(19) United States
(12) Patent Application Publication
(10) Pub. No.: US 2026/0024420 A1
(43) Pub. Date: Jan. 22, 2026

(54) ELECTRONIC JEWELRY BOXES, ILLUMINATED CHARGING VESSELS, AND NETWORK-READY ELECTRONIC JEWELRY FOR USE WITH SMART PHONES AND WEARABLE DIAGNOSTIC DEVICES INCLUDING MANUFACTURING METHODS, ASSEMBLIES, AND SYSTEMS RELATING THERETO

(71) Applicants: CASA DI BOLLELLA, Irvine, CA (US); LIFE JEWEL INTERNATIONAL, Irvine, CA (US); LIFE PATCH INTERNATIONAL, Irvine, CA (US)

(72) Inventor: Donald BOLLELLA, Irvine, CA (US)

(21) Appl. No.: 19/279,804

(22) Filed: Jul. 24, 2025

Related U.S. Application Data
(63) Continuation of application No. PCT/US2024/013297, filed on Jan. 29, 2024.
(60) Provisional application No. 63/441,652, filed on Jan. 27, 2023.
(60) Provisional application No. 63/443,682, filed on Feb. 6, 2023.
(60) Provisional application No. 63/446,780, filed on Feb. 17, 2023.
(60) Provisional application No. 63/456,486, filed on Apr. 1, 2023.
(60) Provisional application No. 63/468,992, filed on May 25, 2023.
(60) Provisional application No. 63/600,833, filed on Nov. 20, 2023.
(60) Provisional application No. 63/608,956, filed on Dec. 12, 2023.
(60) Provisional application No. 63/615,423, filed on Dec. 28, 2023.
(60) Provisional application No. 63/617,847, filed on Jan. 5, 2024.
(60) Provisional application No. 63/620,691, filed on Jan. 12, 2024.

(51) Int. Cl. G08B 6/00; A44C 7/00; A44C 9/00; A44C 15/00; A44C 27/00; A45C 11/16; A47G 7/06; C03B 9/32; C03B 9/347; F21S 8/06; F21V 33/00; G08B 5/36; H02J 50/10; H05B 47/115

(52) U.S. Cl. CPC G08B 6/00; A44C 7/00; A44C 27/00; A45C 11/16; A47G 7/06; C03B 9/32; C03B 9/347; F21S 8/065; F21V 33/0028; G08B 5/36; H02J 50/10; H05B 47/115; A44C 9/00; A44C 15/0035; F21V 2200/10

(57) ABSTRACT

Various electronic jewelry items along with electronic jewelry boxes, cases, and vessels include several embodiments providing network-ready, light-emitting, and recharging functionalities. A number of these embodiments are directed to various system arrangements including smartphones, wearable diagnostic devices, and communications networks as enabled for interactions therewith. Various related methods of manufacturing and using these electronic jewelry items and jewelry boxes, cases, and vessels are also provided.

### CROSS-REFERENCE TO RELATED APPLICATIONS

This application is a continuation of International Application No. PCT/US2024/013297 filed Jan. 29, 2024, entitled ELECTRONIC JEWELRY BOXES, ILLUMINATED CHARGING VESSELS, AND NETWORK-READY ELECTRONIC JEWELRY FOR USE WITH SMART PHONES AND WEARABLE DIAGNOSTIC DEVICES INCLUDING MANUFACTURING METHODS, ASSEMBLIES, AND SYSTEMS RELATING THERETO, which claims domestic and international priority to U.S. Provisional Patent Applications 63/468,992 filed May 25, 2023; 63/456,486 filed Apr. 1, 2023; 63/446,780 filed Feb. 17, 2023; 63/443,682 filed Feb. 6, 2023; and 63/441,652 filed Jan. 27, 2023 with each thereof similarly titled Electronic Jewelry Boxes And Network-Ready Jewelry For Use With Smart Phones And Wearable Diagnostic Devices Including Systems And Methods Relating Thereto with all of the above being incorporated herein by reference.

The International Application PCT/US2024/013297 further claims domestic and international priority to U.S. Provisional Patent Applications 63/620,691 filed Jan. 12, 2024; 63/617,847 filed Jan. 5, 2024; 63/615,423 filed Dec. 28, 2023; 63/608,956 filed Dec. 12, 2023; and 63/600,833 filed Nov. 20, 2023 with each of same as hereto titled Electronic Jewelry Boxes, Illuminated Charging Vessels, And Network-Ready Electronic Jewelry For Use With Smart Phones And Wearable Diagnostic Devices Including Manufacturing Methods, Assemblies, And Systems Relating To Thereto with all of the above being incorporated herein by reference.

And in addition to the above, the International Application PCT/US2024/013297 by common inventorship and common ownership further relates to U.S. Pub. No. 2020/0345533 titled Wearable Patch For Reducing Snoring Activity filed on May 1, 2020 under U.S. application Ser. No. 16/864,863, with priority to U.S. Provisional Application No. 62/842,472 filed on May 2, 2019; the International Application PCT/US2024/013297 also relates to U.S. Pat. No. 10,603,463 titled Individual Wake-Up Alarm Patch filed on Jul. 30, 2018 under U.S. application Ser. No. 16/049,759, with priority to U.S. Provisional Application No. 62/540,126 filed on Aug. 2, 2017; and the International Application PCT/US2024/013297 further relates to U.S. Provisional Application No. 62/972,657 filed Feb. 11, 2020 titled Wearable Patches For Babies; and still further the International Application PCT/US2024/013297 relates to PCT International Publication No. WO 2021/163341 titled Diagnostic Patches And Bracelets For Promoting Personal And Community Health Including Related Processes, Methods, And Systems as filed on Feb. 11, 2021 under International App. No. PCT/US2021/017679, with priority to U.S. Provisional Application Nos. 62/972,657 filed Feb. 11, 2020; 62/972,654 filed Feb. 11, 2020; 62/976,295 filed Feb. 13, 2020; 62/977,691 filed Feb. 17, 2020; 63/019,295 filed May 2, 2020; 63/023,512 filed May 12, 2020; 63/029,622 filed May 25, 2020; 63/042,677 filed Jun. 23, 2020; and 63/050,029 filed Jul. 9, 2020, whereby all of the above are herein incorporated by reference.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the U.S. Patent and Trademark Office file or records, or as it may further appear in English or translation in any other national or international patent offices around the world, but otherwise the copyright owner hereby reserves worldwide all copyright rights whatsoever. In particular, under the Bern Convention for the Protection of Literary and Artistic Works, national implementations thereof, and any additional national, international, regional, or local copyright laws that may apply to this subject matter, the copyright owner hereof expressly claims copyright protection to all material herein amenable thereto including, without limitation, the artwork in the drawing figures and all corresponding copies or protected derivatives thereof as may be rendered in any two or three dimensional form as based on the pictorial illustrations and textual descriptions herein disclosed necessarily by the creators for public benefit upon the publication hereof.

### BACKGROUND OF THIS INVENTION

### 1. Field of the Invention

This invention relates in general to electronic jewelry and, in particular, to network-enabled electronic jewelry. More specifically, but without restriction to the particular embodiments hereinafter described in accordance with the best modes of practice, this invention relates to electronic jewelry boxes, illuminated charging vessels, and network-ready jewelry items for use with smart phones, wearable diagnostic devices, and combinations thereof including systems, methods, assemblies, and processes relating thereto.

### 2. General Discussion and Related Art

While the histories of jewelry making, glass blowing, and stone sculpting date back to ancient times, the modern arts of electronic jewelry and electronic jewelry boxes are limited with such arts being even less well developed as encompassing personal diagnostic devices, smartphones, or networks including both smartphones and wearable diagnostic devices for personal health and wellness.

### SUMMARY OF THE INVENTION

It is, therefore, an objective of the inventors hereof to advance the arts of electronic jewelry and electronic jewelry boxes.

Another purpose of these efforts is to advance the arts of electronic jewelry as used in combination with smartphones.

It is still a further aim hereof to advance the arts of electronic jewelry as used in combination with personal diagnostic devices.

And still another object hereof is to advance the arts of electronic jewelry as used in connection with networks including both smartphones and personal diagnostic devices.

Yet still another goal of the present inventors is to integrate the old-world arts of jewelry making, glass blowing, and stone carving into the modern arts of micro-electronics and network communications to provide attractive wearables and accessories for personal health and wellness applications.

And still a further intension of this disclosure is to apply the teachings hereof to additional arts such as home interior design, architectural design, and lighting design.

These and other objectives, purposes, aims, intentions, and goals hereof are attained in accordance with the present inventions wherein there are provided various methods of retrofitting an existing piece of jewelry for network functionality. In addition to the above methods, the present inventions include various embodiments of network-ready hold-fast or electronic clasp assemblies for use with pre-existing pieces of jewelry.

Another embodiment of the present invention is directed to a network-ready alert assembly for use with a pre-existing piece of jewelry where this assembly includes a vibration member and an adhesive layer. Here a receiver is implemented in the assembly to receive an activation signal from a remote device so that upon receipt of an activation signal by the receiver, the vibration member vibrates to thereby provide an alert to a user wearing the respective pre-existing jewelry item after the alert assembly is attached thereto by applying the adhesive layer to a backside thereof.

In addition to the above jewelry components, certain other aspects of this invention are directed to providing jewelry boxes for compatibility therewith. As such, the present invention is further directed to various embodiments of jewelry box for charging a collection of jewelry items each having network-ready alert functionality and a rechargeable battery in support thereof. Here in these embodiments, the jewelry box includes wall segments forming the box and at least one indicator light implemented to indicate when the charging port has supplied power and when a rechargeable battery of a respective jewelry item having network-ready alert functionality is charged after placing the respective jewelry item in the box.

And as an alternative to providing assemblies for retrofitting with existing jewelry items, the present invention is further directed to various embodiments of a fully enabled network-ready piece of jewelry comprising a body portion intended for wearable display, a vibration member implemented to vibrate in a predetermine manner, and a receiver component implemented in association with said body portion to form a wearable jewelry assembly thereby being enabled to receive an activation signal from a remote device so that when the remote device sends a respective activation signal to said receiver component, the vibration member vibrates to thereby provide an alert to a user of the wearable jewelry assembly.

And described below in detail in conjunction with various use scenarios, any of the electronic jewelry items hereof may be advantageously deployed in a network system for personal notification or also deployed in a system for second-party notification where the network-ready piece of jewelry is worn by a first person and the remote device is a wearable diagnostic patch worn by a second person.

Next being in accordance with other aspects of the present disclosure, there are provided various embodiments of a charging case for a plurality of network-ready hold fast assemblies for use with pre-existing, post-type jewelry items. These charging cases generally include, as described in further detail below, wall segments, individual charging receptacles, and at least one indicator light implemented to activate when a rechargeable battery in the electronic clasp-type assembly is charged after placing same in a corresponding charging receptacle.

And as may be preferred in any of the embodiments of our electronic jewelry boxes or jewelry charging cases, the side walls may be translucent where the indicator lights of the first color are formed integral with at least one of the side walls. And with any of the above, the indicator lights may be formed from light emitting diodes, smooth optical fiber, optical fiber with nicks or off-set optical crystalline lenses or lensing, or any desired combinations thereof so implemented to scatter the light, focus the light, or otherwise disburse the emitted light in different and various patterns where the patterns may be still, repeating, or moving. Thus in any of these embodiments for our jewelry charging cases, the colors of visible light may be selectable from a plurality of different colors provided by a controller integrated in association with the bottom of the box which is accessed by a wireless device such as a smartphone or tablet.

And in any embodiment of our charging case with receptacles, each of the charging receptacles may include a corresponding network-ready hold fast assembly according to the embodiments thereof as described herein to thereby provided a consumer product for ready use with pre-existing jewelry items already in-hand with the consumer or otherwise later purchased from a traditional vendor of such like.

Yet further intentions of the inventors hereof as expressed through certain of our embodiments of the following glass and stone vessels, is to promote the old-world arts of glass blowing and stone sculpting so that these arts are not lost with the proliferation of globalized mass manufacturing of modern consumer goods but rather are integrated with the arts of high reliability micro-electronics and twenty-first century information networks.

Thus still further according to additional aspects of the present invention, there are provided various embodiments of a method of making a glass jewelry box having light display notification and recharging functionality for use with personal electronic items. These methods generally including the steps of providing a mold configured in a pre-determined shape, blowing molten glass into the mold to form a first layer of glass, applying at least one light emitting element onto the first layer of glass, and blowing molten glass onto the first layer of glass to form a second layer of glass to thereby encapsulate the at least one light emitting element between the first and second layers of glass.

And following the above, there are herein provided a wide variety of various embodiments a glass vessel for charging electronic items of jewelry and a smartphone configured to interact therewith as made by these methods.

Next as to further taking traditional glass blowing and stone sculpting into the realm of micro-electronics and clinical diagnostics, there are also provided by additional aspects of this invention a number of embodiments of a method of making a natural stone jewelry box having light display notification and recharging functionality for use with personal electronic items. One principal embodiment of this method includes the steps of selecting a block of stone having a desired translucence property, forming a stone vessel from the block of stone, the stone vessel having an outside surface, an inside surface, and a thickness therebetween that allows some light to pass therethrough, then blowing molten glass into the stone vessel to form a first layer of glass on the inside surface of the stone vessel, applying at least one light emitting element onto the first layer of glass, and then blowing additional molten glass onto the first layer of glass to form a second layer of glass to encapsulate the at least one light emitting element between the first and second layers of glass as thus contained integral with the stone vessel.

Various additional aspects, features, attributes, and alternatives associated with these methods of making glass and stone vessels including micro-electronics for recharging and light display will be provided in further detail herein below.

### BRIEF DESCRIPTION OF THE DRAWING FIGURES

Further objectives of the present invention together with additional features and functionalities contributing thereto as well as advantages accruing therefrom will be apparent from the following description of various preferred embodiments which are shown in the accompanying drawing figures wherein:

FIGS. 1A, 1B, and 1C show various jewelry items that may be deployed in accordance with the teaching hereof as applied thereto;

FIG. 2A is a flow chart showing the steps of a method of retrofitting an existing piece of jewelry in the form of a clasp-and-post type earring to include network functionality according to certain aspects of the present invention;

FIG. 2B is a series of perspective pictorials illustrating each method step of FIG. 2A;

FIG. 3A is a flow chart showing the steps of a method of retrofitting an existing piece of jewelry in the form of a finger ring to include network functionality according to further aspects of the present invention;

FIG. 3B is a series of perspective pictorial illustrations showing each method step of FIG. 3A;

FIG. 4A is a pictorial representation of a charging case including a plurality of pre-assembled electronic jewelry clasps having network functionality for use in place of an existing clasp associated with an existing piece of jewelry such as a clasp-and-post type earring or broach;

FIG. 4B is a cut-away perspective view of the charging case of FIG. 4A showing within the interior thereof charging components provided for re-energizing the various electronic jewelry items associated therewith;

FIGS. 4C to 4F are perspective schematic views of four different exemplary geometric embodiments of the rectangular charging case of FIG. 4A;

FIG. 4G is a perspective view of the circle-shaped embodiment of the charging case of FIG. 4C including a closable top cover therefor;

FIG. 4H is a perspective view of the heart-shaped embodiment of the charging case of FIG. 4D including a closable top cover therefor;

FIGS. 4I-1 and 4I-2 are more detailed perspective views of the butterfly-shaped charging case of FIG. 4E including a hinged top cover and butterfly-shaped charging receptacles;

FIGS. 4J-1 and 4J-2 are more detailed perspective views of the butterfly-shaped charging case of FIG. 4E here having a removable top cover with butterfly-shaped charging receptacles;

FIG. 4K is a further detailed view of the kitten-shaped charging case of FIG. 4F showing a kitten-shaped electronic jewelry clasp, a correspondingly-shaped charging socket, and certain functional animations incorporating the indicator lights thereof;

FIG. 4L is a detailed view of an exemplary sculpted electronic jewelry box hereof as based on certain geometric and functional features of the charging case of FIG. 4K;

FIG. 4M-1 is detailed perspective view of the kitten-configured charging case of FIG. 4L having a hinged top cover shown in a closed position;

FIG. 4M-2 is more detailed perspective view of the kitten-configured charging case of FIG. 4M-1 with the hinged top open showing use thereof with two different electronic jewelry items hereof;

FIG. 4N-1 is an enlarged perspective detailed view of the sculped kitten-configured electronic jewelry clasp as shown in FIG. 4M-2 for use with the kitten-configured charging case thereof;

FIG. 4N-2 is an enlarged perspective detailed view of the sculped kitten-configured electronic earring as shown in FIG. 4M-2 for use with the kitten-configured charging case thereof;

FIGS. 5A to 5D are pictorial side views of different embodiments of a rechargeable network-ready jewelry clasp according to various aspects of the inventions hereof;

FIGS. 5E and 5F are elevation views of two alternate embodiments of the clasp assembly of FIG. 5D with each thereof having a wing-shaped vibration member;

FIGS. 6A to 6D are perspective exploded views corresponding respectively to the claps shown in FIGS. 5A to 5D;

FIG. 7 is an enlarged plan view of an exemplary circuit board layout with principal components that may be utilized to provide functionality to a clasp assembly according to further aspects of this invention;

FIGS. 8 and 9 are elevation views of two finished clasp assemblies using a type of circuit board and components layout as discussed in conjunction with FIG. 7;

FIG. 10A is a flow chart presenting principal steps of a method of converting the circuit board of FIG. 7 into the backside finished goods clasp assembly of FIG. 8 including an exemplary corresponding pictorial representation of certain steps thereof;

FIG. 10B is a flow chart presenting principal steps of a method of making the backside finished goods clasp assembly of FIG. 5E including an exemplary corresponding pictorial representation of certain steps thereof;

FIGS. 11A and 11B taken together present a flow chart showing the principal steps of a method of making a finished goods frontside electronic jewelry item according to further aspects of this invention with same including a representative pictorial illustration for certain selected steps thereof;

FIGS. 12A and 12B as taken together present a flow chart showing the principal steps of the method of FIGS. 11A and 11B as implemented to make a finished inventory jewelry item in the form of an electronic network-ready base pad assembly ready for further down-stream manufacturing with same illustrating a representative pictorial diagram for certain steps thereof;

FIG. 13A is a flow chart showing the steps of a method of retrofitting an existing piece of jewelry in the form of a clip-type earring with network functionality according to yet another additional aspect of the present invention;

FIG. 13B is a series of perspective pictorials illustrating each method step of FIG. 13A;

FIG. 13C is a flow chart showing the steps of a method of fitting a customized earring with a vibration assembly according to still another aspect of the present invention;

FIG. 13D is a series of perspective pictorials illustrating each method step of FIG. 13C;

FIGS. 13E to 13I are assembly views of the type of customized earring of FIG. 13D showing in perspective pictorial illustrations progressive steps of the fabrication thereof;

FIG. 14A is a perspective pictorial representation of a personal collection of pre-existing jewelry items as may be stored in a traditional jewelry box for safe keeping;

FIG. 14B is a pictorial representation of an electronic jewelry box of the present invention including a flat-bottom charging base in accordance with still further aspects of this invention;

FIG. 14C is a pictorial representation of a delivery card including a plurality of pre-assembled vibration pucks according to this invention with each such puck having network functionality for use with an existing piece of jewelry such as a bracelet, necklace, or clip-type earring to thereby provide a personal alert notification when worn on the body of a user thereof;

FIG. 14D is pictorial rendering of an individual vibration puck removed from the delivery card of FIG. 14C showing a peel away film cover supplied over the corresponding adhesive pad of the selected vibration puck;

FIG. 14E is an illustrative pictorial example of placing one of the vibration pucks from the delivery card of FIG. 14C onto one of the items of existing jewelry selected from the traditional jewelry box of FIG. 14A;

FIG. 14F is a perspective pictorial illustration of various selected items of existing jewelry each having a vibration puck attached thereto as placed in the electronic jewelry box of FIG. 14B for re-charging according to still yet further aspects of the present invention;

FIG. 15A is a pictorial representation of a charging case hereof including a plurality of corresponding network-ready earrings made according to certain aspects of the present invention;

FIG. 15B is a schematic perspective representation of a two-piece electronic jewelry item hereof having a front-side piece and a back-side piece with each thereof having a desired grouping of operable components according to further teachings hereof;

FIG. 15C is a pictorial illustration of a stylized two-piece electronic jewelry item of the type of FIG. 15B here having sculpted pieces configured in the form of a ladybug with closed wings;

FIG. 15D is a pictorial illustration of a stylized two-piece electronic jewelry item of the type of FIG. 15B here having sculpted pieces configured in the form of a ladybug with open wings;

FIG. 15E is a pictorial illustration of a stylized two-piece electronic jewelry item of the type of FIG. 15B here having sculpted pieces configured in the form of a lotus flower and lotus leaf;

FIG. 15F is a pictorial illustration of a stylized two-piece electronic jewelry item of the type of FIG. 15B here having sculpted pieces configured in the form of a sun flower and sun flower stalk;

FIG. 15G-1 is a pictorial illustration of a stylized two-piece electronic jewelry item of the type of FIG. 15B here having sculpted pieces configured in the form of a kitten or cat face as the front-side piece and a corresponding cat tail as the back-side piece;

FIG. 15G-2 is a pictorial illustration of a stylized two-piece electronic jewelry item of the type of FIG. 15B here having sculpted pieces configured in the form of a puppy dog or mature canine face as the front-side piece and a corresponding tail as the back-side piece which item may be manufactured by the method of FIG. 28H discussed in detail herein below;

FIG. 15H is a pictorial illustration of a stylized two-piece electronic jewelry item of the type of FIG. 15B here having sculpted pieces configured in the form of a rose blossom and a rose flower stem with sepal;

FIG. 15I is a pictorial illustration of a stylized two-piece electronic jewelry item of the type of FIG. 15B here having sculpted pieces configured in the form of a flowering Bougainvillea vines and branch;

FIG. 15J is an exploded schematic perspective representation of a clip-type two-piece electronic jewelry item hereof having a front-side piece and a back-side piece with each thereof having a desired grouping of operable components according to further aspects hereof;

FIG. 15K is a pictorial illustration of a stylized two-piece electronic jewelry item of the type of FIG. 15J here having sculpted pieces configured in the form of a ladybug with closed wings;

FIG. 15L is a pictorial illustration of a stylized two-piece electronic jewelry item of the type of FIG. 15J here having sculpted pieces configured in the form of a ladybug with open wings;

FIG. 15M is a pictorial illustration of a stylized two-piece electronic jewelry item of the type of FIG. 15J here having sculpted pieces configured in the form of a lotus flower and lotus leaf;

FIG. 15N-1 is a pictorial illustration of a stylized two-piece electronic jewelry item of the type of FIG. 15J here having sculpted pieces configured in the form of a kitten or feline face as the front-side piece and a corresponding cat tail as the back-side piece;

FIG. 15N-2 is a pictorial illustration of a stylized two-piece electronic jewelry item of the type of FIG. 15J here having sculpted pieces configured in the form of a puppy or canine face as the front-side piece and a corresponding tail as the back-side piece which item may be made according to the method of FIG. 28H discussed herein below;

FIG. 15O is a pictorial illustration of a stylized two-piece electronic jewelry item of the type of FIG. 15J here having sculpted pieces configured in the form of a rose blossom and a rose flower stem with sepal;

FIG. 15P is a pictorial illustration of a stylized two-piece electronic jewelry item of the type of FIG. 15J here having sculpted pieces configured in the form of flowering Bougainvillea vines with a back-side branch;

FIG. 16A is a front elevation view of another embodiment of an electronic jewelry box including indicator lights according to yet further aspects hereof;

FIG. 16B is a backside elevation view of the illuminated electronic jewelry box of FIG. 16A showing in perspective as exploded therefrom a sound box and a control unit in accordance with additional aspects this invention;

FIG. 16C is a view similar to FIG. 16B schematically illustrating various additional electronic functionalities that may be provided with the illuminated electronic jewelry boxes according to further embodiments thereof;

FIG. 17A is a perspective view of different electronic jewelry items having network ready functionality according to the teachings hereof;

FIG. 17B is a pictorial perspective representation of a smartphone enabled to interact with the electronic jewelry items of FIG. 17A;

FIG. 17C illustrates two of several different types of wearable personal diagnostic devices that may engage with the smartphone of FIG. 17B or otherwise engage directly with the electronic jewelry items shown in FIG. 17A;

FIG. 18 is a perspective pictorial view of a user of the jewelry devices hereof initiating activation of the devices by interaction with an authorized website so that the devices are duly registered and paired with the user's smartphone;

FIG. 19 is a perspective pictorial illustration of a use scenario hereof representing various night-time applications that may be employed by a user of the electronic jewelry devices hereof as deployed in a private network including a personal smartphone, a wearable bio-patch, or both a smartphone and a wearable bio-patch;

FIG. 20 is a perspective pictorial illustration of another use scenario of this invention showing a particular night-time application where an enabled electronic jewelry item hereof is worn by a first person and a bio-patch is worn by a second person;

FIG. 21 is another perspective pictorial illustration of yet a further use scenario of this invention showing a day-time application where an enabled electronic jewelry item hereof is worn by a first person and a bio-patch is worn by a second person;

FIG. 22 is perspective pictorial illustration of yet another day-time use scenario hereof illustrating how a network-enabled electronic jewelry item of this invention as worn by a first person is deployed for the safety and wellness of a child while attending class at school;

FIG. 23 is a pictorial representation of the electronic jewelry box of FIG. 16C interactively engaged with the smartphone of FIG. 17B;

FIGS. 24A, 24B, and 24C taken together present a flow chart showing the steps of a method of making an illuminated glass charging vessel for electronic jewelry items according to still further aspects of the present invention;

FIGS. 25A, 25B, and 25C provide a corresponding series of perspective pictorials illustrating each of the method steps in FIGS. 24A, 24B, and 24C;

FIGS. 26A and 26B are pictorial schematic illustrations showing interchangeable side mold plates according to this invention as deployed to perform certain preferred embodiments of the methods shown and discussed in conjunction with FIGS. 24A to 24C and 25A to 25C;

FIG. 26C is a table with illustrations showing some representative textures that may be used with the mold plates of FIG. 26A;

FIG. 26D is a table showing some representative examples of plate patterns that may be used with the mold plates of FIG. 26B, along with glass colors and light colors that may be combined to make an illuminated glass charging vessel of the type discussed in conjunction with FIGS. 24A to 24C and 25A to 250;

FIG. 26E is an additional method step and corresponding perspective pictorial representation of rubbing casting sand into the patterned or textured molding plates to thereby provide a unique outside surface on a glass charging vessel made according to certain preferred embodiments hereof;

FIGS. 27A and 27B are pictorial schematic illustrations showing interchangeable front mold plates according to additional aspects of this invention as deployed to perform certain further preferred embodiments of the methods shown and discussed in conjunction with FIGS. 24A to 24C and 25A to 250;

FIG. 28A is a pictorial representation of receiving a number of visual images of a pet as used in the following methods hereof to make a sculpted article of manufacture therefrom;

FIG. 28B represents a method for converting the visual images of FIG. 28A into a three-dimensional digital image file of the pet pictured therein;

FIG. 28C represents a method for using the three-dimensional digital image file of FIG. 28B to operate a CNC machine to make a custom front mold plate of the type presented in FIG. 27B;

FIG. 28D is a perspective schematic view of a glass-blowing mold having as a front plate thereof, the front mold plate made according to the method of FIG. 28C;

FIG. 28E is a perspective view of an illuminated glass charging vessel made according to various teaching hereof as delivered from the mold of FIG. 28D;

FIG. 28F is a flow chart showing the principal steps of receiving the pet photos of FIG. 28A and then making an electronic jewelry box hereof as based thereon;

FIG. 28G is a perspective illustration of the electronic jewelry box made according to the method of FIG. 28F;

FIG. 28H is a flow chart showing the principal steps of receiving the pet photos of FIG. 28A and then making a sculped electronic jewelry piece hereof as based thereon;

FIG. 28I is a perspective illustration of two different types of two-piece electronic earrings made according to the method of FIG. 28H;

FIG. 29A is a schematic perspective illustration of an illuminated glass charging vessel made according to certain further aspects of this invention having optical fiber extension segments protruding up and over a top edge thereof;

FIGS. 29B to 29F are pictorial perspective illustrations of various exemplary embodiments of an illuminated glass charging vessel hereof with each thereof made according to the method of FIGS. 24A to 24C with a different front mold plate as provided by the textures and patterns from the tables of FIGS. 26C and 26D;

FIG. 29G is a pictorial perspective illustration of an illuminated glass charging vessel hereof as made according to the method of FIGS. 24A to 24C using the high bias artwork front mold plate of FIG. 27A;

FIGS. 30A to 30E are schematic representations showing different fringe patterns that may be formed in the optical fiber extension segments discussed in connection with FIG. 29A;

FIGS. 30F to 30H provide three schematic representations showing an exemplary arrangement of light emitting elements and optical fiber bundles according to certain features, attributes, and functionalities hereof;

FIGS. 31A, 31B, and 31C taken together present a flow chart showing the steps of a method of making an illuminated stone charging vessel for electronic jewelry according to yet still additional aspects of this invention as adopting some of the method steps shown in FIGS. 24A, 24B, and 24C;

FIGS. 32A, 32B, and 32C provide a series of perspective pictorial illustrations showing each of the corresponding method steps in FIGS. 31A, 31B, and 31C;

FIG. 33A is an additional method step and corresponding schematic representation of precious or simi-precious gemstones inlayed into a stone charging vessel made according to certain additional preferred embodiments hereof to thereby provide a unique outside surface associated therewith;

FIG. 33B represents a subassembly method of receiving photos and operating a CNC machine to make a custom stone casing for the type of illuminated vessel crafted according to the method of FIGS. 31A to 31C;

FIG. 33C represents a subassembly method of receiving a lighting design specification and operating a CNC machine to form interior sockets and slots configured to position various light sources and lighting elements in a stone casing before continuing with a glass blowing step according to FIG. 31A;

FIGS. 34A, 34B, 34C, 34D, 34E-1, 34F, and 34G provide a series of perspective pictorial illustrations showing seven different exemplary illuminated stone charging vessels made according to various embodiments of hereof;

FIGS. 34E-2 to 34E-4 are detailed perspective assembly views showing different components and supporting electronic circuitry implemented in the vessel of FIG. 34E-1 as an illustration of such components and circuitry that may be so implemented in any of the charging vessels hereof;

FIG. 35A is a side elevation view of a layered stone charging vessel having an inverted stairstep outside surface made according to further aspects of this invention;

FIG. 35B is a side elevation view of a layered stone charging vessel hereof having a smooth outside surface made according to still further aspects of this invention;

FIG. 35C is a side elevation view of yet another layered stone charging vessel hereof having a quarter-round edging detail on each step of an inverted stepped outside surface thereof as made according to yet further aspects of this invention;

FIG. 36A is a side view of three selected frame segments from the stone charging vessel of FIG. 35A before assembly thereof;

FIG. 36B is a top plan view of the frame segments of FIG. 36A showing representative overlap alignments of consecutive frame segments;

FIG. 37A is a perspective assembly view of the frame segments of FIGS. 36A and 36B;

FIG. 37B is a perspective assembly view of alternate frame segments of the type shown in FIG. 37A;

FIGS. 38A, 38B, 38C, and 38D are perspective detail views showing the outside inverted step surface of the stone charging vessel of FIG. 35A with four different inside surface formulations, respectively;

FIG. 38E is a detailed perspective view of a portion of a side wall of a layered stone charging vessel hereof having a smooth outside surface and a stepped inside surface of the type of vessel shown by exterior view in FIG. 35B;

FIG. 38F is a detailed perspective view of a portion of a side wall of a layered stone charging vessel hereof having a quarter-round edging detail on the inside and outside surfaces thereof as shown on the exterior of the type of vessel illustrated in FIG. 35C;

FIG. 38G is a detailed perspective view of a portion of a side wall of a layered stone charging vessel hereof having a smooth outside surface and a louvered inside surface of the type of vessel shown in the exterior view of FIG. 35B;

FIG. 38H is a detailed perspective view of a portion of a side wall of a layered stone charging vessel hereof having smooth inside and outside surfaces as shown on the exterior of the vessel illustrated in FIG. 35B;

FIG. 39A is a detailed perspective partial view of the inside surface of the type of illuminated stone charging vessel shown in FIGS. 35A and 38A showing a representative channel formed therein for receiving a bundle of optical fibers according to certain additional teachings hereof;

FIG. 39B is a view similar to FIG. 39A where a strip of light emitting sources is deployed horizontally along selected inside steps of a stone charging vessel of the inside surface step-type charging vessels hereof;

FIG. 40A is a partial perspective view of three fiber optic bundles deployed horizontally on the inside surface of a stone charging vessel of the types hereof with each respective bundle associated with a corresponding light emitting element;

FIG. 40B is a view similar to FIG. 40A wherein as an alternative to fiberoptic bundles, strips of light emitting sources are deployed horizontally along selected inside steps or louvers of a stone charging vessel of the types hereof suitable therewith;

FIG. 41 is a view similar to FIG. 37 where channels for receiving a respective fiberoptic bundle have been formed in selected individual frame segments before assembly thereof;

FIGS. 42A, 42B, and 42C are partial detailed schematic views of the light channels of FIG. 41 as formulated for receiving a corresponding fiberoptic bundle;

FIGS. 43A, 43B, and 43C taken together provide flow-charting and pictorial illustrations of a series of steps of a method of making a layered stone vessel according to still yet further aspects of this invention;

FIG. 44A flow-charts and illustrates pictorially additional initial steps that may be included in the method of FIGS. 43A to 43C to make a layered stone vessel from two types of natural stone slabs having different material properties;

FIG. 44B flow-charts and illustrates pictorially a subassembly method for making a horizontally laminated stone block from two types of natural stone slabs having different material properties for further processing according to the method of FIGS. 31A to 31C;

FIG. 44C charts and illustrates provision of a vertically laminated stone block from two types of different stone material made according to the subassembly method of FIG. 44B;

FIG. 44D is a perspective view of a vertically stratified stone shell as provided from FIG. 44C for further processing according to the method of FIGS. 31A to 31C;

FIGS. 45 and 46 flow-chart and illustrate pictorially additional steps that may be used in making a layered stone vessel of the types discussed in connection with FIGS. 43A to 43C and FIG. 44A;

FIGS. 47A, 47B, and 47C taken together present a flow chart showing the steps of a method of making an illuminated stone and glass charging vessel from a layered stone vessel hereof according to still further aspects of the present invention;

FIGS. 48A, 48B, and 48C provide a corresponding series of perspective pictorials illustrating each of the method steps in FIGS. 47A, 47B, and 47C;

FIG. 49 charts and illustrates an additional step that may be included in the method of FIGS. 47A to 47C;

FIGS. 50A, 50B, 50C, and 50D are perspective illustrations showing four different layered natural stone electronic jewelry vessels that may be made according to corresponding methods hereof;

FIG. 51 charts and illustrates an additional method step that may be included in making any of the glass or stone vessels hereof where gold leaf is applied by hand;

FIGS. 52A and 52B chart and illustrate additional method steps that may be included in making any of the glass or stone vessels hereof where a gold coating is applied by vapor deposition;

FIG. 53 is a perspective illustration of a segmented insert assembly that may be used in conjunction with any of the layered stone jewelry boxes hereof to separate into individual compartments a smartphone and electronic jewelry items for recharging according to further aspects hereof;

FIG. 54 is a sectional elevation view of a segmented insert assembly of the type of FIG. 53 shown in a layered stone jewelry box made according to the methods hereof;

FIGS. 55A and 55B are schematic representations of two different inside surface formations that may be utilized in making the segmented insert assembly of FIG. 54;

FIG. 56 is a cut-away perspective view of a layered stone jewelry box hereof showing two different types of top covers that may be provided therewith;

FIG. 57A is a schematic elevation view of an illustrative representation of any of the electronic jewelry boxes or vessels hereof showing in an accompanying block diagram a corresponding electronics casing and various electronic functionalities that may be included therewith;

FIG. 57B is a perspective view of a rechargeable illuminated flower vase made by combining selected teachings hereof as including selected options from the functional features presented in the block diagram of FIG. 57A;

FIG. 57C is a simplified block diagram of the features selected from the options hereof to specify the functionalities of a rechargeable illuminated flower vase of the type presented in FIG. 57B;

FIG. 58A is a next-level block diagram of additional functionalities that may be provided in the electronics casing of FIG. 57A including an architectural mode of operation and a security mode of operation;

FIG. 58B is a functional diagram with perspective pictorial of an illuminated charging vessel hereof that may be used as a silent wake-up light alarm;

FIG. 59A is a perspective top view of a representative segmented insert assembly of the type presented in detail in conjunction with FIGS. 53 to 55B;

FIG. 59B is a schematic perspective assembly view of a number of individual charging pads each associated with a corresponding light source where the assembly is configured to comport with the design of the segmented insert assembly of FIG. 59A;

FIG. 60A is a break-away perspective view of an alternate bottom embodiment for any of the illuminated vessels hereof showing a glass-blown jewelry tray formed integral with the blown-glass bottom of the vessel and individual charging pads corresponding to separate compartments in the jewelry tray;

FIG. 60B provides a subassembly method step illustrating the making of the glass-blown jewelry tray of FIG. 60A;

FIG. 61 presents a flow-chart with pictographic illustrations of principal steps of making an illuminated flat glass panel by applying certain teaching hereof to the arts of making sheet glass for windows, doors, partitions, and walls;

FIG. 62A is an elevation view of a representative partition wall made with the illuminated glass panels hereof showing in an accompanying block diagram certain electronic functionalities that may be provided therewith by applying selected teachings here-from to the arts of architectural design and building security;

FIG. 62B is a perspective view of a certain office building that has provided therein the illuminated glass panels hereof showing in the accompanying block diagram of FIG. 62A certain electronic functionalities implemented in the architectural design thereof;

FIGS. 63A to 63D are elevation views of various doors and windows provided with the illuminated glass panels hereof as made by applying certain teachings here-from to the arts of interior design and home security;

FIG. 64 is a block diagram showing certain electronic functionalities that may be provided with the windows and doors of FIGS. 63A to 63D;

FIG. 65 is a detailed cut-away view of an illuminated door of the type in FIGS. 63A and 63B herein showing an example of providing an electronics assembly for operation thereof;

FIG. 66 is a detailed cut-away view of framing a door jamb or window jamb to provide electrical power to the illuminated windows and doors of FIGS. 63A to 63D;

FIG. 67 is a schematic perspective view of a multi-mode glass chandelier that may be provided hereunder by applying selected teachings here-from to the arts interior lighting also showing in an accompanying block diagram a corresponding electronics casing and various electronic functionalities that may be included therewith;

FIG. 68A is an illustration of a glass-blown element for the type of chandelier of FIG. 67 configured in the form of a bird of paradise flower and stalk having light-emitting optical fibers partially encased therein and partially extending therefrom;

FIG. 68B is an illustration of a glass-blown element for the type of chandelier of FIG. 67 configured in the form of a single bird of paradise leaf having light-emitting optical fibers fully encased therein;

FIG. 68C is an illustration of a glass-blown element for the type of chandelier of FIG. 67 configured in the form of a goose-neck branch having a traditional flame-tip light bulb assembly and light-emitting optical fibers partially encased therein and partially extending therefrom; and

FIG. 68D is a schematic illustration of an electronic base assembly according to this invention as provided for the type of chandelier of FIG. 67 herein configured to support the glass-blown lighting elements of FIGS. 68A, 68B, and 68C.

### DETAILED DESCRIPTION OF THE PREFERRED EMBODIMENTS

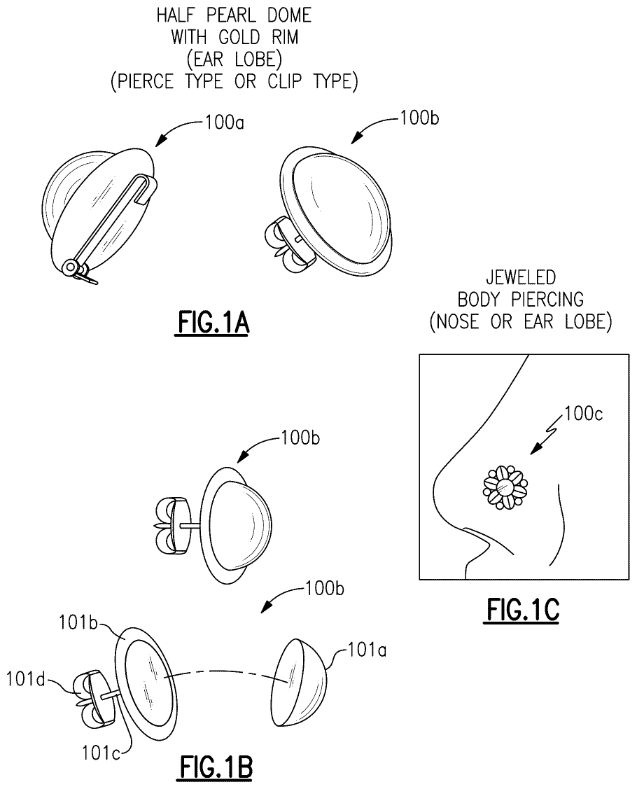

Referring now to FIGS. 1A, 1B, and 1C, there is shown jewelry items 100a, 100b, and 100c. As discussed in further detail below for each item shown as comprising additional aspects of the inventions hereof, jewelry item 100a is in the form of a clip-type pearl earring, jewelry item 100b is in the form of a post-and-clasp-type pearl earring for use on a pierced ear, and jewelry item 100c is in the form of a post-and-clasp-type jeweled nose adornment for use on a nose piercing. In FIG. 1B, jewelry item 100b is shown has having three principal parts including a dome portion 101a, a base portion 101b including a post 101c, and a removable clasp 101d. The jeweled nose adornment 100d of FIG. 1C, as illustrated in this embodiment, includes flower petals, circular features that may be rendered as sepal or flower buds, and a center piece that may be rendered as a flower stigma of other central flower feature. All of the elements of the jeweled nose adornment 100c may be formed from a preferred metal such as gold or may be rendered as a combination of precious metal or mixed precious metals with some features thereof formed by gem stones as is well know in the art of fine jewelry making. As discussed and described in commonly owned U.S. Pub. No. 2020/0345533 titled Wearable Patch for Reducing Snoring Activity filed May 1, 2020 under U.S. patent application Ser. No. 16/864,863 having priority to U.S. Provisional Application No. 62/842,472 filed on May 2, 2019, the jewelry items 100a, 100b, and 100c as also shown here in FIGS. 1A, 1B, and 1C, may be advantageously provided with a variety of electronic and mechanical micro-receivers, components including transmitters, electro-acoustic transducers, microphones, snoring alert transducers, and other such micro-electronic and micro-electro-mechanical components where herein for purposes of simplicity, such components may be generally referred to collectively as electronic and MEMS components where “MEMS” as used herein as a common acronym has its general technical meaning more fully written as “Micro Electronic Mechanical Systems” or “Micro-Electro-Mechanical Systems” with same now including a wide variety of micro and nano-scale electronic and mechanical componentry in various emerging arts across several different technical subject matter areas. And further herein, where needed to fully describe and characterize the inventions hereof, certain particular electronic and MEMS components will be further disclosed and discussed in specific detail for each of the various embodiments hereof as provide below.

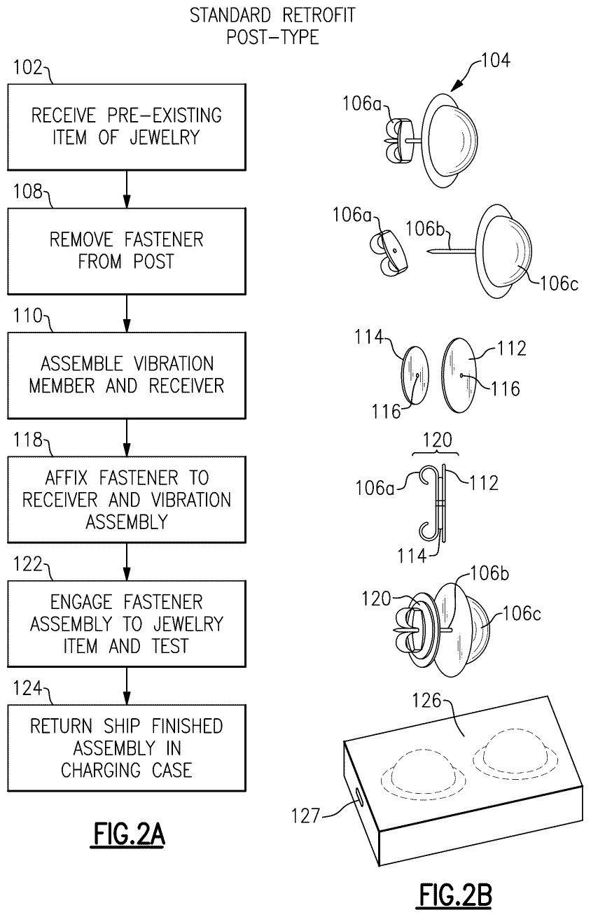

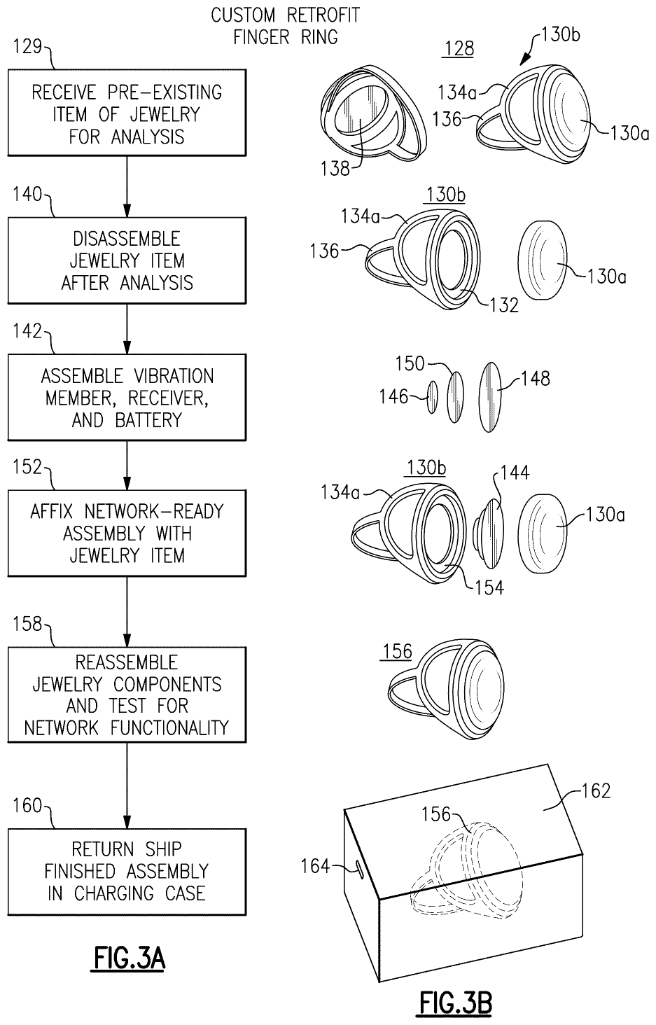

Now turning to one particular aspect of these inventions, the inventors hereof recognize that many members of the public may have a personal inventory of jewelry items that have been long treasured. To address the need as to compatibility with certain related aspects of the present inventions as discussed below in further detail, the inventors hereof provide herein various assemblies and methods of retrofitting an existing item of jewelry to hereby adapt thereto, the various systems, apparatus, processes, and methodologies hereof. Thus, the present invention is further directed to a post-type retrofit method as shown in FIGS. 2A and 2B where FIG. 2A describes a series of steps and FIG. 2B illustrates pictorially each of the corresponding steps in FIG. 2A.

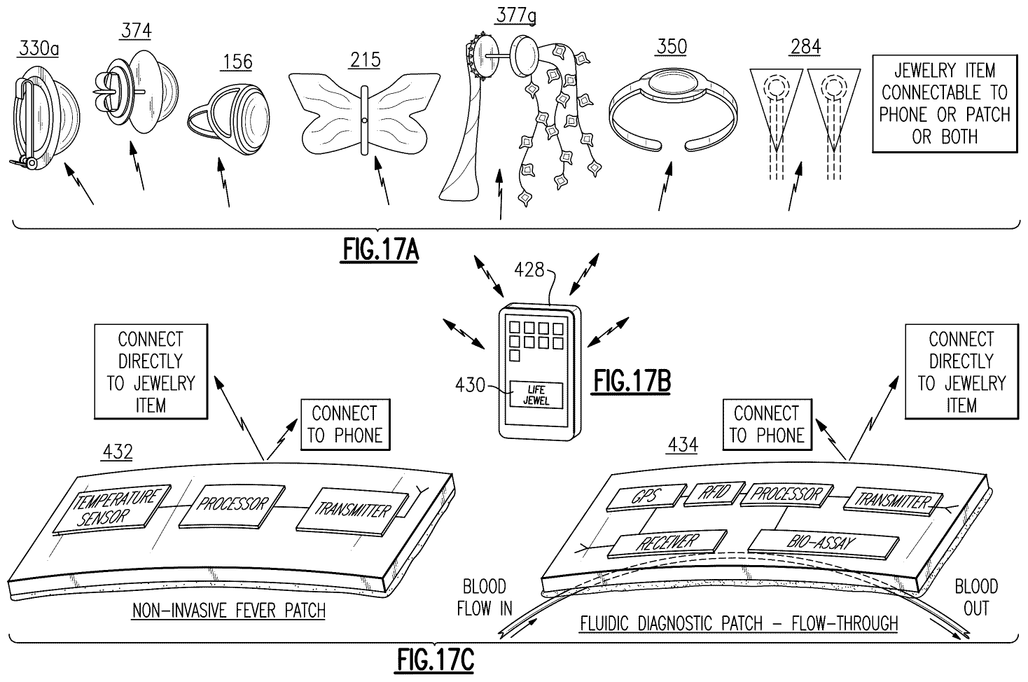

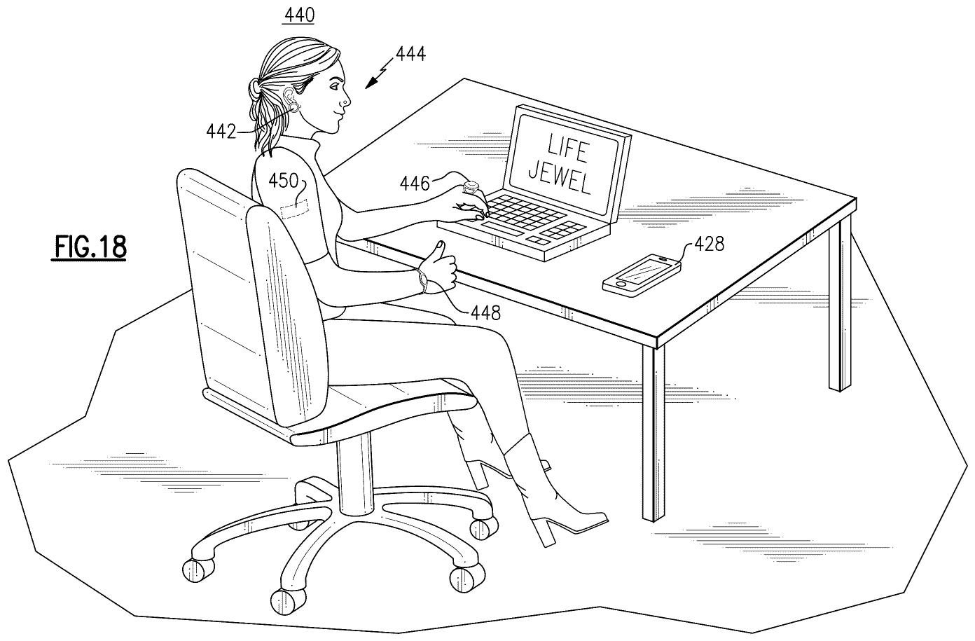

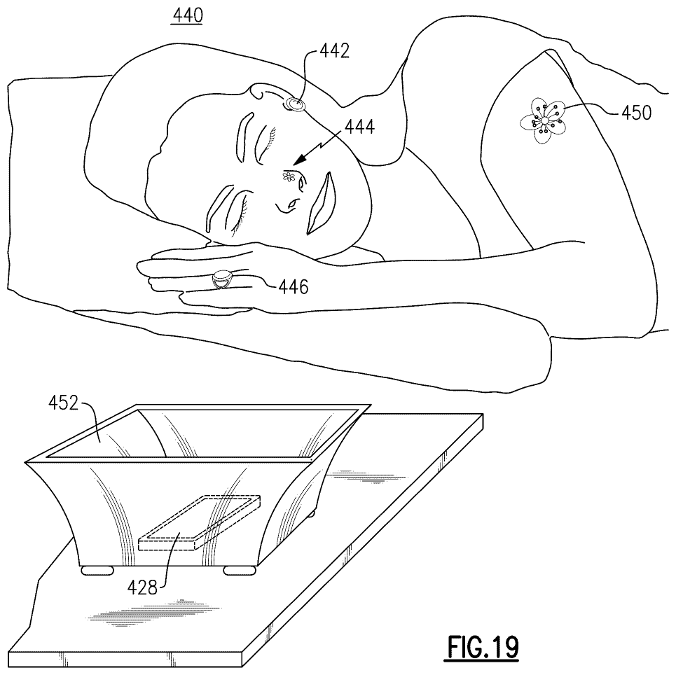

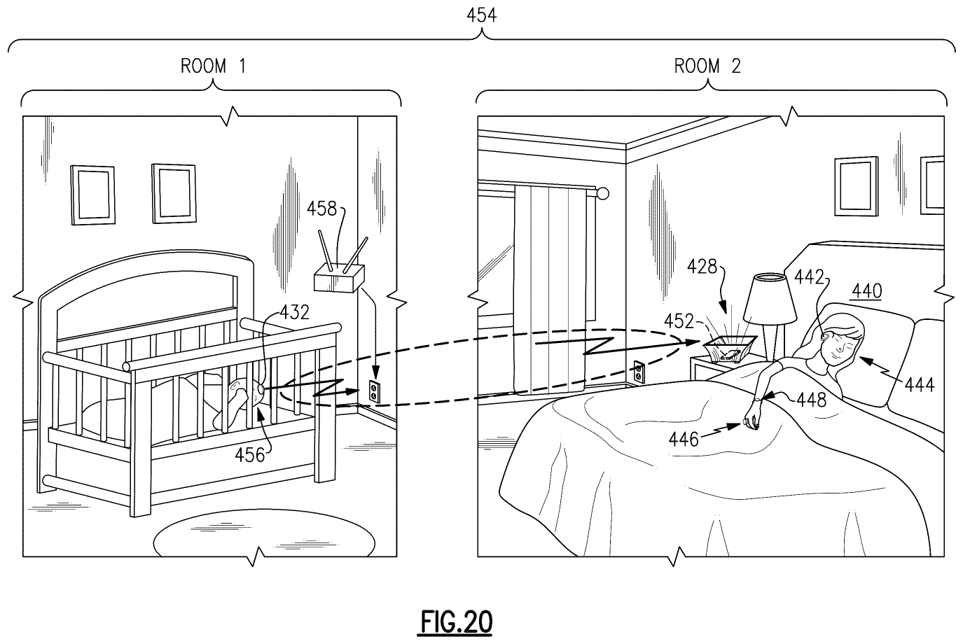

Now with continued reference to FIGS. 2A and 2B, more particularly, there is shown a first step 102 of receiving a pre-existing item of jewelry being here, for example, a post-type pearl earring 104 having a clasp or fastener 106a, a corresponding post 106b sized to mate with the clasp 106a in a manner well-known in the art of jewelry making, and a body portion 106c for display appearances. The earring 104 may be provided from an individual owner thereof who has long treasured the item for personal use, or it may be provided as a new item of manufacture from a customary manufacturer of such jewelry items with some such manufactures being well-known throughout the world of jewelry making. Next at step 108, the clasp 106a is removed and may be inspected for compatibility with the remaining steps of this method. If not suitable, a compatible replacement clasp may be provided in place thereof with such a clasp providing antenna capabilities for communicating wirelessly with a remote device such as a smartphone carried for personal use by an individual wearing the jewelry item as herein discussed, or the remote device may be a wearable personal diagnostic device such as the patch devices referenced and incorporated herein above and further discussed in detail herein below in conjunction with FIGS. 17A, 17B, and 17C. Then next at step 110 in FIG. 2A there is provided in accordance with further aspects of this invention, a vibration member 112 and a receiver 114 with each thereof having a concentric hole 116 formed therein. In this embodiment, each of the vibration member 112 and the receiver 114 include a suitably sized rechargeable battery or other type of rechargeable power cell to perform the functions associated therewith. In other embodiments, there may be provide a single rechargeable battery to power both the vibration member 112 and the receiver 114 with appropriate electrical connections provide there between. Next in step 118, the vibration member 112 and the receiver 114 with each thereof having the concentric hole 116 are affixed in register with a suitable clasp 106a to thereby form a discreetly wearable vibration assembly, vibrating clasp assembly, or vibrating hold fast assembly 120 that may be activated by an alarm function on a smartphone, a notification signal from a bio-patch or a fever patch, or otherwise interact within a local network formed by the vibration assembly 120, a smartphone, and a personal diagnostic patch as herein below described in further detail. And thus further, as to next steps in this method, at step 122, the clasping vibration assembly 120 is engaged with the post 106b and then the full assembly comprising the original pearl earring 104 including its post 106b as maintained integral with the body portion 106c now including the vibrating clasp assembly 120, is electronically tested to ensure its intended functionalities including receiving, vibrating, charging, and recharging are in good working order. Then at step 124, the finished assembly as passed successfully under the testing step 122, is placed in a charging case 126 including a charging port 127 and returned to the original owner thereof or otherwise offered for sale as a new item of manufactured jewelry. Thus with particular reference to FIGS. 2A and 2B, one aspect of this invention is directed to providing a first method of retrofitting an existing piece of jewelry 104 for network functionality where this method comprises the steps of i) providing a vibration member 112 implemented to vibrate in a predetermine manner, ii) providing a receiver 114 in association with said vibration member 112, said receiver 114 implemented to receive an activation signal from a remote device; and iii) assembling the vibration member 112 and the receiver 114 with the existing piece of jewelry 104 to form a wearable assembly so that when the remote device sends a respective activation signal to said receiver, the vibration member vibrates to thereby provide an alert to a user of the wearable assembly. In one particular embodiment of this method where the existing piece of jewelry 104 includes a clasp 106a for engagement with a post 106b, the present invention is further directed to providing a network-ready hold fast assembly 120 for use with a pre-existing piece of jewelry 104 having a post 106b, said network-ready hold fast assembly comprising a clasping component 106a implemented for engaging the post 106b of the pre-existing piece of jewelry 104 in a secure manner and disengaging from the post 106b when desired by a user thereof, ii) a vibration member 112 affixed in assembly with said clasping component 106a and implemented to vibrate in a predetermine manner; and iii) a receiver component 114 affixed in assembly with said vibration member 112 and with said clasping component 106a, said receiver component 114 implemented to receive an activation signal from a remote device so that upon receipt of said activation signal by said receiver component 114, said vibration member 112 vibrates to thereby provide an alert to the user of the assembly when engaged with the post 106b of the pre-existing piece of jewelry 104. And as would be understood given the discussion above, the post 106b of pre-existing piece of jewelry item 104 may be secured through a body piercing of the user thereof, or alternatively, the post 106b of pre-existing piece of jewelry item 104 may be secured through an article of clothing worn by the use thereof.

Next, FIG. 3A presents a flow chart showing the steps of a method of retrofitting an existing piece of jewelry in the form of a finger ring 128 to include network functionality according to further aspects of the present invention, and FIG. 3B is a series of corresponding perspective pictorials illustrating each method step of FIG. 3A. Thus at step 129 of this method, the ring 128 is received for analysis as to suitability with this retrofitting aspect of the present invention. The ring 128 includes a display element 130a and a functional frame portion 130b. The frame portion 130b provides utility and function in its design. As such, the illustrated frame portion 130b includes an annular retainer 132 that functions to hold the display element 130a integral with the ring frame 130b in a manner for display on the finger of a hand that is well known by artisans working in the art of jewelry making. Here as illustrated, the display element 130a is an attractive stone shaped as shown to correspond to the shape of the annular retainer 132. The frame portion 130b herein depicted also includes a pair of opposing D-shaped side members 134a and 134b with 134a shown as illustrated and 134b out of view in this perspective. The function of opposing D-shaped side members 134a and 134b is firstly to hold the annular retainer 132 in its geometrical orientation relative to the frame portion 130b and also to provide lateral support to the ring on the finger when worn to thereby assist by its wide shape and friction on the finger from having the ring stone rotating on the finger toward the palm of the hand. In addition, the opposing D-shaped side members are substantially void of center material mass thus giving an open look which is functionally lighter in weight thus more comfortable over a longer time of use while also providing the wider lateral support functionality to prevent undesired ring rotation. Next in this analysis step 129, it is understood that the ring 128 has a finger loop segment 136 that connects the opposing D-shaped side members 134a and 134b in a closed circle to fit around the finger of a user thereof. Then finally in this analysis, it is determined that this ring 128 has a hollow void or empty space 138 formed by the inside opening in the annular retainer 132 and the back side of the display element 130a. As such, the ring 128 as illustrated is an excellent candidate for this retrofitting method because the hollow void 138 provides an ideal location for fitting the network-ready vibration assembly according to the teachings hereof. Thus at step 140, the ring 128 is disassemble into its two main constituent parts including the display element 130a and the frame portion 130b. Next at step 142, a suitable network-ready vibration assembly 144 is configured for the ring 128. In this embodiment as illustrated for exemplary discussion, the network-ready vibration assembly 144 includes a vibration component 146, a receiver component 148, and a rechargeable battery or power cell 150 as shown at step 142. Next at step 152, the vibration assembly 144 is affixed to the ring preferably by having the outer circumference of the receiver component 148 sized to fit onto an annular seat 154 formed as part of the annular retainer 132 as shown. In this embodiment, the vibration assembly 144 thereby affixes snuggly and becomes integral with a final ring assembly 156 including the original ring 128 now including the vibration assembly 144 where after final assembly the network-enabled ring 156 is tested for intended electronic and mechanical functionalities as shown in step 158 of FIG. 3A. Then finally at step 160, the ring assembly 156 is placed in a suitable charging case 162 having a charging port 164 for connection to a power source for recharging the battery or other type of suitable rechargeable power cell in the ring assembly when needed for use, such as repeated daily use as may be desired.

As similar to the earring 104 of FIG. 2B, the network-enabled ring 156 hereof may be initially received from an individual owner of the original ring 128 who has long treasured the item for personal use, or it may be provided as a new item of manufacture by a customary manufacturer of such jewelry items with some such manufactures being well-known throughout the world of jewelry making. Then after applying the method steps including the testing step 158, the newly enabled network-enabled ring 156 may be returned to the original owner thereof with the charging case 162 or otherwise offered for sale as a new item of manufactured with the charging case 162, or alternatively otherwise returned or offered for sale without the charging case 162 so as to be used with other electronic jewelry cases, boxes, or vessels as also discussed herein below.

Thus here in FIGS. 3A and 3B, there is more specifically provided a method of retrofitting an existing piece of jewelry 128 for network functionality, said method comprising the steps of i) assessing at step 129 a respective existing piece of jewelry 128 for suitability with a desired network functionality; ii) disassembling the respective existing piece of jewelry 128 into separated elements after determining suitability thereof with said desired network functionality; iii) providing a vibration member 146 implemented to vibrate in a predetermine manner; iv) providing a receiver 148 in association with said vibration member 146, said receiver 148 implemented to receive an activation signal from a remote device; and v) assembling the vibration member 146, the receiver 148, and at least some of the separated elements 130a and 130b from the existing piece of jewelry 128 to form a wearable assembly 156 so that when the remote device sends a respective activation signal to said receiver 148, the vibration member 146 vibrates to thereby provide an alert to a user of the wearable assembly 156. And as further shown and discussed, the wearable assembly 156 includes a rechargeable battery 150 in associated with the vibration member 146 and the receiver 148 to form the vibration assembly 144 as integrated as an operable component to created the network-ready wearable assembly 156.

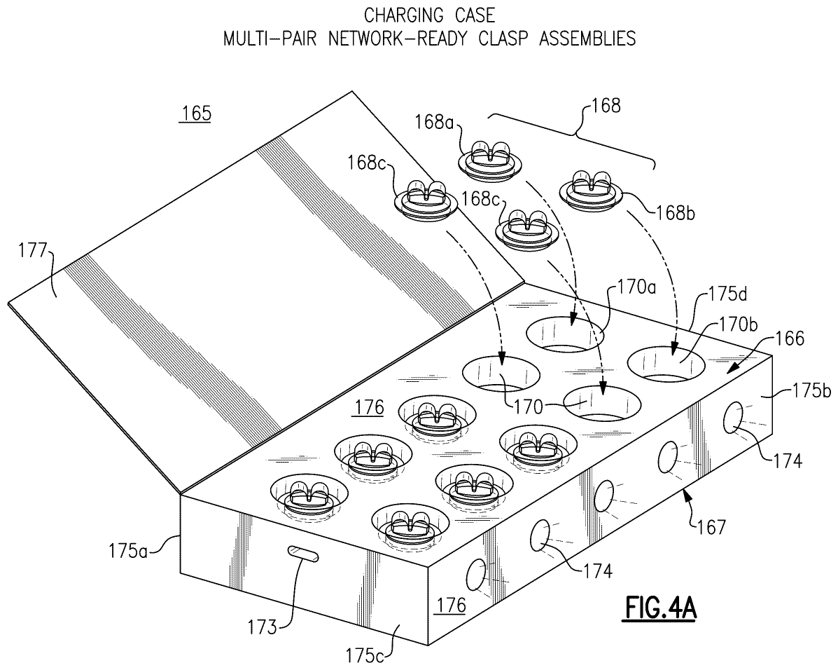

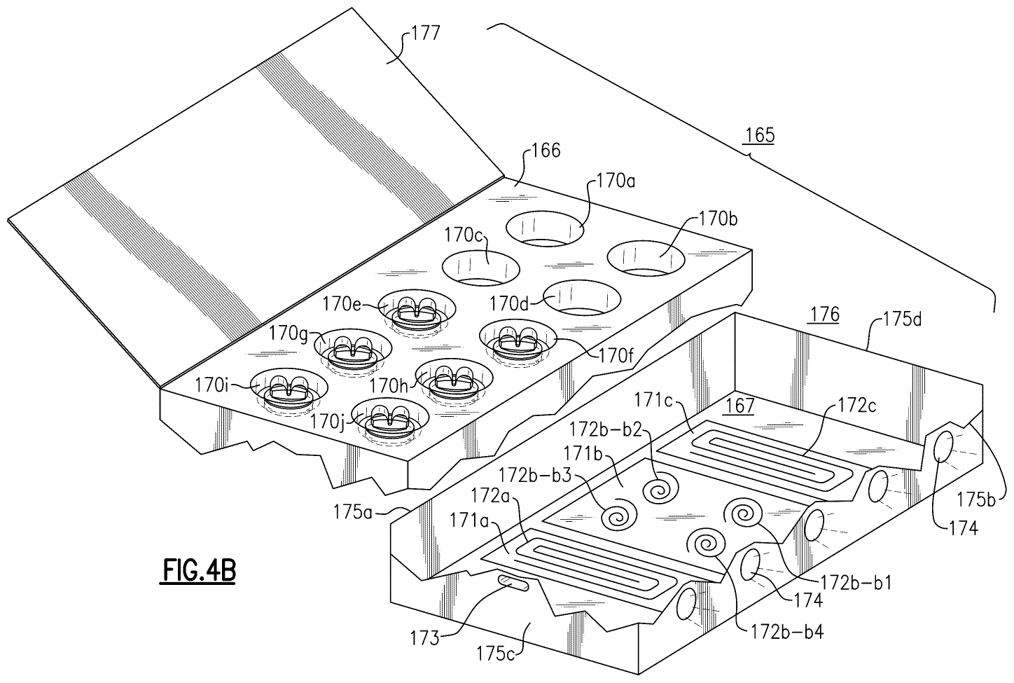

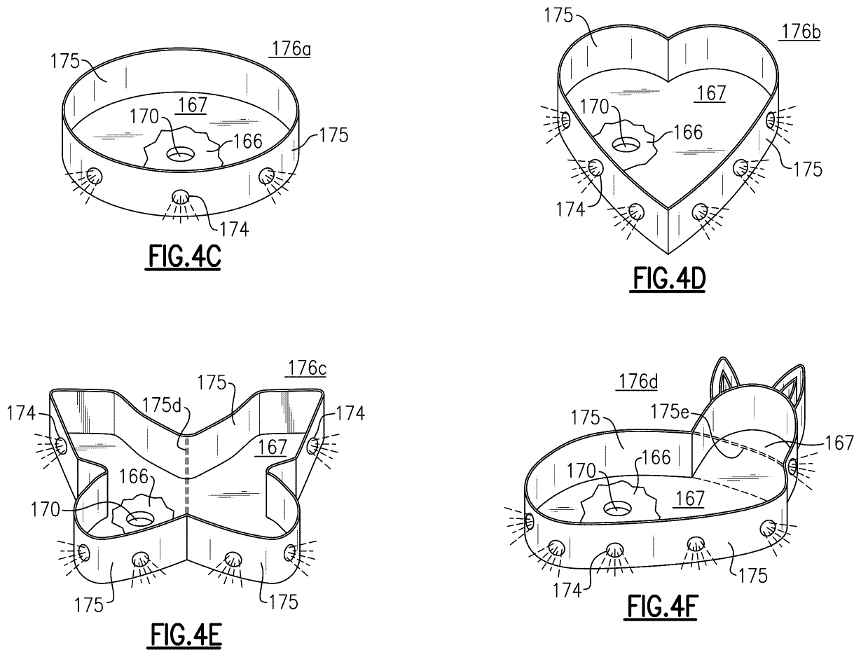

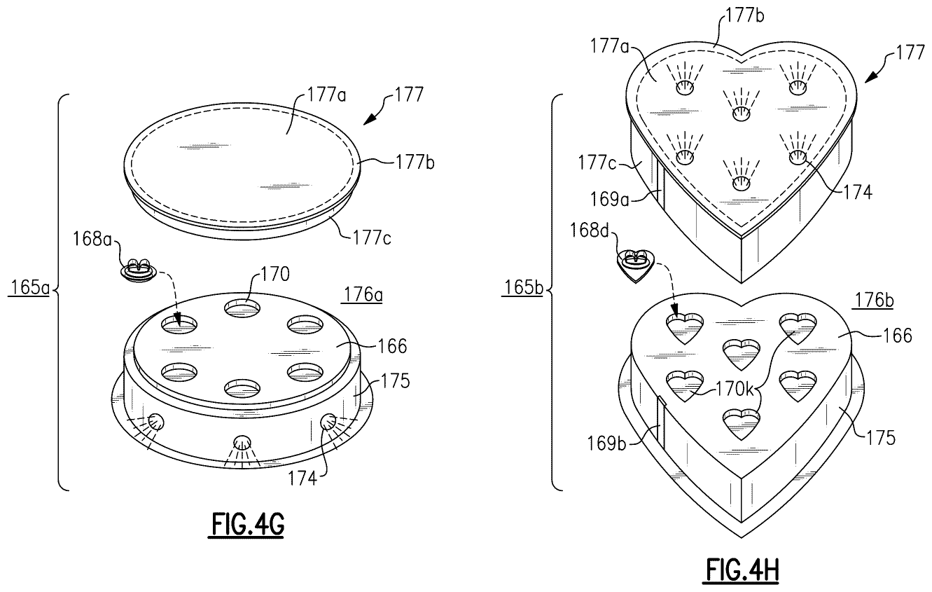

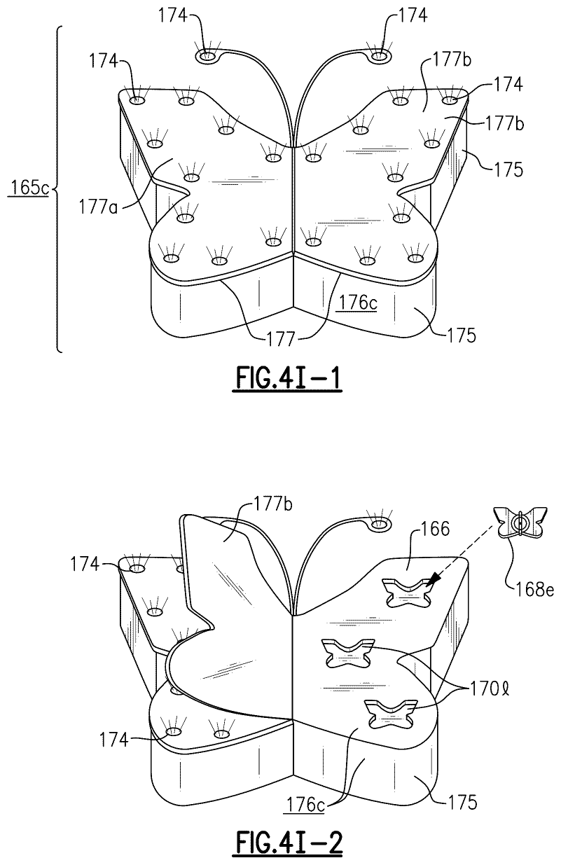

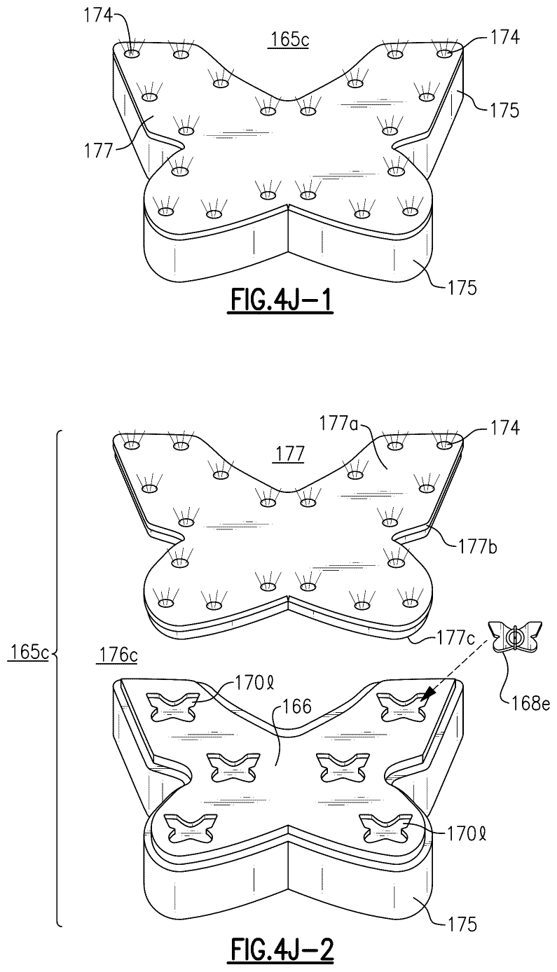

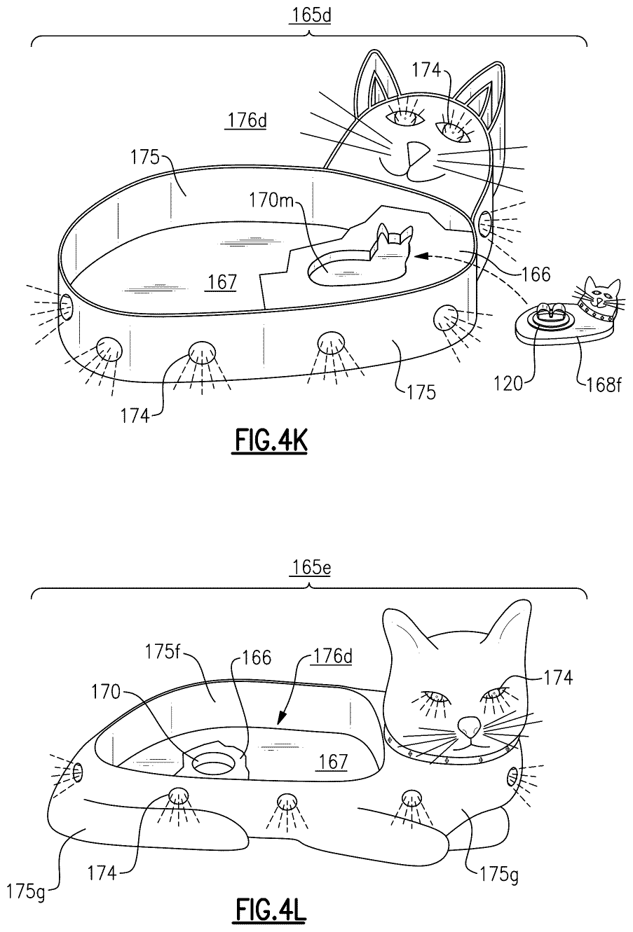

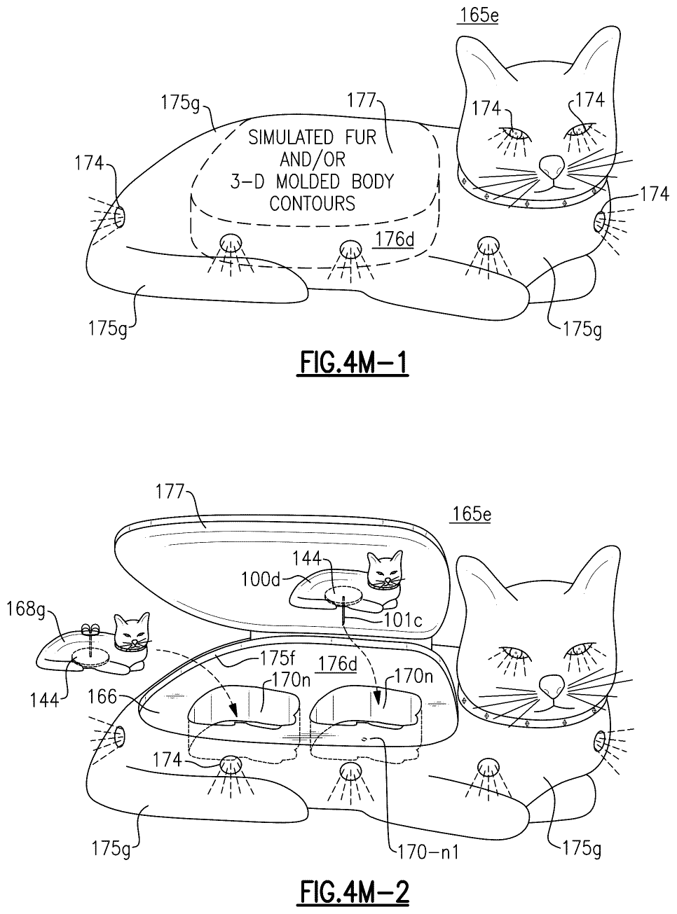

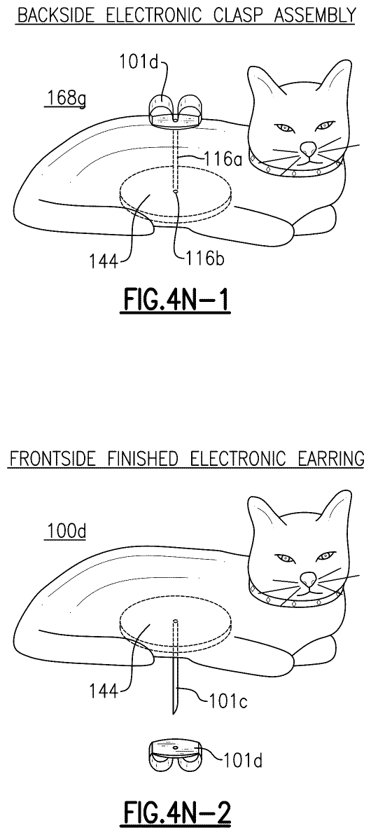

With reference next to FIG. 4A, there is shown a pictorial representation of a charging case or electronic jewelry box 165 having a box top or top 166 and a box bottom or bottom 167 where, as illustrated, the case 165 is configured to accommodate a plurality of pre-assembled electronic clasp pairs including an exemplary clasp pair 168 comprised of a first network-ready clasp assembly 168a and a second network-ready clasp assembly 168b. The paired clap assemblies 168a and 168b are each provided with network functionality according to the teachings hereof as implemented for use in place of an existing traditional clasp associated with an existing piece of jewelry such as a favored pair of post-and-clasp type earrings or with one thereof deployed in use with a broach secured to an article of clothing by post-and-clasp. In some preferred embodiments hereof, the clasp assemblies 168a and 168b may each provide the same functionalities and may then be preferably worn at the same time with, for example, a matching pair of earrings, with one thereof on the left ear and the other thereof on the right ear. In this case where both of the assemblies 168a and 168b are deployed as a pair of concurrent wearable assemblies, each may simultaneously receive a signal from a smartphone or a personal diagnostic patch, where then the left and the right as utilized may provide redundancy as to the reception functionality of the receiver in each of the assemblies 168a and 168b. Further, when the received signal causes an intended vibration in the wearable devices 168a and 168b, as deployed in a paired manner with each on a corresponding left and right ear, then both are enabled to vibrate concurrently to thereby provide a more prevalent notification arrangement. Alternatively, each of the assemblies 168a and 168b may be provided with difference functionalities, where for example, the clasp assembly 168a is worn on the left ear and is enabled to network with a smartphone while concurrently deployed therewith, the clasp assembly 168b is worn on the right ear and is enabled to, separately and independently from 168a, network with a personal diagnostic patch. And as would become apparent to those of skill in the arts of electronic jewelry given the disclosure hereof, in other embodiments hereof as to deployment and use scenarios, each of the clasp assemblies 168a and 168b may be separately used or independently deployed either together or separately on different use occasions including paired earrings, a single earring, a nose piercing, a single broach, paired broaches, paired earrings with a broach, or the like, wherein color coding or other indicia or visual designations may be provided on each assembly as delivered in the case 165 to so indicate its functionality.

With continuing reference to FIG. 4A, the charging case or electronic jewelry box 165 in this embodiment as illustrated includes a number of charging receptacles 170 that may be paired as referenced 170a and 170b. Each of these charging receptacles 170, both paired and unpaired are associated with a corresponding electronic charging component 171 as discussed in more detail in FIG. 4B. Further in FIG. 4A, the electronic jewelry box 165 also includes a charging port or electric supply socket 173, and an indicator light 174 for each of the charging receptacles 170 or for each pair of charging receptacles 170a and 170b as arrayed along a side wall segment 175b of the charging case 165. In this manner, each of the network-ready clasp pairs 168a and 168b has a corresponding charging receptacle 170a and 170b such that when placed therein, a respective corresponding indictor light 174 may be activated in different colors, on-off cycles, light intensities, or any number of desired combinations thereof, to give visual notice of the state of the receptible as empty or occupied, charging, or fully charged as to the intended utility of charging the rechargeable battery or power cell provided in each corresponding network-ready clasp. In other embodiments hereof, the charging case 165 with opposing side walls segments 175a and 175b may include an indicator light corresponding to each individual receptible 170, for example here, by providing a corresponding number of indictor lights 174 on the side wall segment 175a in a similar manner to the indicator lights 174 as illustrated in this view on the side wall segment 175b.

Here as illustrated, opposing side wall segments 175c and 175d do not include any indicator lights 174. And for purposes of particularizing the geometry here, it should be understood that side wall segments 175a, 175b, 175c, and 175d are each straight or flat segments that form a continuous side wall 175 configured in a closed-loop rectangular shape and thus along with the top 166 and the bottom 167 of the charging case 165 (as viewed as planar surfaces) form a three-dimensional box 176 as a geometric form. In other embodiments, however, the side wall 175 may be in the form a continuous smooth curve or circle to form a serpentine or a pill box type configuration, for example, where a single side wall 175 is provided without sharply opposing wall segments or box corners as with the pill box or circular shape or with just one sharp-corner as in the embodiment of providing a heart-shaped side wall 175 with a corresponding heart-shaped top 166. In any of these embodiments, whether the side wall 175 is straight, curved, serpentine, or circular, each charging receptible 170 may preferably have a corresponding indicator light 174 as here shown in the rectangular box configuration of FIG. 4A. In these various differing geometric embodiments of the shape of the box 176, the charging receptacles 170a and 170b, may retain a special pairing or paired orientation, or they may alternatively be an individual charging receptacle for a single clasp assembly 168c having a specific user application, such silent as wake-up alarm and/or snoring detection, as used with a single earring or with a pair of earring where one thereof has the vibrating electronic network ready clasp assembly hereof the other has a traditional clasp. And in certain preferred embodiments of the charging case 165, for example, those designed for travel applications, the box or case 165 may advantageously include a foldable top cover or closable lid 177 to cover the top 166 of the box 165 so that any clasp assembly 168a in its corresponding charging receptacle 170a may be secured therein.

Next with reference to FIG. 4B, there is shown a cut-away view of the bottom 167 of the box 176 comprising the electronic jewelry case 165. Therein show for exemplary purposes are three electronic charging components 171 each consecutively referenced 171a, 171b, and 171c. The charging component 171a is provided with a charging coil 172a implemented on a circuit board with associated electronics that will be described in greater detail herein below. Similarly, the charging component 171b is provided with four individual charging coils 172b-b1, 172b-b2, 172b-b3, and 172b-b4. And finally, the charging component 171c is provided with a single charging coil 172c. Thus as implemented here, the charging component 171a with the single coil 172a is situated to charge electronic clasp assemblies placed in charging receptacles 170i and 170j. Alternatively as shown with charging component 171b, each charging receptacle may be provided with its own corresponding charging coil on a separate circuit with a corresponding indicator light 174. Thus here in regard to charging component 171b as illustrated, charging coil 172b-b1 is situated to charge an electronic clasp assembly placed in charging receptacle 170f, charging coil 172b-b2 is situated to charge an electronic clasp assembly placed in charging receptacle 170e, charging coil 172b-b3 is situated to charge an electronic clasp assembly placed in charging receptacle 170g, and charging coil 172b-b4 is situated to charge an electronic clasp assembly placed in charging receptacle 170h. And further, charging component 171c with its single charging coil 172c is situated to charge an electronic clasp assembly placed in any one or more of charging receptacles 170a, 170b, 170c, and 170d. Thus as would become apparent to those of skill in the electronic arts in view of the present disclosure, an indicator light 174 may be associated with a pair of charging receptacles, here for example charging receptacles 170i and 107j, or an indicator light 174 may be associated with a single charging receptacle here for example where coil 172b-b1 is singularly associated with charging receptacle 170f which is then associated with any electronic clasp assembly placed therein for recharging. Further particularized details relating to such electronic circuitry or micro-circuitry as implemented within the charging components 171a, 171b, and 171c for activating indicator lights 174 are presently considered separate as to the intended scope of the claimed subject matter of this disclosure and thus may be presented as the subject matter of subsequent disclosures extensive hereto.

And now with reference to FIGS. 4C to 4F, there are shown perspective schematic views of four different exemplary geometric embodiments of the rectangular charging case of FIG. 4A. First in FIG. 4C, there is shown a circular box 176a that would serve to form a charging case 165 as described above in FIGS. 4A and 4B with the difference here being in the geometric shape of the box. The circular box 176a has a circular bottom 167 and a corresponding circular side wall 175 which supports the top of the box 166 as shown in a cut-away section with a charging receptacle 170 situated therein as shown. Similarly in FIG. 4D, there is illustrated a heart-shaped box 176b that has a heart-shaped bottom 167 and a corresponding heart-shaped side wall 175 which supports the top of the box 166 as shown in cut-away section with a charging receptacle 170 situated therein as shown. Next in FIG. 4E, there is shown a butterfly-shaped box 176c that has a butterfly-shaped bottom 167 and a corresponding butterfly-shaped side wall 175 which supports the top of the box 166 as shown in cut-away section with a charging receptacle 170 situated therein. The butterfly-shaped box 176c may also include a central or internal divider wall segment 175d as shown to support hinged wings as later described in conjunction with FIGS. 4I-1 and 4I-2. Finally in FIG. 4F, there is shown a kitten or cat-shaped box 176d that has a cat-shaped side wall 175 including cat ears for aesthetic purposes which side wall 175 supports the top 166 of the box 176d as shown in a cut-away section with a charging receptacle 170 situated therein. And as further indicated in FIG. 4F, an appropriately configured bottom 167 is provided within the periphery of cat-shaped side wall 175 and in certain preferred embodiments the cat-shaped side wall 175 may also include an internal divider wall segment 175e as shown to provide assembly functionality and/or structural support to the box. Each of the boxes 176a, 176b, 176c, and 176d in FIGS. 4C to 4F, respectively, include a number of indicator lights 174 with one thereof shown for illustration purposes in association the representative charging receptacle 170 in each thereof.

And according to still further aspects of the inventions hereof as related to the charging cases 165 discussed above in FIGS. 4A to 4B, FIGS. 4G to 4J-2 are perspective views of a closable top cover for each of the differently shaped embodiments of the boxes 176a, 176b, and 176c illustrated above, respectively, in FIGS. 4C to 4E.

Thus in FIG. 4G, the circular box 176a is shown with a circular top cover 177 that is removable from the box 176a as shown. The circular top cover 177 includes a circular top 177a and a circular wall 177c which is provided with a slightly smaller diameter than the circular top 177a to thereby form a grabbable overhang or lip segment 177b. In this manner, the cover 177 is fully removable from the box 176a as shown. And as discussed above in connection with FIG. 4C, here in corresponding FIG. 4G, the box 176a includes the charging receptacles 170 as formed in the top 166 of the box and the side wall 175 includes the indicator lights 174. Here again, the charging receptacles 170 are circular in shape and thus intended to receive the circularly-shaped electronic clasp 168a as shown.

Next in FIG. 4H, the heart-shaped box 176b is shown with a corresponding heart-shaped top cover 177. Here in this heart-shaped embodiment of the charging case 165b, the heart-shaped top cover 177 includes a heart-shaped top 177a and a heart-shaped wall 177c which is provided with a slightly smaller perimeter than the heart-shaped top 177a to thereby form a grabbable overhang or lip segment 177b that is also heart-shaped in view of the heart-shaped wall 177c and corresponding heart-shaped top 177a. In this manner, the cover 177 is fully removable from the box 176b as shown. And as discussed above in connection with FIG. 4D, here in corresponding FIG. 4H, the box 176b includes the charging receptacles 170 as formed in the top 166 of the box, where here, the charging receptacles 170 are in the shape of heart-shaped receptacles 170k as illustrated. Thus as such, this heart-shaped embodiment is intended to be deployed with heart-shaped electronic clasps 168d as shown. Here the heart-shaped receptacles 170k and corresponding heart-shaped electronic clasps 168d are intended by the inventors hereof to create a form of lock-and-key assembly that provides a passive security feature such that unauthorized copies of our heart-shaped electronic clasps 168d are not easily rendered compatible for use with the illustrated heart-shaped box 176b including our heart-shaped receptacles 170k. Additional electronic security feathers relating to the electro-magnetic coupling between the charging components 171 hereof, as shown in FIG. 4B, for example, and our electronic jewelry clasps such as the heart-shaped clasp 168d here in FIG. 4H, are intended to be the subject matter of further disclosures as related hereto.

Further here in this embodiment of FIG. 4H, as an alternative to the above placement of the indicator lights 174, the side wall 175 does not include the indicator lights 174 as shown in FIG. 4D. As such, thus shown here in this alternate heart-shaped embodiment of FIG. 4H with the removable top cover 177, the indicator lights 174 are provided in the top cover 177 as illustrated where electric supply thereto may be proved by an electrical connection 169 including a connector component 169a in the top cover 177 and a corresponding connector component 169b in the side wall 175 of the box 176b, as illustrated here for exemplary purposes.

In a similar manner to the above where in the alternative, the heart-shaped removable top cover 177 of Fig. H includes the indicator lights 174 provided therein, the circular top cover 177 of FIG. 4G may include some or all of the indicator lights 174. In each of these differently-shaped top covers 177, whether circular, heart-shaped, butterfly-shaped, kitten-shaped, or any other desired shape, when removable from their corresponding box 176, the correspondingly shaped removable top cover 177 further includes electric supply thereto which may be provided by the electrical connection 169 with the connector component 169a in the top cover 177 and a corresponding connector component 169b in the side wall 175 of its respective box 176. Alternatively in these removable top cover embodiments, the top cover may be additionally provided with a rechargeable power-storage cell, such as a rechargeable battery, so that when the cover 177 is removed from the box, the indicator lights continue to be illuminated by electric energy provide by the rechargeable power-storage cell integrated with the removable top cover.

Now turning to FIGS. 4I-1 and 4I-2, there is provided therein more detailed perspective views of the butterfly-shaped charging case of FIG. 4E here having a two-piece hinged top cover 177 including a left side hinged wing cover 177a and a right side hinged wing cover 177b. In this manner, the top 166 of the box 176c may be accessed by simply grasping the overhang on the wing 177b and rotating to an open position as shown in FIG. 4I-2. As further shown therein, the top 166 of the box 176c, includes butterfly-shaped charging receptacles 170 configured to receive a butterfly-shaped electronic clasp 168e. Here in this embodiment, the indicator lights 174 are all shown on the wing covers 177a and 177b. As an alternative thereto, the indicator lights 174 may be on the side wall 175 or otherwise on both the wing covers 177a and 177b and the side wall 175. Here in this embodiment, the butterfly-shaped box 176c is shown with a pair of antenna with each thereof including an indicator light 174 which may be illuminated to indicate that power is supplied to the box as needed to provide the charging receptacles 1701 with electric power to change the electronic jewelry clasps 168e when place therein for recharging.

FIGS. 4J-1 and 4J-2 are more detailed perspective views of the butterfly-shaped charging case of FIG. 4E here having a removable butterfly-shaped top cover 177 as discussed above with the removable circular cover in FIG. 4G and the removable heart-shaped cover of FIG. 4H. Here as shown in FIG. 4J-2 the butterfly-shaped charging case 165c is formed by the butterfly-shaped removable cover 177 as configured to fit with the butterfly-shaped box 176c. The removable cover 177 similarly here includes a butterfly-shaped top 177a and a butterfly-shaped wall 177c which is provided with a slightly smaller diameter than the butterfly-shaped top 177a to thereby form a grabbable overhang or lip segment 177b. In this manner, the cover 177 is fully removable from the box 176c as shown. And as further illustrated, the top 166 of the box 176c includes butterfly-shaped charging receptacles or sockets 1701 to accommodate butterfly-shaped electronic earring clasps 168e as discussed above and in further detail herein below regrading the manufacture thereof. Here the indicator lights are shown in the cover 177 but may alternatively be provided in the side wall 175 as shown in FIG. 4E or in combination in both the removable top cover 177 and the side wall 175 of the box 176c.

Next in FIG. 4K, there is provided a further detailed view of the charging case of FIG. 4F showing the kitten or cat-shaped box 176d with a corresponding kitten-shaped charging receptacle 170m configured to accommodate a kitten or cat-shaped electronic jewelry clasp 168f. Here as a further aspect of this embodiment of our inventions, indicator lights 174 are configured as life-like or visually animated cat eyes. The cat-shaped electronic clasp assembly 168f includes the vibration assembly 120 discussed above in FIG. 2B and here may be further adorned with jewels or precious gem stones in the form of a cat collar, for example. Here in FIG. 4K, the top 166 is shown as flat or essentially planar in configuration as to geometric form, and, like above, the box 176d as such may include a flat or planar hinged cover or a flat removable cover to form the corresponding kitten or cat-shaped charging case 165d as illustrated with such hinged or removable covers being kitten or cat-shaped to comport with the top 166 as discussed as to other geometric shaped but not repeated here in the interest of avoiding repetition. As suggested by the animated features of the cat or kitten face represented in FIG. 4K, these features are presented in a stylized manner to appear whimsical, fanciful, or imaginative in their rendering and as such may then be more suitable to a market segment of consumers such as teenagers or pre-teens, as the case may be.

FIG. 4L next presents a detailed view of an exemplary sculpted electronic jewelry box or case 165e according to the teaching hereof as based on some of the geometric and functional features of the kitten or cat-shaped charging case 165d of FIG. 4K. Here in this sculped or contoured three-dimensional configuration, our charging case 165e takes the form of a kitten or cat as rendered with three-dimensional natural features and contours, as may be modeled after a live natural animal such as here illustrated, a natural-featured kitten or cat, where the rendered form here includes a sculped head with ears, eyes, a nose, a mouth, and whiskers; and a resting body portion including here, as illustrated, folded front legs, and a tail wrapped around the body as would be experienced with a usual resting position of a live kitten or cat as may be well-known to cat owners around the world. Here also, the sculpted eyes may advantageously include our indicator lights 174 to provide a sense of animation and functionality to indicate, for example, when the box has supplied power or otherwise to indicated when an electronic jewelry item place therein is fully charged, charging, or otherwise placed or removed from a corresponding charging receptacle 170. As further shown in FIG. 4L, the box 176d as provided in the sculped charging case 165e includes flat bottom 167, interior side wall 175f, and a cut-away section of the top 166 which here is also preferably flat in configuration. And further as to geometric shape, to achieve the desired sculpted three-dimensional form hereof, the exterior of the charging case 165e is provide with contoured exterior walls 175g. Further features, attributes, and functionalities of this sculped cat-shaped charging case are further discussed herein below in conjunction with the following FIGS. 4M-1 to 4N-2.

As such, FIG. 4M-1 is detailed perspective view of the kitten or cat-configured charging case 165e of FIG. 4L having a hinged top cover 177 here shown in a closed position. As discussed just above in FIG. 4L, the exterior side walls 175g in this configuration are not flat or box-shaped as like above in the cat-shaped case 165d of FIGS. 4F and 4K, but here are alternatively rendered in a sculpted three-dimensional form as shown to take the life-like form of a kitten or cat body as may be molded after an image of a live animal. The case 165e here in FIGS. 4M-1 and 4M-2 includes the sculpted head with indicator lights 174 provided in conjunction with the sculped eyes that may be illuminated for any of the functional purposes discussed above. And yet still further according to additional aspects hereof, the case 165e may be provide with simulated or synthetic fur of any desired cat colors to further give life-like animation to interaction with the case 165e under its intended useful applications. And like above with the jeweled electronic clasp 168f of FIG. 4K, here the sculped cat-shaped case 165e configuration may be provided with a jeweled collar as illustrated to further indicate intended purpose and use thereof as a jewelry box. FIG. 4M-2 is a more detailed perspective view of the sculpted kitten-configured charging case 165e of FIG. 4M-1 showing the hinged cover 177 open and formed in the top 166 of the jewel box two representative recharging receptacles or sockets 170n that are here illustrated with a deeper profile to thereby accommodate a corresponding three-dimensional sculpted kitten or cat-shaped electronic jewelry clasp 168g. Also illustrated is a finished jewelry item 100d in the form of a sculpted kitten or cat-shaped earring with a post 101c for use with a pierced ear. Here both the electronic clasp 168g and finished electronic earring 100d are provide with a network-ready vibration assembly 144 of the type discussed above in connection with FIG. 3B and manufacturing step 152 of corresponding FIG. 3A. With regard to the finished electronic earring 100d, its corresponding charging socket or receptacle 170n is further provide with a post-socket 170-n1 to accommodate the post 101c in a fashion that would be well understood by those of skill in the mechanical arts. In this manner, the bottom of the electronic jewelry item 100d may sit flat on the bottom of the receptacle so that an electro-magnetic field generated under the receptacle by a charging coil positioned in association therewith may re-charge the rechargeable battery provided in the network-ready vibration assembly 144 as integrated within the jewelry item 100d in a similar manner as recharging the electronic clasp 168g.

Next in FIG. 4N-1, there is presented a perspective detailed view of the sculped kitten-configured electronic jewelry clasp 168g as shown in FIG. 4M-2. Here in further clarity by way of enlargement, the sculpted kitten-shaped network-ready electronic clasp assembly 168g is shown as having a mechanical hold-fast clasp 101d as here integrated with the cat body by welding or soldering as is well know in the jewelry making arts, and here further provided with the vibration assembly 144 according to the teachings hereof as discussed above and below. As further shown, a central hole 116a is provide fully through the body and a corresponding hole 116b is provide in the vibration assembly 144 so that the post of a respective item of pre-existing jewelry may be thereby secured by sliding the post thereof through the hold 116b, through the hole 116a, and then into the clasp 101d in the usual way. Thus in use, the vibration assembly 144 of the electronic clasp 168g may vibrate to provide the user with a silent personal alert when an intended remote signal is received by the receiver circuitry provide therewith.

As will be discussed in further detail below with regard to additional detailed embodiments of various finished jewelry items hereof, FIG. 4N-2 now simply provides further clarity by way of enlargement of the sculped kitten-configured electronic earring 100d of FIG. 4M-2. Here for initial description, the finished sculpted jewelry item 100b is intended for use as an earing on the front-side of an ear lobe with a traditional simple machinal clasp 101d that secures the post 101c in the usual manner. And thus further here according to the teachings hereof, the front side jewelry item 100d has integrated therein a network-ready vibration assembly 144 of the type discussed above and herein below in further detail as to the manufacture and assembly thereof. As such and thus when in use, the vibration assembly 144 as integrated within the sculped kitten-configured electronic earring 100d may vibrate to provide the user thereof with a silent personal alert when an intended remote signal is received by the receiver circuitry provide therewith.

Thus with specific regard to the embodiments discussed herein above in conjunction with FIGS. 4A to 4N-2, the present invention is further directed to providing a charging case 165 for a plurality of network-ready hold fast assemblies 168a, 168b, 168c for use with pre-existing post-type jewelry items, said charging case 165 comprising i) a side wall 175, a bottom 167, and a top 166 configured to form a box 176; ii) a pre-determined number of charging receptacles 170 formed in the top 166 of the box 176, each of said charging receptacles 170 configured to receive a clasp-type network-ready hold fast assembly 168 with each thereof having a rechargeable battery; iii) a charging component 171 situated in association with each of said charging receptacles 170; iv) a power supply port 173 situated in the box 176 in a pre-determine location; and v) at least one indicator light 174 implemented in association with the box 176 to indicate when the power supply port 173 has supplied power and/or when a respective rechargeable battery associated with a corresponding clasp-type network-ready hold fast assembly 168 is charged after placing same in a corresponding charging receptacle 170. In one preferred embodiment of the charging case 165, the side wall 175, the bottom 167, and the top 166 are configured to form a circular box 176a as shown in FIG. 4C. In another embodiment thereof, the side wall 175, the bottom 167, and the top 166 are configured to form a heart-shaped box 176b as shown in FIG. 4D, and sill yet in a further embodiment of the charging case 165, the side wall 175, the bottom 167, the top 166, the charging receptacles 170, and the closable lid 177 are configured to form a heart-shaped box 176b reminiscent of a heart-shaped box of chocolate candy. And still further according to the various more complicated geometries of the box 176 comprising the charging case 165 of FIG. 4A, the side wall 175, the bottom 167, and the top 166 are configured to form a butterfly-shaped box 176c as shown in FIG. 4E, or similarly in the shape of a four-leaf clover. And more preferably for certain consumer groups as may be desired, the side wall 175, the bottom 167, and the top 166 of the box 176 are configured to form a box 176d in the shape of a kitten or cat, and further when including the closable lid 177 in the rendering, the side wall 175, the bottom 167, the top 166, and the closable lid 177 may be configured together to form a box 176d in the shape of a kitten as rendered in further detail with various desired body and facial features included therewith including simulated fur of a soft and pleasing touch where desired. Thus in this embodiment as represented in FIG. 4J, still further, the closable lid 177 may be additionally configured to include a two- or three-dimensional rendering of a kitten or cat face with two kitten or cat eyes with an indictor light 174 positioned coincidentally with each kitten or cat eye so that when a respective one of the indicator lights is illuminated, the kitten or cat eye associated therewith is illuminated. And likewise for other animal shapes, the side wall 175, the bottom 167, and the top 166 may be configured to form a box 176 in the shape of a puppy dog, and further including the closable lid 177 as further configured to contribute to the form of a box 176 in the shape of a puppy dog with additional body and or facial features including simulated fur of a soft and pleasing touch where desired.

And still further in connection with any of the embodiments of the charging case or electronic jewelry box 165 discussed above in FIGS. 4A to 4F, the at least one indicator light 174 may include a plurality of indicator lights 174, wherein a first number of said plurality of indicator lights 174 emits a first color of visible light and a second number of said plurality of indicator lights 174 emits a second color of visible light, wherein the first number of indicator lights 174 may be one or two, and/or in combination therewith or separately, wherein the second number of said plurality of indicator lights 174 emitting the second color of visible light may also be one or two, or alternatively the first number of lights 174 may be greater than two, and/or the second number of lights 174 with the second color may greater than two. In some thereof, as may be desired, the first color is emitted when the power supply port has power, and/or the second color of light is emitted when the rechargeable battery of the respective jewelry item as placed in a corresponding charging receptacle 170 is charged to a predetermined level of charge. The box 176 comprising the electronic jewelry case 165 may be made form from any suitable material a hard high-quality plastic material this is further suited to forming such boxes. The material may be selected so that the side walls are opaque or in some preferred embodiments, either transparent, semi-transparent, or translucent such that when the side walls are translucent or semi-transparent, the indicator lights of the first color may be formed integral with the side wall 175, FIGS. 4C to 4F, or at least one of the side wall segments 175a, 175b, 175c, and 175d discussed in regard to FIGS. 4A and 4B. Here also then, the second color indicator light 174 may be formed integral with at least one of the side walls with each pair of charging receptacles 170a and 170b, for example, having a different indicator light 174 associated therewith. In the rectangular embodiment of the box 176 of FIGS. 4A and 4B, the indicator lights 174 of the first color may be formed integral with two or more of the side walls, 175175a and 175b, for example, and together or separately, the indicator lights 174 of the second color correspondingly formed integral with two or more of the side wall segments 175a, 175b, 175c, and 175d. Here as may be preferred, the indicator lights 174 of the first color may be situated in an alternating series with the indicator lights 174 of the second color along the two or more side wall segments 175a, 175b, 175c, and 175d of FIGS. 4A and 4B, or side walls 175 shown in FIGS. 4C to 4F. Here also, the indicator lights 174 of the second color may be situated in an alternating series with the indicator lights 174 of the first color along the two or more side walls or side wall segments. In any of these illuminated embodiments, the indicator lights may be formed from light emitting diodes. In any of the above, the first and second colors of visible light may be selectable from a plurality of different colors provided by a controller associated with a power cord implemented in association with the charging port 173, or alternatively, the first and second colors of visible light may be preferably selectable from a plurality of different colors provided by a controller integrated in association with the bottom 167 of the box 176. In any of these illuminated embodiment with two light colors, the indicator lights 174 of the second color may be emitted in a repeating pattern. And finally here, any of the embodiments of the electronic jewelry box 165 shown in any of FIGS. 4A to 4F, may be present at retail with each of said pre-determined number of charging receptacles 170 including any of the electronic clasp assemblies discussed and/or claimed herein.

And still in further embodiments of the inventions discussed in conjunction with FIGS. 4A to 4M-2, the inventors hereof further provide a charging case for a plurality of network-ready hold fast assemblies for use with pre-existing post-type jewelry items, said charging case comprising in one principal embodiment thereof i) a side wall 175, a bottom 167, and a top 166 configured to form a box 176; ii) a pre-determined number of charging receptacles 170 formed in the top 166 of the box 176, each of said charging receptacles 170 configured to receive a clasp-type network-ready hold fast assembly 168 with each thereof having a rechargeable battery or other form of rechargeable power storage cell; iii) a closable lid 177 for covering at least a portion of the top 166 of the box 176 and for covering the charging receptacles 170 formed therein; iv) a charging component 171 situated in association with each of said charging receptacles 170; v) a power supply port 173 situated in the box 176 in a pre-determine location; and vi) at least one indicator light 174 to indicate when the power supply port 173 has supplied power. In one particular embodiment thereof, the side wall 175, the bottom 167, and the top 166 are configured to form a rectangular box 176 with the closable lid 177 configured thereto in a hinged manner so that the charging receptacles 170 are covered and uncovered by a rotating action of the lid 177 relative the box 176. And here for further functionality, the side wall 175 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170. In a further implementation thereof, each of the charging receptacles 170 may preferably include a corresponding indicator light 174 implemented consecutively along the side wall 175. To add a desired variety and security functionality to the complete assembly, each of the charging receptacles 170 is configured in a particular geometric form and the clasp-type network-ready hold fast assemblies 168 intended for use therewith are each configured in a correspondingly compatible geometric form. In this manner, unauthorized clasp assemblies may not be easily used with the charging case hereof. As such, the geometric form of our clasp assemblies 168 and their corresponding charging receptacles 170 may be rectangular, square, circular, heart-shaped, butterfly-shaped, kitten-shaped, or any other desired shapes and configuration as thereby suggested as examples hereof.

Thus, for example, to achieve a desired circular configuration as like that shown in FIGS. 4C and 4G, the side wall 175, the bottom 167, and the top 166 are configured to form a circular box 176a with a circular closable lid 177 configured thereto in a removable manner so that the charging receptacles 170 are covered and uncovered, respectively, by a corresponding replacing and removing action of the circular lid 177 relative the box 176a. And here also, the side wall 175 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170. And still for more particularized functionality, each of the charging receptacles 170 may preferably include a corresponding indicator light 174 implemented consecutively along the side wall 175. And also here, each of the charging receptacles 170 may be advantageously configured in a particular geometric form and the clasp-type network-ready hold fast assemblies 168 intended for use therewith are each configured in a correspondingly compatible geometric form. And thus like above, as desired, geometric form may be selected from a rectangular, square, circular, heart-shaped, butterfly-shaped, kitten-shaped, or any other thereby suggested by these examples in view of the disclosure hereof. And as such, in the preferred embodiment shown in FIG. 4G, the removable top cover 177, the box 176a, and the clasp assembly 168a are all selected to have a corresponding circular form or circular configuration as illustrated.

To achieve a desired heart-shaped charging case configuration as like that shown in FIGS. 4D and 4H, the side wall 175, the bottom 167, and the top 166 are configured to form a heart-shaped box 176b with a heart-shaped closable lid 177 configured thereto in a removable manner so that the charging receptacles 170 are covered and uncovered, respectively, by a corresponding replacing and removing action of the heart-shaped lid 177 relative the box 176b. Here like above, the side wall 175 incudes at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170, and as may preferred, each of the charging receptacles 170 may include a corresponding indicator light 174 implemented consecutively along the side wall 175. Alternatively, the heart-shaped lid 177 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170. And more preferably then, each of the charging receptacles 170 may include a corresponding indicator light 174 implemented consecutively in association with heart-shaped lid 177 as illustrated in the lip 177 of FIG. 4H. In any of the above heart-shaped configurations, each of the charging receptacles 170 may be advantageously configured in a particular geometric form with the clasp-type network-ready hold fast assemblies 168 intended for use therewith each being configured in a correspondingly compatible geometric form, where here also, the geometric form may be rectangular, square, circular, heart-shaped, butterfly-shaped, or kitten-shaped, for example. And as such, in the preferred embodiment of the heart-shaped charging case 165b shown in FIG. 4H, the removable top cover 177, the box 176b, and the clasp assembly 168d are all selected to have a corresponding heart-shaped form or heart-shaped configuration as illustrated.

And thus next to achieve desired butterfly-shaped charging case configurations such as those shown in FIGS. 4E, 4I-1, 4I-2, 4J-1, and 4J-2, the side wall 175, the bottom 167, and the top 166 are configured to form a butterfly-shaped box 176c with a butterfly-shaped closable lid 177 configured thereto in a removable manner so that the charging receptacles 170 are covered and uncovered, respectively, by a corresponding replacing and removing action of the butterfly-shaped lid 177 relative the box 176c as shown in FIGS. 4J-1 and 4J-2. Alternatively the side wall 175, the bottom 167, and the top 166 are configured to form a butterfly-shaped box 176c with a two-piece butterfly-shaped closable lid 177 including wings 177a and 177b configured thereto in a hinged manner so that the charging receptacles 170 are covered and uncovered by a separate rotating action of each piece of the two-piece butterfly-shaped closable lid 177a and 177b relative to the box 176c as shown in FIGS. 4I-1 and 4I-2. And like above, here in the butterfly embodiment, the side wall 175 may include advantageously at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170. Or more preferably, where desired each of the charging receptacles 170 may include a corresponding indicator light 174 implemented consecutively along the side wall 175. Alternatively, the butterfly-shaped lid 177 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170 where in some embodiments, each of the charging receptacles 170 includes a corresponding indicator light 174 implemented consecutively in association with the butterfly-shaped lid 177. To further provide the security discussed above regarding use of only authorized electronic clasp assemblies 168 herewith, each of the charging receptacles 170 may be configured in a particular geometric form and the clasp-type network-ready hold fast assemblies 168 intended for use therewith then are each configured in a correspondingly compatible geometric form, the geometric form then being rectangular, square, circular, heart-shaped, butterfly-shaped, or kitten-shaped.

And now lastly here for these different box shapes, in FIGS. 4F and 4K, the inventors hereof provide a kitten-shaped charging case 165d wherein the side wall 175, the bottom 167, and the top 166 are configured to form a kitten-shaped box 176d with a kitten-shaped closable lid 177 configured thereto in a removable manner so that the charging receptacles 170 are covered and uncovered, respectively, by a corresponding replacing and removing action of the kitten-shaped lid 177 relative the box 176d. Alternatively, the side wall 175, the bottom 167, and the top 166 are configured to form a kitten-shaped box 176d with a kitten-shaped closable lid 177 configured thereto in a hinged manner so that the charging receptacles 170 are covered and uncovered by a rotating action of the kitten-shaped closable lid 177 relative to the box 176d. And similarly to provide a sense of functional animation, the side wall 175 incudes at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170, where in a preferred embodiment thereof, each of the charging receptacles 170 includes a corresponding indicator light 174 implemented consecutively along the side wall 175. Alternatively, the kitten-shaped lid 177 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170 or, like above with the heart-shaped and butterfly-shaped lids, each of the charging receptacles 170 includes a corresponding indicator light 174 implemented consecutively in association with the kitten-shaped lid 177. Also here in the kitten-shaped box 165d, each of the charging receptacles 170 may be configured in a particular geometric form and the clasp-type network-ready hold fast assemblies 168 intended for use therewith then being each configured in a correspondingly compatible geometric form. And as such, for example, said geometric form may be rectangular, square, circular, heart-shaped, butterfly-shaped, or kitten-shaped. And according to further aspects hereof, for a sense or feel of animation and life, the kitten-shaped charging case may include simulated fur configured on at least a portion of the kitten-shaped box 176d or on at least a portion of the kitten-shaped closable lid 177 or on at least a portion of both the kitten-shaped box 176d and the kitten-shaped closable lid 177. And for further functional amination, any of the above kitten or cat-shaped charging cases may further include an indicator light 174 configured as each of two cat eyes provided in a face portion of the kitten or cat-shaped box 176d.

As for any desired wing-shaped charging case, now generally referring to FIGS. 4E, 4I-1 and 4I-2, 4J-1 and 4J-2, the side wall 175, the bottom 167, and the top 166 may be configured to form any desired wing-shaped box 176c with a wing-shaped closable lid 177 configured thereto in a removable manner so that the charging receptacles 170 are covered and uncovered, respectively, by a corresponding replacing and removing action of the wing-shaped lid 177 relative the box 176c. Alternatively, the side wall 175, the bottom 167, and the top 166 are configured to form a wing-shaped box 176c with a two-piece wing-shaped closable lid 177 configured thereto in a hinged manner so that the charging receptacles 170 are covered and uncovered by a separate rotating action of each piece of the two-piece wing-shaped closable lid 177 relative to the box 176c. In any of these generally wing-shaped embodiments, the side wall 175 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170, and preferably where desired each of the charging receptacles 170 may include a corresponding indicator light 174 implemented consecutively along the side wall 175. Alternatively or in combination therewith, the wing-shaped lid 177 incudes at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170. And more preferably in some embodiments thereof, each of the charging receptacles 170 includes a corresponding indicator light 174 implemented consecutively in association with the wing-shaped lid 177. In any of these wing-shaped embodiments, each of the charging receptacles 170 may be advantageously configured in a particular geometric form and the clasp-type network-ready hold fast assemblies 168 intended for use therewith are each configured in a correspondingly compatible geometric form, as for the product securing reasons discussed above. And for particular embodiments of our wing-shaped electronic jewelry boxes, wing-shape box 176c and its corresponding wing-shaped lid 177 may be rendered together to form alternately a butterfly configuration, as illustrated, or otherwise rendered together to form a hummingbird configuration, a bumblebee configuration, a honeybee configuration, a ladybug configuration, a dragonfly configuration, an eagle bird-configuration, a falcon bird-configuration, a hawk bird-configuration. Any in any of these embodiments, the geometric form of the charging receptacle 170 and corresponding shape of the electronic clasp assembly 168 may be butterfly-shaped, hummingbird-shaped, bumblebee-shaped, honeybee-shaped, ladybug-shaped, dragonfly-shaped, eagle-shaped, falcon-shaped, or hawk-shaped.

And finally here, as for any desired animal-shaped charging case 165, now generally referring to FIGS. 4F and 4K as representative thereof, the inventors hereof further provide a charging case wherein the side wall 175, the bottom 167, and the top 166 are configured to form an animal-shaped box 176d with a corresponding animal-shaped closable lid 177 configured thereto in a removable manner so that the charging receptacles 170 are covered and uncovered, respectively, by a corresponding replacing and removing action of the animal-shaped lid 177 relative the animal-shaped box 176d. Alternatively, the side wall 175, the bottom 167, and the top 166 are configured to form an animal-shaped box 176d with an animal-shaped closable lid 177 configured thereto in a hinged manner so that the charging receptacles 170 are covered and uncovered by a rotating action of the animal-shaped closable lid 177 relative to the animal-shaped box 176d. In either of these embodiments, the side wall 175 incudes at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170, and more preferably where desired, each of the charging receptacles 170 includes a corresponding indicator light 174 implemented consecutively along the side wall 175. Alternatively or in combination therewith, the animal-shaped lid 177 may include at least one indicator light 174 associated with a corresponding charging receptacle 170 to indicate when a respective rechargeable battery associated with a corresponding clasp-type network-ready hold fast assembly 168 is charged after placing same in said corresponding charging receptacle 170, and in certain preferred embodiments thereof, each of the charging receptacles 170 includes a corresponding indicator light 174 implemented consecutively in association with the animal-shaped lid 177. In any of these animal-shaped embodiments of our charging case 165 each of the charging receptacles 170 may be configured in a particular geometric form and the clasp-type network-ready hold fast assemblies 168 intended for use therewith are then each configured in a correspondingly compatible geometric form. And for particularized animal shapes as may be desired, the inventors hereof provide that the animal-shape box 176d and its corresponding animal-shaped lid 177 may be rendered together to form a kitten configuration, a cat configuration, a puppy-dog configuration, a dog configuration, a panda bear configuration, a koala bear configuration, a rabbit configuration, a peacock configuration, a lion configuration, a leopard configuration, a panther configuration, or any other desired animal configuration with indication lights 174 animating the figure as illuminated eyes or other such features and accessories. And in certain preferred embodiments thereof, the geometric forms of the electronic clasps 168 and receptacles 170 correspond to a selected animal configuration of the animal-shape box 176d. Any of these animal-shaped embodiments may further include simulated fur configured on at least a portion of the animal-shaped box 176d or on at least a portion of the animal-shaped closable lid 177 or on at least a portion of both the animal-shaped box 176d and the animal-shaped closable lid 177. And further to provided a completed assembly ready for use, any of the above charging cases may be delivered wherein each of the pre-determined number of charging receptacles includes a corresponding network-ready hold fast assembly of a desired type as provided herein. And as for recharging the frontside electronic jewelry items hereof, like the kitten-configured earring of FIG. 4K, and those discussed further herein below, there is provided that any of the charging cases of FIGS. 4A to 4m-2 may advantageously include at least one of their pre-determined number of charging receptacles 170 being further configured to recharge any of the frontside electronic jewelry items hereof.

And even more specifically regarding the sculpted animal-formed jewelry boxes hereof, as exemplified by the sculpted and contoured kitten or cat-shaped charging case 165e of FIGS. 4L to 4M-2, the inventors more particularly provide a method of making an animal-shaped electronic jewelry box 165e, said method comprising the steps of i) receiving at least one specified image of an animal; ii) rendering a sculped form of the animal provided by the at least one specified image thereof; iii) providing a box 176d configured within the sculpted form; iv) providing the box 176d with a plurality of charging receptacles 170, 170n with each thereof configured to receive an electronic jewelry item for recharging 168g 100d; v) integrating at least one indicator light 174 in the sculped form; and iv) providing the box 176d with electronic control circuitry to activate the at least one indicator light and the charging receptacles in a prescribed manner. According to this method the at least one specified image of the animal may include a plurality of specified images of the animal. And with the advent of computer-aided-design (CAD) tools, the rendering step may advantageously include machine reading of the plurality of specified images and creating a three-dimensional model of the animal within a CAD tool, for example. Here then, the three-dimensional CAD model of the animal is used to form the sculped form of the animal, for example with a 3-D printer. And for a further desired final rendering, the sculped form of the animal may be provided with life-like synthetic or simulated fur by applying such relevant established arts thereto. And for an ever more life-like rending of a specific animal, such as a much beloved family pet, the synthetic fur may be colored according to the colors of the animal provided by the at least one specified image thereof. Thus the at least one specified image of the animal may include a traditional paper photograph of the animal in the case of animals or pets who are no longer with their owners, or in the case of currently living animals or pets, the at least one specified image of the animal may include one or more digital images of the animal taken with a mobile phone for example and emailed over the internet to the provider of this method as is well known in the commercial arts of internet commerce.

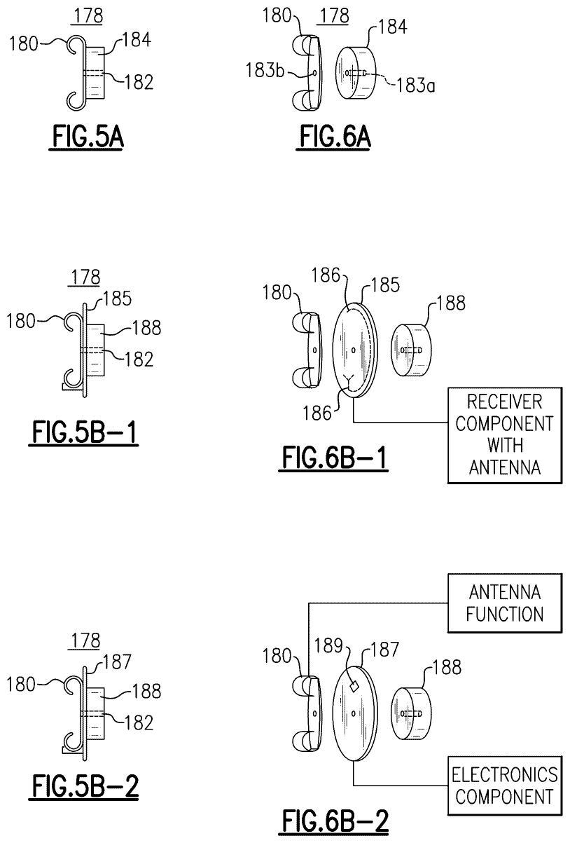

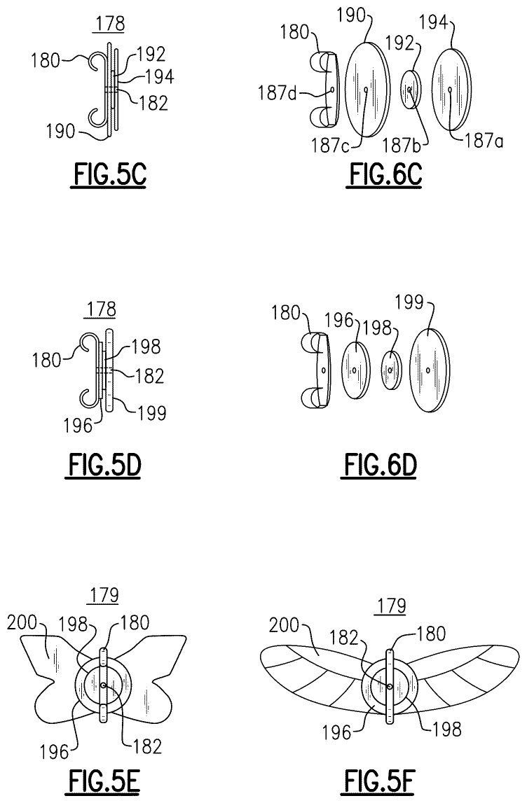

Now moving on to FIGS. 5A to 5D, there is illustrated a series of pictorial side elevation views of different embodiments of a rechargeable network-ready vibrating jewelry clasp 178 that may be provided individually as shown or in combination with a charging case like the recharging case 165 discussed above in connection with FIGS. 4A to 4M-2. In this manner, the clasps 178 may be charged with supply energy and then deployed for use with pre-existing items of jewelry that have been long treasured by an owner thereof. Alternatively, the rechargeable network-ready vibrating jewelry clasps 178 may be provided in combination with a corresponding newly manufactured item of jewelry. Thus in either case, the rechargeable network-ready vibrating jewelry clasp 178 is, by the teachings hereof, ready for wearable interactive use with a smartphone, a personal diagnostic bio-patch or fever patch, or any combination thereof as use in a personal mobile network with each such element being a reporting node, a receiving node, or a communications node in such a configured short-range mobile network that moves in synchronicity in space and time with the movement of the personal space of a person using the configuration as a wearable private network assembly for personal notification purposes.

For purposes of discussion of the individual components of the assemblies in FIGS. 5A to 5D, the perspective exploded views of FIGS. 6A to 6D are provided herein as corresponding respectively to the claps shown in FIGS. 5A to 5D. As such, in the following, FIG. 5A may be taken together with FIG. 6A, FIG. 5B considered with FIG. 6B, and so on to FIGS. 5D and 6D.

Thus with the above at hand, it is shown that each of the embodiments of the rechargeable network-ready vibrating jewelry clasp assembly 178 of FIGS. 5A to 5D includes a clasp 180 having a concentric hole in register with a thru-hole or through-hole 182 which traverses the assembly, as illustrated, to mate in a securable manner with the post of a jewelry item such as an earring. In some embodiments, clasp 180 may serve the dual purposes of securing an item of jewelry to the body and also functioning as an antenna for reception of a signal directed thereto. In the embodiment of FIGS. 5A and 6A, the receiver, the vibration assembly, and the rechargeable battery or other suitable type of rechargeable power cell are provided within a sealed housing herein presented as a vibration puck or vibrating puck 184. Thus in this manner, clasp assembly 178 is formed from two principal parts including the clasp 180 as secured in a fixed manner to the vibrating puck 184.

Thus as illustrated and described in connection with FIGS. 5A and 6A, in this specific preferred embodiment hereof, the inventors provide a network-ready clasp assembly 178 for wearable use with a jewelry item having a post, where this particular network-ready clasp assembly comprises i) a vibrating puck 184 including a central hole formed therethrough, the vibrating puck 184 herein being configured in the form of a sealed casing having therein enclosed in operable association with one another, a rechargeable battery, receiving circuitry, control circuitry, and an electro-mechanical component for providing vibration; and ii) a clasp 180 with a central hole formed therethrough, the clasp affixed to the vibrating puck with the central holes of each of the clasp and vibrating puck in register to form a through-hole configured to receive a post of a jewelry item so that upon receipt of a signal from a remote device, the electro-mechanical component is activated to provide a vibration sensation to a person wearing the assembly with the jewelry item.

Next in FIGS. 5B-1 and 6B-1, an alternate embodiment of the clasp assembly 178 is illustrated where the assembly includes the clasp 180, a receiver assembly or receiver component 185 here shown including an antenna 186, and an alternate embodiment of a vibrating puck as labeled vibration puck 188 here in FIGS. 5B-1 and 6B-1. In this embodiment, the receiver component 185 and antenna 186 are provided in a sealed housing including the receiver circuitry. In this embodiment of FIGS. 5B-1 and 6B-1, the vibration puck 188 is similarly provided in a sealed housing that includes a vibration assembly in accordance with the above discussion and also includes the control circuitry and a power cell such as a rechargeable battery. Then in final steps of manufacture assembly, the clasp 180, the receiver component 185, and the vibration puck 188 are affixed to one another in register as illustrated to form the through-hole 182 thereby configuring the clasp assembly 178 to receive the post of an existing item of jewelry such as an earring to be worn on the ear of a user in a manner as described herein. And thus here to activate the vibration assembly, there is provided an electrical connection between the receiver component 185 and the vibrating puck 188 such that when the receiver circuitry receives a notification signal from a personal diagnostic device or a smartphone, the control circuitry will enable the vibration assembly to vibrate thus providing a silent notification in a discreet manner to a person utilizing the clasp assembly 178 as a wearable smart device. Thus in this embodiment, there is provided a network-ready clasp assembly 178 for wearable use with a jewelry item having a post. In this specific embodiment, the network-ready clasp assembly 178 includes i) a vibrating puck 188 including a central hole formed therethrough, the vibrating puck 188 being configured in the form of a sealed casing having therein enclosed in operable association a rechargeable battery with control circuitry and an electro-mechanical component for providing vibration; ii) a receiver component 185 including a central hole formed therethrough, said receiver component having sealed therein receiving circuitry operably associated with said control circuitry in said vibrating puck, and said receiver component being affixed to said vibrating puck with the central holes of each thereof being in register with one another; and iii) a clasp 180 with a central hole formed therethrough, said clasp affixed to said receiver component with the central holes of each of the clasp 180, the receiver component 185, and the vibrating puck 188 in register to form a through-hole configured to receive the post of a jewelry item so that upon receipt of a signal from a remote device, the electro-mechanical component is activated to provide a vibration sensation to a person wearing the clasp assembly with the jewelry item.

Alternatively as represented in FIGS. 5B-2 and 6B-2, another embodiment of the clasp assembly 178 may be provided where the receiver circuitry and control circuitry are implemented in an electronics component 187 provided in a sealed housing with a contact pad 189. In this embodiment of FIGS. 5B-2 and 6B-2, the vibration puck 188 is similarly provided in a sealed housing which includes the vibration assembly and a rechargeable power cell such as a rechargeable battery adapted hereto. In this embodiment of the clasp assembly 178 of FIGS. 5B-2 and 6B-2, the clasp 180 as shown in the accompanying text box may serve as an antenna as an alternative to, or in addition to, the antenna 186 discussed above in the embodiment of FIG. 6B-1. Thus here, the clasp 180 as shown in FIG. 6B-2 electronically connects to the receiver circuitry in the electronics assembly 187 via the contact pad 189. Then in final steps of manufacture assembly, the clasp 180, the electronics component 187, and the vibration puck 188 are affixed to one another in register as illustrated to form the through-hole 182 thereby configuring the finished clasp assembly 178, like those above, to receive the post of an existing item of jewelry. And thus similarly here to activate the vibration assembly, there is provided an electrical connection between the electronics component 187 and the vibrating puck 188 such that when the receiver circuitry receives a notification signal from a personal diagnostic device or a smartphone, the control circuitry, through the electrical connection between the electronics component 187 and the vibrating puck 188, enables the vibration assembly to vibrate to thereby give a user of the device personal notification as to an external event or condition. Thus here in this alternate embodiment, there is provided by the inventors hereof a network-ready clasp assembly 178 for wearable use with a jewelry item having a post wherein the network-ready clasp assembly 178 comprises i) an electronics component including a central hole formed therethrough, said electronics component configured in the form of a sealed casing having therein enclosed receiving circuitry in operable association with control circuitry; ii) a vibrating puck including a central hole formed therethrough, said vibrating puck having sealed therein a rechargeable battery and an electro-mechanical component operably associated with said control circuitry in said electronics component, said electronics component being affixed to said vibrating puck with the central holes of each thereof being in register with one another; and iii) a clasp with a central hole formed therethrough, said clasp affixed to said electronics component with the central holes of each of the clasp, the electronics component, and the vibrating puck in register to form a through-hole configured to receive a post of a jewelry item so that upon receipt of a signal from a remote device, the electro-mechanical component is activated to provide a vibration sensation to a person wearing the clasp assembly with a respective jewelry item.

With reference next to FIGS. 5C and 6C, there is shown yet another alternate embodiment of the clasp assembly 178 according to this invention. Here in this embodiment, the assembly 178 includes the clasp portion 180, a components housing 190, a spacer 192, and a vibration member or vibration component 194 each with a central hole and securely assembled together in register to form the thru-hole 182 as illustrated. Here, the rechargeable battery and electronic circuitry for receiving and controlling are preferably provided within the housing 190 in a sealed manner with an electrical connection through the spacer 192 so that the vibration member 194 may be activated with a provided electric supply and then vibrate in a mechanical manner to thereby provide personal silent notification to a user thereof according to the teaching hereof. In additional embodiments hereof, the spacer 192 may include any selected portion of the electronics providing the desired functionalities and utilities hereof or may be only a conduit for electrical supply to the vibration member 194. In these later cases, the spacer 192 may be preferably formed from a supple material such as rubber to thereby provide some freedom of deflection among the elements of the clasp assembly 178 such that the vibration member 194 may vibrate with fewer rigid forces acting thereon. In a more preferred embodiment, the spacer 192 is circular is shape and the diameter thereof is substantially smaller than the diameter of the vibration member 194. In this manner, the distal portions of the vibration member 194 are free of solid backing and thus are more easily caused to vibrate given a supplied mechanical displacement as driven by a supplied power provide from the rechargeable battery thus giving a longer life and increased mechanical effectiveness to vibration functionality of the vibration member 194 after placing same in a fully charged condition. Thus here as discussed in conjunction with FIGS. 5C and 6C, the inventors hereof further provide a network-ready clasp assembly 178 for wearable use with a jewelry item having a post, with this particular embodiment of our network-ready clasp assembly comprising i) a components housing 190 including a central hole formed therethrough, the components housing configured in the form of a sealed casing having therein enclosed a rechargeable battery, receiving circuitry, and control circuitry; ii) a vibration member 194 including a central hole formed therethrough, the vibration member having sealed therein an electro-mechanical component implemented in operable association with the control circuitry; iii) a spacer 192 including a central hole formed therethrough, the spacer having an electrical connection from the control circuitry in the components housing 190 to the electro-mechanical component in the vibration member 194 and being affixed therebetween with each of the central holes of the components housing, spacer, and vibration member in register with one another; and iv) a clasp 180 with a central hole formed therethrough, the clasp affixed to the components housing with the central holes of each of the clasp, the components housing, the spacer, and the vibration member in register to form a through-hole configured to receive a post of a jewelry item so that upon receipt of a signal from a remote device, the electro-mechanical component is activated to provide a vibration sensation to a person wearing the clasp assembly with a respective jewelry item.

With the advancing state of the art of micro-electronics, it is herein proposed that the electronic circuitry required to provide our desired functionality to the clasp assembly 178 overall may be provided in a package that is considerably smaller than the desired sizing of the vibration member and also considerably smaller than the sizing of the rechargeable battery needed to drive the assembly over its daily use scenarios as intended by the provisions hereof. Thus there is provided a further preferred configuration of the clasp assembly 178 in FIGS. 5D and 6D wherein the assembly 178 includes the clasp 180, a rechargeable battery 196, an electronics housing 198, and a circular vibration member 199. Here also in this embodiment of FIGS. 5D and 6D, each of the clasp portion 180, the rechargeable battery 196, the electronics housing 198, and the vibration member 199 are provided with a central hole and securely affixed together in register to form the thru-hole 182 as illustrated. Here, the electronics housing is sized considerably smaller than the vibration member 199 thereby serving also as a spacer between the rechargeable battery 196 and the circular vibration member 199. In this manner, the distal portions of the vibration member 199 are mechanically free of any solid backing material and thus with less mass are more easily vibrated with a given needed supply power to activate an intended state of vibration. Thus here in FIGS. 5D and 6D, the inventors provide a network-ready clasp assembly 178 for wearable use with a jewelry item having a post, this network-ready clasp assembly comprising i) a rechargeable battery 196 implemented in a sealed casing including a central hole formed therethrough; ii) a vibration member 199 having a diameter thereacross with a central hole formed therethrough, the vibration member including sealed therein an electro-mechanical component; iii) an electronics housing 198 having a diameter thereacross with a central hole formed therethrough, said electronics housing including sealed therein receiving circuitry and control circuitry and being affixed between the rechargeable battery and vibration member with each of the central holes of the rechargeable battery, the electronics housing, and the vibration member in register with one another; and iv) a clasp 180 with a central hole formed therethrough, said clasp affixed to said rechargeable battery 196 with the central holes of each of the clasp, the rechargeable battery, the electronics housing, and the vibration member in register to form a through-hole configured to receive a post of a jewelry item so that upon receipt of a signal from a remote device, the electro-mechanical component is activated to provide a vibration sensation to a person wearing the clasp assembly with a respective jewelry item. In this embodiment, the diameter across the vibration member is greater than the diameter across the electronics housing, where preferably the diameter across the vibration member is at least twice that of the diameter across the electronics housing so that a desired distal position of the vibration member is free of backing material such that it may more freely vibrated when so activated.

Thus given the desired utility of providing a vibration member with reduced peripheral mass, more freedom to vibrate, less mechanical stiffness, or any preferred combination thereof as correlated to needed input energy over a desired use scenario, as implemented in a clasp assembly of the present invention, the inventors hereof further provide herein certain alternate preferred configurations of the vibration member 199 of FIGS. 5D and 6D as next illustrated in FIGS. 5E and 5F.

Now with continuing reference to FIGS. 5E and 5F, there are presented elevation views of two alternate embodiments 179 of the clasp assembly of FIG. 5D with each thereof having a wing-shaped vibration member 200 as an alternative to the circular vibration member 199 shown in FIGS. 5D and 6D. As such, the wing-shaped vibration member 200 of the clasp assembly 179 of FIG. 5E is configured in the form of a pair of butterfly wings wherein the clasp 180 servers as a centering mass along with the battery and the electronics with the distal portions of the wing-shaped vibration member 200 free of any backing material at the distal regions thereof to thereby better provide vibration in the distal regions with a given power supply afforded by a suitably sized rechargeable battery or power cell as correlated to a preferred daily use scenario. And similarly, in FIG. 5F, the wing-shaped vibration member 200 is configured in the form of a pair of humming bird wings wherein the clasp 180 also serves as a centering mass along with the battery and the electronics which are sized to have a concentrated mass within a predetermined radius from the thru-hole 182 while the wing span of the butterfly or humming bird wings is provided with an overall length longer than the diameter of the concentrated mass created by the clasp, battery, and electronic components thus allowing the distal portions of the wing-shaped vibration member 200 to be free of any backing material to thereby vibrate more easily given a supplied unit of input energy. In these configurations, the casings of the wings may be formed from any suitable material and are preferably formed from gold or silver of a desired thickness where the necessary MEMS components for causing vibration are secured therein according to the teachings of our related inventions as referenced and incorporated herein. And as would be readily apparent to those skilled in these arts given the disclosures hereof, the shape of the vibration member 200 would not be limited to the butterfly shape of FIG. 5E or the hummingbird wing shape of FIG. 5F but may be extended to include any other desired shapes or formations from nature or other indicia, symbols, or visual configurations as desired which for these functional purposes have an overall diameter greater than a diameter of the concentrated mass created by centering the clasp, battery, and electronics as discussed above.

Thus more particularly in connection with FIGS. 5E and 5F, certain preferred embodiments of the present inventions are directed to a network-ready clasp assembly 179 for wearable use with a jewelry item having a post where the network-ready clasp assembly includes i) a rechargeable battery 196 implemented in a sealed casing including a central hole formed therethrough, ii) a wing-shaped vibration member 200 having a diameter thereacross and a central hole formed therethrough, said wing-shaped vibration member 200 including sealed therein an electro-mechanical component, iii) an electronics housing 198 having a diameter thereacross and a central hole formed therethrough, said electronics housing 198 including sealed therein receiving circuitry and control circuitry with each of the central holes of the rechargeable battery 196, the electronics housing 198, and the wing-shaped vibration member 200 in register with one another; and iv) a clasp 180 with a central hole 182 formed therethrough, said clasp 180 affixed to a back side of the assembly 179 with the central holes of each of the clasp 180, the rechargeable battery 196, the electronics housing 198, and the wing-shaped vibration member 200 in register to form a through-hole in the assembly configured to receive a post of a jewelry item so that upon receipt of a signal from a remote device, the electro-mechanical component is activated to provide a vibration sensation to a person wearing the clasp assembly with a respective jewelry item. In one embodiment hereof, the wing-shaped vibration member 200 is formed in the shape of butterfly wings, and in another embodiment the wing-shaped vibration member 200 is formed in the shape of hummingbird wings. Alternatively, the wing-shaped vibration member 200 may be formed in the shape of honeybee or bumblebee wings and the clasp 180 and/or the electronical housing 198 taken together or separately, may be formed in the shape of a honeybee or bumblebee body. Similarly, the wing-shaped vibration member 200 may be formed in the shape of eagle wings and the clasp 180 and/or the electronical housing 198 taken together or separately, may be correspondingly formed in the shape of the head of an eagle, or still when the wing-shaped vibration member 200 is formed in the shape of eagle wings, the clasp 180 and/or the electronical housing 198, taken together or separately, may be formed in the shape of the head and body of an eagle. And thus as further alternative embodiments, the wing-shaped vibration member 200 may be formed in the shape of at least one peacock tail feather, two peacock tail feathers in a cross-x configuration, or the wing-shaped vibration member 200 may be formed in the shape of a plurality of peacock tail feathers in an open fan configuration. Still further, the wing-shaped vibration member 200 may be formed in the shape of a plurality of peacock tail feathers in an open fan configuration and the clasp 180 and/or electronics housing 198 taken together or separately are then formed in a shape of a peacock head and body. In yet further embodiments of the electronic clasp assembly 179 of FIGS. 5E and 5F, the wing-shaped vibration member 200 may be formed in a shape of open ladybug wings and the clasp 180 and/or the electronics housing 198 taken together or separately are then formed in a shape of a ladybug head and body, or the wing-shaped vibration member 200 may be formed in the shape of open dragonfly wings and then the clasp 180 and electronics housing 198 taken together or separately are formed in the shape of a dragonfly head and body. And still further, the wing-shaped vibration member 200 may be formed in the shape of falcon or hawk wings and then the clasp 180 and electronics housing 198 taken together or separately are formed in the shape of the head and body of a falcon or hawk, respectively.

And further, as would be apparent to those developing skills in these new arts of electronic jewelry for personal notification purposes given the disclosures and teaching hereof, the line-up of components from the clasp 180 to the vibration member 194 may be adjusted to include four, three, or two separate main components, as illustrated across the different embodiments of FIGS. 5A to 5F, for example, and the principal functional elements including the rechargeable battery, the control circuitry, the receiver circuitry, the antenna, and the different MEMS elements forming the vibration member, may be integrated on a single components board in a single integrated circuit chip, or they may be alternatively implemented on a single components board in two or more integrated circuit chips, or yet still they may be implemented on two or more components boards including two or more integrated circuit chips provided within the clasp 180 (the antenna as provided therein, for example), the vibration puck 184, the receiver assembly 185, the vibration puck 188, the components housing 190, the spacer 192, the vibration member 194, or the electronics housing 198, or any various combinations and consolidations thereof as may be desired. Thus the present inventions should not be considered limited to the precise detailed embodiments discussed in conjunction with FIGS. 5A to 5F but may be fairly considered to include all various thereof as coming within the spirit and scope of the claims presented herein below.

For purposes of convenient nomenclature as to classification of some of the inventions hereof, the above embodiments of the clasp assembly 178 in FIGS. 5A to 5C, as a class, group, or product type grouping, may be referred to as “stacked clasp assemblies” or “puck-type clasp assemblies” since the principal electronic and electro-mechanical vibration components are assembled in a stack when viewed along an axis that is colinear with the thru-hole 182 or otherwise provided or partly provided in the form of a puck in a lineup along such an axis. In the case of the configurations of FIGS. 5E and 5F where the wing-shaped vibration member 200 has a length thereacross of about twice or more as compared to the other electronic components of the assembly, as here illustrated, then for purposes of nomenclature as to classification of these configurations, as a class, group, product type, or product grouping, our term “dual-form clasp assemblies” may be applied for convenience of discussion. Thus as such, the configuration of FIGS. 5D and 6D, depending on the diameter of the vibration 199 relative to the diameters of the electronics housing 198 and the battery 196, may be referred to as a “stacked clasp assembly” or a “dual-form clasp assembly” as the term may apply in view of the widths of these components when taken relative to one another as viewed along a longitudinal axes taken along a width of the assembly including the thru-hole 182.

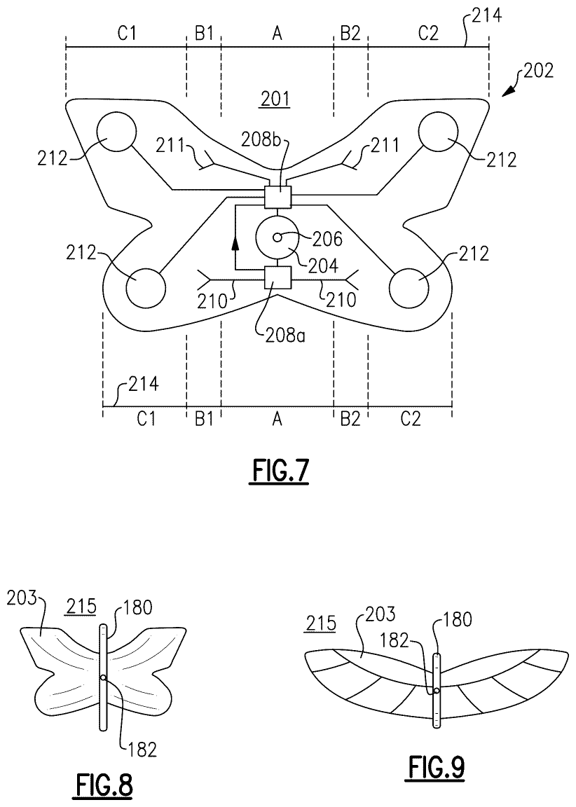

Thus as proposed herein above and discussed in further detail below in conjunction with FIGS. 7 to 10B, any of the electronic network-ready clasp assemblies 178 disclosed in FIGS. 5A to 5D and 6A to 6D, may be advantageously configured in certain particular two-dimensional geometric shapes or further so configured as three-dimensional forms for not only aesthetic or artistic purposes but also for utilitarian functional purposes such as device security, energy management and conservation, and personal silent notification as such notification herein relates to the kinematics or mechanical dynamics of vibrating objects. More particularly, the vibrating puck 184 discussed above in conjunction with FIGS. 5A and 6A, may be heart-shaped or butterfly-shaped to so comport with the shape of a charging receptacle or further conformed, for example, into a solid three-dimensional form of a sleeping kitten in a front view with a flat vibrating back or bottom side that gently presses against the back of the ear lobe when deployed with an existing item of jewelry to give silent personal notification to a user thereof. In a similar manner, the receiver assembly 185 of FIGS. 5B-1 and 6B-1 while shown disk-shaped for simplicity in these figures, may readily be configured into a two-dimensional heart-shape form or wing-shaped form, for example, with such forms and shapes also applied to the vibrating puck 188 thereof as discussed. Further in kind, the electronics assembly 187 and vibrating puck 188 of FIGS. 5B-2 and 5B-2, along with the components housing 190 and vibration member 194 of FIGS. 5C and 6C, and also along with the circular or disk-shaped vibration member 199 of FIGS. 5C and 6C, may be rendered into particular shapes and forms to achieve to achieve not only the artistic purposes but also therewith various functional and utilitarian advantages associated therewith. Thus as such, and as discussed above, the vibration members 200 of the electronic clasp assemblies 179 of FIGS. 5E and 5F are illustrated as wing-shaped in the form of butterfly wings as shown in FIG. 5E or hummingbird wings as in FIG. 5F.

And further yet as an alternative to the above stacked clasp assemblies and dual-form clasp assemblies, whether rendered in simple or more complex shapes and forms, the inventors hereof provide a layout arrangement where the principle electronic and electro-mechanical components hereof are provided in a planar layout on a circuit board with the components being subject to layout rules, preferences, and optimized placements on the circuit board that may have a specific shape to aid in functionality while also providing an emotional sentiment when worn as an item of jewelry.

Thus with reference next to FIGS. 7 and 8, there is schematically illustrated an enlarged plan view of an exemplary circuit board assembly 201 with principal components that may be utilized to provide desired functionality to the clasp assembly of FIG. 8. Here in this embodiment, a circuit board 202 of a desired shape is firstly provide with a rechargeable battery 204 centrally positioned therein as illustrated. As further shown, the circuit board 202 is generally configured in the shape of butterfly wings to comport with the desired shape of a corresponding vibration member 203 as illustrated in FIG. 8. As shown in FIG. 7, both the circuit board 202 and the rechargeable battery 204 are provided with a central hole 206 that comports to form the thru-hole 182 shown in FIGS. 8 and 9. Next as schematically illustrated, the circuit board 202 may be provided with a number of integrated circuit chips 208. Here in this particular embodiment, two integrated circuit chips 208 are provided including a first integrated circuit chip 208a and a second integrated circuit chip 208b where the integrated circuit chip 208a is implemented to include receiver circuitry and the integrated circuit chip 208b includes controller circuitry. And as further shown, the integrated circuit chip 208a is provide with an antenna 210 as implemented to receive a closed air signal from a smartphone, personal diagnostic device such as a fever patch or bio-patch, or both a smartphone and a wearable personal diagnostic device as discussed herein below in further detail in connection with FIG. 17C. In embodiments of the circuit board assembly 201 where the control circuitry 208b is further implemented to send a reporting signal, a transmission antenna 211 may be implemented therewith to thereby send a desired information signal to a remote device.

Then as next illustrated in FIG. 7, the integrated circuit chip 208b is connected to four vibrating disks 212 which are located on the periphery of the circuit board 202 as shaped in this particular embodiment in the form of miniature butterfly wings. The circuit board 202 may be made of a rigid material having a consistent thickness and stiffness thereacross or it may include a gradient of stiffness across the wingspan. Thus in this manner, for convenience of discussion, a stiffness indicator bar 214 is placed above and below the circuit board 202 in FIG. 7 for purposes of graphically illustrating this aspect of the present invention. Here then according to these teachings, in a central region A of the circuit board 202 containing the battery 204 and the integrated circuit chips 208, the circuit board 202 is provided with a high stiffness to secure the battery and integrated circuit chips in a flat and rigid manner. Then across the regions labeled B1 and B2, the circuit board 202 transitions to a lower stiffness in corresponding regions C1 and C2 at an outer or distal portion of each of the wings as illustrated by the stiffness indicator bar 214. Thus in this manner, when the antenna 210 receives a signal from a smartphone or bio-patch, the receiver chip notifies the controller chip 208b, which in turn activates the vibrating disks 212 to pulse with vibration over a selected period of time thereby giving a user of the device a silent personal notification. And according to advantages hereof, in the embodiments where the circuit board 202 includes lower stiffness in distal portions including the vibrating disks 212, for a given unit of input energy from the battery 204 to a vibrating disk 212, displacement of a distal portion along an axis normal to the circuit board centered through a respective disk, may be increased due to the corresponding reduction of circuit board stiffness in the area adjacent to the vibrating disk. Thus in this manner, a given charge of the battery as sized for this device may provide a use scenario over a longer time period. Thus as discussed in regard to FIG. 7, there is provided a circuit board assembly 201 for use with an item of jewelry having a post, with this circuit board assembly comprising i) a circuit board 202 configured to receive a plurality of components, said circuit board including a central hole and having a preselected shape, ii) a rechargeable battery 204 including a central hole 206, said rechargeable battery affixed to the circuit board with the central holes of the circuit board and rechargeable battery in register with each other; iii) at least one electromechanical vibration component 212; receiving circuitry 208a for receiving a signal from a remote device; and iv) control circuitry 208b operably associated with said at least one electromechanical vibration component, the receiving circuitry, and the rechargeable battery. Here as shown, the at least one electromechanical vibration component 212 is placed at a distal location on the circuit board 202. And as illustrated by the stiffness indicator bar 214, the circuit board 202 may be provided with a stiffness gradient including a lower stiffness associated with the at least one electromechanical vibration component 212 and a higher stiffness associated with the rechargeable battery 204, receiving circuitry 208a, and control circuitry 208b.

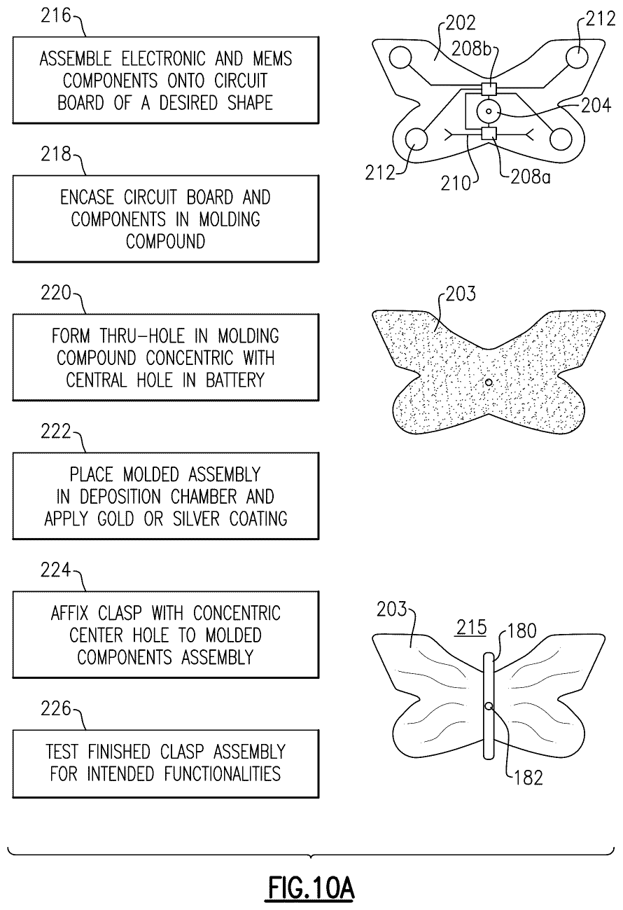

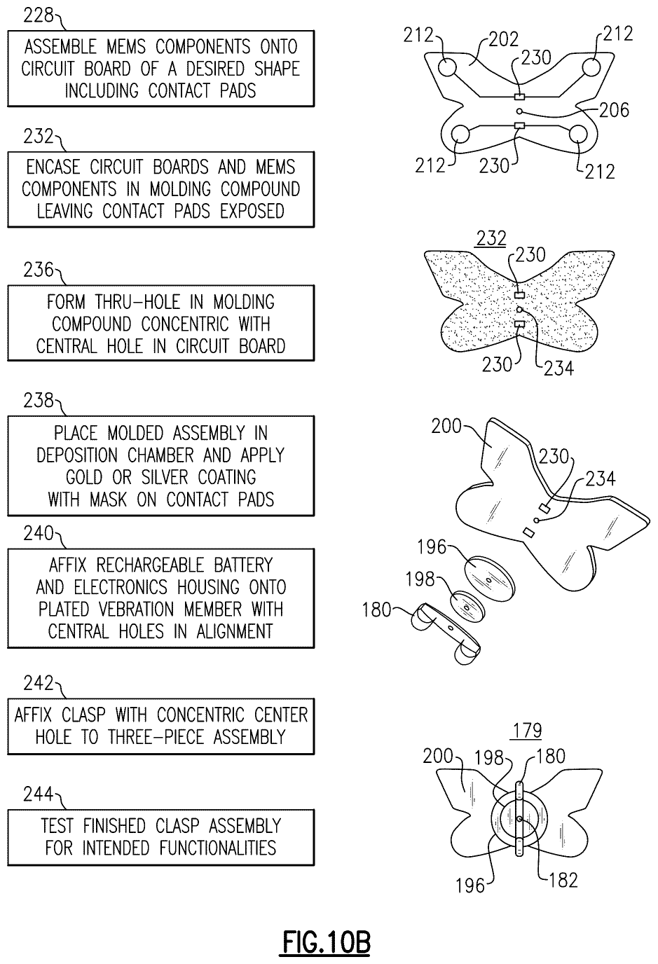

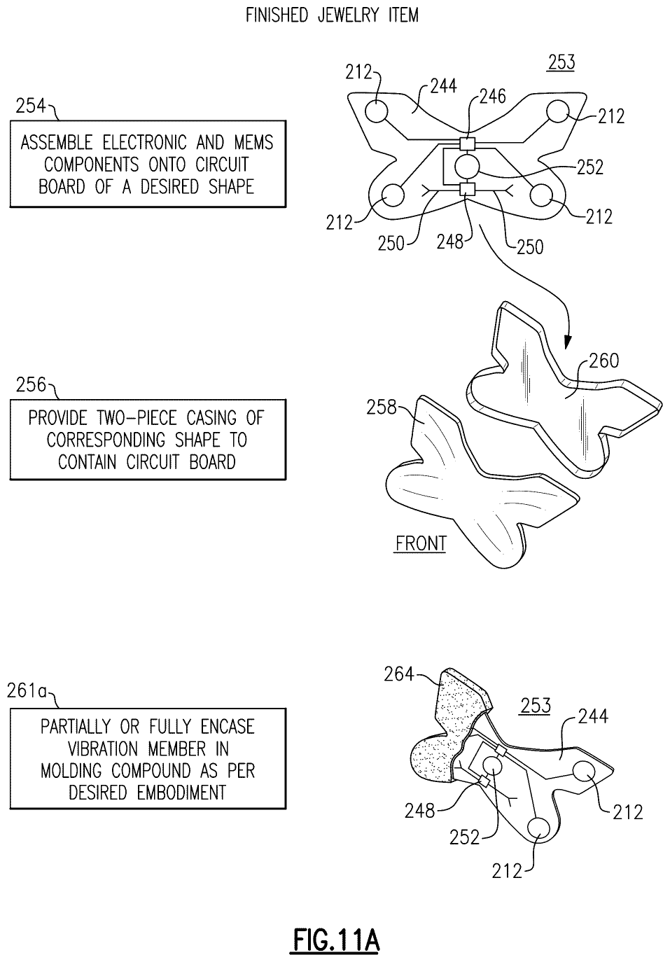

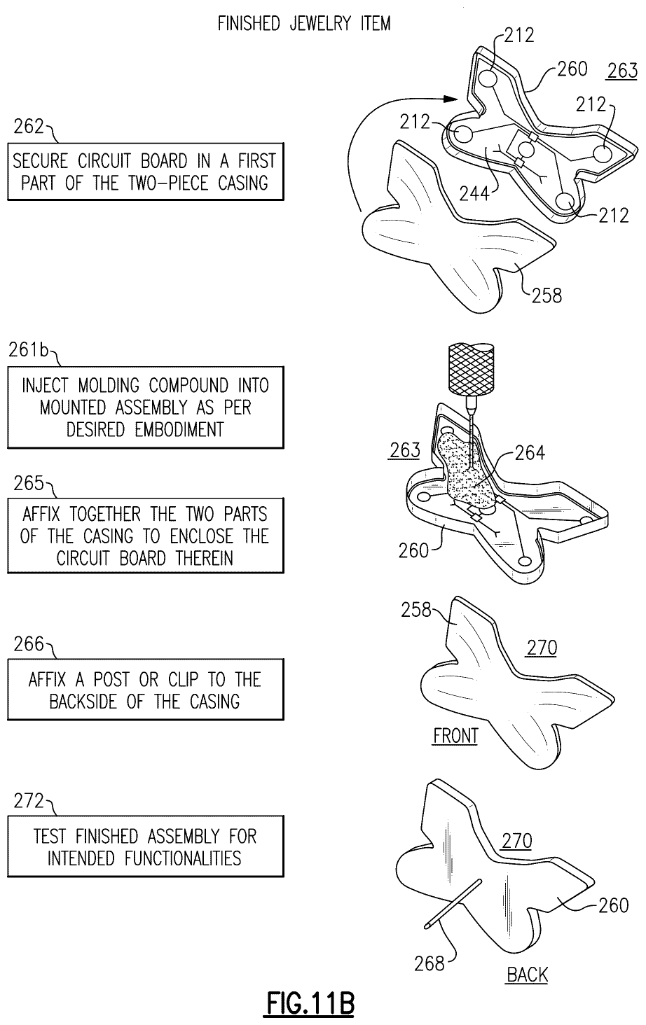

Thus with the above circuit board assembly 201 in hand, as shaped in the form of the butterfly wings of FIGS. 7 and 8 or in the form of the hummingbird wings presented in FIG. 9, finished clasp assemblies 215 of FIGS. 8 and 9 may be used with a long-treasured pre-exiting item of jewelry according to the teachings hereof. Thus as more particularly in connection with FIGS. 7, 8, and 9 and as claimed herein below, the inventors hereof further provide a network-ready clasp assembly 215 for wearable use with a jewelry item having a post, this network-ready clasp assembly including i) a wing-shaped vibration member 203 having a diameter thereacross, said wing-shaped vibration member 203 including at least one electro-mechanical vibrating component 212, ii) a rechargeable battery 204 implemented within the wing-shaped vibration member, iii) receiving circuitry 208a and control circuitry 208b implemented within the wing-shaped vibration member 203 in operable association with said rechargeable battery 204, iv) a hole formed through said wing-shaped vibration member 206 and 182, and v) a clasp 180 with a hole formed therethrough 182, said clasp 180 affixed to a back side of the wing-shaped vibration member 203 with the holes of the clasp and the wing-shaped vibration member in register to form a clasp assembly 215 with a through-hole 182 configured to receive a post of a jewelry item so that upon receipt of a signal from a remote device, the at least one electro-mechanical vibrating component 212 is activated to provide a vibration sensation to a person wearing the clasp assembly with a respective jewelry item. As illustrated, in many of the preferred embodiments of the network-ready clasp assembly 215, the wing-shaped vibration member 203 includes a circuit board 202, and the receiving circuitry 208a and control circuitry 208b are implemented on the circuit board 202. Further, the rechargeable battery 204 may be preferably implemented in association with, or otherwise affixed on, the circuit board 202. And in embodiments implemented for receiving a wireless signal, the receiving circuitry 208a may advantageously include a receiving antenna 210. In certain embodiments where sending a signal is desired, the control circuitry 208b may be operably associated with a transmission antenna 211 implemented to send an information signal to a remote device. In certain preferred embodiments of the circuit board assembly 201, the circuit board 202 has a gradient of stiffness providing a central region of stiffness A and distal regions of stiffness C1 and C2, as illustrated. In some embodiments thereto, the central region of stiffness A of the circuit board 202 has a greater stiffness than a stiffness associated with the distal regions C! and C2 of the circuit board 202 and the at least one electro-mechanical vibrating component 212 is located within a selected distal region of regions C1 or C2, upper or lower, of the circuit board 202. More particularized embodiments of any of the above may be presented wherein the circuit board 202 of FIG. 7, as implemented in any of the types of finished clasp assemblies 215 represented in FIGS. 8 and 9, is fully or partially encased in a molding compound according to the method steps 218 and 261 discussed below in more detail in connection with FIGS. 10A and 11A. And still in further detailed embodiments hereof as illustrated in FIG. 7, any of the above may be provided wherein the battery 204 has a central hole 206 and is affixed in register with a central hole in the circuit board to thereby provide a centrally located weighted center of gravity associated with the wing-shaped vibration member 203 as illustrated across FIGS. 7, 8, and 9. To provide the desired vibration alert in a discreet manner in these embodiments, at least two electro-mechanical vibrating components 212 are provided with one thereof positioned in a first distal region C1 of the circuit board 202 and the other positioned in a second distal region C2 of the circuit board 202. In an augmented embodiment thereof, we provide at least four electro-mechanical vibrating components 212 with a first thereof positioned in a first distal region (C1-Upper) of the circuit board 202, a second of the electro-mechanical vibrating components 212 positioned in a second distal region (C1-Lower) of the circuit board 202, a third of the four electro-mechanical vibrating components 212 positioned in a third distal region (C2-Upper) of the circuit board 202, and a fourth of the four electro-mechanical vibrating components positioned in a fourth distal region (C2-Lower) of the circuit board 202, as shown in detail in FIG. 7 in the butterfly wing configuration thereof. Thus with the above in hand, any thereof may be provided wherein the wing-shaped vibration member 203 is formed in the shape of butterfly wings as shown in FIGS. 7 and 8, or the wing-shaped vibration member 203 may be formed in the shape of hummingbird wings, as represented in FIG. 9, or the wing-shaped vibration member 203 may be formed in the shape of honeybee wings (not pictured but herein disclosed and discussed) and the clasp 180 then is formed in the shape of a honeybee body, or the wing-shaped vibration member 203 may be formed in the shape of at least one peacock tail feather (not pictured but herein disclosed and discussed), or still, the wing-shaped vibration member 203 may be formed in the shape of a plurality of peacock tail feathers (not pictured but herein disclosed and discussed) in an open fan configuration, or in addition thereto, the wing-shaped vibration member 203 is formed in the shape of a plurality of peacock tail feathers in an open fan configuration and the clasp 180 is then formed in the shape of a peacock head and body, and yet still in addition thereto in the alternative, as desired, the wing-shaped vibration member 203 are represented in FIGS. 8 and 9, may be formed in the shape of bumblebee wings and the clasp 180 then formed in the shape of a bumblebee body, and perhaps for other age groups or selected market segments, for example, the wing-shaped vibration member 203 may be preferably formed in the shape of open ladybug wings (not pictured but herein disclosed and discussed) and then the clasp 180 formed in the shape of a ladybug head and body, or yet still, depending on the addressable market, the wing-shaped vibration member 203 may be alternatively formed in the shape of open dragonfly wings and the clasp 180 then formed in the shape of a dragonfly head and body, or still further in the alternative, the wing-shaped vibration member 203 may be formed in the shape of eagle wings (not pictured but herein disclosed and discussed) and the clasp 180 then formed in the shape of the head of an eagle, or similarly but still in the alternative, the wing-shaped vibration member 203 is formed in the shape of eagle wings and then the clasp 180 is formed in the shape of both the head and body of an eagle, or still as may be preferred in the alternative, the wing-shaped vibration member 203 is formed in the shape of falcon or hawk wings and the clasp 130 is formed in the shape of the head and body of a falcon or hawk. And as for the circuit board 202, in alternate embodiments thereof, the distal regions of stiffness C1 and C2 may be provided with a greater stiffness than a stiffness associated with the central region A of the circuit board 202 and then the at least one electro-mechanical vibrating component 212 is located within the central region A of the circuit board 202, and here in this reverse stiffness configuration of the circuit board 202, the circuit board 202 may be fully or partially encased in a molding compound, as discussed below in further detail in conjunction with manufacturing steps 218 of FIG. 10A steps and 261a and 261b of FIGS. 11A and 11B.

Now with reference to FIG. 10A, there is shown a flow chart presenting principal steps in a method of converting the circuit board of FIG. 7 into the finished goods clasp assembly 215 of FIG. 8 including an exemplary corresponding pictorial representation for a certain initial, intermediate, and final steps thereof. Thus at step 216, the shape of butterfly wings is selected for the shape of the circuit board 202 which is then provided with component parts including the rechargeable battery 204 with its concentric central hole, the integrated circuit chips 208a and 208b, the antenna 210, and the vibrating disks 212 as discussed above. Then at step 218, the circuit board is encased in molding compound and thereafter at step 220 a thru-hole in the molding compound is formed in register with the central hole of the rechargeable battery 204 to thereby create a vibration member 203 here in this embodiment including the battery 204, the electronic circuit chips 208a and 208b, the antenna 210, and the vibrating disks 212 all therein in this embodiment encapsuled in the molding compound according to step 218. Next at step 222, the vibration member 203 as an item of goods in process is placed in a vapor deposition chamber where a layer of gold (Au) or silver (Ag) is applied thereto. Then at step 224, the clasp 180 with its central hole is concentrically affixed to the vibration member 203 to form the thru-hole 182 of a final clasp assembly 215 according to this method. And finally at step 226 before shipping as a manufactured jewelry item, the clasp assembly 215 is electronically and mechanically tested to verify that its provided functionalities of receiving, controlling, recharging, and vibrating are in good working order for all prescribed daily use scenarios and that all such should remain in good working order with high reliability for the prescribed service life of the assembly as presented for public use. Thus here, it is intended by the inventors hereof that the component parts utilized to provide the desired electronic and mechanical functionalities for our inventions are of the highest reliability as developed and sourced from their respective industries so that the electronic jewelry items and the electronic jewelry boxes, cases, and vessels disclosed herein have a useful service life of good working order that is co-extensive with the service life expected of jewelry items and jewelry boxes generally.

Thus according to FIG. 10A, there is provided a method of making a clasp assembly 215 for use with an existing jewelry item having a post, this particular method comprising the steps of i) providing a circuit board 202 configured to receive a plurality of components, said circuit board including a central hole and having a preselected shape; ii) attaching to the circuit board in operable associate with one another receiving circuitry 208a, control circuitry 208b, at least one electromechanical vibration component 212, and a rechargeable battery 204 including a central hole 206, said rechargeable battery 204 affixed to the circuit board with the central holes 206 of the circuit board and rechargeable battery in register with each other; iii) encasing the circuit board in a molding compound to form a vibration member 203; iv) forming a hole in the vibration member that is concentric with the central holes of the circuit board and rechargeable battery to thereby form a thru-hole in the vibration member; v) applying a metal coating onto the vibration member; and vi) attaching a clasp 180 with a central hole to the vibration member so that the central hole of the clasp 180 is concentric with the thru-hole 182 in the vibration member 203 as illustrated here in FIG. 10A.

FIG. 10B is a flow chart presenting principal steps of a method of making the backside finished goods clasp assembly 178 of FIG. 5E including an exemplary corresponding pictorial representation for certain steps thereof. Thus here in FIG. 10B at an initial step 228, the butterfly-shaped circuit board 202 is selected and placed thereon are four separate vibrating disks 212 which are electrically connected to a pair of contract pads 230 which here in this embodiment are located proximate the central hole 206 of the circuit board 202. Next at step 232, the circuit board 202 including the vibrating disks 212 are encased in molding compound leaving the contact pads 230 exposed to form a molded vibration subassembly 232 having a central hole 234 formed therein at step 236. Then at step 230, the contact pads 230 are masked and the molded vibration subassembly 232 is placed in a deposition chamber wherein a coating of gold (Au) or silver (Ag) is applied thereto thereby transforming the molded vibration subassembly 232 into the functional wing-shaped vibration member 200 of the rechargeable network-ready clasp assembly 179 as illustrated in FIG. 5E and here shown again at the bottom of FIG. 10B. Thus to complete this method of making the finished-goods clasp assembly 179 of FIG. 5E, at steps 240 and 242 shown here in FIG. 10B, the clasp 180, the electronics housing 198, and rechargeable battery 196 are affixed to the vibration member 200 with each of their central holes in alignment and affixed in register to form the thru-hole 182 to receive the post of a pre-existing jewelry item such as an earring or broach of the traditional post-and-clasp type. And as with the above method of FIG. 10A, at step 244 here in FIG. 10B, before shipping as a manufactured jewelry item, the finished clasp assembly 179 is electronically and mechanically tested to verify that its provided functionalities of receiving, controlling, recharging, and vibrating are in good working order for all prescribed daily use scenarios and that all such should remain in good working order with high reliability for the prescribed service life of the assembly as presented for public use.

Thus as shown and discussed in connection with our network-ready jewelry embodiments of FIGS. 5D, 5E, 5F, and 6D, there is hereby provided here in FIG. 10B another method of making an electronic clasp assembly 178 or 179 for use with an existing jewelry item having a post. This method comprises the steps of i) providing a circuit board 202 configured to receive a plurality of components, said circuit board 202 including a central hole 206 and having a preselected shape, ii) attaching to the circuit board 202 at least one electromechanical vibration component 212 having an operational connection to a contact pad 230, iii) encasing the circuit board 202 in a molding compound to form a vibration member 232 while maintaining the contact pad in an accessible condition, iv) forming a hole 234 in the vibration member 232 that is concentric with the central hole 206 of the circuit board 202 to thereby form a thru-hole in the vibration member, v) applying a metal coating onto the vibration member 232 while maintaining the contact pad 230 in a functional condition, vi) providing electronic circuitry 198 and a rechargeable battery 196 in an operable assembly having a central hole 182, vii) attaching the operable assembly to the vibration member with the holes of each thereof in register and the contact pad providing an electrical connection there between, and viii) attaching a clasp with a central hole to the operable assembly so that the central hole of the clasp is concentric with the central hole in the operable assembly and the thru-hole in the vibration member.

Next with reference next to FIGS. 11A and 11B, there is shown a flow chart presenting the principal steps of a method of making a frontside electronic jewelry item according to further aspects of this invention which includes a representative pictorial illustration for certain steps thereof. As with the electronic backside clasp assembly 215 of FIGS. 8, 9, and 10A, “backside” thereto applying because the assembly 215 is intended for use on the back side of the ear of a user thereof when engaged with a pre-existing item of jewelry including an earring or otherwise employed, for example, on the back side of an article of clothing when used with a broach. But notwithstanding the backside application of such a clasp assembly, it is intended by the inventors hereof that certain preferred embodiments thereof be endowed with aesthetic features, such as butterfly wings, hummingbird wings, honeybee wings, ladybug wings, dragonfly wings, bumblebee wings, or eagle wings, for example, or any other such functional shapes found in nature, that also serve the functional aspects of this invention including mechanical vibration for personal notification purposes being experienced by a user as reminiscent of such animated living things found in nature. Thus in some contrast therewith, “frontside” is applied to the jewelry item of these FIGS. 11A and 11B since in an earring configuration, this item is intended to be worn on the front side of the ear of a user thereof and thus has additional intended display properties for decorative, aesthetic, artistic, or distinctive personal purposes such as have long been associated with the wearing of traditional jewelry items for millennia and thus herein continuing such a tradition with the advent of electronic jewelry. Now with the above in hand and with continuing reference first to FIG. 11A, there is first shown a circuit board 244 here again, for exemplary purposes, having a butterfly shape like the circuit board 202 in FIG. 7. The circuit board 244 is similarly provided with four vibrating disks 212, two exemplary electronic microchips 246 and 248, an antenna 250, and a rechargeable battery 252 which here need not include a central hole like the backside batteries 196 and 204 discussed in FIGS. 10A and 10B. The components in this embodiment of FIGS. 11A and 11B may be configured in an operable assembly to perform a number of desired functionalities such as wirelessly receiving an airwave signal from a remote device, internally processing electronic signals, activating the vibrating disks to provide a vibrating sensation to a person wearing the item, sensing snoring activity, sensing body temperature, and wirelessly sending information signals to a remote electronic device such as a smartphone. Thus for example, the exemplary microchip 248 could be configured as a signal receiving circuit assembly, and microchip 246 could be configured as a signal transmitting circuit assembly for wirelessly sending information signals in which case an additional transmission antenna may be provided therewith like the transmitting antenna 211 in FIG. 7 discussed above. Additional electronic or MEMS components may be included for applications directed to detecting snoring or body temperature. And notwithstanding any such further included functionalities, the circuit board 244 when including the vibrating disks 212 along with corresponding components for enabling same, result in forming a desired operable vibrating or vibration member 253. Thus at step 254 of this method, a desired shape of circuit board is selected and specific electronic and MEMS components are assembled onto the circuit board according to a particular design and corresponding layout parameters. Next at step 256, a corresponding two-piece casing is formed to comport with the shape of the circuit board 244. The two-piece casing as illustrated here includes a front cap portion 258 which provides front side or front surface also referenced 258 and a back housing 260 which similarly provides a back side or back surface 260. Next at step 262, FIG. 11B, the circuit board 244 is secured into the back housing 260, without any molding compound, to thereby form a mounted assembly 263 such that when the vibrating disks 212 are activated, a vibration is transmitted to the back housing 260 so that a person wearing the finished assembly in use feels a vibration sensation on their ear lobe according to the teachings hereof. In a more particularized embodiment hereof, prior to the above step 262, in a step 261a, FIG. 11A, the fully assembled and operable vibration member 253 may be partially or fully encased or encapsuled in a molding compound 264 as discussed above at step 218 in FIG. 10A to thereby secure the electronic and MEMS components in an integrated robust package as may be desired in certain preferred embodiments hereof before affixing same into the back housing 260 at step 262 of FIG. 11B. As an alternative to step 261a, at step 261b of FIG. 11B, molding compound 264 is injected into the mounted assembly 263 including the back housing 260 with the assembled circuit board comprising the vibration member 253 secured therein as shown. As such, when the vibration member 253 including the circuit board 244 with its functional components is secured in the backside casing 260, whether fully encapsulated in molding compound, partially enclosed in molding compound, or otherwise without any molding compound, the mounted assembly 263 as shown at step 262 of FIG. 11B, is in condition for further processing and assembly. Thus returning to the method hereof in the next step in FIG. 11B, at step 265, the front cap 258 is affixed to the back housing 260 to form a closed casing with the vibrating member 253 secured therein. Next at step 266, a post 268 is attached to the back housing 260 to thereby complete a finished jewelry item 270 according to this method. And as would be apparent to those skilled in the arts of jewelry making, the front face of the front cap 258, either before or after affixing same to the back housing 260, may be adorned with any desired type of ornamentation suitable to distinguish the item from a consumer marketing perspective. Finally at step 272, FIG. 11B, the finished jewelry item 270 is tested for intended electronic and mechanical functionalities before certifying same ready for shipment into distribution channels for retail sales. The casing pieces 258 and 260 including the post 268 may be made preferably from gold (Au), platinum (Pt), or silver (Ag), and more preferably, in a solid configuration or alternatively as a plated item or may be made from any other suitable material as optimized for reliable performance of fine jewelry including high reliability electronics, where such other material may be plated with any precious metal as desired. As illustrated in FIG. 11B, the back side of the finished jewelry item 270 is shown with a post 268 for use as a broach on an article of clothing or with a body piercing such a pierced ear. Alternatively, as would be apparent to those skilled in the relevant arts, a clip type fastener may be employed in place of the post 268 for use by persons without pierced ears in the case where finished jewelry item 270 is configured as an earring.

Thus in accordance with the teachings of FIGS. 11A and 11B, the inventors hereof further provide a method of making a finished electronic jewelry item 270 having network functionality. A first embodiment of this method includes the steps of i) providing a circuit board 244 configured to receive a plurality of components, said circuit board having a preselected shape, ii) attaching to the circuit board in operable associate with one another, receiving circuitry 246, 248, control circuitry 246, 248, at least one electromechanical vibration component 212, and a rechargeable battery 252 to form a finished circuitry board assembly, iii) mounting the finished circuit board assembly in a first part 260 of a jewel case to form a mounted assembly 263, iv) enclosing the mounted assembly 263 with a second part 258 of the jewel case to form a respective jewelry item 270 having a front side and a back side, and v) attaching a clip or post 268 to the back side of the respective jewelry item to form a corresponding wearable jewelry item 270. Alternatively and in addition thereto, the inventors hereby further provide a method of making a finished electronic jewelry item 270 having network functionality, where here in this embodiment, the method comprises the steps of i) providing a circuit board 244 configured to receive a plurality of components, said circuit board having a preselected shape, ii) attaching to the circuit board 244 in operable associate with one another, receiving circuitry 246, 248, control circuitry 246, 248, at least one electromechanical vibration component 212, and a rechargeable battery 252 to form a circuitry board assembly, iii) at least partially encasing the circuit board assembly 244 in a molding compound to form a vibration member 253, iv) mounting the vibration member 253 in a first part 260 of a jewel case to form a mounted assembly 263, v) enclosing the mounted assembly 263 with a second part 258 of the jewel case to form a respective jewelry item having a front side and a back side, and vi) attaching a clip or post 268 to the back side of the respective jewelry item to form a corresponding wearable jewelry item 270. And in an alternative embodiment of the just-above two methods, the inventors hereof provide a third embodiment hereof where there is provided a method of making a finished jewelry item 270 having network functionality including the steps of i) providing a circuit board 244 configured to receive a plurality of components, said circuit board having a preselected shape, ii) attaching to the circuit board 244 in operable associate with one another, receiving circuitry 246, 248, control circuitry 246, 248, at least one electro-mechanical vibration component 212, and a rechargeable battery 252 to form a finished circuitry board assembly 253, iii) mounting the finished circuit board assembly 253 in a first part 260 of a jewel case to form a mounted assembly 263, iv) injecting molding compound into the mounted assembly 263 to thereby encase the finished circuit board within the first part of the jewel case 260, v) enclosing the mounted assembly 263 with a second part 258 of the jewel case to form a respective jewelry item having a front side and a back side, and vi) attaching a clip or post 268 to the back side of the respective jewelry item to form a corresponding wearable jewelry item 270.

The above vibrating jewelry item 270 of FIGS. 11A and 11B is a finished goods item ready for wear as both an item of fine jewelry and a wearable personal notification device. As such, for alternate channels of distribution including traditional providers of fine jewelry with many thereof being well known throughout the world, the inventors hereof further provide a network-ready base pad implemented in the form of a fine jewelry item as a partially finished goods item for further ornamentation as intended for provision by such well known fine jewelry makers.

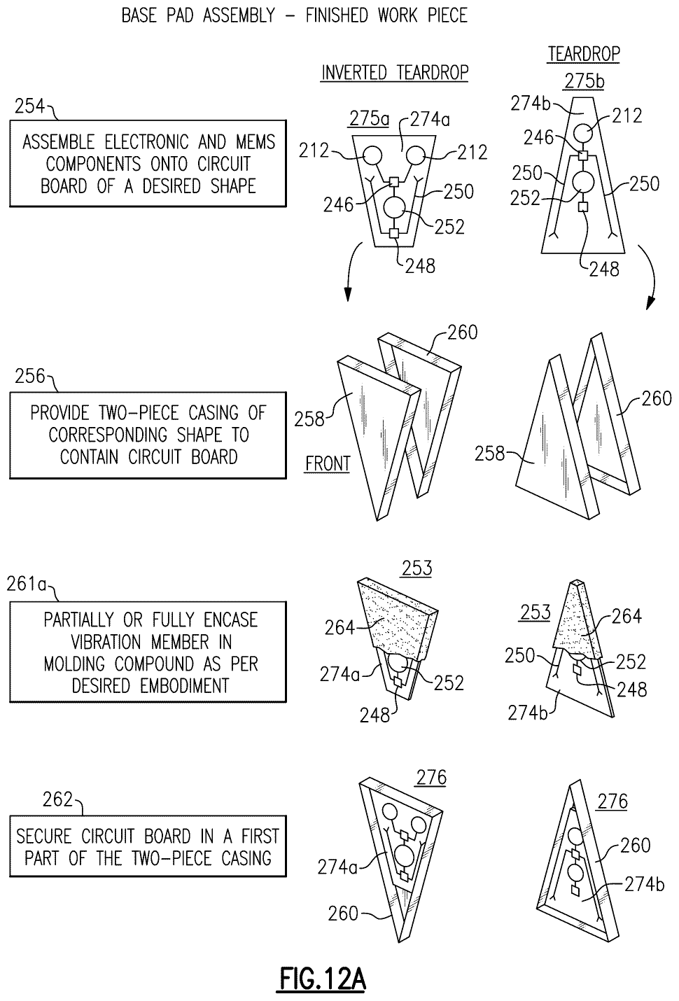

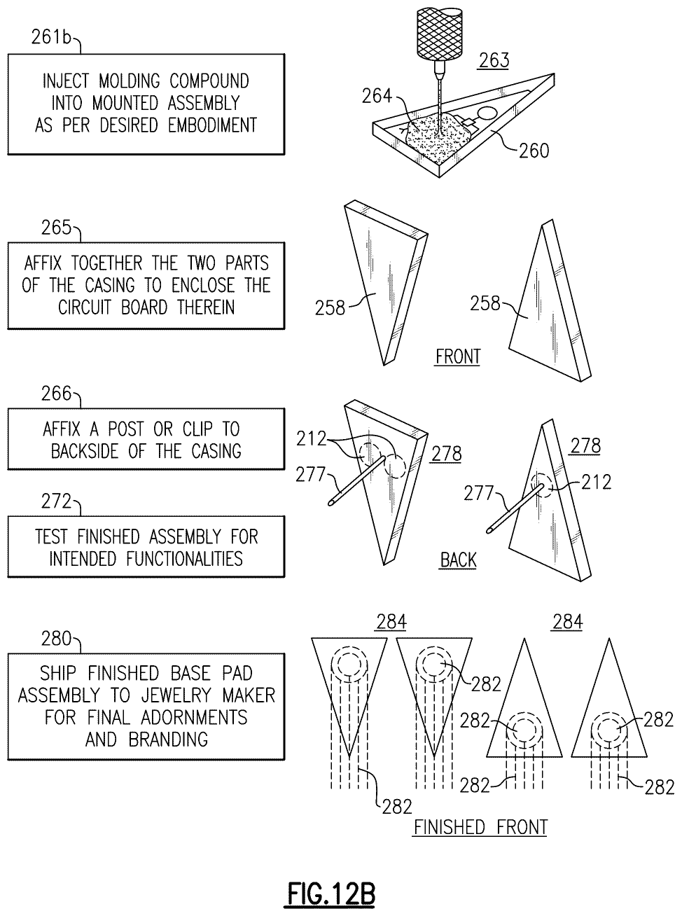

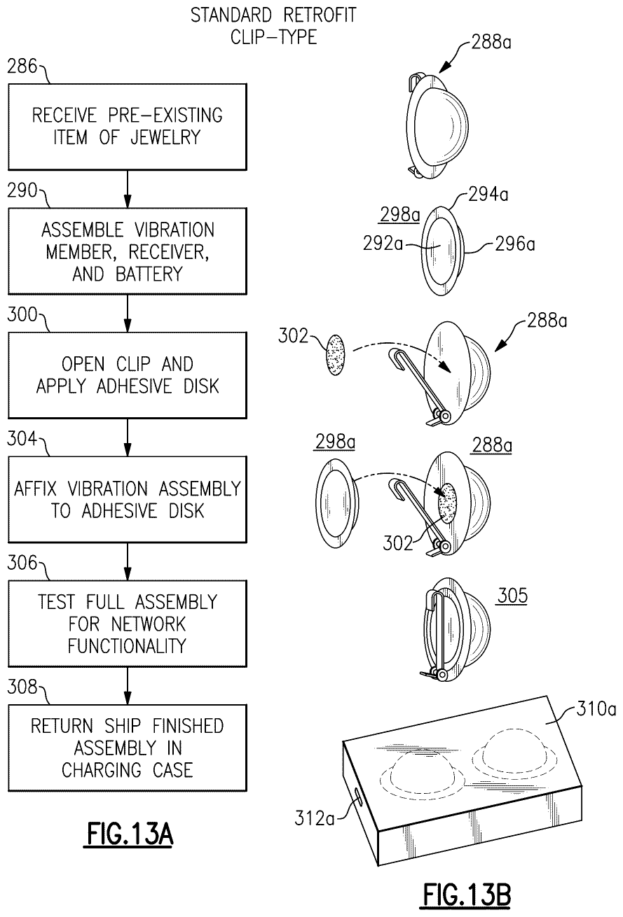

Thus to achieve this objective of providing a fully-enabled network-ready electronic base pad assembly for down-stream ornamentation by traditional providers of fine jewelry, the inventors hereof provide the method and base pad item of FIGS. 12A and 12B which taken together present a flow chart showing certain principal steps of the methods of FIGS. 11A and 11B as implemented to make a finished electronic inventory item in the form of a network-ready base pad assembly completed for further down-stream manufacturing where, like above, FIGS. 12A and 12B, further present a representative pictorial diagram for certain principal steps thereof for purposes of providing further visual clarity to these principal steps of manufacturing. Thus here in FIG. 12A, like above in FIG. 11A, this method starts with step 254 where electronic and MEMS components are assembled in an operable manner on a circuit board 274a or 274b of a desired shape. Here as illustrated, the circuit boards 274a and 274b are formed in the shape of an isosceles triangle with preferably a tip end flattened for better handling in some embodiments thereof thereby forming with more particularly, as shown here, an elongated trapezoidal shape. For purposes of further particularization and detailed description of these inventions as disclosed herein, the circuit board 274b is directed to a teardrop shape and the circuit board 274a is directed to an inverted teardrop shape with the components layout for each thereof being particularly suited to its corresponding circuit board configuration. Thus here as illustrated, the circuit board 274a is provided with two vibrating disks 212, two exemplary electronic microchips 246 and 248, an antenna 250, and a rechargeable battery 252 with each thereof being implemented in an operable assembly to form a corresponding a vibrating circuit board assembly or vibration member 275a. In a similar but alternate configuration, circuit board 274b as configured for this illustrated teardrop embodiment includes a single vibration disk 212 and along therewith in operable association, the two exemplary electronic microchips 246 and 248, the antenna 250, and the rechargeable battery 252 to form a corresponding vibrating circuit board assembly or vibration member 275b. Next at step 256, there is provided a corresponding the two-piece casing including the front cap portion 258 and the back housing 260 for each of the circuit board assemblies 275a and 275b. Here to comport with the shape of the circuit boards 274a and 274b, the front cap portion 258 and the corresponding back housing 260 are shaped in the form of an isosceles triangle to render an appealing teardrop or inverted teardrop base pad assembly. Next at step 262, FIG. 12B, the circuit board assemblies 275a and 275b are respectively secured into the back housing 260, as shown, without any molding compound, to thereby form a respective mounted assembly 276 such that when the vibrating disks 212 are activated, a vibration is transmitted to the back housing 260 so that a person wearing the finished assembly in use feels a vibration sensation on their ear lobe according to the teachings hereof. In a more particularized embodiment hereof, prior to the above step 262, in a step 261a, FIG. 12A, the fully assembled and operable vibration member 275a or 275b may be partially or fully encased or encapsuled in molding compound 264 as discussed above at step 218 in FIG. 10A to thereby secure the electronic and MEMS components in an integrated robust package as may be desired in certain preferred embodiments hereof before affixing same into the back housing 260 at step 262 of FIG. 12B. As an alternative to step 261a, at step 261b of FIG. 12B, molding compound 264 is injected into the mounted assembly 276 including the back housing 260 with the assembled circuit board comprising the vibration member 275a secured therein as shown. As such, when the vibration member 275a or 275b including its respective circuit board 274a or 274b with its functional components is secured in the backside casing 260, whether fully encapsulated in molding compound, partially enclosed in molding compound, or otherwise without any molding compound, the mounted assembly 276 as shown at step 262 of FIG. 12B, is in condition for further processing and assembly. Thus returning to the method hereof in the next step in FIG. 12B, at step 265, the front cap 258 is affixed to the back housing 260 to form a closed casing with the vibrating member 275a, 275b secured therein. Next at step 266, a post 277 (or clip) is attached to the back housing 260 to thereby complete a finished electronic work piece jewelry item, partially finished retail jewelry item, or base pad assembly 278 according to this method. Here at step 266 in the inverted teardrop configuration, the post 277 is preferably positioned centrally on a horizontal axis through the centers of the two vibrating disks 212 and further centrally between the two disks 212 as illustrated. In this manner, a weighted center is determined and the post 277 is positioned relative thereto for comfort in wearability. Further with the position of the post 277 in the inverted teardrop configuration as shown centrally between the two vibrating disks 212 along the referenced horizontal axis, the two vibrating disks 212 are positioned closely and more tightly again the ear lobe so that a lower power level is needed to provide the desired vibration sensation to a person wearing this item as a pierced earring for notification purposes. In a similar manner in the teardrop configuration, the post 277 is preferably positioned along an axis through the center of the single vibration disk 212 that is perpendicular to the back surface of the back housing 260 as illustrated. Here then by design configuration, the single vibrating disk 212 is positioned directly atop the ear lobe being in tighter contact therewith so that even a slight vibration by the single disk 212 in this teardrop configuration may be felt by the user as a desired notification signal or sensation. And as would be apparent to those skilled in the arts of jewelry making, the front face of the front cap 258, either before or after affixing same to the back housing 260, may be adorned with any desired type of ornamentation suitable to distinguish the item from a consumer marketing perspective. Next at step 272, FIG. 12B, the finished base pad assembly 278 is tested for intended electronic and mechanical functionalities before certifying same ready for shipment to a jewelry maker for final adornments and branding as shown next at step 280. Thus here at step 280 where the finished base pad assembly 278 is in the hands of a fine jewelry maker, adornments 282 for illustrative example may be added to the electronic base pad to thereby create a finished jewelry item 284 from the maker so that such a fine jewelry maker may provide its distinctive style and branding to the base pad to thereby distinguish the finished retail jewelry item 284 in the marketplace from other jewelry makers. Also here, after shipment at step 280, the post 277 or clip may be added by the fine jewelry maker as part of finishing the item 284. And like with the above, the casing pieces 258 and 260 including the post 277 may be made preferably from gold (Au), platinum (Pt), or silver (Ag), and more preferably, in a solid configuration or alternatively as a plated item or may be made from any other suitable material as optimized for reliable performance of fine jewelry including high reliability electronics, where such other material may be plated with any precious metal as desired. And here further in this base pad method, the casing pieces 258 and 260 may be first provided by the fine jewelry maker that later further provides the adornments 282 so that the fine jewelry maker's distinctive styling is associated with the casing pieces before further assembly by the electronics manufacture that provides the circuit board assemblies 275a and 275b, for example. Thus here, a good working relationship between the fine jewelry maker and the electronic manufacture is desired to thereby jointly produce the finished items intended by these inventions as discussed with reference to FIGS. 11A to 12B and more particularly with the methods discussed here in conjunction with FIGS. 12A and 12B. And in this collaboration even further, the weighted center of the finished jewelry item 284 relative to the center of gravity of the finished base pad assembly 278 may be of consideration for a desired feel and wearability. For example, if the combined weighted center of gravity of the finished jewelry item 284 is near or at the location of the post 277, then the “swing” of the earing when worn will be of a certain feel, here less swing with a feeling of tightness to the ear where such a desired user experience of tightness to the ear may be achieved more easily in our inverted teardrop design presented in FIGS. 12A and 12B which same being one intended aspect hereof given the weight and location of the electronic and MEMS components arranged on the circuit board 274a. On the other hand, when the center of gravity of the finished jewelry item 284 is made lower than the location of the post 277, here more amenable to the teardrop design of FIGS. 12A and 12B, then when worn, the finished jewelry item 284 may be thereby provided with an increased feel of dangling swing by pendulum motion with body and head movement. Thus as such, this increased feel of swing may be preferred by some such that placement of the components on the circuit board 275b considering their weight combined with the weight, length, and placement of the adornments 282 on the base pad assembly 278 is of important consideration to jointly achieve a certain desired user experience of wearability with pendulum swing along with providing the vibration sensation from the vibrating disk or disks 212 as a personal notification.

Thus in accordance with the teachings of FIGS. 12A and 12B, the inventors hereof provide a method of making a partly finished jewelry item 278 having network functionality, this method comprising the steps of i) providing a circuit board 274a or 274b configured to receive a plurality of components, said circuit board having a preselected shape; ii) attaching to the circuit board in operable associate with one another, receiving circuitry 248, control circuitry 246, at least one electromechanical vibration component 212, and a rechargeable battery 252 to form a finished circuitry board assembly 275a and 275b, respectively, iii) mounting the finished circuit board assembly 275a or 275b in a first part 260 of a jewel case to form a mounted assembly 276; and iv) enclosing the mounted assembly 276 with a second part 258 of the jewel case to form a respective partly finished jewelry item 278 having a back side and a front side configured to receive a desired additional adornment. This item 278 may then be shipped to a maker of fine jewelry where a further method hereof may be practiced. Thus the inventors hereof further provide a method of making a finished jewelry item 284 having network-ready functionality, this method comprising the steps of i) receiving a partly finished jewelry item 278 made by the above method; ii) attaching a selected adornment 282 to the front side 258 of the jewel case; and iii) attaching a clip or post 277 to the back side 260 of the jewel case to form a corresponding finished jewelry item 284 for wearable use. According to further aspects of this method as discussed above for swing feel, the components on the circuit board 274a or 274b may be advantageously arranged to provide a desired center of gravity associated with the finished circuitry board assembly 275a and 275b, respectively, relative to the clip or post, and further, the length, weight, and/or placement of the adornment may be similarly selected in consideration of a desired a desired center of gravity associated with the finished jewelry item 284 relative to the clip or post 277 and the center of gravity of the finished circuitry board assembly.

Alternatively, we provide a method of making a partly finished jewelry item 278 having network functionality, where this embodiment comprises the steps of i) providing a circuit board 274a or 274b configured to receive a plurality of components, said circuit board having a preselected shape, ii) attaching to the circuit board 274a or 274b in operable associate with one another, receiving circuitry 248, control circuitry 246, at least one electromechanical vibration component 212, and a rechargeable battery 252 to form a finished circuitry board assembly 275a or 275b, respectively, iii) at least partially encasing the circuit board assembly 275a or 275b in a molding compound to form a vibration member 253, iv) mounting the vibration member 253 in a first part 260 of a jewel case to form a mounted assembly 276, and v) enclosing the mounted assembly 276 with a second part 258 of the jewel case to form a respective partly finished jewelry item 278 having a back side and a front side configured to receive a desired additional adornment 282. And like above, the item 278 may be the subject of a further method of making a finished jewelry item having network-ready functionality, here comprising the steps of i) receiving a partly finished jewelry item 278 made by the above method, ii) attaching a selected adornment 282 to the front side 258 of the jewel case; and iii) attaching a clip or post to the back side 260 of the jewel case to form a corresponding finished jewelry item 284 for wearable use. And also here like above, for a desired swing feel, the components on the circuit board 274a or 274b may be arranged advantageously to provide a desired center of gravity associated with the finished circuitry board assemblies 275a and 275b, respective, relative to the clip or post, and further, the length, weight, and placement of the adornment or adornments 282 at step 280 may be selected in consideration of a desired a desired center of gravity associated with the finished jewelry item 284 relative to the clip or post 277 and the center of gravity of the finished circuitry board assemblies 274a and 274b, respectively.

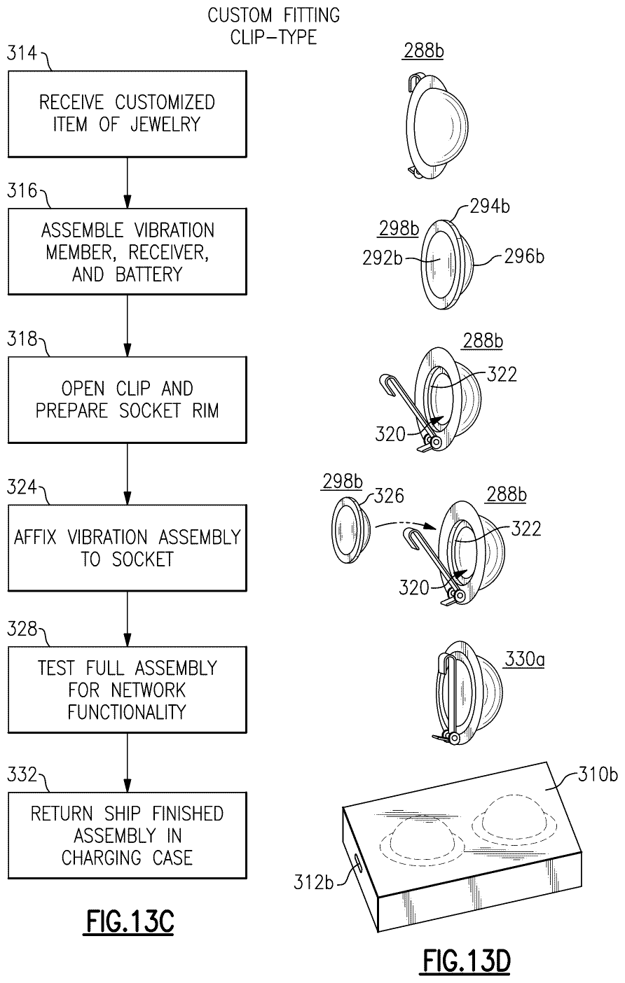

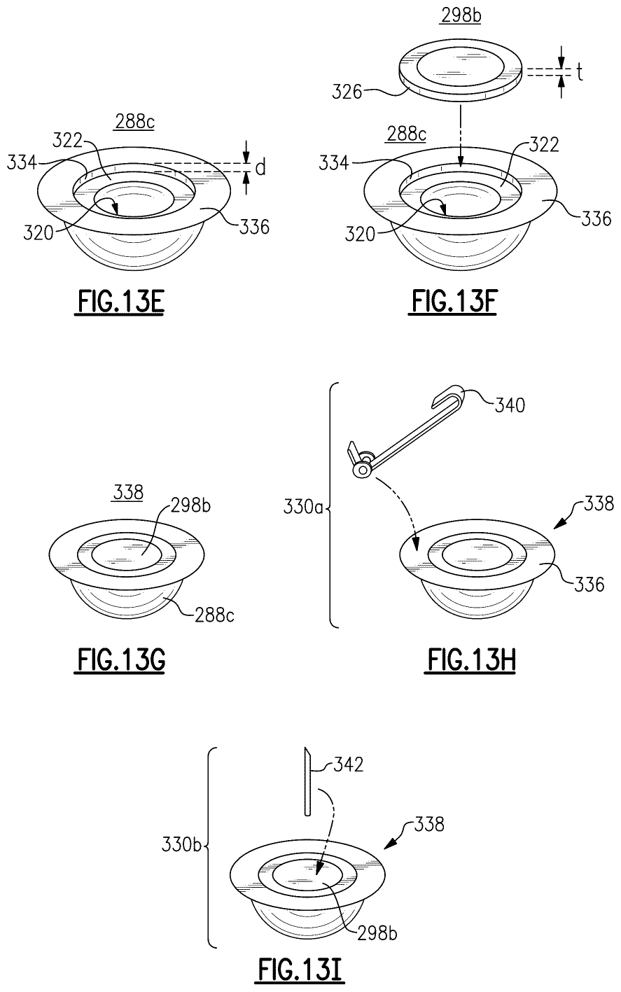

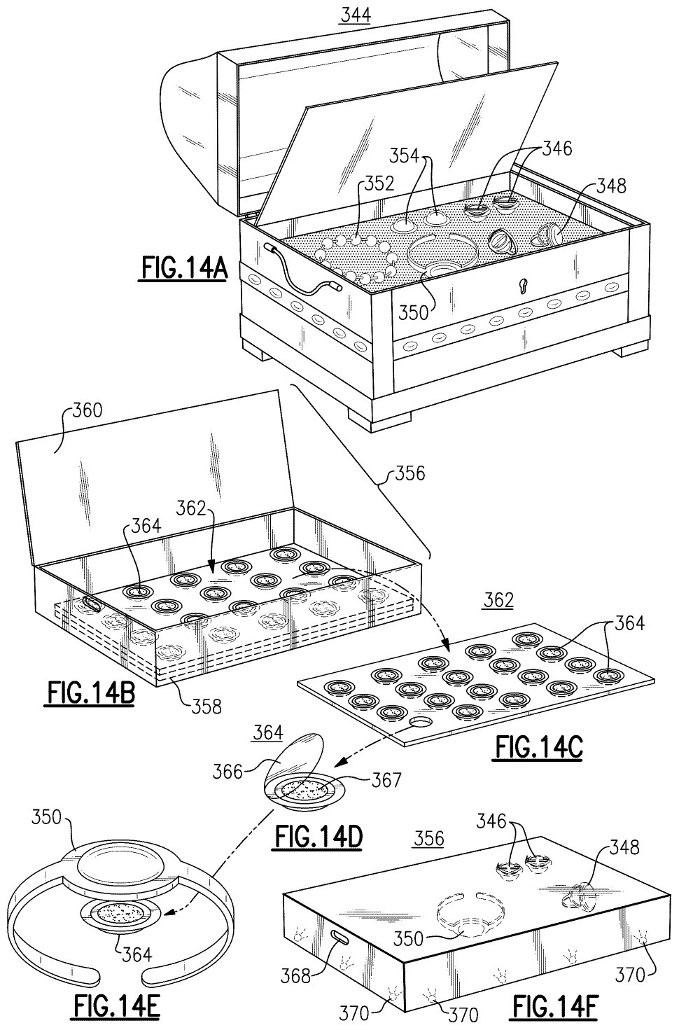

Next with reference to FIG. 13A, there is depicted a flow chart showing the steps of a method of retrofitting an existing piece of jewelry in the form of a clip-type earring hereby enabled with network functionality according to yet another additional aspect of the present invention, and FIG. 13B is a series of perspective pictorials illustrating each corresponding method step of FIG. 13A. Thus at step 286 of FIG. 13A, a pre-existing item of jewelry 288a is received as shown in FIG. 13B. Here as shown, the pre-existing item of jewelry 288a is a clip-type half dome pearl earring but may be any pre-existing item of jewelry duly suited to the steps hereof. Next at step 290, a vibration member 292a, a receiver 294a, and a rechargeable battery 296a are assembled in association as discussed above in detail to form here an operable vibration assembly 298a. Next at step 300, an adhesive disk 302 is applied to the back-side of the jewelry item 288a as shown. The vibration assembly 298 may be the vibration assembly 298a here in FIGS. 13A and 13B as configured for use with the adhesive disk 302 and the pre-existing jewelry item 288a, or the vibration assembly 298 may be a vibration assembly 298b configured for use with a customized jewelry item 288b that has provided therein a socket or recess, as discussed next below in conjunction with FIGS. 13C and 13D.

Now with continuing reference to FIG. 13A, next at step 304, the vibration assembly 298a is affixed to the adhesive disk 302 on the jewelry item 288a for form a finished electronic jewelry item 305. Then at step 306, the finished electronic jewelry item 305, as including a respective vibration assembly 298a, is tested for network and electrical functionalities before proceeding to an in-service use application. And further where desired in certain extended embodiments hereof, at step 308, the finished jewelry item or items may be paired with a suitable charging case 310a including a charging port 312a adapted to receive any desired electrical connection fitting on a charging cord for plug-in use in a manner well known in the art of providing electric charger cords or cables.

The above method of FIGS. 13A and 13B, may be provided to the public by a trained and authorized jewelry expert situated in a jewelry store as supplied with the needed inventory items for performing same on a customer's item of pre-existing jewelry. Or this method may be practiced at home by the owner of the item of pre-existing jewelry when similarly supplied with the needed items for performing same on the owner's selected item of pre-existing jewelry from their personal collection thereof.

In addition to the above, the inventors hereof provide that a jewelry maker may advantageously collaborate with the electronics manufacture that provides a vibration assembly of the types herein disclosed, to co-design from both an artistic perspective and a technical perspective, a custom piece of jewelry specifically intended to receive a network-ready vibration assembly particularly configured to comport therewith and thereto. As such, FIG. 13C of this invention is directed to a flow chart showing the steps of a method of fitting a customized clip-type earring with a vibration assembly according to still yet other aspects hereof while FIG. 13D provides a corresponding series of perspective pictorials illustrating each method step of FIG. 13C. Thus first at step 314 of FIG. 13C, a customized item of jewelry 288b is received from a manufacture thereof. Here for illustrative purposes, the customized item of jewelry 288b is in the form of a clip-type half dome pearl earring but may be any customized item of jewelry duly configured to comport with the vibration assembly hereof. Next at step 316, a vibration member 292b, a receiver component 294b, and a rechargeable battery 296b are assembled together to form the vibration assembly 298b particularly suited to the customized item of jewelry 288b. Next at step 318, the clip is opened to give access to a socket 320 having a support flange or socket flange, rim, or lip 322 and the socket rim is prepared for receiving the vibration assembly 298b. Here, depending on the material comprising the socket and support rim, for example gold or silver, this preparation may include cleaning the support rim with a solvent and application of soldering flux. Then next at step 324, the vibration assembly 298b is affixed into the socket or recess 320 of the jewelry item 288b to thereby form a finished electronic jewelry item 330 including network-ready functionally. As further shown, the vibration assembly 298b is provided with a corresponding rim or lip 326 as configured in this particularized jewelry assembly whereby the rim 326 formed on the vibration assembly 298b seats on the corresponding support flange or rim 322 of jewelry item 288b such that the back of the vibrating assembly 298b is flush and flat with the back-side of the jewelry item 288b for purposes of comfort in wearability here in this half dome pearl earring embodiment. And here again as depending on the materials employed, where gold or silver is preferred for both the socket flange and casing of the vibration assembly, silver solder may be used at a lower temperature to solder the vibration assembly to the socket 320 of the customized jewelry item 288b thus forming the finished item 330. And then similarly here in this customized assembly, at step 328, the finished jewelry item 330, as now including a respective vibration assembly 298a is tested for network and electrical functionalities before proceeding to an in-service use application. And further where desired in certain extended embodiments hereof, at step 332, the finished jewelry item or items may be paired with a suitable charging case 310b including a charging port 312b adapted to receive any desired electrical connection fitting on a charging cord for plug-in use in a manner well known in the art of providing electric charger cords or cables.

Next with reference to FIGS. 13E to 13I, there are provided assembly views in further detail of the type of customized earring of FIG. 13D. Thus first in FIG. 13E, a partially completed item of jewelry 288c is receive for further assembly. Here the partially completed item of jewelry 288c is in the form of a half dome pearl earring without including a clip or post at this stage of the present method. As shown in further detail by enlargement, partially completed item of jewelry 288c includes at this stage the receiving socket 320 and the socket flange 322 where the socket flange 322 is mechanically connected to and the structurally supported by an annular ring 334 which in turn is mechanically supported by an exterior trim flange 336. As shown by dashed projection lines in FIG. 13E, the annular ring 334 has a depth “d” projecting into the socket 320 that is selected in measurement relative to the thickness of the vibration assembly 298b as next discussed. Thus next in FIG. 13F, the vibration assembly 298b is illustrated as including the rim or lip 326 thereof and by the dashed projection lines is provided with a thickness “t”. Thus preferably, the depth d of the annular ring is substantially equal to the thickness t of the vibration assembly at the rim portion 326 so that when the assembly 298b is fitted into the socket 320, the back side of the assembly is flush with the back surface of the trim flange 336. Thus in assembly, the vibration assembly 298b is fitted into the socket 320 such that the rim 326 of the vibration assembly 298b seats on the socket flange 322. Then when all of the rim 326 of the assembly 298b, socket flange 322, annular ring 334, and trim flange 336 are formed from compatible metals, such as silver or gold, the vibration assembly 298b may be soldered into the socket 320 to thereby form an assembled partially finished electronic jewelry item 338 as next shown in FIG. 13G. Thus as simply illustrated in FIG. 13G, the partially finished electronic jewelry item 338 includes the vibration assembly 298b as now rigidly integrated with the partially completed item of jewelry 288c. And next to form the finished electronic jewelry item of FIG. 13B, a clip 340 is applied to the trim flange 336 in a manner well know in the art of jewelry making as shown in FIG. 13H. And alternatively to achieve a post and clasp type earring, FIG. 13I show a post 342 applied to the center of the back side of the vibration assembly 298b as secured with the partially finished jewelry item 338 to thereby form a finished electronic jewelry item 330b as illustrated.

Thus with reference to FIGS. 13A and 13B, the inventors hereof further provide a method of retro-fitting an existing piece of jewelry 288a with network functionality, said method comprising the steps of i) receiving a respective existing piece of jewelry 288a; ii) identifying a target surface on the respective existing piece of jewelry 288a; iii) providing a vibration member 292a, receiver circuitry 294a, and a rechargeable power cell 296a integrated together to form a vibration assembly 298a that comports with the target surface of the respective existing piece of jewelry 288a; iv) applying an adhesive disk 302 to the target surface of the respective existing piece of jewelry 288a; and v) attaching the vibration assembly 298a to the adhesive disk 302 to form a finished electronic jewelry item 305 that when worn as a wearable decorative item, is enabled by the receiver circuitry 294a to receive a signal from a remote wireless device and activate the vibration member 292a to thereby provide a silent notification to a person wearing the finished electronic jewelry item 305. And according to additional aspects hereof, this method may further include providing an electronic jewelry box 310a for recharging the rechargeable power cell of the finished electronic jewelry 305.

And further with reference to FIGS. 13C to 13I, the inventors hereof also provide a method of custom fitting a partially finished piece of jewelry with network functionality, where this method comprises the steps of i) receiving a partially finished piece of jewelry 288c having a socket 320; ii) providing a vibration member 292b, receiver circuitry 294b, and a rechargeable power cell 296b integrated together to form a vibration assembly 298b that comports with the socket 320 of the respective partially finished piece of jewelry 288c; iii) securing the vibration assembly 298b into the socket 320 of the respective partially finished piece of jewelry 288c to form a partially finished electronic jewelry item 338 having a front side and a back side; and iv) attaching a clip 340 or post 342 to the back side of the partially finished electronic jewelry item to form a finished electronic jewelry item 330a, 330b that when worn as a wearable decorative item, is enabled by the receiver circuitry 294b to receive a signal from a remote wireless device and activate the vibration member 292b to thereby provide a silent notification to a person wearing the finished electronic jewelry item 330a, 330b. And like above, this method may further include providing an electronic jewelry box 310b for charging the rechargeable power cell 296b of the finished electronic jewelry items 330a and 330b.

With continuing discussion now with reference to FIG. 14A, there is shown therein a perspective pictorial representation of a personal collection of pre-existing jewelry items as may be stored in a traditional jewelry box 344 for safe keeping. Here in the box 344 for illustrative purposes, there is shown a pair of clip type earrings 346, a ring 348, a wrist bracelet 350, a pearl bracelet 352, and a pair of post and clasp type earrings 354 as designed for use with a pierced ear in this example of an item of pre-existing jewelry. With application of this aspect of the present invention, some of the pre-existing jewelry items in the box 344 may be suited hereto and some may not be so suited. As such, an inventory of the items may be taken and those items that are suitable hereto may be selected for retro-fitting application according to the following.

FIG. 14B is a pictorial representation of an electronic jewelry box 356 with a closable cover 358 having a flat-bottom charging base or charging pad 358 and a closable cover 360 in accordance with still further aspects of this invention. As also shown in FIGS. 14B and 14C, the box 356 is provided with a delivery card 362 of pre-assembled vibration disks or pucks 364 of the type of this invention as discussed above in connection with FIGS. 13A and 13B. Here in this embodiment, each pre-assembled vibration puck 364 includes a peel-away cover 366 that when removed from the puck 364, gives exposure to a sticky adhesive pad 367. Thus in use, an owner of the collection of pre-existing jewelry items shown in FIG. 14A may examine each thereof and determine that the bracelet 350 is suited for application of the vibration puck 364 for the intended purposes hereof, and then to achieved same, simply removes the peel-away cover 366 and applies the puck 364 to the back side of the bracelet 350 as shown. Thus in this manner, there is provided a simple and cost effective way of outfitting a pre-existing item of jewelry with the functionality of certain aspects of this invention which here include enabling the jewelry item to receive an alert signal for a remote device such as a mobile phone where upon in a prescribed manner the puck vibrates in a silent fashion to thereby provide the user thereof with a personal private notification.

Application of a wearable vibration assembly as a skin patch may be found in commonly assigned related issued patents and pending patent applications as referenced and incorporated herein and thus as such, the vibration puck 364 hereof may thus be considered as an alternative thereto as applied to a pre-existing item of jewelry rather than deployed as a vibrating skin patch.

Thus continuing with the subject matter of this disclosure, next in FIG. 14C there is shown a pictorial representation of a delivery card including a plurality of pre-assembled vibration pucks according to this invention with each such puck having network functionality for use with an existing piece of jewelry such as a bracelet, necklace, or clip-type earring to thereby provide a personal alert notification when worn on the body of a user thereof. Thus as to use hereof, here in FIG. 14D there is shown a pictorial rendering of an individual vibration puck 364 removed from the delivery card 362 of FIG. 14C showing a peel away of a film cover 366 supplied over a corresponding adhesive pad 367 of the selected vibration puck 364. Next in FIG. 14E, there is illustrated a pictorial example of placing one of the vibration pucks 364 from the delivery card 362 of FIG. 14C onto the bracelet 350 as selected from the traditional jewelry box 344 of FIG. 14A. Thus in use, when the braceleted 350 is worn as an item of jewelry including the applied vibration puck 364, a remote signal received by receiver circuitry provide in the puck may activate the puck to vibrated as discussed above to thereby give a silent notification alert to the person wearing the bracelet as augmented herewith to include such desired functionality.

Lastly as relating to this embodiment of the electronic jewelry box 356, FIG. 14F shown perspective pictorial illustration of the box 356 including various selected items of existing jewelry including the ring 348, the clip-type earring 346, and the bracelet 350 each including a vibration puck attached thereto as then placed in the electronic jewelry box 356 of FIGS. 14B and 14F for re-charging by the charging pad 358 according to these aspects hereof. And as discussed in detail above in connection with our related electronic boxes in FIGS. 4A to 4M-2, the box hereof in FIG. 14F may advantageously include an electric supply of charging port 368 and one or more indicator lights 370.

Thus more generally as to the vibration assembly 298a of FIG. 13B and the pre-assembled vibration disks or pucks 364 of FIGS. 14B to 14E, there is hereby provided and claimed, a network-ready alert assembly 298a, 364 for use with a pre-existing piece of jewelry having a backside, the network-ready alert assembly comprising a vibration member 292a having a first surface and a second surface opposed thereto, said vibration member 292a being implemented to vibrate in a predetermine manner; an adhesive layer 302, 367 applied to said first surface of said vibration member 292a and implemented for attachment to the backside of a respective pre-existing piece of jewelry 288a, 350; and a receiver component 294a affixed in assembly with said second surface of said vibration member 292a, said receiver component 294a implemented to receive an activation signal from a remote device so that upon receipt of said activation signal by said receiver component 294a, said vibration member 292a vibrates to thereby provide an alert to a user wearing the respective pre-existing piece of jewelry 288a, 350 after the network-ready alert assembly is attached thereto by applying the adhesive layer 302, 367 to the backside thereof. And in a related embodiment thereto, this is also provide a network-ready alert assembly 298a, 364 for use with a pre-existing piece of jewelry having a backside, where here the network-ready alert assembly 298a, 364 includes a receiver component 294a having a first surface and a second surface opposed thereto, said receiver component 294a implemented to receive an activation signal from a remote device; an adhesive layer 302, 367 applied to said first surface of said receiver component 294a and implemented for attachment to the backside of a respective pre-existing piece of jewelry 288a, 350; and a vibration member 292a being affixed in assembly with said second surface of said receiver component 294a and implemented to vibrate in a predetermine manner so that upon receipt of said activation signal by said receiver component 294a, said vibration member 292a vibrates to thereby provide an alert to a user wearing the respective pre-existing piece of jewelry 288a, 350 after the network-ready alert assembly 298a, 364 is attached thereto by applying the adhesive layer 302, 367 to the backside thereof.

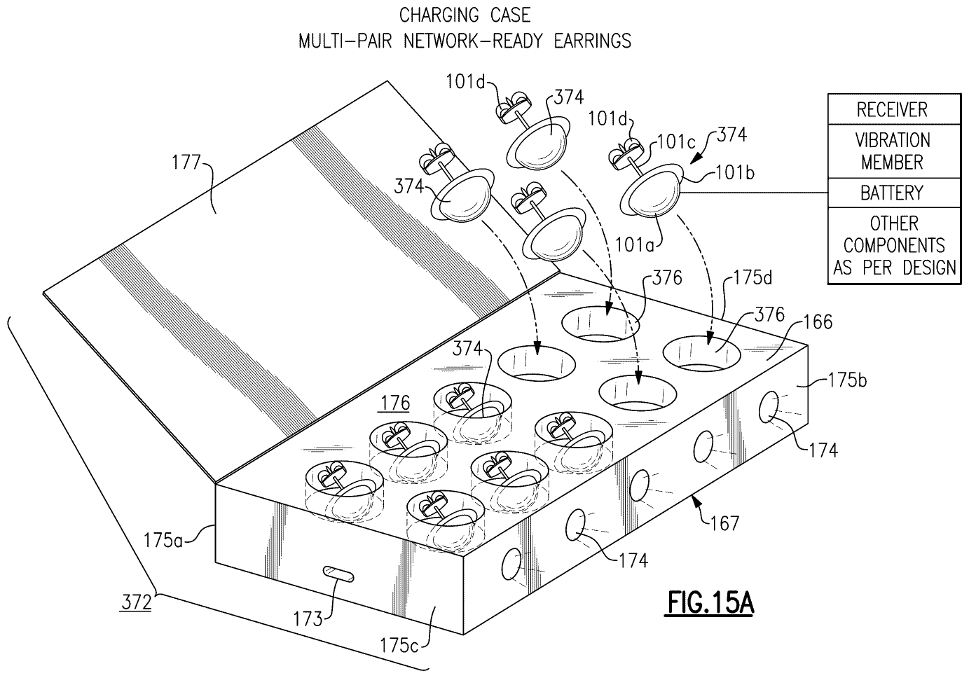

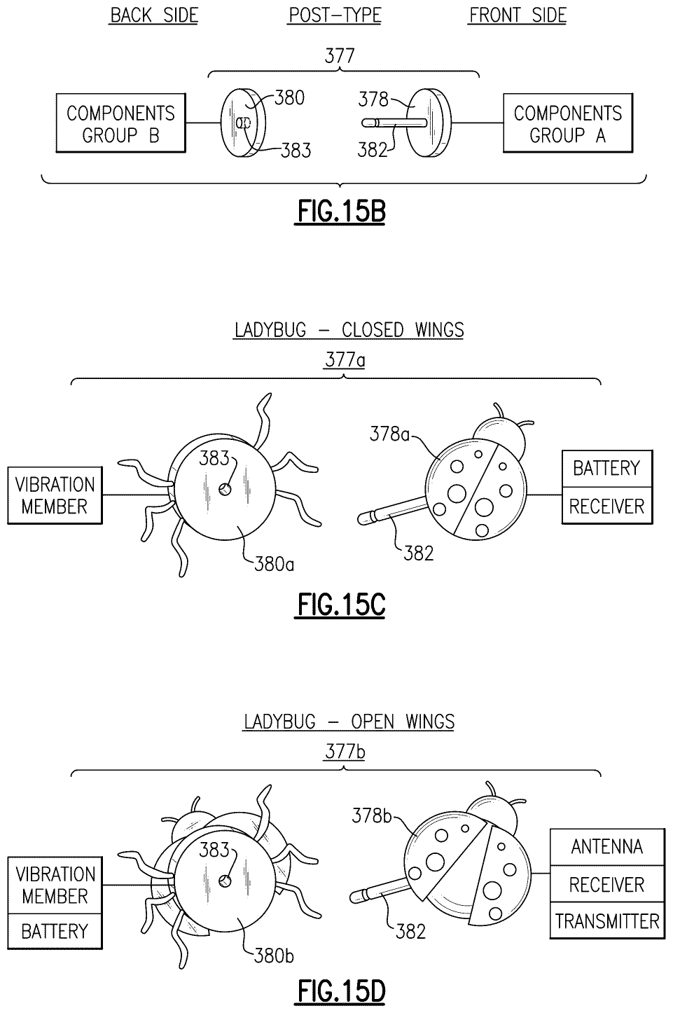

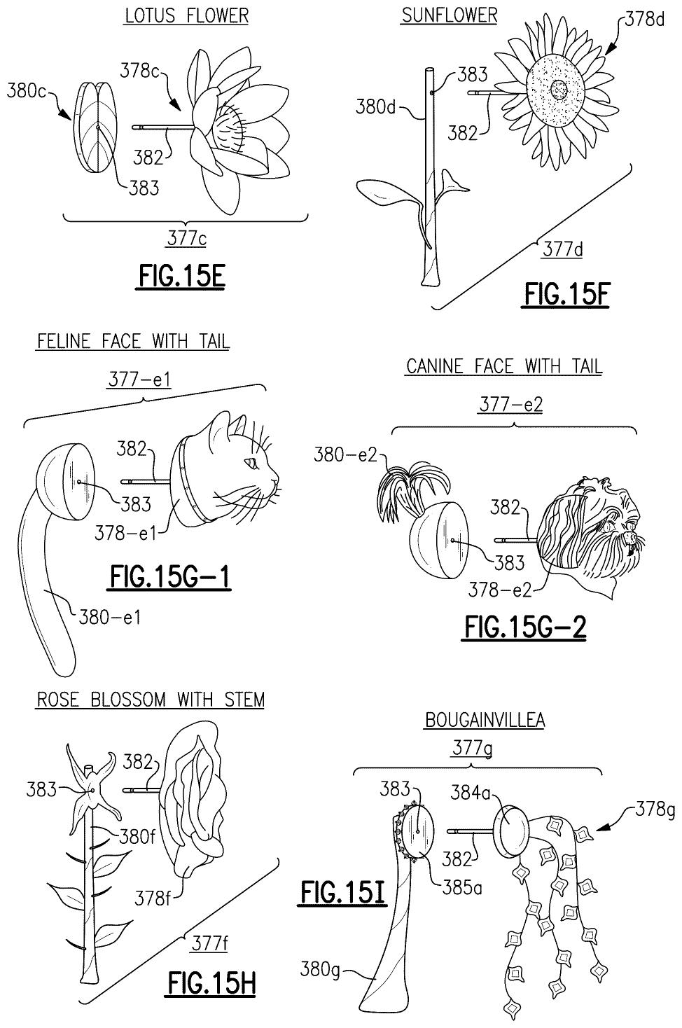

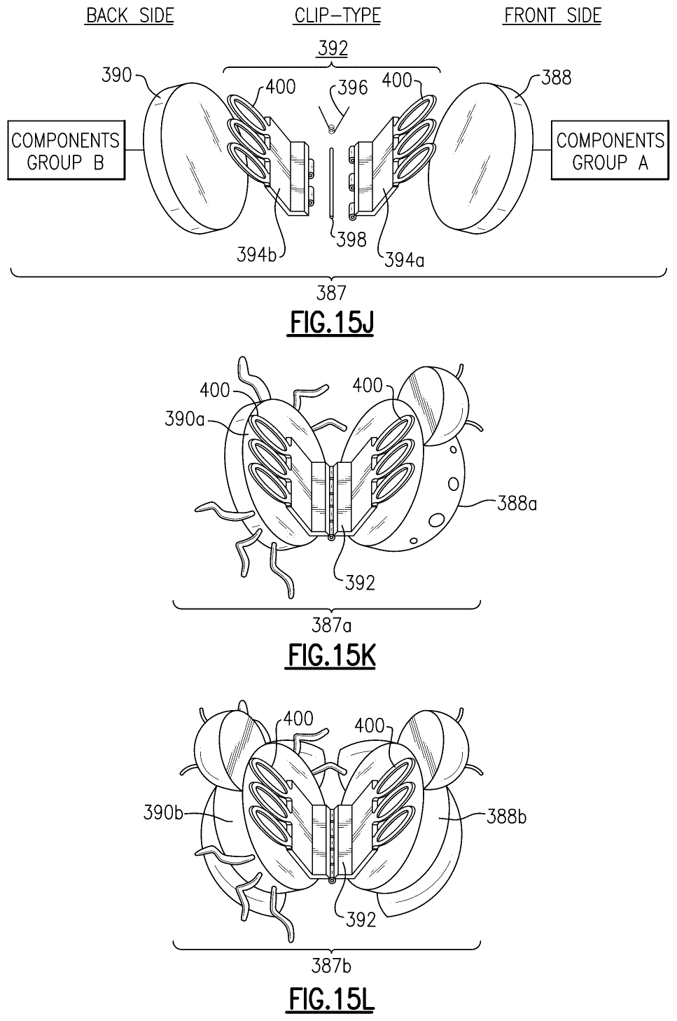

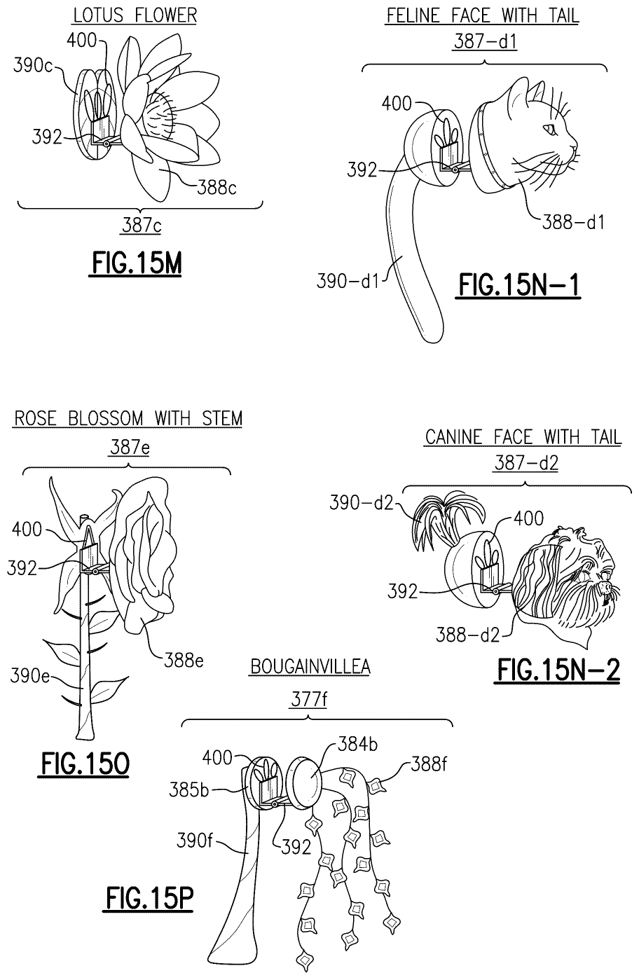

Moving on to FIG. 15A now, there is shown a pictorial representation of an alternative embodiment of an electronic jewelry charging case 372 hereof including a plurality of corresponding network-ready electronic earrings 372 made according to certain aspects of the present invention. Like the charging case 165 discussed above in FIGS. 4A and 4B, the charging case 372 here in FIG. 15A includes a closable top cover 177, indicator lights 174, and a power supply port 173. As illustrated, the case 372 here in FIG. 15A, like the charging case 165 of FIGS. 4A and 4B, includes in substantial part a box 176 having four side wall segments 175a, 175b, 175c, 175d, a top 166, and a bottom 167 and is here configured as a rectangular box but may be configured as any desired geometric shape including the circular form, heart-shape, butterfly-shape, kitten or cat shaped configurations discussed in detail in FIGS. 4C to 4F and 4G to 4M-1 which details need not be repeated here as same are readily applied to the box 372 hereof in FIG. 15A. As such, however, charging receptacles 376 here in charging case 372 are specifically configured to charge completed electronic jewelry items such as the electronic earrings 374 illustrated here in FIG. 15A for exemplary purposes, rather than the clasp or hold-fast assemblies 168 discussed above in FIGS. 4A to 4M-2 which are, as described in detail above, intended for use with an existing piece of jewelry having a post. The electronic earring 374 is provided with receiver circuitry, a vibration member, and a rechargeable power cell such as a rechargeable battery, as designed, manufactured, and assembled by the methods hereof, for example, by the methods of FIGS. 11A and 11B with collaboration by the jewelry maker and the electronics provider, as adapted thereto and thus as such may not need repeating here for this specific jewelry item. And as illustrated, the electronic earring 374 includes a solid post 101c and a traditional mechanical clasp 101d such that all the electronic and MEMS components of the type herein provided, are housed in the dome portion 101a or the dome position and the base portion 101b as initially discussed above in FIG. 1B and shown again here in the case of the electronic earring 374 here in FIG. 15A.

As an alternative to providing all the operational components hereof in a single jewelry item, the inventors of this disclosure further provide herein a two-piece electronic earring where both pieces have significant atheistic or artistic design features that are stylized as companion parts to an integrated whole from a conceptual design perspective.

Thus to achieve this aspect of the present inventions, FIG. 15B provides an initial schematic representation of a two-piece electronic jewelry item 377 hereof having a front-side piece 378 and a back-side piece 380 with each thereof having a desired grouping of operable components according different embodiments thereof. For example, Group A of the front-side piece 378 may include a rechargeable battery and electronic circuitry for receiving and sending signals, and Group B may include a MEMS vibration member like those discussed above for providing silent notification to a user thereof when worn as an item of jewelry. As further illustrated in FIG. 15B, a post 382 is provided on the front-side piece 378 and a corresponding hole 383 is provided on the back-side piece 380. As has been recently developed in the arts of jewelry making, a mechanical clasp for use with pierced earring, like the clasp 101d discussed above in FIG. 1B, that grips the post 101c by means of friction between the edge of hole in the clasp and the post, may be replaced with a hole that has configured therein a rubber O-ring or silicone bead such that when the post is inserted into the hole, a smooth-running, soft-touch binding frictional force is provided between the sliding post and the rubber or silicone as opposed to the metal-on-metal grabbing feel between a metal post and a traditional metal clasp. As such, this recent advancement allows for eliminating the traditional metal clasp in favor of a fully decorative jewelry piece that may be used on the back of the ear to secure the post of the front piece in a smoother feeling manner with increased aesthetic aspects to the back-side piece. Thus as such, here in FIGS. 15B to 15I, such a silicone bead is utilized in the hole 383 to thereby eliminate the need for a traditional clasp and thus provide the opportunity to stylize and sculpt the back-side piece 380 in a variety of astatically pleasing different configurations. And as such then, throughout the entirety of this disclosure, more generally, the terms “hold-fast assembly”, “clasp”, “clasp assembly”, “clasping component”, and the like may be taken to include or comprise both the traditional mechanical clasp or the more recent smooth sliding, soft material type provided internal to the receiving hole for the post such as the hole 383 here in FIG. 15B as provide in the back-side piece 380.

Thus as one example of a stylized two-piece jewelry item according to these aspects of this invention, FIG. 15C provides a pictorial illustration of a stylized two-piece electronic jewelry item 377a of the type of FIG. 15B here having sculpted pieces configured in the form of a ladybug with closed wings. As such, a front-side piece 378a is configured as a ladybug body with a head portion where the wings are closed, and a corresponding back-side piece 380a is configured in the form of the bottom of the ladybug body with legs extending therefrom. Here the dome portion of the ladybug body in the front-side piece 378a is well suited to house a stack of components, such as a rechargeable power cell or battery and receiver circuitry, and the essentially flat form of the back-side piece 380a is suited to housing a vibration disk or a rechargeable battery with a central hole like those discussed above.

Another example of a stylized two-piece jewelry item according to these aspects of this invention is presented in FIG. 15D which provides a pictorial illustration of a stylized two-piece electronic jewelry item 377b of the type of FIG. 15B here having sculpted pieces configured in the form of a ladybug with open wings. As such, a front-side piece 378b is configured as a ladybug body with a head portion where the wings are open, and a corresponding back-side piece 380b is configured in the form of the bottom of the body with legs extending therefrom here also showing open wings on the back-side piece 380b such that from both the front-side and back-side views of the two-piece item when worn, the appearance presents as a top view of a ladybug in flight thus giving further life and a sense of whimsical animation to the item when worn and when experiencing the vibration thereof when in use. Here in FIG. 15D, the dome portion of the ladybug body in the front-side piece 378b, like above, is well suited to house a stack of components, and the essentially flat form of the back-side piece 380b is suited to housing a vibration disk or a rechargeable battery with a central hole like those discussed above in FIGS. 6A to 6D. Yet another example of a stylized two-piece jewelry item according to these aspects of this invention is presented in FIG. 15E which provides a pictorial illustration of a stylized two-piece electronic jewelry item 377c of the type of FIG. 15B here having sculpted pieces configured in the form of a lotus flower and lotus leaf. As such, a front-side piece 378c is configured as a lotus flower with a central portion including stamen and a carpel or seedpod, and a corresponding back-side piece 380c is configured in the form of a lotus leaf as would be experienced in nature as floating on a top surface of still waters where lotus flowers would typically be found. Here the central portion of the lotus flower the front-side piece 378c, like the above ladybug bodies, is well suited to house a stack of micro-components, and the essentially flat form of the back-side piece 380c as configured as a floating lotus leaf is suited to housing a vibration disk or a rechargeable battery with a central hole like those discussed above in FIGS. 5A to 5D and 6A to 6D.

Still yet a further example of a stylized two-piece jewelry item according to these aspects of this invention is presented in FIG. 15F which provides a pictorial illustration of a stylized two-piece electronic jewelry item 377d of the type of FIG. 15B here having sculpted pieces configured in the form of a sun flower and a sun flower stalk or stem. As such, a front-side piece 378d is configured as a sun flower with a central portion including disk floret or seed cluster, and a corresponding back-side piece 380d is configured in the form of the stalk or stem as would be experienced growing in a natural setting. Here the central portion of the sun flower in the front-side piece 378d, like above, is well suited to house a stack of micro-components, and the slender long form of the back-side piece 380d configured as a stem is suited to housing an antenna assembly as implemented to receive a wireless signal from a remote device such as a mobile phone, or an electronic bio-patch, or both a mobile phone and an electronic bio-patch.

And further, as an example of a two-piece jewelry item stylized as a favored pet or animal, according to further aspects of this invention, there is provided in FIG. 15G-1 a pictorial illustration of a stylized two-piece electronic jewelry item 377-e1 of the type of FIG. 15B here having sculpted pieces configured in the form of a kitten or cat. As such, a front-side piece 378-e1 is configured as a kitten or cat head with facial details, and a corresponding back-side piece 380-e1 is configured in the form of a cat tail which here includes a stylized body portion that would naturally be associated therewith. Here the head part of the front-side piece 378-e1, like above, is well suited to house a stack of micro-components, and the slender long form of the back-side piece 380-e1 configured as a cat tail is suited to housing an antenna assembly and receiver circuitry as implemented to receive a signal from a remote device such as a mobile phone, or an electronic bio-patch, or both a mobile phone and an electronic bio-patch, while the associated body portion here included in the back-side piece 380-e1 may be further suited to housing a vibration disk or a rechargeable battery with a central hole like those discussed above in FIGS. 5A to 5D and 6A to 6D.

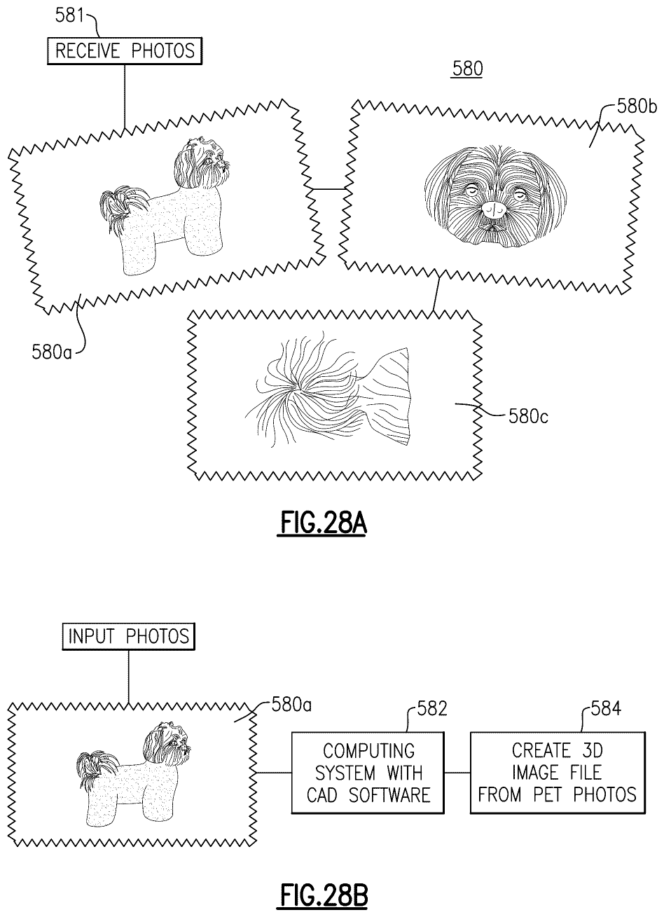

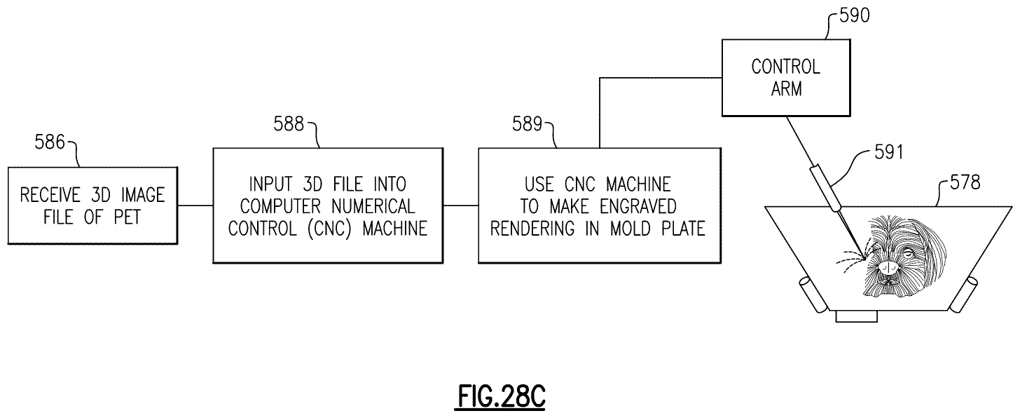

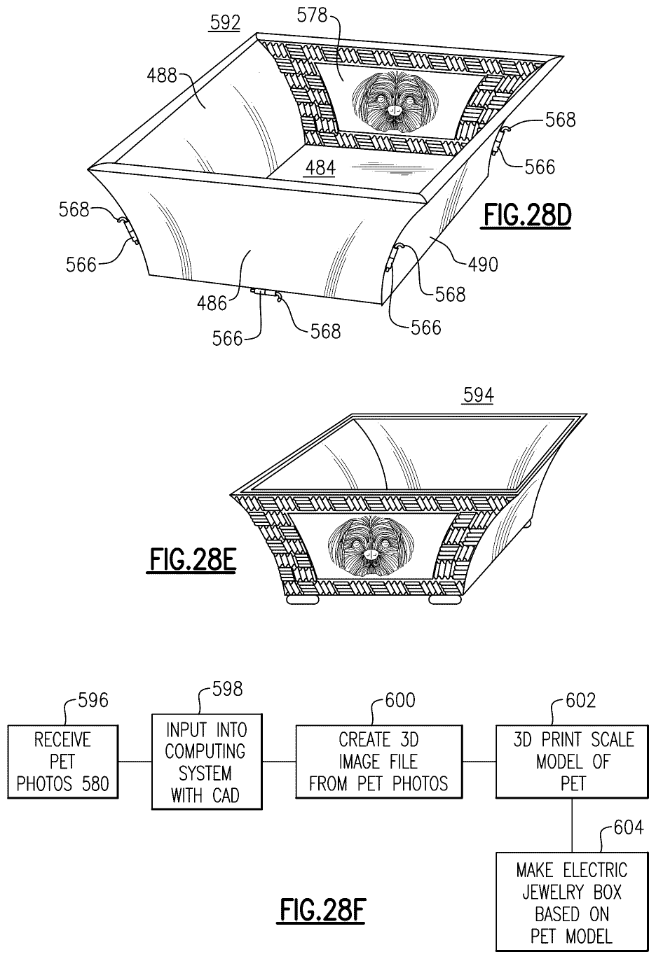

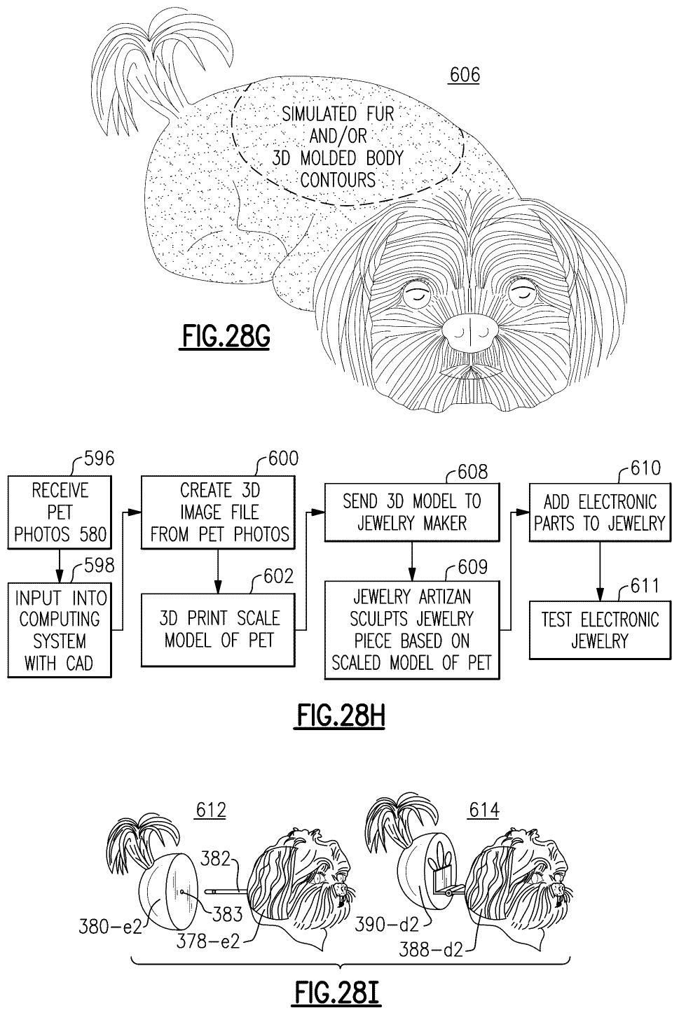

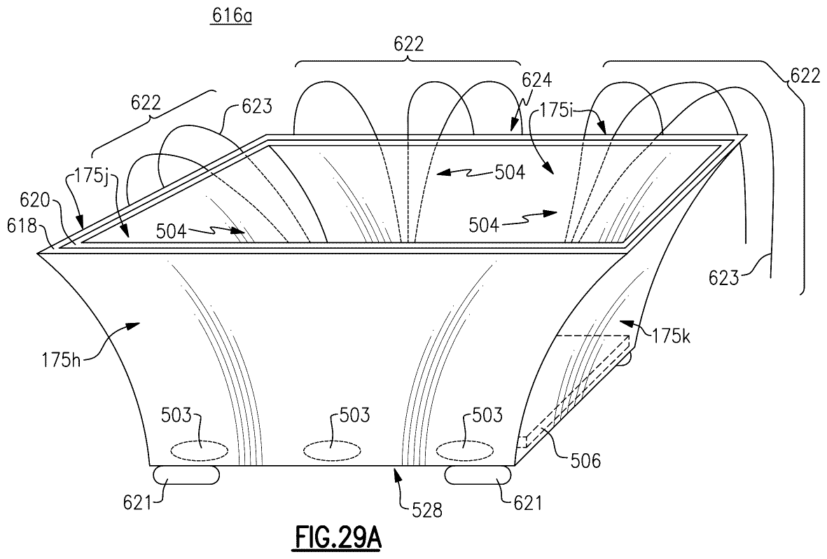

FIG. 15G-2 is a pictorial illustration of a stylized two-piece electronic jewelry item of the type of FIG. 15B here having sculpted pieces configured in the form of a puppy or mature canine face as the front-side piece and a corresponding tail as the back-side piece that may be manufactured by the method of FIG. 28H discussed in detail herein below. Thus here, as a counter-part to the above feline sculpting, as another example of a two-piece jewelry item stylized as a favored pet or animal, according to further aspects of this invention, there is provided in FIG. 15G-2 more particularly a pictorial illustration of a stylized two-piece electronic jewelry item 377-e2 of the type of FIG. 15B here having sculpted pieces configured in the form of a puppy or mature dog of any desired breed. As such, a front-side piece 378-e2 is configured as a puppy or adult canine head with facial details of a particular breed, and a corresponding back-side piece 380-e2 is configured in the form of a corresponding tail of the same breed which here includes a stylized body portion that would naturally be associated therewith. Here the head part of the front-side piece 378-e2, like above, is well suited to house a stack of micro-components, and the slender long form of the back-side piece 380-e2 configured as a canine tail is suited to housing an antenna assembly and receiver circuitry as implemented to receive a signal from a remote device such as a mobile phone, or an electronic bio-patch, or both a mobile phone and an electronic bio-patch, while the associated body portion here included in the back-side piece 380-e2 may be further suited to housing a vibration disk or a rechargeable battery with a central hole like those discussed above in FIGS. 5A to 5D and 6A to 6D.

FIG. 15H is a pictorial illustration of a stylized two-piece electronic jewelry item 377f of the type of FIG. 15B here having sculpted pieces configured in the form of a rose blossom and a rose flower stem with sepal. Here, like above, a front-side piece 378f is configured as a rose blossom, and a corresponding back-side piece 380f is configured in the form of a rose flower stem including here the sepal portion of such a flower as would be experienced growing in a natural setting or otherwise present with each rose blossom in a bouquet of long-stem cut roses for display in a vase. Here the rose blossom part in the front-side piece 378f, like above, is well suited to house a stack of micro-components, and the slender long form of the back-side piece 380d configured as a stem is suited to housing an antenna assembly as implemented to receive a signal from a remote device such as a mobile phone, or an electronic bio-patch, or both a mobile phone and an electronic bio-patch, while the sepal flower part here included with the back-side piece 380f is suited to housing a vibration disk or a rechargeable battery with a central hole like those discussed above in FIGS. 5A to 5D and 6A to 6D.

And lastly as a further example of these electronic jewelry items, FIG. 15I provides a pictorial illustration of a stylized two-piece electronic jewelry item 377g of the type of FIG. 15B here having sculpted pieces configured in the form of flowering Bougainvillea vines and a supporting branch or trunk structure as would be found rooted in the ground in a typical home garden or public park. Here, a front-side piece 378g is provided with a central button 384a which is stylized as a typical jewelry part configured to receive a variety of different adornments in a similar fashion to the teardrop and invented teardrop base pad assemblies 278 discussed above in detail in conjunction with FIGS. 12A and 12B. Here the jeweled central button 384a in the front-side piece 378g, is particularly configured to house a stack of micro-components, and the slender long form of the back-side piece 380g configured as a Bougainvillea branch or trunk is suited to housing an antenna assembly as implemented to receive a signal from a remote device such as a mobile phone, or an electronic bio-patch, or both a mobile phone and an electronic bio-patch. Here as also illustrated, the back-side piece 380g includes a stylized disk-shaped part or back-side button 385a that is particularly configured to house a vibration disk, a rechargeable battery, or both a vibration disk and a rechargeable battery, or the receiver circuitry including an antenna which may be directed up and down the branch portion for improved reception, where each of these components is provided with a central hole, where needed to accommodate the post, like those discussed above in FIGS. 6A to 6D. And as above, the back-side piece 380g may be also readily manufactured by adapting thereto the steps of the method of making the teardrop or invented teardrop base pad assemblies 278 discussed in detail above in conjunction with FIGS. 12A and 12B. Thus here, like the base pad assemblies 278, the frontside piece 378g including the central button 384a along with the back-side piece 380g with its attached stylized disk-shaped part or button 385a may be provided in collaboration from an electronics manufacture to a traditional jewelry maker for adding any desired form of adornments as provided at step 280 of the method of FIG. 12B. Here for purposes of illustration and example, the adornments on the front-side central button 384a include flowering Bougainvillea vines which may be rigid in structure or alternately may be made from fine chain links as is known in the art of traditional jewelry making to thereby provide movement or articulation to the vines so that they are thereby rendered to increase a feel or sensation of dangling swing by pendulum motion with body and head movement, and the length of the vines may be of any desired length to promote such a dangling, swinging sensation. Further as to adornments on the jeweled button 384a, the half-dome shown may be configured from natural pearl or cultured pearl, or alternatively in a precious metal such as gold, platinum, or silver, and the circular or annular rim shown on the button 384a also may be configured is any such desired precious metal. In a similar manner, the branch portion of the back-side piece 380g is preferably made from gold, platinum, or silver, while the stylized disk 385a may be made with such precious metal material and as illustrated here further bejeweled with adornments such as traditional cut diamonds in a reverse setting to form a spikey or spiked collar as shown. Here then, artistic stylization by use of such adornments in thereby provided on both the front-side piece 378g and the back-side piece 380g, where the traditional post with clasp on the back-side of the ear when worn is not necessarily intended for display with aesthetic or visual appeal.

And as would be appreciated by those of skill in the electrical and mechanical arts, given the disclosure hereof, there would be proposed several suitable means, when desired, for providing electric current between the front-side and back-side pieces 378 and 380 such as, for example, by providing a two-conductor co-axial post 382 so that electric current from a rechargeable battery in one piece may be directed via the post to the other piece to thereby power electronic components provide therein. Alternatively, a battery may be provided in both pieces, or an electro-magnetic (EM) field may be produced by one piece to activate current flow in a coil of the second piece or otherwise activate or facilitate reception or transmission of an antenna assembly. Further, other forms of a two-conductor post may include co-linear segments extending along the length of the post with each extending segment separated from the other by a suitable insulating material where each separate conducting segment electrically mates with a corresponding conductor or conducting surface provided in a receiving socket provided in the back-side piece. Further, these electronic posts may not necessarily be round in cross-section but may be of any desired geometric shape to form a type of “keyed post” to facilitate the two-conductor pathways between two operational pieces where the receiving hole in the back-side piece conforms in shape to the cross-section of the post thus creating a type of lock-and-key connection between the two pieces. As such, nonetheless, further detailed subject matter as to providing any desired two-conductor pathway from a battery in one piece to components in the other piece is considered extensive to the presently intended scope of the claimed subject matter of this disclosure and thus may be presented as the subject matter of subsequent disclosures as filed adjunct hereto.

Now with reference next to FIG. 15J, there is provided an exploded schematic perspective representation of a clip-type two-piece electronic jewelry item 387 having a front-side piece 388 and a back-side piece 390 with each thereof having a desired grouping of operable components as illustrated with the front-side piece 388 having a Group A subassembly of components, and back-side piece 390 having a Group B subassembly of components. Here in these clip-type embodiments, a spring or clip assembly 392 is utilized to affix together the front-side and back-side pieces 388 and 390. As such, the clip assembly 392 includes a front-side hinge plate 394a, a back-side hinge plate 394b, a hinge pin 398, and a spring 396, as an example of such a clip assembly that would be understood by those in the mechanical arts as applied by those of skill in the jewelry making arts. Each of the hinge plates 394a and 394b include a solid portion as shown and metal lace fingers 400 that provide mechanical support to there corresponding front-side piece or back-side piece. Here, front-side may be taken as right-side, and back-side may be taken as left-side, as presented, left to right in the illustration, such that depending on the sculped renderings of the adornments of each of the two pieces, the finished clip-type two-piece electronic jewelry item 387 hereof may be worn in a reversible manner thus effectively doubling its display effect, as compared to a traditional post-and-clasp or clip-type earing where if worn backwards or in a reverse manner, may not be suitable for intended display purposes. More recent mechanical arts have advanced the clip-type earing such that a snap fit is provide whereby the spring-loaded assembly is further provide with a detent that snaps or clicks into a corresponding mechanical receptacle under compressive spring force to provide comfortable as to wearability. The details of such know mechanical subassemblies are presently considered separate as to the intended scope of the claimed subject matter of this disclosure and thus may be found in current publications as adjacent hereto.

FIG. 15K is a pictorial illustration of a stylized two-piece electronic jewelry item 387a of the type of FIG. 15J here having sculpted pieces configured in the form of a ladybug with closed wings. Thus as shown, the front-side piece 388a includes a ladybug body with sculpted features and the back-side piece 390a similarly includes sculpted features as legs to the body. Each of the pieces 388a and 390a serve as components housings for components as discussed above and as such these details are not repeated here in the interest of avoiding duplicative disclosure. And as illustrated, the metal lace fingers 400 are configured in this embodiment to include three such finger for each piece that spread up and out to cover each of the planar back portions of each of the pieces in substantial part.

FIG. 15L is a pictorial illustration of a stylized two-piece electronic jewelry item 387b of the type of FIG. 15J here having sculpted pieces configured in the form of a ladybug with open wings. Thus as shown, the front-side piece 388b includes a ladybug body with open wings as sculpted features and the back-side piece 390b similarly includes the same sculpted features. Like above, each of the pieces 388b and 390b serve as a components housing for electronic and MEMS components as discussed above and as such these details are not repeated here. And as illustrated, the metal lace fingers 400 are configured in this embodiment to include three such fingers for each piece that spread up and out to cover in substantial part each of the back planar surfaces of each of the pieces. Here in this embodiment, the two-piece electronic jewelry item 387b is configured as reversable or reverse-wearable since the sculpted features on both the front-side and back-side are the same. Thus when worn either way, the same aesthetic features are presented. This repeating ladybug design is only one example of a reversable or reverse-wearable two-piece electronic jewelry item according to this aspect of the present inventions. As such, many other designs where the sculpted features on both sides are the same or different would be suitable to achieve these aspects hereof. For example, for a given pair of earrings according to these teachings, one side may be sculped as a honey bee and the other side sculpted as a flower where each sculpture has a high aesthetic value intended for display while still serving as a housing for electronic and MEMS components. In this case, when worn, one ear may present the honey bee on the front side and the other ear may present the flower on the front side where the honey bee and flower are partners in nature to achieve pollination.

FIG. 15M is a pictorial illustration of a stylized two-piece electronic jewelry item 387c of the type of FIG. 15J here having sculpted pieces configured in the form of a lotus flower and lotus leaf. Thus as shown, the front-side piece 388c includes as sculpted features a lotus flower and the back-side piece 390c includes corresponding sculpted features in the form of a lotus leaf. Each of the pieces 388c and 390c serve as components housings for components as discussed above and as such these details are not repeated here as to avoid unnecessary duplication. And as illustrated, the metal lace fingers 400 are configured in this embodiment to include three such fingers for each piece that spread up and out to cover each of the planar back portions of each of the pieces in substantial part. As illustrated on the leaf piece, the middle finger is shorter to accommodate the natural notch in a lotus leaf while the left and right fingers extend there-above to provide further support as may be desired.

FIG. 15N-1 is a pictorial illustration of a stylized two-piece electronic jewelry item 387-d1 of the type of FIG. 15J here having sculpted pieces configured in the form of a kitten, cat, or feline face as the front-side piece 388-d1 and a corresponding cat tail and associated body portion as the back-side piece 390-d1. Like above, each of the pieces 388-d1 and 390-d1 serve as a components housing for electronic and MEMS components as discussed above and thus such details are not repeated here. And as illustrated, the metal lace fingers 400 are configured in this embodiment to include three such fingers for each piece that spread up and out to cover in substantial part each of the planar back portions of each of the pieces. And here, while perhaps not first recognized, this cat body two-piece item according to the teachings hereof may be considered, as desired, as a reversable or reverse wearable earing. Where in this case, when worn, one ear may present the face of the cat or kitten on the front side and the other ear may present the tail portion on the front side so that when worn in this manner for display, such suggests a cat or kitten wrapping around the back of the neck of the person wearing this pair of earrings in this manner.

FIG. 15N-2 is a pictorial illustration of a stylized two-piece electronic jewelry item 387-d2 of the type of FIG. 15J here having sculpted pieces configured in the form of a puppy or canine face as the front-side piece 388-d2 and a corresponding canine tail and associated body portion as the back-side piece 390-d2 which two-piece item may be made according to the method of FIG. 28H discussed herein below in further detail. Now as to the item 387-d2 in here in FIG. 15N-2, like above, each of the pieces 388-d2 and 390-d2 serve as a components housing for electronic and MEMS components as discussed above and thus such details are not repeated here. And as illustrated, the metal lace fingers 400 are configured in this embodiment to include three such fingers for each piece that spread up and out to cover in substantial part each of the planar back portions of each of the pieces. And here, while perhaps not first recognized, this canine body two-piece item according to the teachings hereof may be considered, as desired, as a reversable or reverse wearable earing. Where in this case, when worn, one ear may present the face of the puppy or dog on the front side and the other ear may present the corresponding tail portion on the front side so that when worn in this manner for display, such suggests a puppy or dog wrapping around the back of the neck of the person wearing this pair of earrings in this manner.

Next in FIG. 15O, there is presented a pictorial illustration of a stylized two-piece electronic jewelry item 387e of the type of FIG. 15J here having sculpted pieces configured in the form of a rose blossom and a rose flower stem with sepal. Thus as shown, the front-side piece 388e includes as sculpted features a rose blossom and the back-side piece 390e includes corresponding sculpted features in the form of a rose stem with sepal further stylized with stem leaves and thorns. Like above, each of the pieces 388e and 390e serve as a housings for electronic and MEMS components as discussed above, and here as illustrated on the back-side piece 390e, there is provide a single metal lace finger 400 to comport with the shape of the sepal. Here also, this item may be considered reverse wearable such that when worn, one ear may present the rose blossom on the front side and the other ear may present the stem and septal on the front side so that when worn in this manner for display, a vase of two roses may be suggested with each thereof presenting in an opposite direction.

FIG. 15P is a pictorial illustration of a stylized two-piece electronic jewelry item 387f of the type of FIG. 15J here having a sculpted front-side piece 388f configured in the form of flowering Bougainvillea vines and a back-side piece 390f sculpted as a corresponding branch to from an electronic clip-type earring with a botanical motif. In a similar manner as to the post-type for a pierced ear discussed in conjunction with FIG. 15I, here in FIG. 15P, the front-side piece 388f also includes a jeweled central button 384b which is stylized as a typical jewelry part configured to receive a variety of different adornments in a similar fashion to the teardrop and invented teardrop base pad assemblies 278 discussed above in detail in conjunction with FIGS. 12A and 12B. Here the jeweled central button 384b in the front-side piece 388f, is particularly configured to house a stack of micro-components, and the slender long form of the back-side piece 390f configured as a Bougainvillea branch or trunk is suited to housing an antenna assembly as implemented to receive a signal from a remote device such as a mobile phone, or an electronic bio-patch, or both a mobile phone and an electronic bio-patch. Here as also illustrated, the back-side piece 390f includes a stylized disk-shaped part or jeweled back-side button 385b that is particularly configured to house a vibration disk, a rechargeable battery, or both a vibration disk and a rechargeable battery, or the receiver circuitry including an antenna which may be directed up and down the branch portion for improved reception. And as above, the back-side piece 390f may be also readily manufactured by adapting thereto the steps of the method of making the teardrop and invented teardrop base pad assemblies 278 discussed in detail above in conjunction with FIGS. 12A and 12B. Thus here, like the base pad assemblies 278, the frontside piece 388f including the central button 384b along with the back-side piece 390f with its attached stylized or bejeweled disk-shaped part or button 385b may be provided in collaboration from an electronics manufacture to a traditional jewelry maker for adding any desired form of gem stone or precious metal adornments as provided at step 280 of the method of FIG. 12B. Here for purposes of illustration and example, the adornments include flowering Bougainvillea vines on the front side piece 388f which may be rigid in structure or alternately may be made from fine chain links as is known in the art of traditional jewelry making to thereby provide movement or articulation to the vines so that they are thereby rendered to increase a feel or sensation of dangling swing by pendulum motion with body and head movement, and as shown further adornments on the back-side piece 390f may include diamonds, rubies, emeralds or other gem stones set on the edge of the disk 385b. Thus here in FIG. 15P, like above in FIG. 15I, with the mixed motifs of sculpted botanical forms and traditional jeweled items including precious gem stones such as diamonds, rubies, or emeralds, for example, as set in gold or silver, there is thus herewith provided a stylized or sculpted mixed motif two-piece electronic jewelry item according to the teachings hereof.

As such, as shown in detail in FIGS. 15B to 15P, the inventors hereof further provide a two-piece electronic jewelry item for use as an earing, said two-piece electronic jewelry item comprising a decorative front-side piece 378, 388 configured as a first components housing; a decorative back-side piece 380, 390 configured as a second components housing and configured to operate in association with the front-side piece 378, 388 to form a wearable jewelry assembly 377, 387; and a plurality of circuit components integrated within the first and second components housings to form a components assembly implemented to receive a wireless electronic signal from a remote device and thereby provide a vibration sensation to a person using the wearable jewelry assembly as an earing. Here in one embodiment, the components assembly includes a receiver, a vibration member, and a rechargeable power cell, and also the receiver may include an antenna. In further embodiments hereof, the components assembly may further include a transmitter configured to send a wireless signal to a remote device. And for hearable applications, the components assembly further includes a microphone configured to detect a sound. In such hearable embodiments, the sound detected may be a snoring sound made by a person using the wearable jewelry assembly as an earing while sleeping in an adjustable bed. In system implementations thereof, the wireless signal sent by the transmitter causes the adjustable bed to change to a position that causes the sleeping person to reduce the snoring sound. As to further system configurations, the remote device associated with the transmitter may be the same device as the remote device associated with the receiver, or the remote device associated with the transmitter may be a different device than the remote device associated with the receiver. In specific embodiments regarding the location of specific components, the antenna is provided in the back-side piece 380, 390 and the receiver, the vibration member, and the rechargeable power cell are provided in the front-side piece 378, 388, alternatively, the antenna and the receiver are provided in the back-side piece 380, 390 and the vibration member and the rechargeable power cell are provided in the front-side piece 378, 388, still further in alternate embodiments, the antenna, the receiver, and the vibration member are provided in the back-side piece 380, 390 and the rechargeable power cell is provided in the front-side piece 378, 388, yet also where desired given the form of the sculped adornments, the rechargeable power cell and the vibration member are provided in the back-side piece 380, 390 and the antenna and the receiver are provided in the front-side piece 378, 388. In another embodiment, the rechargeable power cell, the vibration member, the antenna, and the receiver are all provided in the front-side piece 378, 388, or as may be preferred as based on the geometry of the decorative portion of back-side piece, the rechargeable power cell, the vibration member, the antenna, and the receiver are all provided in the back-side piece 380, 390.

As to proposed sculpted features, the front-side piece 378, 388 may be configured to form a top portion of a ladybug body with closed wings and the back-side piece 380, 290 then configured to form a bottom portion of a ladybug body with legs, or the front-side piece 378, 388 is configured to form a top portion of a ladybug body with open wings and the back-side piece 380, 380 is configured to form a bottom portion of a ladybug body with legs, which may have corresponding open wings, and head to form the same view from both sides of the item when worn as an earing. Still further as to sculpted features according to this aspect of these embodiments, the front-side piece 378, 388 may be configured to form a lotus flower and the back-side piece 380, 390 is configured to form a lotus leaf, or the front-side piece 378, 388 is configured to form a sun flower and the back-side piece 380, 390 is configured to form a sun flower stalk or stem, or still, the front-side piece 378, 388 is configured to form a face of an animal and the back-side piece 380, 390 is configured to form a back-side portion of the animal with a tail, here, the animal may be a cat or kitten, a dog or puppy, a panda bear, or any other desired animal. And still, in the alternative as to preferred floral renderings, the front-side piece 378, 388 may be configured to form a rose blossom and the back-side piece 380, 390 then configured to form a rose stem with a sepal portion with components housed within the sepal portion, or still, the front-side piece 378, 388 is configured in the form of flowering vines and the back-side piece 380, 390 is configured to form a branch portion as supporting growth of the flowering vines, where here, the flowering vines may be in the form of Bougainvillea, or any other desired flowing vine.

As to all the above two-piece electronic jewelry items 377 for use with a pierced ear as discussed in conjunction with FIG. 15B to FIG. 15I, the front-side piece 378 includes a post 382 and the back-side piece 380 includes a corresponding hole 383 configured to securely receive the post in a removable manner.

And as to each of the two-piece electronic jewelry items 387 of FIGS. 15J to 15P, the front-side piece 388 and the back-side piece 390 are affixed together by spring-hinge assembly 392 to form a clip-type earing.

And as would be appreciated by those of skill in the electrical and mechanical arts, given the disclosure hereof, there would be proposed several suitable means, when desired, for providing electric current between the front-side and back-side pieces 388 and 390 such as, for example, by providing a two-conductor wire through the hinge assembly 392 so that electric current from a rechargeable battery in one piece may be directed via the wire to the other piece to thereby power electronic components provide therein. Alternatively, a battery may be provided in both pieces, or an electro-magnetic (EM) field may be produced by one piece to activate current flow in a coil of the second piece or otherwise activate or facilitate reception or transmission of an antenna assembly. Further, other forms of a two-conductor wires may be proposed to conduct low voltage current from the front-side piece to the back-side piece through the hinge assembly 392. As such, nonetheless, further detailed subject matter as to providing any desired two-conductor pathway from a battery in one piece to components in the other piece via a hinge assembly is considered extensive to the presently intended scope of the claimed subject matter of this disclosure and thus may be presented as the subject matter of subsequent disclosures as filed adjunct hereto.

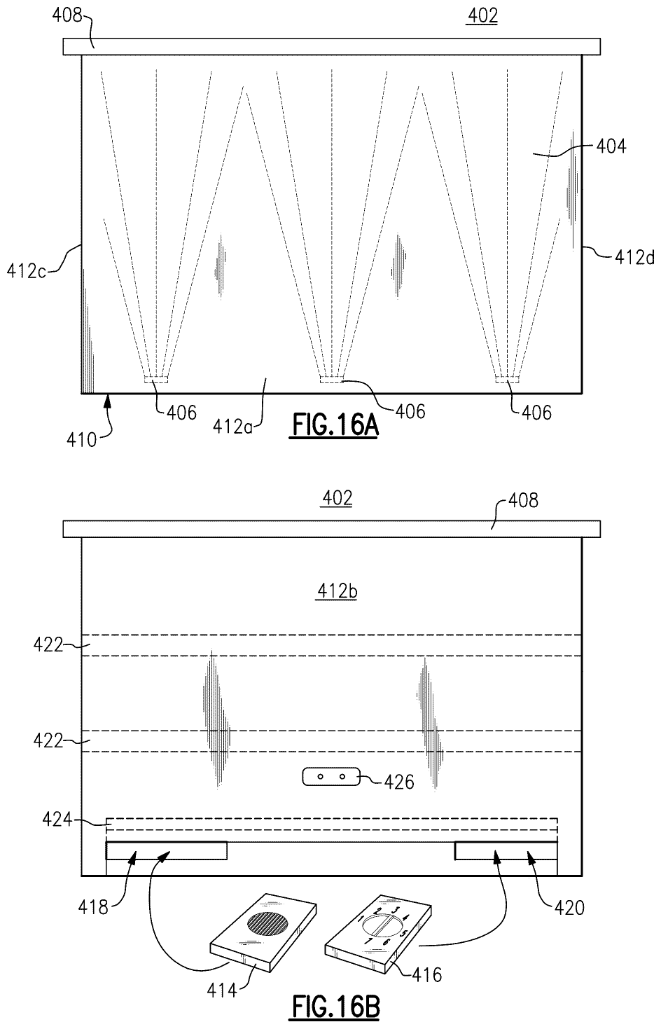

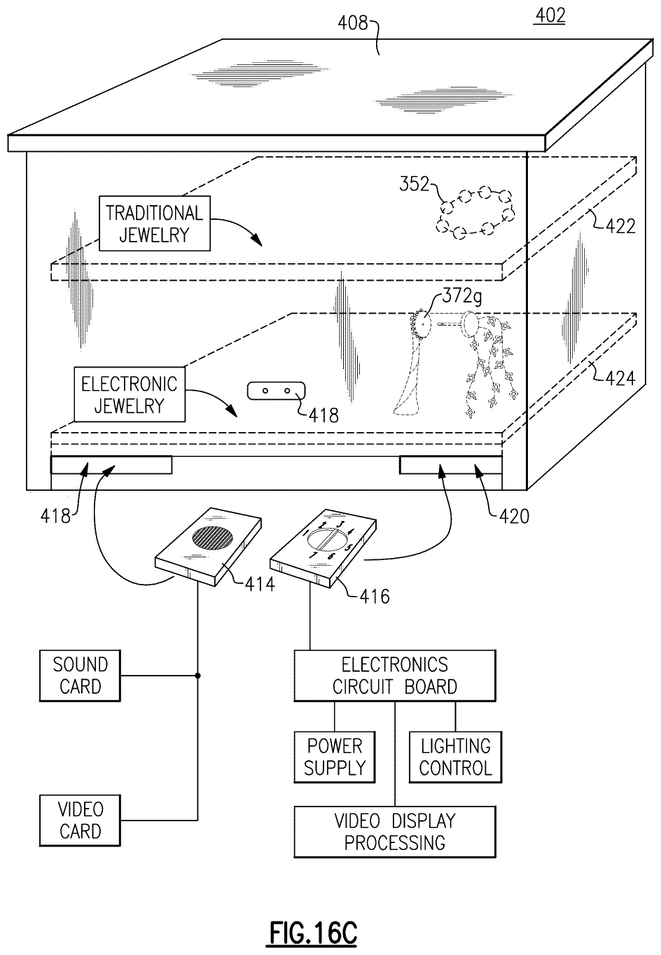

Now as to the continued subject matter hereof, with reference next to FIG. 16A, there is shown a front elevation view of another embodiment of an electronic jewelry box 402 including a front face 404 with indicator lights 406 that project light thereinto. As to the principal structural elements of the box 402, like the box 176 in FIGS. 4A and 15A, the box 402 here in FIGS. 16A to 16C includes a hinged cover 408, a bottom 410, and four side walls 412 including a front side wall 412a, a back side wall 412b, a left-hand side wall 412c, and a left-hand side wall 412d. Next, FIG. 16B presents a backside elevation view of the electronic jewelry box 402 of FIG. 16A showing in perspective as removed therefrom a sound box 414 and a control unit or controller 416 in accordance with additional aspects this invention. The electronic jewelry box 402 is provided with a first hide-away pocket, compartment, or cubby hole 418 for the sound box 414 and a second hide-away pocket, compartment, or cubby hole 420 for the control unit 416. In this manner, both the sound box 414 and the control unit 416 are easily accessed for repair, upgrades, or replacement. FIG. 16B also shows in dashed-line traditional jewelry box removable trays 422 and an electronic charging pad 424 of the type discussed in detail above in FIGS. 4B and 14B. A plug socket or charging port 426 is provided to receive power from an electric supply cord in the usual way so that the indicator lights 406, the sound box 414, the control unit 416, and the charging pad 424 are thereby supplied with electric power as needed for their respective functionalities. FIG. 16C is a view similar to FIG. 16B schematically extended in perspective and illustrating various additional electronic functionalities that may be provided with these illuminated electronic jewelry boxes according to further embodiments thereof. First as illustrated, the traditional removable jewelry tray 422 includes the pearl bracelet 352 of FIG. 14A which has not been rendered with electronic functionalities hereof and thus as a traditional piece of jewelry is stored for use on the traditional jewelry tray 422 in the usual manner. Meanwhile, on the charging pad 424 for further illustration of the intended functionalities of the electronic jewelry boxes hereof as used in conjunction with the electronic jewelry items hereof, as disclosed and claimed in combination and separately herein, the stylized two-piece electronic jewelry item with flowering Bougainvillea vines 377g of FIG. 15I is shown again here on the charging pad 424 of FIG. 16C as intended for recharging and later functional use for any of the use scenarios herein presented and discussed. And as would be readily appreciated by those of skill in the relevant arts, given there disclosure hereof, the indicator lights 406 may be rendered in the form of electronically controlled projection lights such that a video may be projected on the front face 404 of the box 402.

For purposes of clarity here with regard to the electronic jewelry box 402 of FIGS. 16A to 16C, front face 404 comprises the front wall 412a as a structural element as well as any electronics and lighting elements that may be integrated therewith so as to provide a lively or animated light display or light show where activation of any included indicator lights provides not only a pleasing, soothing, visual experience from an artistic perspective (that may be enhanced with corresponding soothing sounds including sound in the form of speech) but also such light activation has the added functionality as a communications tool that may indicate the status of electronic items placed into the box or otherwise when the box has supplied power. As to providing sounds from the sound box 414, pre-recorded phrases such as “your jewelry box has power” or “your favorite pair of earrings are fully charged” may be included as added animation and functionality for certain market segments as desired. Further technical details for providing such sound functionality are beyond the scope of the present disclosure and as such may be the subject matter of subsequent disclosures filed in companionship herewith.

Thus with reference to FIGS. 16A to 16C, there is further herein provided a jewelry box 402 for charging a collection of jewelry items each having network-ready alert functionality and a rechargeable battery in support thereof, where here the jewelry box 402 includes side walls 412, a bottom 410, and a top 408 implemented to open and close relative to the side walls 412 with one of the side walls being a back side wall 412b relative to the open and close functionality of the top 408, where the side walls 412, the bottom 410, and the top 408 are implemented to form the box 402. Further included in the box 402 is a charging pad 424 situated in association with the bottom of the box and a charging port or plug socket 426 implemented in the back side wall 412b of the box 402. And still further, the box 402 includes at least one indicator light 406 implemented to indicate when the charging port 426 has supplied power and/or when a rechargeable battery of a respective electronic jewelry item (377g, for example) is charged after placing the jewelry item in the box. In certain embodiments thereof, the at least one indicator light 406 includes a plurality of indicator lights 406. Then where desired, a first number of said plurality of indicator lights 406 may emit a first color of visible light and a second number of said plurality of indicator lights 406 then emits a second color of visible light. And as to the desired illumination of the box 402, the first number colored lights 406 may be one, two, or more than two; and the second number colored lights 406 may be one, two, or more than two as well. In any of these embodiments, the first color may be emitted when the charging port has power, and the second color may be emitted when the rechargeable battery of a respective jewelry item (for example, 377g) as placed in the box 402 is charged to a predetermined level of charge. In certain preferred embodiments of the above, the side walls 412 are translucent and the indicator lights 406 of the first color are formed integral with at least one of the side walls and similarly, when the side walls 412 are translucent, then the indicator lights of the second color may be formed integral with at least one of the side walls as shown in the front face 404 in FIG. 16A. And thus depending on the desired level of illumination as associated with liveliness or animation of the box 402, the indicator lights 406 of the first color may be formed integral with two or more of the side walls, and in a similar fashion, the indicator lights 406 of the second color also may be formed integral with two or more of the side walls 412. Given these various options for our electronic jewelry box 402, a variety of arrangements may be provided such as where the indicator lights 406 of the first color are situated in an alternating series with the indicator lights 406 of the second color along one, two, or more then two side walls, or the indicator lights 406 of the second color are situated in an alternating series with the indicator lights 406 of the first color along one, two, or more than two side walls. In any of these above embodiments, the indicator lights 406 may be formed from light emitting diodes. And as illustrated, the first and second colors of visible light may be selectable from a plurality of different colors provided by a controller 416 associated with a power cord implemented in association with the charging port 426 in the backside 412b of the box, or the first and second colors of visible light may be selectable from a plurality of different colors provided by the controller 416 as integrated in association with the bottom 410 of the box 402 such as in the cubby hole or hide-away pocket 420. Thus with the introduction of the controller 416, a variety of light shows may be projected on the front face 404 of the box 402, thereby giving the box a sense of liveliness and animation. And as to this aspect of the present invention, the indicator lights 406 of the second color may be emitted in a repeating pattern, where, for example, the repeating pattern is projected as a water fountain pattern, or the repeating pattern is projected as an exploding fireworks pattern. In more particular embodiments of any of the above electronic jewelry boxes 402, the added functionality of sound may be desired and as such is herein provided. Thus to achieve same, there is further provided the sound box 412, as discussed above, that plays a selected sound clip when the second color is emitted, for example. And still in further animated embodiments hereof, the controller 416 or sound box 414 working in conjunction therewith, may be provided with a video card that plays a selected video clip as projected on the front face 404 when the second color is emitted, or at any other programmed time such as when a user of the box is getting set to fall asleep for the nighttime rest. In these embodiments, the controller 416 may project any of the repeating patterns in a random order, and where preferred, any of these projected repeating patterns may be projected in a slower, smoothly moving manner to help induce as sense of calm for transitioning a person for nighttime sleep after busy day of daytime activities.

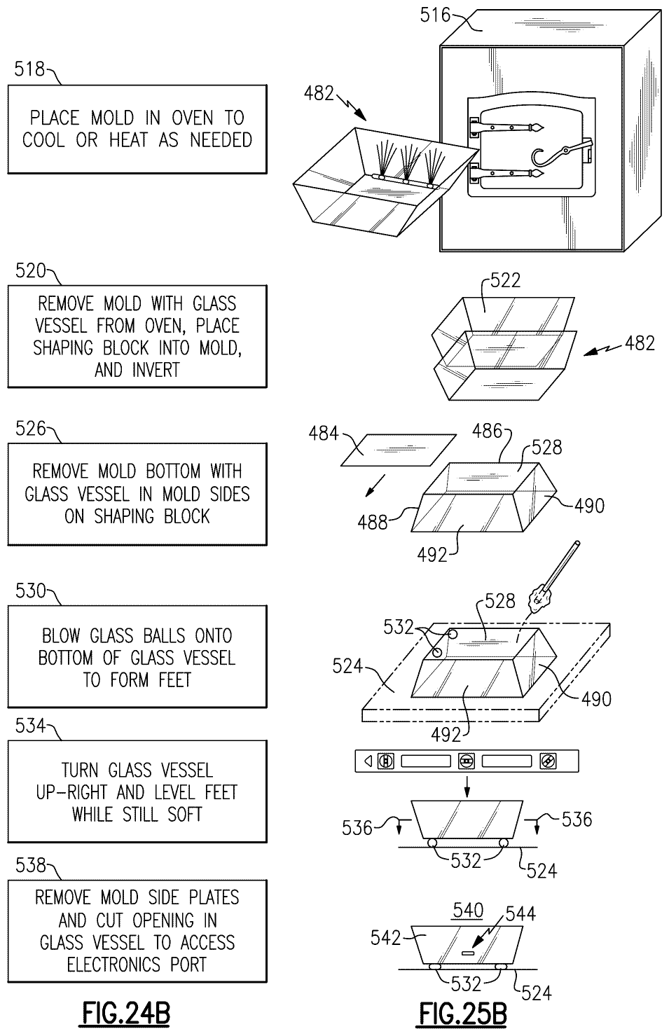

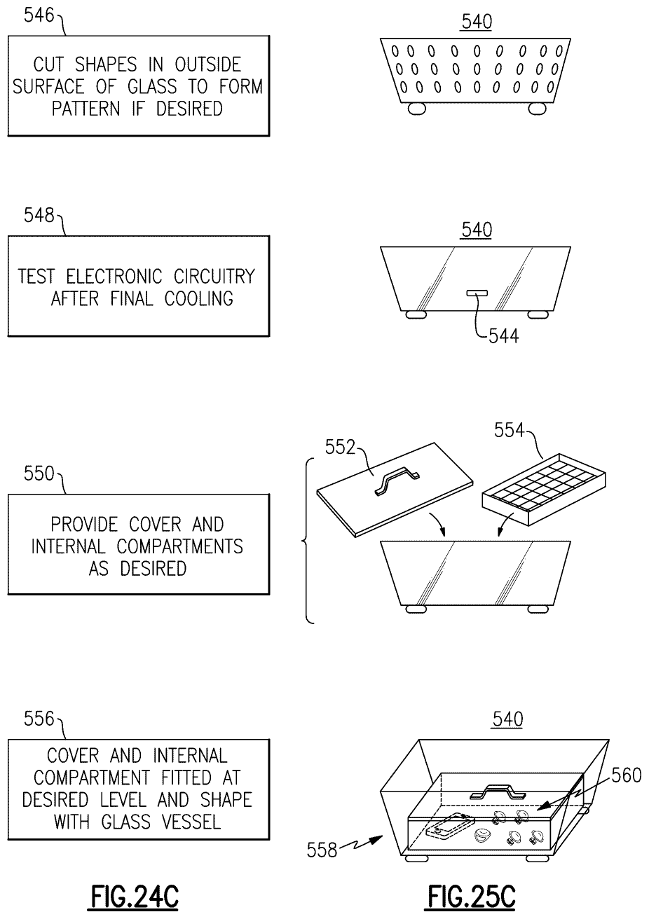

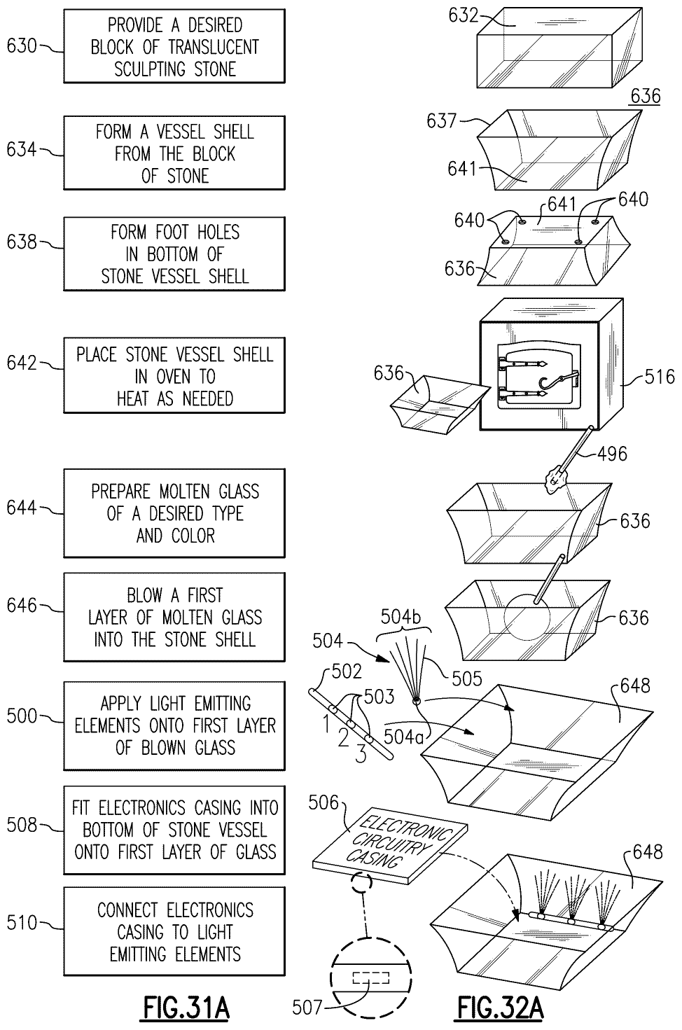

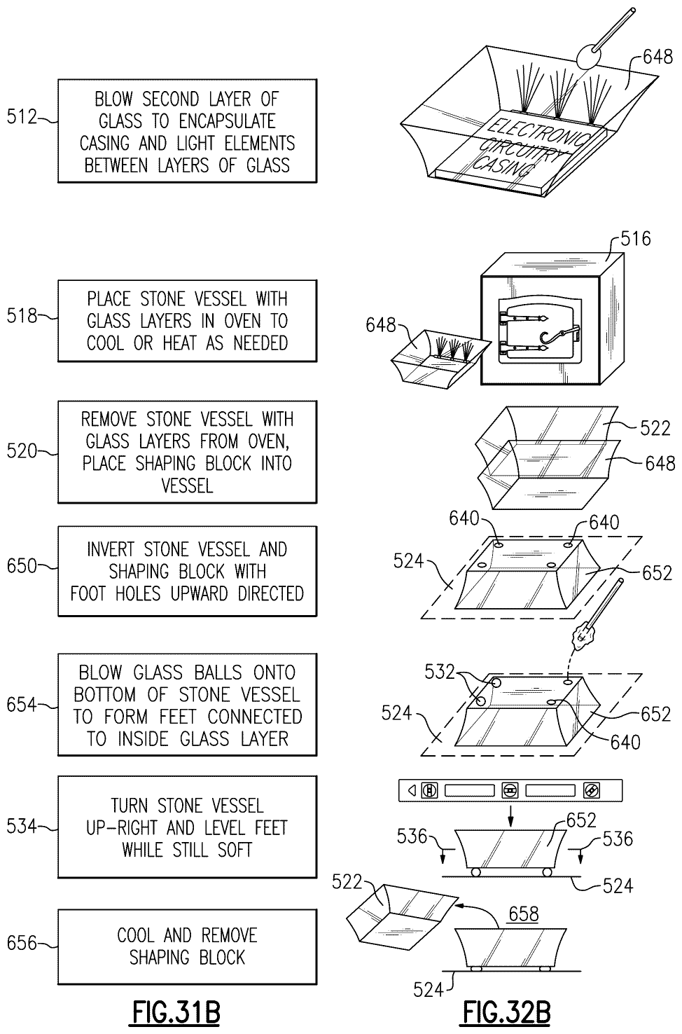

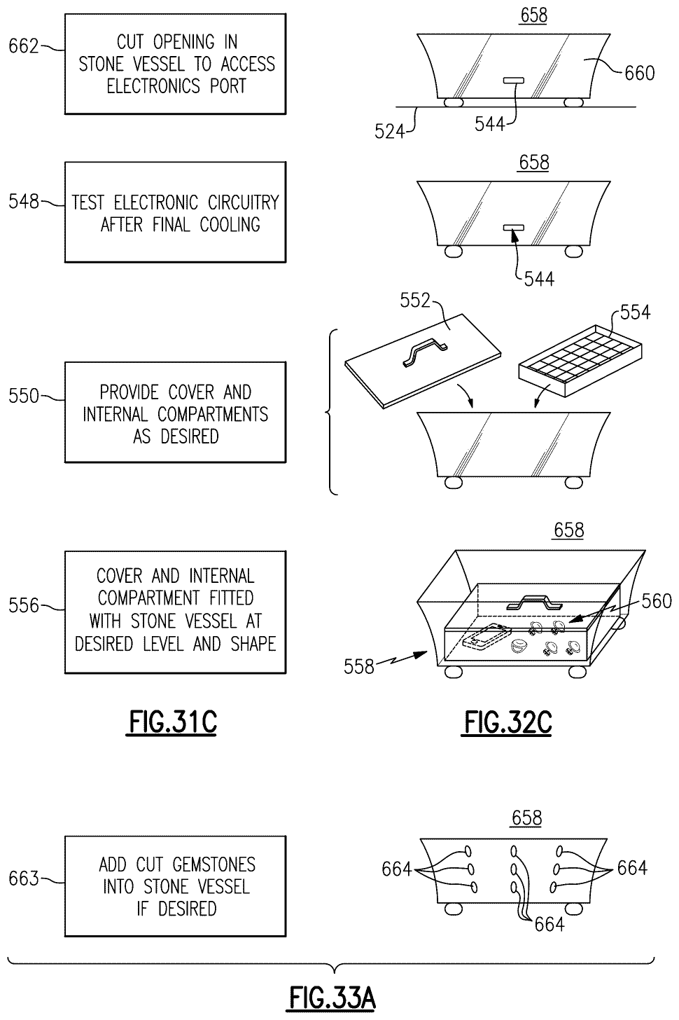

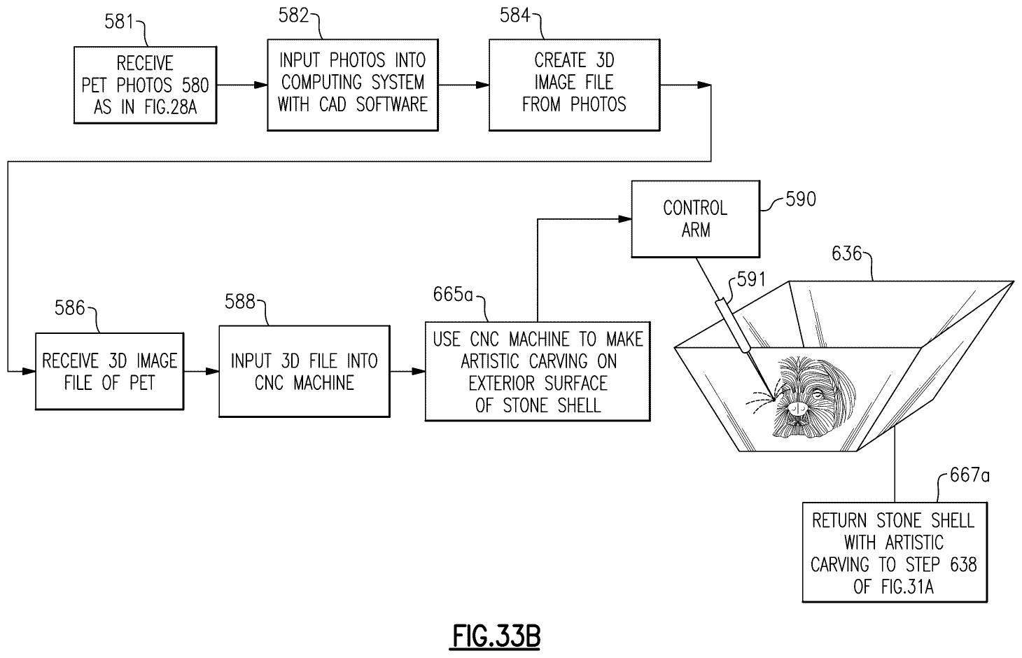

The electronic jewelry box 402 of FIGS. 16A, 16B, and 16C may be made from any desired manufactured material that is translucent and formable into workable sheets or alternatively, for certain markets segments that may be so interested, the box 402 may be more preferably made from sculpting a natural stone material such as Onyx, Alabaster, or Makrana marble of the type from India which may further preferably include some selectedly placed inlayed precious or simi-precious stones as has been well known for hundreds of years by specialized stone artisans working with these materials. In any of these stone sculpted embodiments, the stone vessel may include gilding where desired to provided further unique characterization to each such stone vessel made according to these methods as exemplified in the methods discussed below in detail in conjunction with FIGS. 31A to 31C, FIGS. 32A to 32C, FIGS. 43A to 43C, FIGS. 47A to 47C, and FIGS. 48A to 48C; and as to our blown glass illuminated charging vessels as discussed in detail in connection with FIGS. 24A to 24C, FIGS. 25A to 25C, and FIGS. 26A to 30H.

But before proceeding to discussing in detail these methods of making our blown glass and sculpted stone illuminated charging vessels for electronic jewelry and mobile phones, according to still further teachings hereof, readers are next directed to the following disclosure of our system level assemblies and various use scenarios associated therewith.

Thus now with reference to FIG. 17A, there is shown a perspective view of some of the above electronic jewelry items having network-ready functionality according to the teachings hereof as presented here again for exemplary purposes which include the finger ring assembly 156 of FIG. 3B, the butterfly-shaped finished clasp assembly 215 of FIGS. 8 and 10A, the finished inverted teardrop earrings 284 of FIG. 12B, the finished electronic jewelry item 330a of FIG. 13D as configured in a half-dome clip-on pearl earring, the wrist bracelet 350 of FIG. 14E, the electronic earrings 374 of FIG. 15A, and the stylized two-piece electronic jewelry item 377g of FIG. 15I. As discussed above, each of these electronic jewelry items includes a receiver or receiver circuitry that enables the jewelry item to receive a wireless signal from a remote device such as a mobile phone or an electronic bio-patch of the types discussed above and incorporated herein by reference. And as discussed above in conjunction with this receiving functionality, each of these electronic jewelry items further includes a vibration member that when activated by the received signal from the remote device, causes a vibration sensation on a person wearing the jewelry item as intended. This vibration sensation then serves as a discreet silent alert signal to the person wearing the jewelry item, which alert may be a silent wake-up alarm so that a sleeping partner is not awoken by a sounding alarm, or where the vibration sensation is a timed notification to take a medication, or a timed notification for a parent to call a child daughter or son at a certain time, or to leave work to pick up such a child from school. Thus at a system level, these electronic jewelry items may be used advantageously in concert with a smart phone such as the exemplary smart phone 428 illustrated in FIG. 17B as enabled with a “Life Jewel” app 430 which is currently intended as the subject matter of additional companion disclosures related hereto.

As to the health and wellness applications hereof, FIG. 17C exemplifies two of several different types of personal diagnostic devices that may engage with the smart phone of FIG. 17B or otherwise engage directly with the electronic jewelry items shown in FIG. 17A. One such device is a non-invasive fever patch 432 discussed in further detail in commonly owned U.S. Provisional Application No. 62/972,657 filed Feb. 11, 2020, titled Wearable Patches for Babies as referenced and incorporated herein above, and the other illustrated wearable diagnostic device is a representative fluidic diagnostic patch 434 of the flow-through type as discussed in further detail in commonly owned U.S. Provisional Application No. 62/700,981 filed Jul. 20, 2018, titled Bio-Patch Having Artificial Capillaries to Provide Continuous Blood Flow. Thus when in use in a supporting system infrastructure, the electronic jewelry items hereof may advantageously interact smart phones and bio-patches. In these health and wellness applications including a bio-patch, the vibration sensation of our electronic jewelry items may then also serve as a discreet silent alert signal to the person wearing the jewelry item as reporting a detected change in a health or wellness condition of that person or of a second person wearing the bio-patch. In these applications, the detected health condition may be a change in blood sugar level, an elevated body temperature, entering a window of fertility, confirmation of conception as to a desired pregnancy, increased levels of lactose acid in the blood stream, alcohol blood levels, or other such health, wellness, or lifestyle biological or medical conditions as disclosed in our various commonly assigned bio-patch applications and patents as incorporated herein above and further below.

Thus with the above at hand for purposes hereof, next described are various use or deployment scenarios of an electronic jewelry item according to the present inventions as shown deployed within a supporting wireless communications infrastructure and being thereby wirelessly engaged with a smart phone, a wearable diagnostic patch, or as engaged with both a smart phone and a wearable diagnostic patch. As such, FIG. 18 is a perspective pictorial view of a person 440 using the electronic jewelry hereof initiating activation of the jewelry by interaction with an authorized website so that the jewelry items are duly registered and paired with the user's smartphone 428 as referenced above in FIG. 17B. As such, the person 440 is shown with an electronic earring 442 being representative of any of the types discussed above, an electronic nose piercing 444 of the type illustrated above in FIG. 1C, an electronic ring 446 of the type discussed above in FIGS. 3A and 3B, an electronic bracelet 448 being, for example, such of the type in FIGS. 14A to 14E, and a bio-patch 450 which in this case for purposes of illustration may be taken as a human fertility patch.

Thus once the above devices are duly registered and activated, in comes FIG. 19 showing a perspective pictorial illustration of a use scenario hereof representing various night-time applications that may be employed by the user 440 of the electronic jewelry devices hereof as deployed in a private network including the personal smartphone 428, the bio-patch 450, or both the smartphone 428 and the bio-patch 450. For example, the nose piercing 444 may detect a snoring sound, send a signal to the phone 428, which in turn sends a signal to the bed, which bed is then adjusted to gently coax the sleeping person into a different sleeping position to reduce the snoring sound so a partner sleeping next thereto is not awaken by the snoring sound while such person 440 remains in a sleeping state as well. Further technical details relating to such a snoring application and the electronic and mechanical apparatus associated therewith are provided in the above commonly owned and incorporated U.S. Pub. No. 2020/0345533 titled Wearable Patch For Reducing Snoring Activity as filed on May 1, 2020 under U.S. application Ser. No. 16/864,863, having priority to U.S. Provisional Application No. 62/842,472 as filed on May 2, 2019, and thus as such, those snoring application technical details need not be repeated herein.

Another night-time application represented in FIG. 19 is that of our silent wake-up alarm. Thus here in such a wake-up application, the smartphone 428 is set for a wake-up time, for example 6:00 am to start the workday, and then at the set time, a wireless signal is sent from the phone 428 to the earring 442, the nose piercing 444, and/or the ring 446 which signal will, upon receipt at the jewelry item, cause the jewelry item vibrate to gently wake up the sleeping person 440 without making the noise of a sounding alarm that could otherwise disturb a partner sleeping next thereto who desires to sleep later into the morning. Further technical details relating to such a silent alarm wake-up application and the electronic and mechanical apparatus associated therewith are provided in the above-referenced commonly owned and incorporated U.S. Pat. No. 10,603,463 titled Individual Wake-Up Alarm Patch as filed on Jul. 30, 2018 under U.S. application Ser. No. 16/049,759, having priority to U.S. Provisional Application No. 62/540,126 as filed on Aug. 2, 2017, and thus as such, those silent alarm wake-up technical details need not be repeated herein.

Yet another night-time application represented in FIG. 19 is that of our human fertility patch. Thus here in such a human fertility application, the bio-patch 450 is configured to detect a change in female hormones associate with ovulation as an indicator of a condition of fertility which may have only a window of between 12 to 24 hours as to presenting an opportunity for fertilization. Thus upon movement of a mature egg down the fallopian tube from the ovary to be placed in position for fertilization, hormones associated therewith are detected by the bio-patch 450, where upon such detection, a wireless signal is sent from the bio-patch 450 to the phone 428, and then promptly in turn the phone 428 sends a wireless signal to the earring 442, the nose piercing 444, and/or the ring 446 which signal will, upon receipt at the jewelry item, cause the jewelry item to vibrate to thereby wake up the sleeping person 440 so that she may then take any desired steps towards conception and fertilization. Further details relating to such a human fertility application including apparatus and methods associated therewith are provided in our currently unfiled disclosure docket number LPCH-17118-PROV titled Human Fertility and Pregnancy Bio-Patches Including Personal Computing Devices and Methods, Processes, and Medical Protocols Relating Thereto as dated Jul. 10, 2017 under the inventor's signature, and thus as unfiled and otherwise unpublished, such details will not be presented herein. However, for those of our readers who may be interested in our fertility applications as adjacent to the above human application, attention is directed to commonly assigned UK Patent No. GB2585577 titled Bio-Patch and Related Methods for Detecting Fertility Condition in Cows as granted on Dec. 7, 2022 which is a counterpart British nationalization of International Application PCT/US2019/018375 filed Feb. 17, 2019 having priority to our U.S. Provisional Application 62/631,645 as filed on Feb. 17, 2018.

And lastly now here in FIG. 19, there is shown a thematic representation of a charging vessel 452 according to further aspects of the inventions disclosed herein as relating to our glass-blown illuminated charging vessels discussed in detail below in connection with FIGS. 24A to 24C, FIGS. 25A to 25C, and FIGS. 26A to 30D, and our sculpted stone vessels with blown glass as made according to the methods discussed below in detail in conjunction with FIGS. 31A to 31C, FIGS. 32A to 32C, FIGS. 43A to 43C, FIGS. 47A to 47C, and FIGS. 48A to 48C.

But for now continuing with use of our electronic jewelry as deployed in active wireless networks, FIG. 20 is a perspective pictorial illustration presenting another use scenario of these inventions showing a particular night-time application where an enabled electronic jewelry device hereof is worn by a first person and a bio-patch is worn by a second person. Thus here in FIG. 20 to set the stage of the scenario, there is first provided a home-living network arrangement 454 having a first room such as a nursery for an infant child 456 and a second room such as a bedroom for the mother of the child. Here in this scenario as illustrated, the sleeping person 440 may be taken as the mother of the sleeping child 456. Next as illustrated in Room 2 of this scenario, one of our sculpted-stone or blown-glass charging vessels 452 is on a nightstand securely charging the mother's phone 428 as shown laying flat on the bottom of the charging vessel 452. Meanwhile in Room 1, the sleeping infant child 456 is wearing a baby fever patch 432 of the type discussed above in FIG. 17C. And now lastly to set the scene in Room 2, the sleeping mother 440 is shown wearing an electronic earring 442, an electronic nose piercing 444, an electronic ring 446, and an electronic bracelet 448 of the types discussed above. Thus in this arrangement, if the sleeping child 456 starts to develop an elevated temperature, the fever patch 432 will detect such and then send a wireless signal to the mother's phone 428 where upon then promptly in turn the phone 428 sends a wireless signal to the earring 442, the nose piercing 444, the ring 446, and/or the bracelet 448 which signal will, upon receipt at the jewelry item, cause the jewelry item to vibrate to thereby wake up the sleeping mother 440 so that she may then take any desired steps towards attending to her child. And lastly here as to room configuration regarding the network system, Room 1 may be equipped with a signal boosting device 458 that thereby receives a signal from the fever patch 432 and relays that signal to the mother's phone 428 in Room 2. Alternatively as presented in FIGS. 17A to 17C, the fever patch 432 may communicate directly with any of the illustrated electronic jewelry items including the electronic earring 442, the nose piercing 444, the ring 446, and/or the bracelet 448, where then the phone 428 in not an intermediate node needed in the network. Thus in this alternate network configuration, the fever patch 456 communicates with the booster box 458, and then the booster box 458 communicates the patch signal directly to the jewelry items for activation of the jewelry item to thereby provided the above-discussed vibration sensation on the sleeping mother.

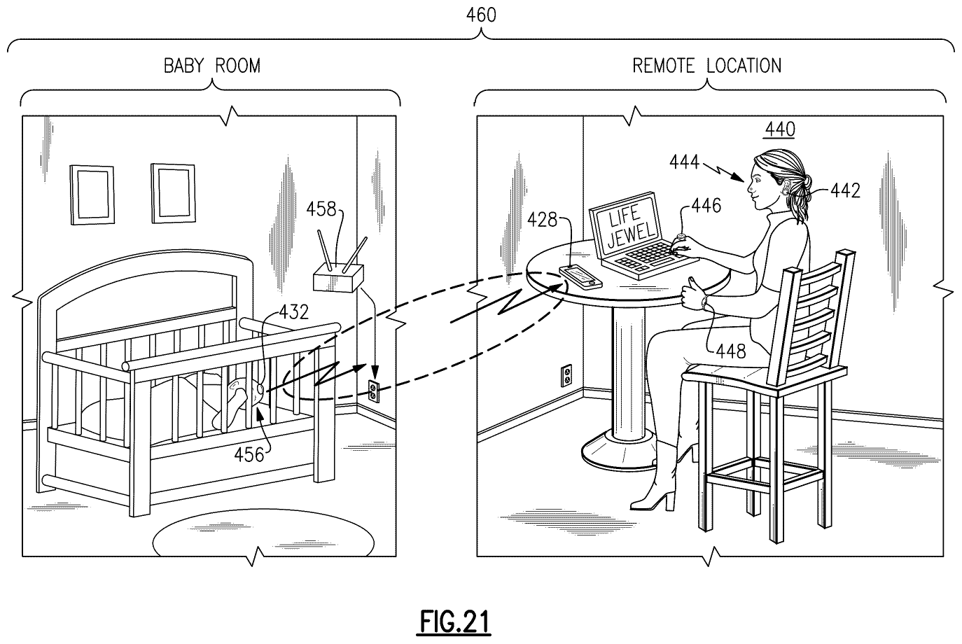

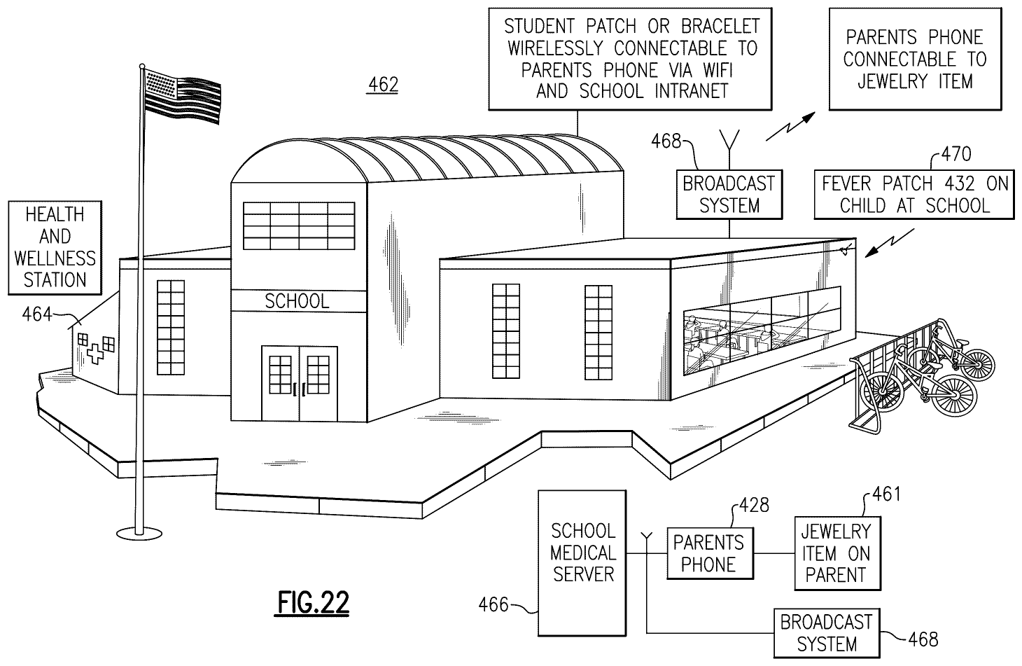

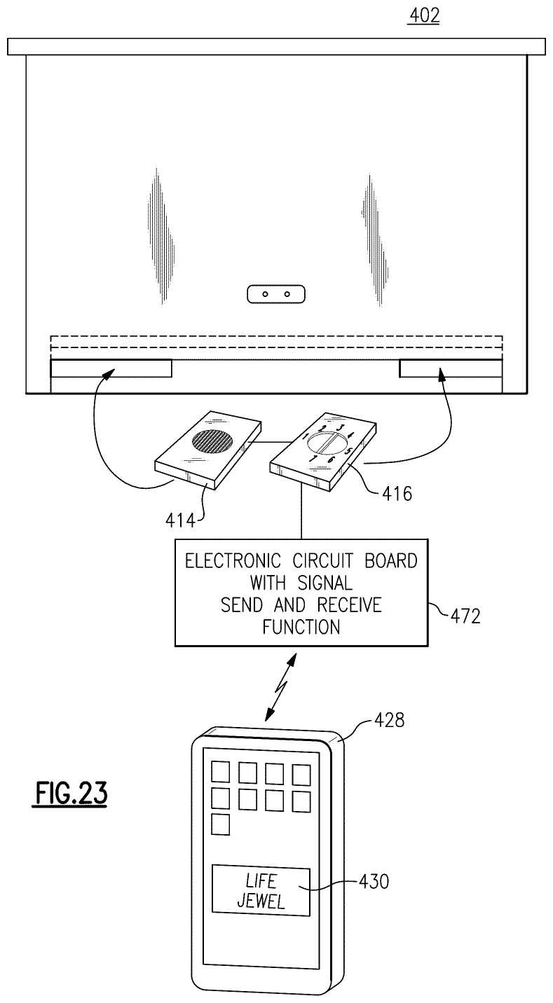

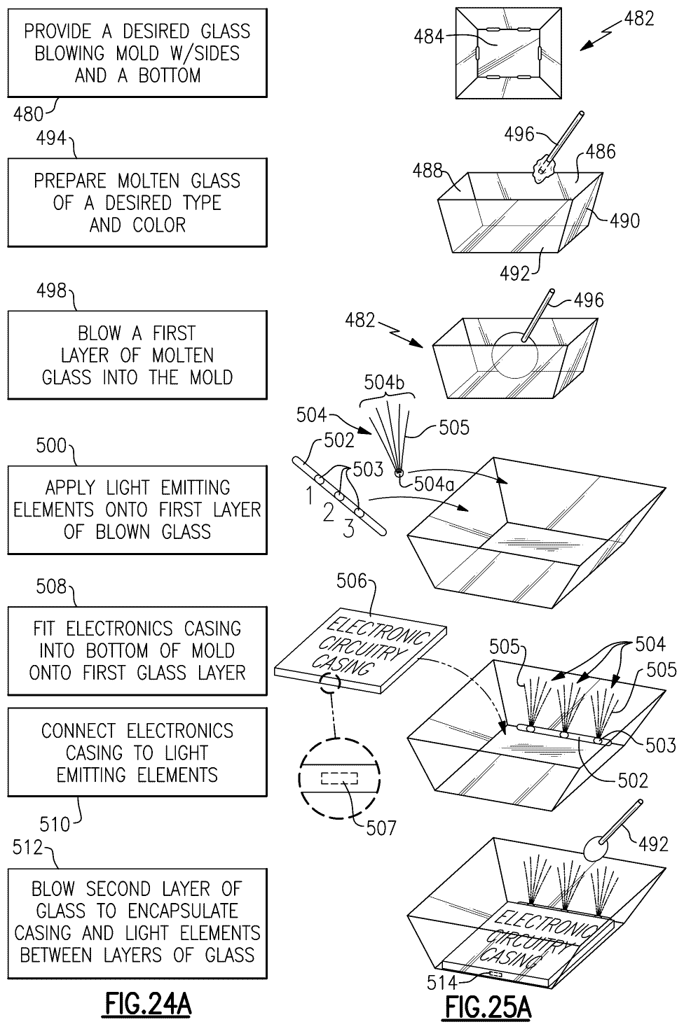

FIG. 21 is another perspective pictorial illustration of yet a further use scenario of these inventions showing a day-time application where an enabled electronic jewelry device hereof is worn by a first person and a bio-patch is worn by a second person. In this scenario, there is provided a home-and-remote-location network arrangement 460 including a Baby Room and a Remote Location, which Remote Location may be an employer's place of business or a coffee shop, for example. Here in the arrangement 460 of FIG. 21, the scene in the Baby Room is set the same as Room 1 in FIG. 20, while the person 440, here again taken as the mother of the infant child 456, is shown working at a bistro table that may be at a coffee shop or at an employer's place of business such as an employee cafeteria. And like the scene in Room 2 of FIG. 20, the mother 440 here at the bistro table is shown as wearing the electronic network-ready earring 442, nose piercing 444, ring 446, and bracelet 448, and has handy at the table her trusted mobile phone 428 in a well charged condition. Thus in this scenario, like above, if the sleeping child 456 starts to develop an elevated temperature, the fever patch 432 will detect such and then send a wireless signal to the mother's phone 428 where upon then promptly in turn the phone 428 sends a wireless signal to the earring 442, the nose piercing 444, the ring 446, and/or the bracelet 448 which signal will, upon receipt at the jewelry item, cause the jewelry item to vibrate to thereby call the mother's attention to her infant child so that she may then take any desired steps towards attending to her child. And also here, as to room configuration regarding the network system, the baby Room may be equipped with the signal boosting device 458 that thereby receives a signal from the fever patch 432 and relays that signal to the mother's phone 428 at the Remote Location.

Daily modern life is filled with a constant bombardment of video images and electronic sounds of beeping, chirping, or dinging sounds from all sorts of electronic devices and as such our sensory perception thereof may be diminished, muted, distracted, or otherwise blocked out so we may stay focused on tasks at hand. Further, an average person may receive hundreds of emails and texts per day. Thus here in such an active or overloaded communications environment, our vibrating electronic jewelry provides a completely different form of sensory communication so as to be easily and really distinguished from our daily engrossed experiences with common electronic devices and video screen forms of textual communications as receive by ever tiring eyes and ears as the busy workday progresses. Thus here most advantageously, the person 440 may configure his or her electronic jewelry to receive only certain priority signals, or even only one type of priority signal such as a distress signal from a child's bio-patch or other wearable communications device. In this manner, if the earring or the bracelet of the person 440 vibrates, they know immediately, by a different sensory perception, with that being a vibrating skin touch rather than by overloaded sensory perception of sights and sounds by eyes and ears, that the skin touch vibration feel as produced by our electronic jewelry, is caused uniquely by a signal from their child, or that signal is from, alternatively, another person of their caring such as a life partner with whom a pregnancy is desired as discussed above in the human fertility scenario of FIG. 19. And further as an advantage of the vibrating skin touch form of communication provided by our electronic jewelry, such notification is imperceptible to other persons who may be in the immediate vicinity, as such, this vibrating touch is received privately and discreetly, without the highly noticeable visual sight of a person staring at a mobile phone to scroll through text messages or email while attention may be needed elsewhere or having to wear a pair of ear buds a or a single ear bud at work during a meeting in a conference with colleagues present, for example. Further, the electronic jewelry hereof may be considered substantially preferable over other electronic commutation devices where such jewelry is an attractive display item of personal fashion, that may be preferred as more appropriate in certain settings such as at movie theater, attending religious services, or while attending a performing arts event such as a Broadway show or orchestral performance, with such electronic jewelry further being a preferred pairing with the clothing attire that may be desired at such events.

Thus with the above at hand as to the vibrating skin-touch sensation of our above electronic jewelry serving as a method of silent and discreet sensory communication or notification as facilitated over a local communications network, the electronic jewelry items hereof may thus be easily extended into use scenarios over wider-area networks that take advantage of existing global telecommunications infrastructure. As such and in adjacency therewith, one of the inventors hereof along with certain other co-inventors have provided a post-pandemic school building that implements new health and wellness methods, apparatus, and systems within the traditional modern school building to allow school children to continue to attend in-person classes at school during pandemic conditions so that their educational development is not adversely affected as was demonstrably shown with some of the remote-learning policies that were mandated during the COVID-19 worldwide pandemic of the years 2020 to 2023. Such health and wellness methods, apparatus, and systems for school buildings are fully disclosed in commonly assigned PCT International Publication No. WO 2021/163341 titled Diagnostic Patches And Bracelets For Promoting Personal And Community Health Including Related Processes, Methods, And Systems as filed on Feb. 11, 2021 under International App. No. PCT/US2021/017679 which has been incorporated herein so as to provide a technical backdrop for the next use scenario of our electronic jewelry items as discussed above in FIGS. 1A to 17A.

Thus with the above published patent application as technical backdrop, FIG. 22 presents a scenario setting illustrating yet another use application hereof showing how a network-enabled electronic jewelry device 461 of these inventions as worn by a first person such as a parent, may be deployed for the health, safety, and wellness of a child while attending class at school. First to set the scene, a school building 462 is provided with a health and wellness station 464 that includes a secure medical server 466. Upon registration of the child at school, the parent's phone 428, as used in the above scenarios of FIGS. 18 to 21, is here registered with the school and is thereby allowed to connect in a secure manner to a secure broadcast system 468. Next at system block 470, the child attending classes has been provided with a bio-patch such as the fever patch 432 discussed above in FIG. 17C. Then during the school day, if the child develops an elevated temperature, the fever patch 432 reports the elevated temperature the school medical server 466, which server in turn relays a packet of information for that student to the broadcast system 468, which broadcast system then securely sends a wireless communications single including the packet of information to the parent's phone 428, which phone, like above in the scenarios of FIGS. 18 to 21, sends a wireless signal to the jewelry item 461, which jewelry item then vibrates to provide a silent discreet alert notification to the person wearing the jewelry item, here in this scenario, that person being the parent of the child that has developed the elevated temperature at school, where upon sensing the vibration, the patent may then take any desired steps towards attending to their child. And here like above, the jewelry item 461 may be of the form of any of the electronic jewelry items hereof such as the finger ring assembly 156 of FIG. 3B, the butterfly-shaped finished clasp assembly 215 of FIGS. 8 and 10A, the finished inverted teardrop earrings 284 of FIG. 12B, the finished electronic jewelry item 330a of FIG. 13D, the wrist bracelet 350 of FIG. 14E, the electronic earrings 374 of FIG. 15A, and the stylized two-piece electronic jewelry item 377g of FIG. 15I as discussed above in further connection with FIG. 17A.

Thus generally with regard to the electronic jewelry items of FIG. 17A, as may be deployed across any of the use scenarios of FIGS. 18 to 22, there is provided a network-ready piece of jewelry such as any one of the jewelry items 156, 215, 284, 330a, 350, 374, 377, for example, which all generically include i) a body portion intended for wearable display by a user 440 thereof, said body portion including the from-side and/or the back-side pieces of our two-piece electronic jewelry items; ii) a vibration member implemented to vibrate in a predetermine manner and positioned in an affixed manner relative said body portion such that the user 440 may feel a vibration when said vibration member vibrates in said predetermined manner; and iii) a receiver component implemented in association with said body portion to form a wearable assembly thereby being enabled to receive an activation signal from a remote device so that when the remote device sends a respective activation signal to said receiver component, the vibration member vibrates to thereby provide an alert to a user 440 of the wearable assembly. Thus when worn for advantageous use according to the teachings hereof, the above network-ready piece of jewelry may be deployed within a system for personal notification comprising the jewelry item wherein the remote device is a smartphone 428 associated with the user 440 of the wearable assembly, or wherein the remote device is a wearable diagnostic patch 432, 434 worn by the user 440 of the wearable assembly. There is also provided a system for second-party notification where the above network-ready piece of jewelry is worn by a first person 440 wherein the remote device is a wearable diagnostic patch 432, 434 worn by a second person 456, 470.

In a similar manner to the above, there are more specific embodiments of a network-ready piece of jewelry of the post and clasp type 284, 374, and 377, for example, that comprise i) a body portion or front-side piece intended for wearable display by a user 440 thereof, said body portion or front-side piece having a post, ii) a clasping component or back-side piece implemented for engaging the post of said body portion or front-side piece in a secure manner and disengaging from the post when so desired by a user 440 thereof, iii) a vibration member affixed in assembly with said clasping component or back-side piece and implemented to vibrate in a predetermine manner, and iv) a receiver component affixed in assembly with said vibration member and with said clasping component to form a wearable assembly with said body portion or front-side piece, said wearable assembly thereby being enabled to receive an activation signal from a remote device so that upon receipt of said activation signal by said receiver component, said vibration member vibrates to thereby provide an alert to the user 440 of said wearable assembly. And in connection therewith, like above, there is further provided a system for personal notification comprising such a network-ready piece of electronic jewelry wherein the remote device is a smartphone 428 associated with the user 440 of the wearable assembly, or wherein the remote device is a wearable diagnostic patch 432, 434 worn by the user 440 of the wearable assembly. And in various alternate system implementations, there is provided a system for second-party notification comprising the above post-type network-ready piece of jewelry as worn by a first person 440 wherein the remote device is a wearable diagnostic patch 432, 434 worn by a second person 456, 470.

And still further in connection with the general discussion of FIGS. 17A to 22, as further supported and described there-above, there is also provided a network-ready piece of jewelry, for example 284 or 377, comprising i) a body portion or front-side piece intended for wearable display by a user 440 thereof, said body portion or front-side piece having a post, ii) a clasping component or back-side piece implemented for engaging the post of said body portion or front-side piece in a secure manner and disengaging from the post when so desired by a user 440 thereof, iii) a vibration member affixed in assembly with said body portion or front-side piece and implemented to vibrate in a predetermine manner, and iv) a receiver component affixed in assembly with said body portion or front-side piece to form a wearable assembly with said clasping component or back-side piece, said wearable assembly thereby being enabled to receive an activation signal from a remote device so that upon receipt of said activation signal by said receiver component, said vibration member vibrates to thereby provide an alert to the user 440 when wearing said wearable assembly. Here also in association therewith, there is further provided a system for personal notification comprising such a network-ready piece of jewelry wherein the remote device is a smartphone 428 associated with the user 440 of the wearable assembly, or the remote device is a wearable diagnostic patch 432, 434 worn by the user 440 of the wearable assembly. And as further extended, like above, there is also provided a system for second-party notification comprising such a network-ready piece of jewelry as worn by a first person 440 wherein the remote device is a wearable diagnostic patch 432, 434 worn by a second person 456, 470.

And still further according to this related subject matter hereof, in view of the discussion of FIG. 15B as to a co-axial post for conducting electric current from a front-side body portion to a back side clasping component, there is more particularly provided a network-ready piece of jewelry comprising i) a body portion intended for wearable display by a user 440 thereof, said body portion having a post, ii) a rechargeable battery implemented within said body portion and electrically engaged with said post of said body portion, iii) a clasping component implemented for engaging the post of said body portion in a secure manner and disengaging from the post when so desired by a user 440 thereof, iv) a vibration member affixed in association with said clasping component and implemented to vibrate in a predetermine manner, said clasping component and said vibration member forming a vibrating clasp, and v) a receiver component affixed in association with said body portion to form a wearable assembly with said vibrating clasp, said wearable assembly thereby being enabled to receive an activation signal from a remote device so that upon receipt of said activation signal by said receiver component, said vibration member vibrates to thereby provide an alert to the user 440 of said wearable assembly. And here also, there is further provided a system for personal notification comprising the above network-ready piece of jewelry wherein the remote device is a smartphone 428 associated with the user 440 of the wearable assembly, or wherein the remote device is a wearable diagnostic patch 432, 434 worn by the user 440 of the wearable assembly. And also in the extension thereof, there is also provided a system for second-party notification comprising the above network-ready piece of jewelry as worn by the first person 440 wherein the remote device is a wearable diagnostic patch 432, 434 worn by a second person 456, 470.

And still further as to these aspects of the present invention, there is alternatively provided a network-ready piece of jewelry comprising i) a body portion intended for wearable display by a user 440 thereof, said body portion having a post, ii) a clasping component implemented for engaging the post of said body portion in a secure manner and disengaging from the post when so desired by a user 440 thereof, iii) a vibration member affixed in association with said body portion and implemented to vibrate in a predetermine manner, said body portion and said vibration member forming a vibrating body, and iv) a receiver component affixed in association with said clasping component to form a wearable assembly with said vibrating body, said wearable assembly thereby being enabled to receive an activation signal from a remote device so that upon receipt of said activation signal by said receiver component, said vibration member vibrates to thereby provide an alert to the user 440 of said wearable assembly. And then again here, like above, there is provided in conjunction therewith a system for personal notification comprising this type of network-ready piece of jewelry wherein the remote device is a smartphone 428 associated with the user 440 of the wearable assembly or alternately within the system, the remote device is a wearable diagnostic patch 432, 434 worn by the user 440 of the wearable assembly. And then as an extension thereof, there is also provided a system for second-party notification comprising this particular network-ready piece of jewelry as being worn by a first person 440 wherein the remote device is a wearable diagnostic patch worn 432, 434 by a second person 456, 470.

And lastly for now across the specific subject matter of FIGS. 17A to 22, as supported by the discussion and additional drawing figures provide there-above, there is provided a network-ready piece of jewelry comprising i) a body portion having a backside with at least a portion thereof including a flat area, ii) a vibration member having a first surface and a second surface opposed thereto, said vibration member being implemented to vibrate in a predetermine manner, iii) an adhesive applied to said first surface of said vibration member and thereby attached to the flat area on the backside of the body portion, and iv) a receiver component affixed in assembly with said body portion thereby forming a wearable jewelry assembly, said receiver component implemented to receive an activation signal from a remote device so that upon receipt of said activation signal by said receiver component, said vibration member vibrates to thereby provide an alert to a user 440 wearing said wearable jewelry assembly. And here also in conjunction therewith, there is provided a system for personal notification comprising this particular network-ready piece of jewelry wherein the remote device is a smartphone 428 associated with the user 440 of the wearable jewelry assembly, or wherein the remote device is a wearable diagnostic patch 432, 434 worn by the user 440 of the wearable jewelry assembly. And then again also here as extended, there is further provide a system for second-party notification comprising this specific network-ready piece of jewelry being worn by a first person 440 wherein the remote device is a wearable diagnostic patch 432, 434 worn by a second person such as the infant child 456 discussed in FIGS. 20 and 21 or the child at school as discussed in connection with the system block 470 of FIG. 22.

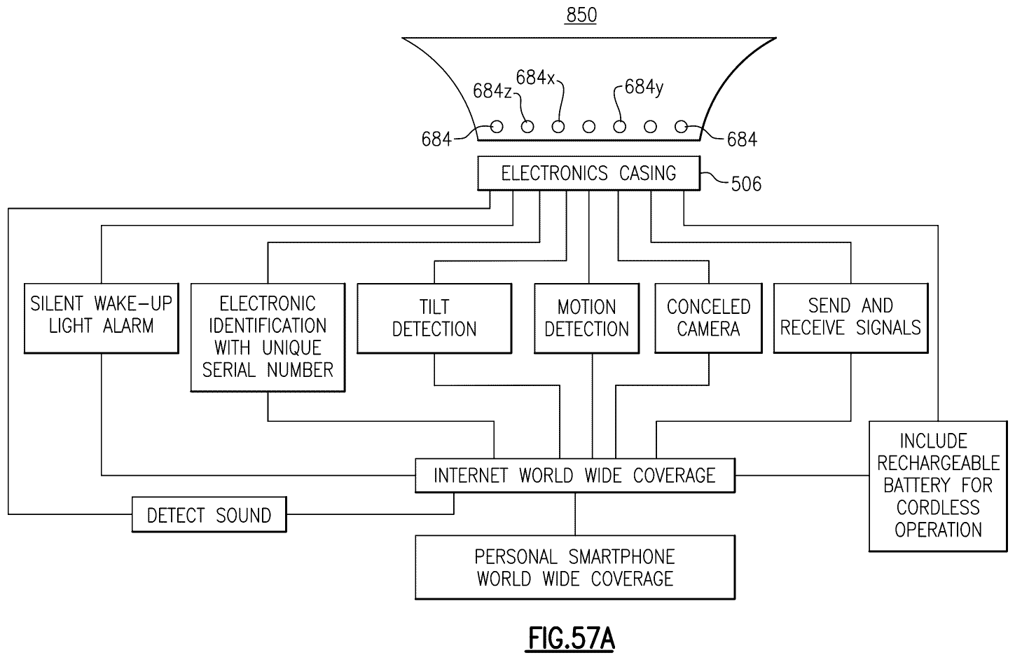

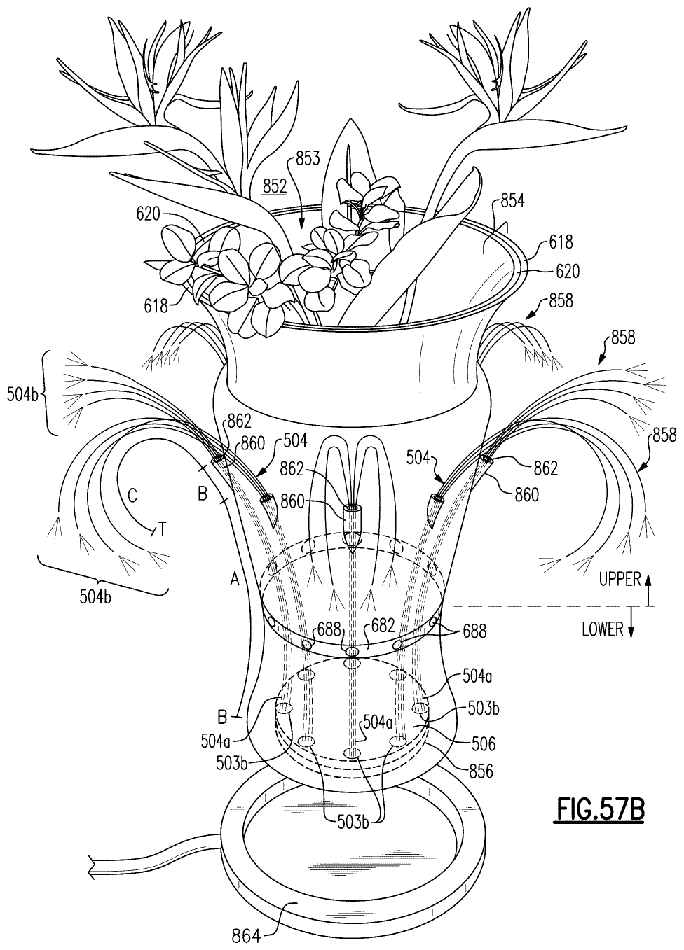

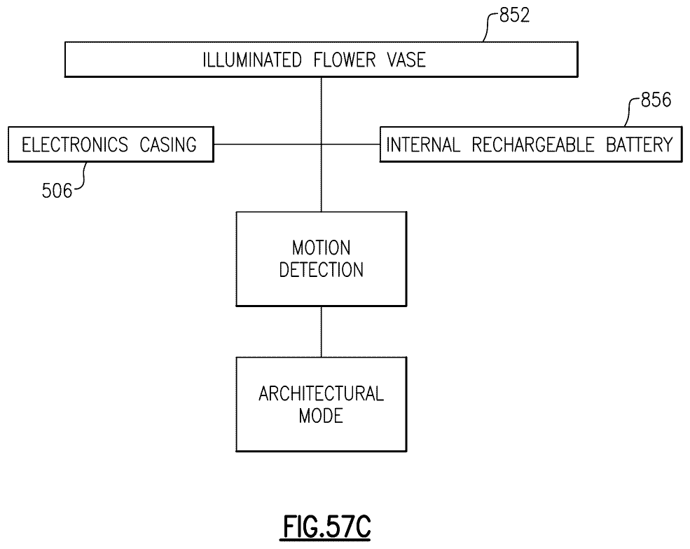

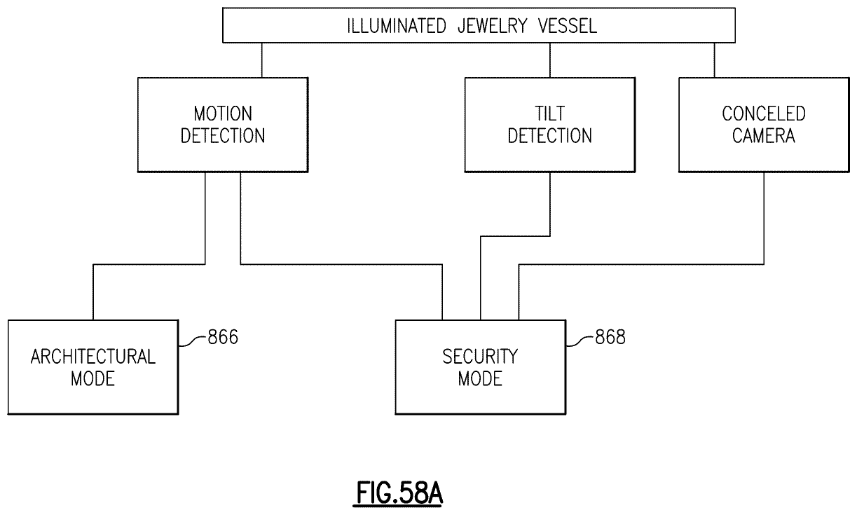

Now taking a break in direction from the above, it may be said generally that the modern smartphone has become a ubiquitous daily tool that, among other things like taking photos, listening to music, sending and receiving texts and emails, searching the Internet, and of course, making phone calls, further enables a person to remotely control a variety of different electro-mechanical systems implemented for domestic use. Such domestic systems currently include, for example, lawn watering systems, front door surveillance systems, house door locking systems, home lighting systems both interior and exterior, home heating systems, and systems developing in the automotive industry relating to daily use of motor vehicles such as electric motor vehicles and their charging systems. Thus in this developing trend as applied hereto, FIG. 23 is a pictorial representation of the jewelry box 402 of FIG. 16C interactively engaged with the smartphone 428 of FIG. 17B which includes our proposed “Life Jewel” app 430. As illustrated, the controller 416 in this embodiment is operably connected to the sound box 414 and is further provided with an electronic circuit board or commutations module 472 that enables functionalities in the controller 416, as connected to the sound box 414, to be remotely accessed by the smartphone 428. Thus the “Life Jewel” app 430 here in this embodiment includes further functionality that enables the phone 428 to remotely interact with the electronic jewelry box 402 in a variety of useful ways as discussed herein below in further detail in connection with FIGS. 57 and 58. The particular technical details of the module 472 and the app 430 as augmented to interact with our electronic jewelry box 402 are beyond the intended scope of this disclosure and thus are more appropriately presented in related companion disclosures intended for filing adjacent hereto.

Next with reference to FIGS. 24A, 24B, and 24C there is presented when taken together a flow chart showing the steps of a method of making a glass jewelry vessel according to yet still further aspects of the present invention and in FIGS. 25A, 25B, and 25C there is illustrated a corresponding series of perspective pictorials showing each of the method steps in FIGS. 24A, 24B, and 24C.

Thus now with reference to FIG. 24A taken in tandem with FIG. 25A, there is first presented a step 480 of providing a desired glass blowing mold having a number of sides and a bottom. Here, a five-piece mold 482 has been selected where the five-piece mold 482 includes a bottom panel or plate 484 and four side panels or plates 486, 488, 490, and 492 where each of the side plates is configured to taper outwardly relative to extending from the bottom plate 484 upwardly to the open top as illustrated. At step 494, molten glass of a desired type and color is prepared and by use of a traditional glass-blowing tube or pipe 496, a first layer of glass is blown into the mold 482 at step 498. Next at step 500, light emitting elements 502 and 504 are applied onto the first glass layer. Here, preferably, the first layer of glass is still in a semi-soft state such that the light emitting elements 502 and 504 may be pressed into the first layer of glass. As illustrated schematically, light emitting element 502 is a strip of LED lights including three light sources or indicator lights 503 numbered 1, 2, and 3, and light emitting element 504, as shown schematically, includes a bundle of optical fibers 504 comprised of individual optical fibers 505 as may be bundled together at one end thereof to form an input end 504a where at the other end of the bundle 504, the individual fibers are loose and free such that they may be placed as desired to form an output end 504b of the bundle 504 as illustrated here and discussed in further detail below. Herein the indictor lights 503 may function in a similar manner as to the indictor lights 174 provided in the electronic charging cases and boxes 165 and 372 discussed above in detail in connection with FIGS. 4A to 4M-2 and 15A and in a similar manner to the indictor lights 406 provided in the above electronic jewelry box 402 as used with traditional and electronic jewelry as discussed in connection with above FIGS. 16A to 16C and thus as such, discussion of this functionality of the indictors lights 503 need not be repeated here.

Next provided in this manufacturing method as a supplied input item, is an electronic circuitry casing or electronics casing 506 here including a knock-out or marking 507 which marks the location of an electric socket in the casing as discussed below in further detail. The electronics provided in the casing 506 may be of any desired functionality including at least electronic functionality for controlling at least the light emitting elements 502 and 504 and for providing operability to a charging pad implemented therein for recharging personal electronic items such as smartphones and electronic jewelry. The type of charging pad provide therein is of the type discussed above in detail in connection with FIGS. 4B, 16B, and 16C as implemented in various embodiments across all the charging cases and electronic jewelry boxes hereof as discussed above in FIGS. 2A and 2B, FIGS. 3A and 3B, FIGS. 4A to 4M-2, FIGS. 13A and 13B, FIGS. 13C and 13D, FIGS. 14B to 14F, FIG. 15A, FIGS. 16A to 16C, and FIG. 23, and as further discussed below as included with our blown-glass and sculpted-stone illuminated charging vessels for smartphones and electronic jewelry. As discussed above as to the electronics manufacturer working in collaboration with a traditional jewelry maker to provide the electronic jewelry items hereof, such an electronics manufacturer may also be suited to working in collaboration with the inventor-artisans hereof who have designed and created the blown-glass and sculpted-stone vessels hereof. As such, the particular technical details of the electronic circuitry provided in the casing 506 are considered beyond the intended scope of this disclosure and thus will be provided in companion disclosures as may be filed adjacent hereto.

Thus upon receipt from the electronics manufacture, or prior thereto, the electronics casing 506 may be certified as tested for full functionality as designed before placing same into inventory as an input item to the present method.

Moving along now to step 508 next in FIG. 24A, the casing 506 is fitted onto the first blown glass layer and at step 510, the casing is electrically connected to the light emitting elements 502 and 504. Here also, an electronics and lights testing may be performed before proceeding to the next step, if desired in the present method. And also, like above, the first glass layer may still be soft when placing the casing 506 thereon or it may be in a cooler more solidified state. Then at step 512, a second glass layer is blown into the mold 482 to thereby encapsulate the casing 506 and the lighting elements 502 and 504 between the two layers of glass. Then during any of the above steps or the steps hereof following, next as per FIGS. 24B and 25B, the mold 482 may be place in an oven 516 under step 518 to heat or cool the contents thereof at that time in the method as may be desired to avoid cracking, promote annealing, slowly change from a higher temperature to a lower temperature for further processing, slowly change from a lower temperature to a higher temperature for further processing, or more quickly cool or heat the glass so as to promote a crackled effect in one or both of the glass layers as all of such is well known in the creative glass blowing arts where electronics, like those here, may not be known or included within the item there traditionally at hand.

Now with continuing reference to FIGS. 24B and 25B, at step 520, the mold 482 is removed from the oven 516 and a shaping block 522 is placed into the mold onto the interior surface of the second layer of glass and may be gently pressed there-into and there-against to give a certain final desired shape to the interior shape of the glass vessel here under formation. The shaping block 522 is thusly important here for at least two principal reasons; first, the shaping block 522 shapes the interior of the glass vessel to thereby yield more precise tolerances as to the interior dimensions such that manufactured inserts such as jewelry trays or compartment dividers may be better ensured to fit as intended therewith and then second as to the importance of the shaping block 522 in this method, to provide structural support to the glass vessel inside the mold during further processing and final cooling to reach a room-temperature final solidified state. And then as also indicated at step 520, this now temporarily-assembled, shaping-block-on-glass-in-mold assembly is inverted and placed on the top surface of a work bench 524. Next so as to provide attractive and functional feet to our glass-blown vessel, at step 526 with the vessel here being at a desired temperature for further processing as removed from the oven 516 to achieve same, the bottom plate 484 of the mold is removed to expose the exterior surface of the bottom 528 of the glass vessel. Meanwhile, the side plates 486, 488, 490, and 492 at this stage of the process remain connected and in tack against the blown glass on the sides so as to provide lateral support to the corresponding glass side walls of the vessel while still pressing against the shaping block 522 that remains there-under in the vessel while atop the work bench 524 as illustrated in the schematic pictorials here in FIG. 25B at this stage of the present method. So here next at step 530, glass balls 532 in a semi-molten state may then be applied to the exposed glass bottom 528 while the side plates 486, 488, 490, and 492 continue to hold the assembly in lateral support. Here the glass vessel may be allowed to begin to cool to thereby start its approach to a final state of solidification while as is well known in the creative material arts of glass blowing, a torch or blow-torch of the oxyacetylene type or other type may be use to provide localized heating at the location of the feet to be applied here as initially semi-molten balls of glass 532. In this manner, a better bonding may be achieved between the semi-molten glass balls 532 and the heated or re-heated localized areas on exterior surface of the bottom 528 of the glass vessel. Then within a timing tolerance as to the pliability of the applied glass balls 532, at step 534, the assembly is turned up-right on the work bench 524 and while the glass balls 532 are in a certain desired pliable state, a slight downward pressure as indicted by pressure arrows 536, is applied to the assembly so that the feet may be squeezed or squashed a bit, thus deforming same into semi-oblong pucks or disks with rounded side edges in an artistic manner to thereby hand form custom feet for each vessel such that when placed in use, the top of the vessel will set level with a night-stand, for example, as illustrated above in FIG. 19 as to the vessel 452 shown therein in connection with our discussion of certain use scenarios of the electronic jewelry items hereof as deployed within a wireless network infrastructure. And now here again in FIGS. 24B and 25B, at step 538, the side plates are removed to thereby release from the mold a semi-finished version of a glass vessel 540 made according to this method such that in a back side 542 thereof, an opening 544 may be formed to thereby access an electronics port or socket previously provided in the electronics casing 506 discussed above. Here the opening 544 in the glass vessel 540 and also a corresponding opening as marked by the knock-out or marking 507 in the electronics casing 506 may be achieved by glass and ceramic grinding tools well know in the art, or may be otherwise formed in the glass by cutting the glass while in a semi-molten state or otherwise by re-heating locally by either of, or a combination of both of, these techniques, and then with delicate hand grinding tools in use, a corresponding opening in the casing 506 at the location of the knock-out 507 may be formed to expose the electrical socket previously provided therein.

Thus as discussed in FIGS. 24A and 24B along with the corresponding pictorials in FIGS. 25A and 25B, these aspects of the present invention are directed to a method of making a glass jewelry box or changing vessel 540 having light display notification and recharging functionality for use with personal electronic items, the method comprising the steps of providing a mold 482 configured in a pre-determined shape to form a glass vessel; blowing molten glass into the mold 482 to form a first layer of glass having an outside surface and an inside surface; applying at least one light emitting element 502, 504 onto the inside surface of the first layer of glass in a selected location; and blowing molten glass onto the inside surface of the first layer of glass to form a second layer of glass to thereby encapsulate said at least one light emitting element 502, 504 between the first and second layers of glass.

Now with referenced next to FIGS. 24C and 25C, to continue with further aspects of this method, at step 546, when the glass vessel 540 is removed from the mold 482, it may be of a desired temperature or material state to have cut herein on the exterior surface thereof, various desired artistic shapes which is well know in the arts of making leaded-glass crystal stemware, decorative bowls, serving plates, flower vases, and the like. Alternatively, as know in the art, the vessel 540 here at this stage so as to perform the glass cutting step 546 may be place in the oven 516, FIG. 25B, to bring the temperature, material state, or both thereof, to a condition being optimal for such decorative glass cutting.

Next at step 548, after all the above manufacturing steps are performed, particularly those where heat is applied to the vessel 540 as a work-in-process item, the electronic circuitry contained within the electronics casing is tested using test equipment connected to the socket in the casing via the opening 544 to ensure full functionality thereof. And as would be understood by those of skill in the relevant arts, such a testing step or steps as to the electronic functionality, may be performed at any desired time during fabrication and assembly to ensure the good functioning thereof throughout the steps of this method of making such an item as the vessel 540.

Then to prepare for consumer use, at step 550, FIG. 24C, a cover 552 and a jewelry tray 554 with compartments may provided as illustrated in FIG. 25C. Then at step 556, the cover 552 and tray 554 may be fitted to desired tolerances in assembly thereof to be tailored as to a desired level of height and shape for fitting within the interior of the vessel 540 as discussed in further detail herein below in connection with FIGS. 53 to 56. Here, since the shaping block 522, FIG. 25B, was used to form the interior dimensions of the vessel 540 given the shape of the mold 482, tailoring needs for the cover 552 and tray 554 will be minimal or even unnecessary when the cover 552 and tray 554 are specified or “spec'd” as to the particular shaping block used in manufacturing. And lastly here, FIG. 25C shows a smartphone 558 and some of the electronic jewelry 560 hereof in dashed line in the bottom interior of the vessel 540 as representing the charging or recharging thereof in accordance with the teachings hereof.

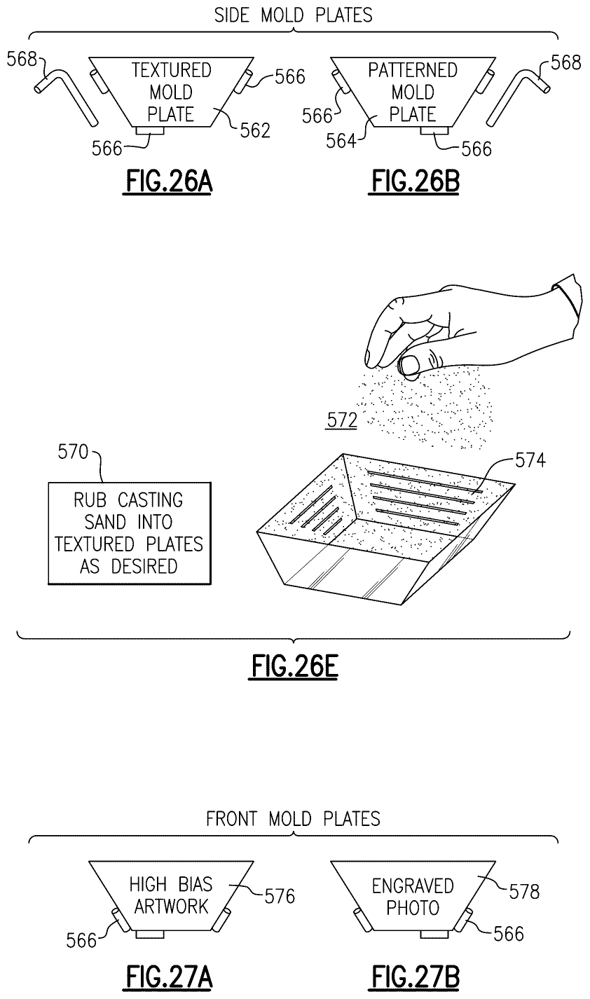

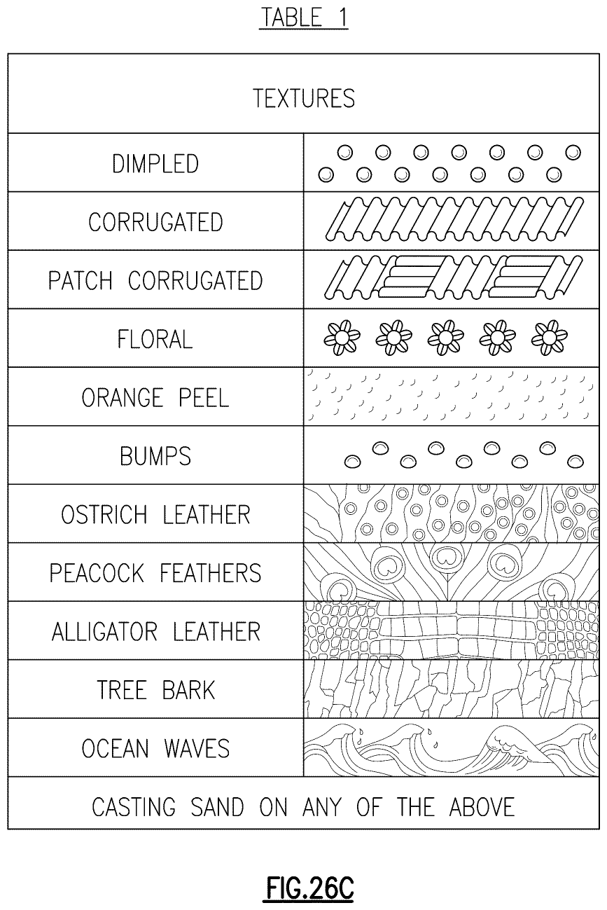

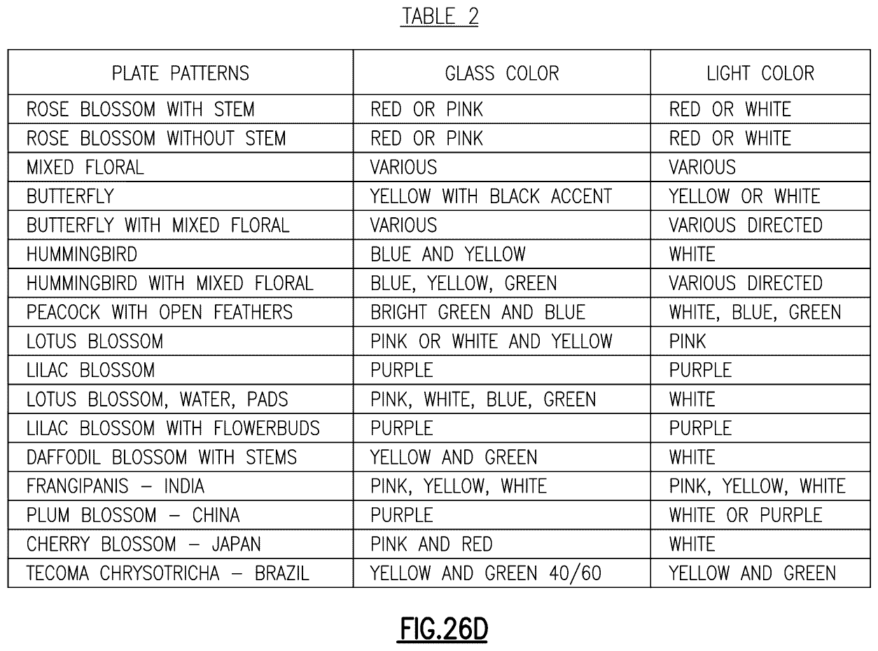

FIGS. 26A and 26B are pictorial schematic illustrations showing interchangeable side mold plates 562 and 564 of the type of side plates 486, 488, 490, and 492 discussed above in FIG. 25A to form the glass vessel 540 thereof, where here, according to further aspects of this invention, the side plates 562 and 564 are deployed to form certain preferred embodiments of a glass vessel of the type 540 by applying the methods of FIGS. 24A to 24C and 25A to 25C to thereby produce a more customized and even unique glass vessel according to these additional components and features as used in the various methods hereof for making an illuminated blown-glass charging vessel for charging mobile phones and electronic jewelry according to the various interrelated aspects of this disclosure. Thus now with more particularity, the side mold plate 562 of FIG. 26A is a textured mold plate, and the side mold plate 564 of FIG. 26B is a patterned mold plate. Each of the mold plates 562 and 564 have a series of hinge segments 566 that may be assembled to form a mold with a removeable bottom plate by use of a simple quick-release hinge pin 568 where such mechanical aspects of providing a mold for making glass blown items are well understood by those of skill in such mechanical arts.

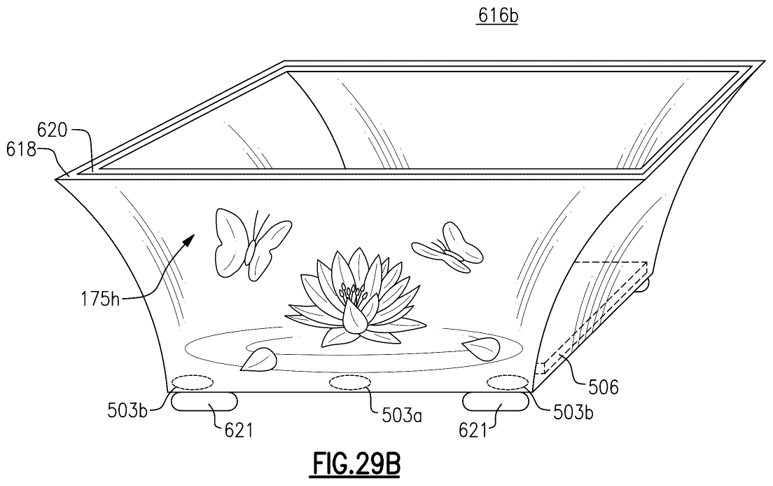

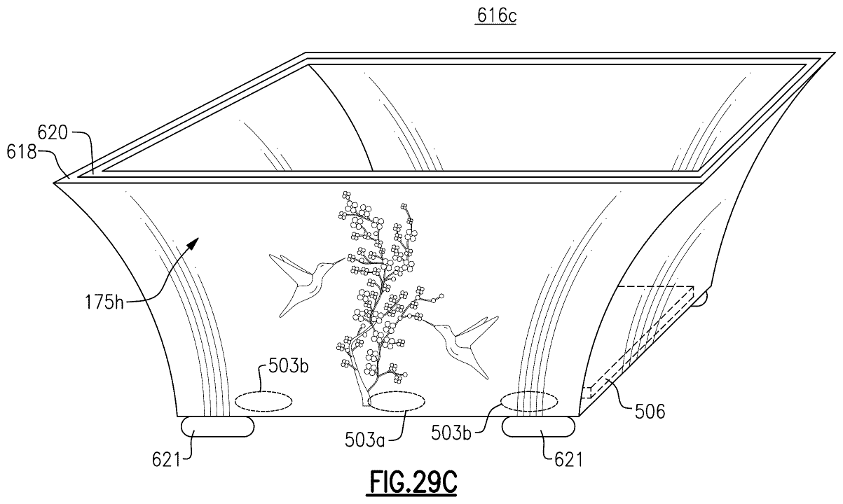

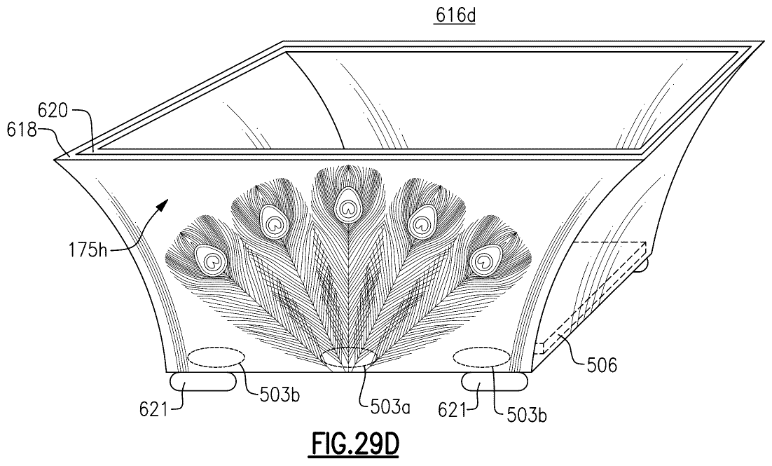

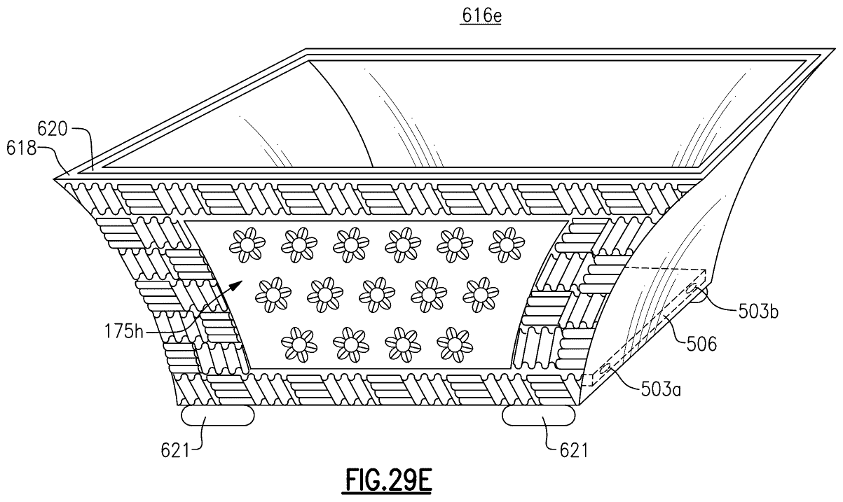

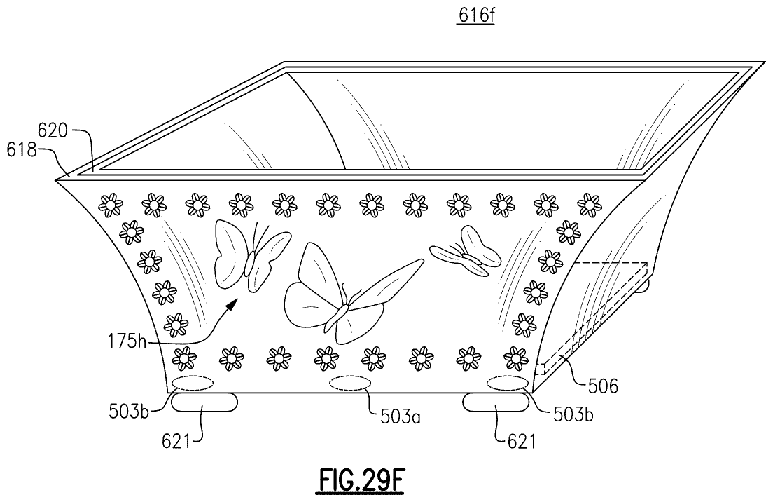

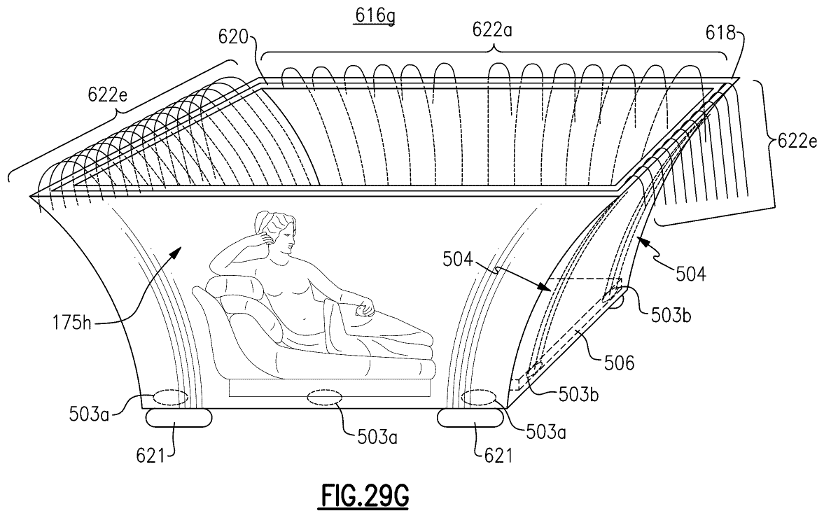

Examples of textures that may be engraved into or embossed onto the side mold plate 562, FIG. 26A, for either positive bias relief, or negative bias relief, or a combination of both positive and negative bias relief, as may be desired, are provided in an illustrated manner presented in FIG. 26C as Table 1 hereof. As shown, these examples of textures include dimpled, corrugated, patch corrugated, floral, orange peel, bumps, ostrich leather, peacock feathers, alligator or crocodile leather, tree bark, and ocean waves, to list a few where other such textures as may be desired on the exterior surface of our glass-blown vessels for a certain look, feel, or target market, may be easily provided in addition to those listed according to the teaching hereof as applied to these methods of making a textured glass vessel by the methods of FIGS. 24A to 24C and 25A to 25C. Such additional textures may be provided in a boarder or frieze around each side plate as in some of the below embodiments, for example, see FIGS. 29E and 29F, and may extend well beyond the list of Table 1 as desired to include, for example, long-familiar boarder or frieze designs from Greek or Roman architecture or from later renown design epochs such as from the Italian Renaissance or from the style-design period of Louis XVI such designs as in particular including, for example, the Ribbon-and-Reed or Egg-and-Dart motifs as may be used as boarders in the molding plates for making the glass vessels hereof.

FIG. 26D is a Table 2 hereof presenting visually by way of organized brief textual description, some representative examples of plate patterns that may be used with the mold plate 564 of FIG. 26B, along with corresponding glass colors, and selected light colors particularly suited to the patterns so as to make an illuminated glass charging vessel, as discussed above in conjunction with the methods of FIGS. 24A to 24C and 25A to 25C, by carefully curating the overall sensory experience with the vessel as animated by the shapes formed on the sides of the vessel, the colors of glass used to make the layers of blown glass comprising the vessel, the colors of light emitted from the light elements as encapsulated between the glass layers, and then further in some embodiments, the gently movable extensions of optical fibers that may project out from a top edge of the vessel, with all thereof being further discussed below in additional embodiments hereof. And like above with the textures, these patterns may be engraved into or embossed onto the side mold plate 564 of FIG. 26B, for either positive bias relief, or negative bias relief, or a combination of both positive and negative bias relief, as may be desired. Thus, provided in Table 2 of FIG. 26D are some selected examples of molding plate patterns, glass colors, and light colors according to these aspects of this disclosure. Here then first listed, is a pattern including a rose blossom with stem. In this rendering of our vessel, the glass color may be selected as red or pink with the first layer forming the exterior of the vessel in darker red glass, for example, and the second blown glass layer forming the interior of the vessel formed in pink glass. Thus in this embodiment with rose blossoms and stems formed on the exterior sides of the vessel, red or white indicator lights may be encapsulated therein to provide the animated effect of a rose garden as illuminated by sunlight which may be rendered more vibrant by activation of the lights being turned on or off, or otherwise controlled by smoothly changing the intensity of the lights, or by implementing any other light show arrangements as may be provided by the electronics associated therewith.

The next listed pattern in Table 2 is rose blossom without a stem. In this manner, the pattern created on the exterior of the glass vessel by such a mold plate 564, FIG. 26B, would be a series of randomly placed rose blossoms with some type of flat glass spacings provided there between. As to these flat spacing between the blossoms, they may be textured according to Table 1 such that the space between the blossoms may be sanded, dimpled, or orange peel texture, for example, to thereby create a more sensory experience by touch when feeling the vessel by touch-of-hand. Here also, preferably, red and pink glass may be used with preferably dark red glass on the exterior of the vessel and pink colored glass on the interior of the vessel. And like above, the encapsulated LED lights here may be of white color or to enhance the animation effect and the lighting mood that may be set in a room where the vessel will be placed into use, the lights here may be a deeper ruby red color.

The next example pattern that may be formed on the mold plate 564 of FIG. 26B, as provided in Table 2 of FIG. 26D may be that of a mixed floral pattern including roses, daisies, and daffodils, for example, or any desired combination of mixed floral patterns. Here in keeping with our curating aspects for selecting surface shapes, glass colors, and light colors to provide a sensory experience of a natural motif such as botanicals existing in nature, the glass colors and the light colors may be thus selected accordingly.

In keeping with the proposed botanical or garden motifs for our glass blown vessels, next listed in the Table 2 of FIG. 26D. is a butterfly pattern. As is understood under common life experience, butterflies existing in nature around the world at different locations occur in a wide variety of differing colors with each butterfly perhaps exhibiting two or three distinct colorations at most, in the main. Thus depending on the type consumer market that may vary according to country of sale or use, for example, a Brazilian rainforest motif verses an English garden setting in the mid-west of the U.S., various colors such as the Monarch butterfly colors of orange and black with some white speckling, may be selected or otherwise avoided in preference of yellows and black accents as suggested here in Table 2. Thus when the butterflies on the vessel are in yellow and black as shown, the proposed glass color and color of lights corresponding to the animated features hereof may preferably include yellow light, for example, as directed at the portion of glass colored yellow while white light may then be directed at the smaller portions of black glass utilized simply as an accent color.

Moving next down the list of patterns in Table 2 of FIG. 26D, to Frangipanis that may be associated with India, Plum Blossom of China, Cherry Blossom of Japan, and Tecoma Chrysotricha (Golden Trumpet Tree) or Paisagens of Brazil, it is understood by the inventors hereof that these natural motifs, as in some cases tied uniquely to certain geographical locations across the globe, may have cultural, literary, or spiritual symbolism associated therewith. For example, Chrysanthemums in China may be associated with the Chinese poet Tao Yuanming as a symbol of humility, while Cherry Blossoms (Sakura) in Japan symbolize a sense of springtime renewal and optimism tempered with a sense of the transience of life as a theme from Buddhism. Thus our glass vessel as rendered with cherry blossom patterns, particularly when animated with pink, white, and accents of red color glass and light, may be taken as preserving the transience of life over the useful life of our vessel. In a similar manner, as to cultural ties to a particular country as based on botanicals native to that country, the Tecoma Chrysotricha of Brazil may be provided on our vessel in its natural colors of yellow and green both as to glass colors and light colors with the design rendered so that about forty percent of the visual experience is in yellow color and about sixty percent of the visual experience with the vessel is in green color to thereby emote not only an feeling of natural botanicals associated with this national flower, but also emote tacitly a sense of national pride that may thereby be associated with the colors of the Brazilian flag or from those same colors as used on the uniforms of the national football or soccer team.

As such, suffice it to say here that our glass vessels may be carefully and thoughtfully curated to provide an overall sensory experience with the vessel as informed and animated by the shapes molded on the sides of the vessel, by the colors of glass used to make the layers of blown glass comprising the vessel, by the colors of light emitted from the light elements as encapsulated between the glass layers, and then further in some embodiments, as so informed and animated by the gently movable extensions of optical fibers that may project out from a top edge of the vessel, to thereby emote a feeling of cultural, literary, or spiritual heritage associated with the symbolism thereby provided by these curated renderings.

FIG. 26E is an additional method step and corresponding perspective pictorial representation of rubbing casting sand into the textured or patterned side plates 562, 562 of FIGS. 26A and 26B to thereby provide a unique outside surface on a glass vessel made according to certain preferred embodiments by the methods presented above in detail in FIGS. 24A to 24C and 25A to 25C. Thus here in FIG. 26E, there is provided an additional method step 570 that may be applied where desired according to these aspects of this invention where at this step, before blowing the first layer of glass, casting sand may be applied to the mold as comprised of textured or patterned plates, where desired. As such, the schematic pictorial illustrates a mold 572 having formed thereon a lined pattern 574. The lined pattern 574 is representative of any of the above textures or patterns discussed in conjunction with the textured mode plates 562 of FIG. 26A, or the patterned mold plates 564 of FIG. 26B. As such, in practicing the above method step 570 of applying casting sand onto the textured or patterned side plates 562, 564, before the first layer of glass is blown into the mold, a unique exterior surface of the glass vessel is thereby attained. Here, as would be understood by those of skill in the art, the casting sand may play its traditional role of providing particulate matter between the mold surface and the first blown glass layer such that the item of blown glass is more easily removed or released from the mold plates as not having been mechanically adhered thereto since the particulate matter provides a releasing factor to the process. In addition, this step provides an added aspect of this invention when a more generous amount of casting sand may be randomly applied to the side plates such that the texture or pattern is thereby obscured to some greater extent. As an advantage hereof according to these aspects of this invention, the textures and patterns provided by the mold plates are thereby rendered more distinctive in character such that while repeating this sanding step for each glass vessel as made from the same set of side plates, each glass vessel may be considered unique from the next made, with the same plates, when the casting sand is randomly applied as to its then added features to the pattern or texture thereby rendering each exterior surface of each glass vessel that much different from the next using the same plates to thereby create a uniquely rendered version of the glass vessel as made by repeating use of the same plates.

FIGS. 27A and 27B are similarly pictorial schematic illustrations showing interchangeable front mold plates according to additional aspects of this invention as deployed to perform certain further preferred embodiments of the methods shown and discussed above in conjunction with FIGS. 24A to 24C and 25A to 25C. Here first shown in FIG. 27A is a front mold plate 576 that has engraved or embossed artwork provided thereon. For example, the artwork may have a biblical theme such as Moses Parting the Red Sea, the Last Supper, or the Crucifixion, or the artwork may be a facsimile reproduction of well-known sculped statuary such as Michelangelo's David as being emblematic of the city of Florence, Italy representing youthful beauty, or the white marble rendering of Pauline Bonaparte as Venus Victrix by the Italian sculptor Antonio Canova which resides in the Galleria Borghese in Rome, Italy, for all the world to visit and behold. Thus in practicing the methods of making our glass vessels as discussed above, an added advantage thereto may be provided by making front mold plates in high bias which capture in facsimile reproduction, the imagery of such well-known sculped statuary or that which may be used from renown oil painting found on exhibit in various museums around the world which may be reproduced in this manner under use policies relating thereto.

And further in keeping with providing unique features to our illuminated glass vessels for charging personal electronic items as made by use of molds for forming imagery on the exterior surface of blown glass, the inventors hereof further provide methods and related components for taking a visual image and transferring or replicating same in bias relief on to the exterior surface of a glass vessel made according to the teaching disclosed herein. Thus in FIG. 27B, there is shown a front mold plate 578 that has engraved thereon, an imaged from a digital photo or a traditional paper photograph.

Now with the above at hand as to the custom mold plate 578 of FIG. 27B, then next turning to FIG. 28A there is provided therein a pictorial representation of receiving a number of visual images 580, here being of a pet as used in the following methods hereof to make a sculpted article of manufacture therefrom. Thus at a common step for these methods, there is provided a step 581 of receiving a number of visual images 580. These images in the typical scenario would be digital images taken with a smartphone, for example, or otherwise taken by a professional photographer with a specialized digital camera, or they may be traditional black-and-white or color photographs on photopaper. Here in this embodiment as to pet photos, at least three shots or poses of the pet have been prescribed which include an image of the body or a body shot, pic, or view 580a, then a close-up pic of the head or face 580b, and lastly here in this example, a clear shot of the hindquarters of the animal 580c including a good view of the shape and nature of the tail. Here as to pet type, a full-breed Shih Tzu canine is represented in the black-line sketchings of FIG. 28A for an example of distinctiveness as to facial features and tail shape which will be discussed as important featured elements of the methods below.

FIG. 28B represents a method for converting all or some selection of the visual images 580 of FIG. 28A into a three-dimensional digital image file of the pet pictured therein. Here at step 582, the images are scanned or otherwise transferred into a digital photo imaging system that may advantageously include computer-aided-design functionality, then at step 584 a three-dimensional or 3D image file of the featured pet is created.

Now returning to our illuminated blown-glass vessels as made according to the methods discussed above in conjunction with FIGS. 24A to 27B, here next in FIG. 28C there is provided an illustrated flow chart of the principal steps of a method for using the three-dimensional digital image file created at step 584 of the method of FIG. 28B as used here to operate a CNC machine to make a custom front mold plate 578 of the type presented in FIG. 27B. Thus first here at step 586, the 3D image file of the pet is received, or one such image file of several is selected, and then next at step 588, the selected 3D image file of the pet is digitally input into the operating system of a CNC machine, a type of automated manufacturing machine that includes an operating system having “Computer Numerical Control” or CNC functionality. Then at step 589, the CNC machine is used to make an engraved rendering of the selected image of the pet in the front mold plate 578, here that rendering being a close-up of the face of the canine as illustrated, And as would be understood by those of skill in the relevant art, a CNC machine includes a control arm 590 that has a cutting, welding, grinding, or other type of shaping or work tool 591 attached thereto that may be controlled to make cuts or shapes in a work piece using a water jet, laser light, or a sharpened hard-metal end-mill tool while accurately performing such in great detail down to very small dimensions. Thus here in this method according to these further teachings hereof, the control arm 590 and shaping tool 591 are deployed to make the engraved rendering of the canine face from the image 580b of FIG. 28A, into the inside surface of the front mold plate 578 as illustrated.

Thus next in FIG. 28D there is provided a perspective schematic view of a glass-blowing mold 592 having as a front plate thereof, the front mold plate 578 as made according to the method of FIG. 28C. Here, like above as to the five-piece mold 482 of FIG. 25A, the mold 592 is a five-piece mold including the bottom plate 484 and the three side plates 486, 488, and 490 where here in FIG. 28D, the side plate 492 of FIG. 25A has here been replaced with the engraved front mold plate 578. Thus as would be readily understood here in view of the above, the mold 592 including the front mold plate 578 as having engraved therein the rendering of the canine face from the image 580b of FIG. 28A, may be used as a custom glass-blowing mold in practicing the steps of the method presented in FIGS. 24A to 24C.

Thus to further exemplify the above visually for the benefit of those interested herein, FIG. 28E provides a perspective view of a custom illuminated glass charging vessel 594 made using the front mold plate 578 as discussed above in reference to FIGS. 28C and 28D. Here the custom vessel 598 includes the canine face image as a molded glass feature on the front side of the vessel which is also shown as including a molded boarder formed from the patch corrugated texture presented in Table 1 of FIG. 26C as discussed in conjunction therewith and generally with the textured mold plate 562 of FIG. 26A.

Thus in view of the above in connection with FIGS. 28A to 28E as discussed so far above, it should be apparent from the teachings hereof that the photos represented above in FIG. 28A needed not be limited to photos of a beloved family pet, but may include here personal photos of any beloved person, place, or thing as submitted and received as initial input for these methods of making a custom front mold plated as represented by the front mold plate 578 in FIGS. 28C and 28D. This grouping of personal photos thus, for further example, may be that of a first born infant child, a child lost prematurely to an unexpected death, a parent, or other such beloved person. The grouping of photos may also be of a beloved place such as a temple garden in Kyoto Japan having raked small stones with large rocks interspaced there-among, glimpses of temple buildings, various garden plantings such as towering bamboo stalks with new-growth green shoots of buds and new leaves, perhaps being planted and growing along a stone pathway, and with all of such perhaps with an image of a single person in amongst the photos submitted. And as represented above in FIG. 28A, the subject of the submitted photos may be that of any beloved family pet. Then, with the 3D image file thereof, any desired type of custom front plate 578 may be created by these methods for a one-time or limited special production use to make just one or some restricted number of special production glass vessels such as the glass vessel 594 shown in FIG. 28E.

Thus as to FIGS. 28A to 28E, the inventors hereof further provide a method of making a custom mold plate for making therefrom a customized blown-glass illuminated charging vessel, said method comprising the steps of i) receiving a collection of personal photo images; ii) creating an image file from the photo images; iii) inputting the image file into a CNC machine and therewith cutting a mold plate having an image from the image file engraved thereon; and iv) using the mold plate with the engraved image to make a glass vessel according to the method of FIGS. 24A to 24C. And in a particular embodiment thereof, the photo images may include selected images of a beloved family pet such as the Shih Tzu shown in FIG. 28A.

Next as to making custom electronic jewelry items as to further integrated teachings hereof, FIG. 28F presents a flow chart showing the principal steps of making a custom electronic jewelry box according to yet still further aspects of these inventions. Here at an initial step 596, a collection of photos is received and for purposes of this method will be taken as the images 580 of the pure-breed Shih Tzu canine discussed above in FIG. 28A. Next at step 598 back in FIG. 28F, the photos 580 are input into a CAD system for integration and further digital processing and with this processing then next at step 600, a 3D image file of the pet canine is created from the photos 580. Then at step 602, by use of a 3D printer, a 3D scale model of the pet is formed. And finally at step 604, the 3D scale model of the pet canine is sent to a skilled artisan for rendering the jewelry box according to specifications provided therewith. Such a skilled artisan, craftsperson, or expert may include those with combined expertise across the arts of taxidermy, custom doll-making, and scale-model renderings as to architectural designs as coordinated by the wholesale provider of the box in collaboration with the electronics provider with further technical specifications provided as to the desired electronic functionalities of electronic jewelry boxes as discussed above in detail in conjunction with FIGS. 4A to 4M-2.

The resulting from the method of FIG. 28F, FIG. 28G provides a perspective illustration of a custom-made electronic jewelry box or charging case 606 in a canine version as made uniquely, solely, and expressly for the owner of the beloved pet illustrated in the images of FIG. 28A. Thus here, like the feline version of the charging box 165e in above FIG. 4M-1, the sculpted canine jewelry box 606 illustrated here in FIG. 28G includes sculpted exterior side walls in the likeness of the canine pictured in the submitted photos, the corresponding hinged top cover, with thereunder provided a box with charging receptacles for recharging electronic jewelry items hereof as discussed above in detail and thus not repeated here. Further, the electronic jewelry box or charging case 606 in this canine motif, may include the indictor lights where such may be deployed, like above, to animate the eyes of this canine rendering.

Thus according to these aspects of this invention as discussed in conjunction with FIGS. 28F and 28G, the inventors hereof further provide a method of making a custom electronic jewelry box, said method comprising the steps of i) receiving a collection of photo images of a pet; ii) creating a three-dimensional image file of the pet from the photo images; iii) using the image file to 3D print a scale model of the pet; and iv) using the scale model of the pet to make a custom electronic jewelry box of the types illustrated in FIGS. 4M-1, 4M-2, and 28G.

FIG. 28H is a flow chart showing the principal steps of receiving the pet photos 580 of FIG. 28A and then making a sculped electronic jewelry piece hereof as based thereon. Here in this method, like above in the method of FIG. 28F, initially at step 596, a collection of photos is received and for purposes of this method will be taken again as the exemplary images 580 of the full-breed Shih Tzu canine discussed above in FIG. 28A. Next at step 598 back in FIG. 28H, the photos 580 are input into a CAD system for integration and further digital processing and with this processing then next at step 600, a 3D image file of the pet canine is created from the photos 580. Then at step 602, by use of a 3D printer, a 3D scale model of the pet is formed, here the scale model may preferably be of the same size as the jewelry item to be made therefrom, or may be larger than the final jewelry item to thereby show finer detail. Then next at step 608 of this method, the scale model made at step 602 is delivered to a jewelry maker and then at step 609 hereof, a jewelry artisan makes a sculpted jewelry piece based on the scaled model according to specifications provided therewith to thereby render the jewelry piece in the likeness of the pet as provided in the images 580 received at step 596 hereof. Such a skilled jewelry artisan may include those currently engaged with traditional jewelry makers who now in collaboration with the electronics manufacture implements further technical specifications provided as to the desired electronic functionalities of these electronic jewelry items as discussed above in detail in conjunction with FIGS. 15A to 15I. As such, the finished sculpted jewelry pieces from step 609 are then sent to a test and assembly facility where there, electronic parts are fitted with the sculpted jewelry piece as provided at step 610 and discussed above in detail, and then finally at step 611 of the method here in FIG. 28H, the finalized electronic jewelry item is tested for operational functionality for sending and receiving wireless signals, where so enabled, tested for vibrating upon receipt of a certain personal notification signal when so enabled, and tested for detecting a snoring sound, when so enabled, with such functionalities discussed above in detail and thus not repeated here.

As such, relative to providing a custom version of our electronic jewelry as made uniquely, exclusively, and expressly for the owner of the beloved pet, as illustrated in the canine images of FIG. 28A, for example, as to these aspects of this invention, where further the jewelry item is rendered in the likeness of that same beloved pet, now FIG. 28I so provides a perspective illustration of two different types of custom two-piece electronic earrings made according to the method of FIG. 28H that then may be offered exclusively to only the owner of the pet for their exclusive use and display as hereby intended and enabled. Thus in this manner, the owner of such a beloved pet may take advantage of these methods as provided in the open market place to thereby easily procure a unique jewelry item emblematic or otherwise in memory of a beloved pet which jewelry item may be further gifted to a child on a special occasion such as a quinceañera or wedding, where such child may have grown up over many years while living in the same household with such a beloved pet over such a number of years together.

Thus according to these aspects of this invention as here discussed in conjunction with FIGS. 28H and 281, the inventors hereof further provide a method of making a custom two-piece electronic jewelry item of the type discussed above in FIGS. 15B to 15P where this particular method includes the steps of i) receiving a collection of photo images of an object; ii) creating a three-dimensional image file of the object from the photo images; iii) using the three-dimensional image file to 3D print a scale model of the object; and iv) using the scale model of the object to make a two-piece electronic jewelry item in the likeness of the object. And in a preferred embodiment thereof, the photo images may be of a beloved family pet such as the full-breed Shih Tzu pictured in the images of FIG. 28A.

Now moving on to FIG. 29A, there is provided a schematic perspective illustration of an illuminated glass charging vessel 616a made according to the method of FIGS. 24A to 24C, as discussed above in detail. Here the vessel 616a has optical fiber bundles 504 encased or encapsulated between a first layer of glass 618 and a second layer of glass 620 as prescribed above at step 512 of FIG. 24A. And then here further, according to additional teachings hereof, the above step 512 is performed by the glass blowing artisan in accordance with lighting specifications for this embodiment to form a fringe pattern 622 of optical fiber extension segments 623 protruding up and over an upper or top edge 624 of the vessel 616a. In this manner, the indictor lights 503 may be positioned to provided light into an input end of each bundle of optical fibers 504 such that in use, light will travel up the bundle, thereby illuminating the vessel, and then extend along the extensions 623 to provide a subtle light show of animated light given the preset or otherwise programable functionalities of the indictor lights 503. Further, the extensions 623 may be formed from a flexible optic fiber such that the extensions 623 are gently movable by touch of hand or otherwise gently movable by application of moving air such as created by a ceiling fan, wind from an open window, or conditioned air moving out from a wall register in proximity to placement of the vessel in use. And here, as provided by the above method of making, the vessel 616a may advantageously include the electronics casing 506 as positioned in the bottom thereof to enable the desired functionalities of illumination and charging as discussed herein.

And as would be understood readily in view hereof, from a structural perspective, it may be said that the vessel 616a of FIG. 29A is formed by side walls 175 and a substantially planar bottom 528 formed integral therewith as may be understood across all common types of glass-on-glass vessels 616 disclosed herein and here in particular comprised of four side walls including side wall 175h as being a front side wall of vessel 616a, side wall 175i as being a back side wall thereof, side wall 175j as being a left-side side wall of the here illustrated vessel 616a, and side wall 175k as being a right-side side wall thereof as such individual wall references may commonly apply to any of the vessels 616 hereof without the need of repeating same therewith for purposes of visual clarity in the following FIGS. 29B to 29G. And further, like the above electronic jewelry boxes 165 of FIGS. 4A to 4K, the glass-on-glass vessels 616 here in FIGS. 29A to 29G, need not be limited to four side walls but may have any desired number of straight or curved side walls and corresponding geometric configurations from round with one closed loop side wall 175, such as shown in FIG. 4C and applied hereto as may be desired, or heart shaped with one such curvilinear side wall 175, as shown in FIG. 4D and as may be applied hereto, or even butterfly-shaped as to complex shapes, such as shown in FIG. 4E and applied hereto by application of skilled artisans from the glass-blowing arts, or as to regular polygon shapes from triangular including three side walls 175, square or rectangular including four side walls 175, or pentagonal including five such side walls 175, or any further such as would be understood in view hereof. And now here in these preferred embodiments, all the vessels 616 are rectangular in configuration having four side walls 175h, 175i, 175j, and 175k, as shown, that extend up from the bottom 528 and taper outwardly as so provide by their respective side mold plates 486, 488, 490, 492, and bottom mold plate 484, discussed above in detail in connection with FIGS. 25A and 25B.

Next in FIG. 29B, there is provided a pictorial perspective illustration of an illuminated glass charging vessel 616b with supporting feet 621 made according to the method of FIGS. 24A to 24C having a front side wall 175h as formed from a corresponding front mold plate of the type 576 of FIG. 27A, here including a mix of lotus blossom and butterfly motifs as selected from Table 2 of FIG. 26D. Here, indicator light or light source 503a, as encapsulated between the two different layers of glass 618 and 620, may be of a pink color, to illuminate the lotus blossom rendering which may be formed in pink color glass, while the indicator lights or light sources 503b may be of white light to illuminate the butterfly renderings which may be formed from yellow color glass with black glass accents, as provided in Table 2. And here also, as provided by the above method of making, the vessel 616b may advantageously include the electronics casing 506 as positioned in the bottom thereof to enable the desired functionalities of illumination and charging as discussed herein as to the light sources 503a and 503b as well as the charging pads that may be included therein for recharging personal electronic items such as a mobile phone or electronic jewelry when placed in the vessel 616b.

And then next in FIG. 29C for purposes of further illustration, there is provided a pictorial perspective schematic rendering of an illuminated glass charging vessel 616c made according to the method of FIGS. 24A to 24C with a front side wall 175h as formed from a corresponding front mold plate including a mix of cherry blossom and hummingbird motifs as selected from Table 2 of FIG. 26D. Here, indicator light or light source 503a, as encased between the two different layers of glass 618 and 620, may be white in color, to illuminate the cherry blossoms rendering which may be formed in pink and red color glass, while the indicator lights or light sources 503b, also so encased between the layers of glass 618 and 620, may also be of white light to illuminate the hummingbird renderings which may be formed from blue and yellow color glass, as provide in Table 2. Here as with any such feet 621, any one of more of the of the illimitation lights may be positioned to project light downwardly such that ambient light will illuminant the surface supporting the feet 621 given the gap created between the bottom of the vessel and the surface supporting the feet. And here also, as provided by the above method of making, the vessel 616c may advantageously include the electronics casing 506 as positioned in the bottom thereof to enable the desired functionalities of illumination and charging as discussed herein as configured to control the light sources 503a and 503b as well as providing the charging pads that may be included therein for recharging personal electronic items such as a mobile phone or electronic jewelry when placed in the vessel 616c.

Then for purposes of still further illustration, in FIG. 29D there is provided a pictorial perspective schematic rendering of an illuminated glass charging vessel 616d made according to the method of FIGS. 24A to 24C having a front side wall 175h as formed from a corresponding front mold plate including the peacock feather motif as selected from Table 2 of FIG. 26D. Here, indicator light or light source 503a, as encapsulated between the two different layers of glass 618 and 620, may be green in color, to illuminate the center portion of the fan of peacock feathers which may be formed in bright green and blue color glass, while the indicator lights or light sources 503b, as so also encased between the layers of blown glass 618 and 620, may be blue color to illuminate the left and right portion of the fan of peacock feathers which may be formed from the same bright green and blue color glass, as provide in Table 2. And here also, light may be directed out from the bottom of the vessel 616d as supported by the feet 621. And like above, as provided by the method of making, the vessel 616d may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or electronic jewelry when placed in the vessel 616d.

Still yet for an example of textures selected from Table 1 of FIG. 26C, next in FIG. 29E there is provided a pictorial perspective schematic rendering of an illuminated glass charging vessel 616e made according to the method of FIGS. 24A to 24C having a front side wall 175h as formed from a corresponding front mold plate including a mix of patch corrugated and floral textures as selected from Table 1 of FIG. 26C. Here, indicator lights or light sources 503a and 503b, as encapsulated between the two different layers of glass 618 and 620, may be of any desired set, fixed, or programmable color or colors, and the glass color of the vessel may be of any corresponding desired single color or multiple colors. And here also, like any of the above, light may be directed out from the bottom of the vessel 616e as supported by the feet 621. And here also, like above, as provided by the method of making, the vessel 616e may advantageously include the electronics casing 506 as positioned in the bottom thereof, as illustrated, to thereby enable the desired functionalities of illumination and charging as discussed herein where the electronics casing 506 is configured to control the light sources 503a and 503b as well as provide the charging pads for recharging personal electronic items such as a mobile phone or electronic jewelry when placed in the vessel 616e for recharging after a day's use of same.

And still further, as to mixing textures and patterns on the exterior surface of the first layer of blown glass, FIG. 29F provides a pictorial perspective schematic rendering of an illuminated glass charging vessel 616f made according to the method of FIGS. 24A to 24C having a front side wall 175h as formed from a corresponding front mold plate including a mix of the butterfly pattern selected from Table 2 of FIG. 26D, and the Floral texture from Table 1 of FIG. 26C. Here, indicator light or light source 503a may be in any desired color, to illuminate the center butterfly which may be formed in any desired corresponding color of glass, while the indicator lights or light sources 503b may be of the same or different color as the color of the center light 503a to illuminate the left and right butterfly renderings and floral boarder motif which may be formed from the same or different color glass, as provide in Table 2 or as otherwise desired. And like above according to the method of making the vessel 616f, the indictor lights and light sources for illumination 503a and 503b, as controlled by the internal electronics, which in turn may be operated remotely by an app on a smartphone, are encapsulated between the two different layers of glass 618 and 620 to thereby protect same from ambient exposure thus adding functional service life thereto. And like above, with illumination lights so directed, a cushion of light may be provided as extending out from the bottom of the vessel 616f as supported by the feet 621. And here too, as provided by the above method of making, the vessel 616f may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or electronic jewelry when placed in the vessel 616f for recharging after a day's use thereof.

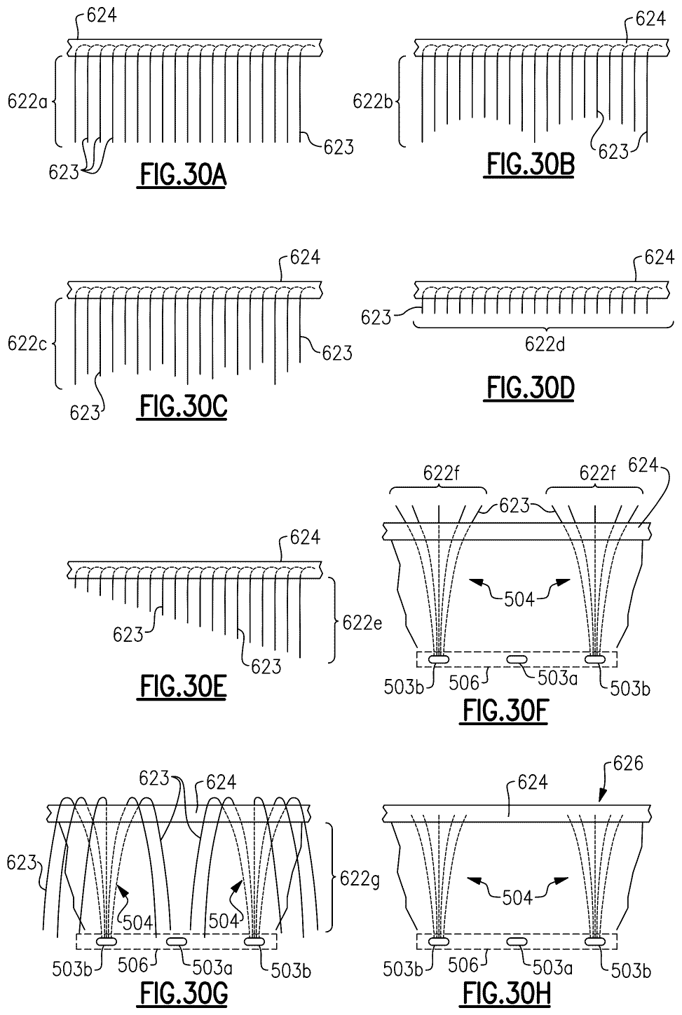

Lastly as to these examples, FIG. 29G provides a pictorial perspective schematic rendering of an illuminated glass charging vessel 616g made according to the method of FIGS. 24A to 24C having a front side wall 175h as formed from a corresponding front mold plate of the type 576 of FIG. 27A which for purposes of illustration here includes therein a high bias relief rendering of a renown sculpture, here being rendered in the form of the well-known and beloved Pauline Bonaparte as Venus Victrix by the Italian sculptor Antonio Canova which resides in the Galleria Borghese in Rome, Italy, for exemplary purposes. Such a front mold plate, where authorized and approved, when needed, may be made by the method of making the front mold plate 578 and corresponding vessel 594 of FIGS. 28A to 28E where here authorized photos of the Venus Victrix are deployed in place of the beloved pet photos 580 of FIG. 28A. As to illuminating the charging vessel 616g in this example embodiment, illumination lights 503a are provided on the front side of the vessel, as encapsulated internally therein between the glass layers, to illuminate the molded artwork while indicator lights 503b, as represented in dashed-line on the right side of the vessel as drawn here, are positioned to provide light into fiber optic bundles 504 as encapsulated between the layers of glass 618 and 620, as per the above method of FIGS. 24A to 24C, which fiber optic bundles 504 here illustrated include extension segments to form a fringe 622a on the backside of the vessel and an angle-cut fringe 622e of extensions of optical fiber on the sides thereof as discussed below in further detail, respectively, in FIGS. 30A and 30E. And here too, since the feet 621 support the vessel 616g above a supporting surface providing an air gap there between, downwardly directed illumination lights may provide the effect of the vessel sitting atop of a cushion of light. And here also, as with the above glass-on-glass vessels 616, the vessel 616g may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or electronic jewelry when placed in the vessel 616g for recharging.

Thus given the methods above of making custom mold plates to thereby further make our molded illuminated glass charging vessels for personal electronic items as particularized for certain consumer groups, as based on national origin, for example, the inventors hereof are presented with the opportunity to provide a library of research results relating to flower types, such as the Frangipanis, Butea monosperma, Ascocenda Princess Mikasa, Ganges Primrose, Lotus, Jasmine, Hibiscus, Sun Flower, Orchids, and Jungle Germanium of India, for example, along with a catalog of native natural scenes including such native flora and fauna that would naturally interact therewith such as butterflies, hummingbirds, and peacocks, for example, which natural scenes may be augment with national historical architectural treasures such as world-heritage temples or shrines, or otherwise molded with impressions of renown works of art as part of any particular country's cultural heritage, where such national and nature images when artfully comingled may provide an added subtle resonance for a user thereof as being from or of that national heritage when visually experiencing such colorful illuminated symbolism of their national, cultural, or spiritual heritage as integrated within such a colorfully animated illuminated glass-blown charging vessel of the types herein disclosed by the inventors hereof. And then further with the methods of FIGS. 28A to 28E of making a custom mold plate as particularized uniquely for a single consumer, any of the above scenes as engraved on a mold plate may have integrated therein a personal image such as that of a particular loved-one or of a beloved family pet, for example.

Thus the inventors hereof further provide a method of making a custom mold plate for making therefrom a customized blown-glass illuminated charging vessel including the steps of i) providing a library of flora and fauna of a particular geographical region; ii) providing a catalogue of national historical architectural treasures associated with said particular geographical region; iii) providing an index of literary works and symbolism associated therewith where said literary works are associated with said particular geographical region; iv) providing an annotated photo album of renown artistic works associated with said particular geographical region; v) providing a software tool that allows selecting images from said library, said catalogue, said index, and said photo album and integrating the selected images into a panel rendering, said software tool then creating an image file of the panel rendering; vi) transferring the image file to a CNC machine and cutting a molding plate having the panel rendering engraved thereon; and vii) using the molding plate with the engraved panel rendering to make any of the above glass vessels. And here for making a unique glass vessel for the sole use of a single consumer, the software tool is further enabled to intake personal photo images and thereby allow such personal images to be among those selected for integration into the panel rendering.

Now turning to FIGS. 30A to 30G, therein provided are schematic representations showing different fringe patterns 622 that may be formed by the optical fiber extensions or extension segments 623 discussed in connection with FIGS. 29A and 29G while FIGS. 30F to 30H show exemplary arrangements of light emitting elements and optical fiber bundles that may be used in combination with any of the glass vessels discussed above in FIGS. 29A through 29G, or any other embodiment of a glass vessel made according to the methods discussed in conjunction with FIGS. 24A to 24C and 25A to 25C.

Thus as first illustrated schematically in FIG. 30A, each of the glass vessels 616 in FIGS. 29A to 29G, and those made according to the above methods, has top edge 624 that may be used as fulcrum or pivot point that enables extension segments 623 of the encased optical fibers to project out therefrom and thus by designed fitting and thoughtful placement from the artisan applying these elements during the making of the vessel at the time of glass blowing, such extension segments 623 in these embodiments are further provided to extend outwardly over the top edge 624 as illustrated to form a fringe 622a of optical fibers as akin to bangs in a Pageboy or Bob hair cut. As such, the optical fibers here utilized for these functional and artistic purposes may be specially formulated as to their chemical and physical properties so as to mimic hair types from bushy and curly, to flowing and wavy, to flat and straight. Further, in these schematic drawings, each extension line 623 may be taken as to represent a single optical fiber or a bundle of two or more optical fibers. Thus in the fabrication hereof, artisans having special training akin to a professional hair stylist, may trim the extensions with specialized cutting tools to thereby give the vessels a look of a Pageboy or Bob haircut style which styles may more particularly include the Angled Bob, Blunt Bob, French Bob, or Wavy Bob, for example. And then alternatively in other embodiments, these optical fiber extensions 623 may be styled in the form of cat whiskers, dog whiskers, or in the fashion of walrus whiskers. Thus in FIG. 30A, a fringe pattern 622a is formed by such extensions 623 all having a similar length as illustrated. Next in FIG. 30B, the fringe pattern 622b has a repeating pattern in the form of an architectural pattern used in frames and moldings, while the fringe pattern 622c in FIG. 30C, is formed by random lengths of the optical fiber extensions 623. In FIG. 30D, the extensions 623 are comparatively short and thus when each extension line in the schematic drawing here represents a bundle of many optical fibers, and the physical nature of the fibers in the bundled extensions is that of bushy and curly, the extensions 623 may be stylized in a fringe pattern 622d as an illuminated fur collar where here the color of light is preferably white and the color of the optical fibers is either clear or otherwise a frosted white. And then in such an embodiment hereof, the color of the glass forming the first blown layer may also be of a translucent frosted white formation thus giving the vessel and furry collar extensions as illuminated with animated white light, a wintery frosted look of snow falling on white fur. Next in FIG. 30E, the fringe 622e is cut at an angle thus giving the feel of an architectural progression or that of an Angled Bob hair style, where here again the fringe pattern 622e may be styled by the artisan where the nature of fibers is bushy and curly, flowing and wavy, or flat and straight as depending on the number of fibers provided and the physical and chemical properties of the glass comprising the optical fiber in these embodiments. Next as to further detail of these aspects of the present invention, shown schematically in FIG. 30F is a light source 503a that may be for illuminating artwork on the vessel, upwardly illuminating the side wall of the vessel for purposes of providing ambient lighting, downwardly projecting light to cast a halo of light out from under the feet of the vessel, or otherwise may be an indicator light of the types discussed above. Here further, light sources 503b are positioned to project light in to a corresponding optical fiber bundle 504 as illustrated and the extensions 623 are cut short as shown in the fringe pattern 622f so as to be in the nature of bristles in a bristle brush or a three-day growth of stubbled whiskers forming a stubbled fringe or mustache around the top edge 624. Here the color of the glass comprising the fibers may be of a translucent red color or a translucent black color and in the former case, the color of the light source 503b may be ruby red, while the color of the light source in the latter case may preferably be white.

And here finally as to exemplary illustrations of fringe patterns 622 formed by the extensions 623, in FIG. 30G, the extensions 623 forming the fringe pattern 622g projecting out and over the top edge 624 are comparatively long such that when the vessel is placed on a table, credenza, sideboard, or nightstand for use, the extensions 623 may terminate just above the surface supporting the feet of the vessel. Here also, the extensions 623 forming the fringe pattern 622g may be bushy and curly, flowing and wavy, or flat and straight as depending on the number of fibers provided and the physical and chemical properties of the glass comprising the optical fiber, such that the styling of the extensions may be in the nature of a Blunt Bob, a French Bob, or a Wavy Bob, for example, where the length of each fiber is the same as the others in the bundle or of slightly differing lengths and then depending on the spacing of the bundles along the top edge 624 and the tightness of the fibers in each bundle, the extensions 623 may be stylized in the nature of a straight cut pony tail or a natural-growth pony tail where each of the fibers while comparatively long have slightly varying lengths.

As such, any of the various embodiments of the above fringe patterns 622 of FIGS. 30A to 30G may be deployed as desired on any of the above two-layer glass-on-glass vessels 616 or variations thereof as specifically designed and curated to achieve the intended effects and functionalities thereof. Further, these fringe patterns 622 are not limited to use with the above glass-on-glass vessels but may be similarly deployed in any of the glass-on-stone vessels hereof as discussed below with reference therein back here to FIGS. 30A to 30G so as not to unnecessarily reintroduce and repeat the same below.

Thus further in view of the above, it should be understood that the intension of the inventors here is to provide a subtle life-like animation to our glass-on-glass and glass-on-stone vessels (that is not robotic in nature) by further prescribing the nature of styling the shape, look, and light show associated with each of the optical fiber fringe patterns 622 discussed above, and as further deployed below, particularly as the functionality of the electronics in the electronics casing 506 provided in a concealed manner in the bottom of the vessel is prescribed to control the color, intensity, and on/off function of the light sources 503b as they project light into the bundles 504 and then along and exiting from the extensions 623.

And here finally in FIG. 30H, each and all of the individual fibers in each of the fiber bundles 504 do not extend out from the top edge 624 but rather terminate just below a top surface 626 of the top edge 624 such that light emitted from the ends of the encapsulated fibers will project straight up, through the top edge 624, and out of the top surface 626 thereby forming sports of light in the top surface 626 in the nature of stars shining in a clear night sky or seeds on the surface of a strawberry where in the first case, the shining nighttime stars are achieved by making the color of the glass forming the top edge 624 a translucent black color and the light provided by the light source 503b white in color, and in the second case of seeds or achenes on the surface of a strawberry, the color of the glass forming the top edge 624 is a translucent strawberry red color and the light provided by the light source 503b may be black, yellow, or white in color with yellow preferred particularly when the spots of glass in the edge projecting down to the terminus of each fiber may be made clear of color in a field of otherwise strawberry-red glass comprising the top edge 624. And across FIGS. 30F, 30G, and 30H, the electronics casing 506 is therein referenced to denote that the lights sources 503a and 503b are operably connected to control circuitry provided therein to achieve the desired illumination and notification-by-illumination aspects hereof as discussed above and in further detail herein below.

And so here more generally across all these embodiments of FIGS. 30A to 30H, by prescribing the number of fibers in the bundle, the spacing between the bundles as positioned along the top edge, the physical and chemical properties of the glass comprising the optical fibers, the color of the glass comprising the fibers, the color of the glass comprising the vessel, the color of the light sources, the length and styling of the extensions, if any, and the functionality of the electronics controlling the light sources, a very wide variety of stylized looks may be achieved to further illuminate the vessels with each so tailored to provide both functional and sensory engagement therewith.

Now generally across FIGS. 24A to 30H regarding various embodiments of our glass-blown illuminated vessels, the inventors hereof thus provide a method of making a glass jewelry box having light display notification and recharging functionality for use with personal electronic items, said method comprising the steps of i) providing a mold 482 configured in a pre-determined shape to form a glass vessel 540; ii) blowing molten glass into the mold 482 to form a first layer of glass 618 having an outside surface and an inside surface; iii) applying at least one light emitting element 502, 503, 504 onto the inside surface of the first layer of glass 618 in a selected location; and iv) blowing molten glass onto the inside surface of the first layer of glass to form a second layer of glass 620 to thereby encapsulate said at least one light emitting element 502, 503, 504 between the first and second layers of glass 618, 620. Here in this method, the mold 482 has a bottom mold plate 484 that forms a corresponding bottom area 528 in the first layer of glass 618, and so as to provide the desired functionality to the glass vessel 540, this method includes the further step of providing a ceramic casing 506 having electronic circuitry secured therein, and fitting the ceramic casing 506 onto the inside surface of bottom area 528 of the first layer of glass 618 before the step of blowing molten glass to form the second layer of glass 620 and so as to make needed electrical connections, we provide the further step of connecting the at least one light emitting element 502, 503, 504 to the electronic circuitry in the ceramic casing 506 before the step of blowing molten glass to form the second layer of glass 620. And since the molten glass and oven temperature may be within certain rangers so as to provide workability to the vessel during the steps of this method, the ceramic casing 506 is preferably formed from a high-temperature ceramic material having heat resistant properties that protect the electronic circuitry from the temperatures associated with the steps of blowing molten glass and heating or cooling same in an oven 516. As such, this method may advantageously include the further step of placing the glass vessel 540 into an oven 516 to cool down so the glass layers avoid cracking while cooling down from the higher temperatures associated with blowing molten glass, to a lower temperature for further processing. And then to further work on the vessel 540 during this fabricating process, this method may advantageously further include the step of removing the glass vessel 540 from the mold 482 or removing the bottom plate 484 from the mold 482 while the side plates 486, 488, 490, 492 remain connected to each other with the vessel 540 therein, before reaching a temperature associated with solidification. Then at an appropriate time in this process, the further step of inverting the glass vessel 540 and applying molten balls of glass 532 to the outside surface of the bottom area 528 of the first layer of glass 618 to form feet 621 for the glass vessel 540 to stand on when in use. And where desired for certain embodiments of the vessel 540, this method may further include the further step 546 of cutting shapes into the outside surface of the first layer 618 of glass to form a desired pattern of cut glass on the outside surface of the first layer of glass 618. And for the desired operational functionality in any of these glass vessels 540, the electronic circuitry may advantageously include circuitry for charging rechargeable batteries when placed in the glass vessel 540 during use thereof, and further to provide the various illumination effects and light show notifications hereof, the electronic circuitry may further include circuitry for providing power to the at least one light emitting element and for controlling the lighting effects thereof. Thus, the at least one light emitting element may be comprised of at least one light emitting diode 503, at least one optical fiber 505 or bundle of optical fibers 504, laser light, or a combination thereof. And thus in practicing this method, the useful article as embodied in the vessel 540 may be placed in use to charge a mobile phone, used to charge any of the electronic jewelry items claimed or disclosed herein that include a rechargeable battery, or used to charge both a mobile phone and electronic jewelry items placed therein together at the end of a day's use thereof. And as discussed above so as to provide silent notification and engagement with a person using the vessel, two or more light emitting diodes 503 may be deployed where each has a different function as to illumination or notification. Thus in some embodiments, each of the light emitting diodes 503 may be associated with at least one optical fiber 505, further each of the light emitting diodes 503 may be associated with a corresponding cluster of optical fibers 504 bundled together at an input end 504a thereof. Then in placement of the fibers during the process of the method, each cluster of optical fibers 504 may be contained within the first and second layers of glass 618 and 620, respectively, such that both the input end 504a and the output end 504b are both encapsulated between the layers 618 and 620 and further wholly contained therein. And in alternate embodiments of the vessel, as illustrated in FIGS. 29A and 29G, the glass vessels 616a and 616g including the upper or top edge 624 may have a selected number of fibers from at least one cluster of optical fibers each extend out from the upper edge to form the fringe patterns 622, as illustrated and discussed above. And in fabrication of same per the method of FIGS. 24A to 24C, a selected number of fibers from each cluster of optical fibers 504 may extend out from the upper edge 624 to thereby form a corresponding number of extension segments 623 as discussed in detail in FIGS. 30A to 30G. And to provide animation in certain embodiments by some gentle movement, at least some of the extension segments 623 may be of sufficient length to be movable by touch of hand or movement of air directed thereon such as in the fringe pattern 622e of FIG. 30E, or alternatively, all of the extension segments 623 may be of sufficient length to be movable by touch of hand or movement of air directed thereon such as in the fringe pattern 622g discussed above in conjunction with FIG. 30G. And in some preferred embodiments hereof, all of the extension segments 623 are of substantially the same length as in the fringe patterns 622a, 622d, 622f, and 622g shown respectively in FIGS. 30A, 30D, 30F, and 30G, or alternately, the extension segments 623 may be of randomly differing lengths as in the fringe pattern 622c discussed in connection with FIG. 30C, or still in the alternative, the extension segments 623 may be cut to form a repeating fringe pattern such as, for example, the fringe pattern 622b of FIG. 30B.

In making any of the above blown glass vessels, the inside surface of the mold 482 use to make same may include a textured surface, such as including, for example, any one or more of the textures provided in Table 1 of FIG. 26C including a repeating corrugated pattern, an irregular corrugated pattern, a floral pattern, an orange peel pattern, a dimpled pattern, a random pattern of bumps, a repeating pattern of bumps, a pattern of shapes projecting into the textured surface, a pattern of shapes projecting out from the textured surface, or a pattern of shapes with some thereof projecting into the textured surface and others thereof projecting out from the textured surface. With use of any of these molds, as may be desired, in practicing the above method of making the glass jewelry box, the method may further include the step of randomly scattering casting sand on the textured surface before performing the step of blowing molten glass to form said first layer of glass to thereby form irregularities on the outside surface of the first layer of glass, as discussed in detail in conjunction with FIGS. 26C and 26E, or this sanding step may be performed by evenly scattering casting sand on the textured surface before performing the step of blowing molten glass into the mold to form said first layer of glass to thereby form irregularities on the outside surface of the first layer of glass. And when two artisans are engaged in making the vessel, one blowing the glass and another to assist therewith, this sanding step may include randomly or evenly scattering casting sand on to the textured surface as performed by a first artisan, while in concert and synchronicity therewith a second artisan performs the step of blowing molten glass to form said first layer of glass.

And as to providing feet 621 for the vessels, the method of FIGS. 24A to 24C may preferably use a mold 482 that includes a removable bottom panel or plate 484 further including the step of removing the bottom panel or plate 484 from the mold 482 before the glass reaches a temperature associated with solidification, then in fabrication, the method includes the further step of inverting the glass vessel and applying molten balls of glass 532 to the outside surface of the bottom area 528 of the first layer of glass 618 to form feet 621 for the glass vessel to stand on when in use.

And as would be understood by those of skill in the mechanical arts, when an inside surface of the mold 482 includes a smooth surface, a corresponding surface on the outside surface of the first layer of glass 618 is smooth when the glass vessel 540 or 616 is removed from the mold and in a similar manner, when an inside surface of the mold 482 includes a textured surface, a corresponding surface on the outside surface of the first layer of glass 618 is thereby textured when the glass vessel 540 or 616 is removed from the mold, and in these embodiments, as discussed above, the textured surface may be formed from a texture selected from the group including wavey, corrugated, dimpled, bumpy, scalloped, orange peel, projecting shapes, recessed shapes, or any combination thereof. And as to the formation of the mold, the mold 482 of FIG. 25A is comprised of five interchangeable plates including four side plates 486, 488, 490, 492 and a bottom plate 484, and as to making a glass vessel therefrom to serve as a glass jewelry box for recharging the electronic jewelry items hereof, for offering a variety thereof, the four side plates 486, 488, 490, 492 may each include different textures, patterns, or combinations thereof, or for simplicity of design, the four side plates 486, 488, 490, 492 may each include the same texture or pattern. And then again to provide more variety among the vessels made in the fabrication process according to the methos of FIGS. 24A to 25C, the four side plates 486, 488, 490, 492 may be randomly selected from a group of interchangeable textured or patterned side plates so that when making a plurality of glass vessels 540 hereby, a variety of different side textures and patterns are achieved therein. Then in practicing this aspect of the present method, after a time of use, a selected number of the side plates may be retired from the group, and separate therefrom or in combination therewith, after a time of use of the prior side plates in the group, a selected number of new side plates may be added to the group. And as discussed above in detail, some of the patterned side plates, like the molding plate 576 of FIG. 27A, may include engravings of original commissioned works of art or artwork of world renown, such as the image of Pauline Bonaparte as Venus Victrix by the Italian sculptor Antonio Canova which sculpture resides in the Galleria Borghese in Rome, Italy, and as then rendered here according to these teaching on the front side of the vessel 616g of FIG. 29G, as discussed above. And in addition thereto, one of the side plates may a front mold plate, such as the mold plate 578 of FIG. 27B, including an engraving formed from a customer photo, as discussed in detail in connection with FIGS. 28A to 28E in making the vessel 594 shown in FIG. 28E.

And further as to the teachings hereof regarding the method of FIG. 24A to 24C, as would be understood by those of skill in the mechanical and glass blowing arts, in view of the disclosure hereof, this method may advantageously further include the step of inserting a shaping block 522 into the mold 482 after performing at least one of the steps of blowing molten glass so that an inside portion of the glass vessel 540 is thereby sized to receive a manufactured insert having compartments for recharging a mobile phone, recharging items of electronic jewelry, or recharging both a mobile phone and at least one item of electronic jewelry placed therein together.

Thus by the above, the inventors hereof provide a wide variety of glass charging vessels 540, 594, and 616 made according to the various embodiments of the methods of FIGS. 24A to 24C, 25A to 25C, 26A to 26E, 27A, 27B, and 28A to 28E, wherein when a smartphone is placed therein for recharging, the at least one light emitting element 502, 503, 504 may be configured to emit a light of a first color to thereby provide silent notification by illumination to a user of the vessel and the smartphone. And further, as discussed above in detail, any of these glass vessels may be further enabled electronically such that when an item of electronic jewelry is placed therein for recharging, the at least one light emitting element 502, 503, 504 emits a light of a second color. In combination therewith, when the vessel includes at least one optical fiber cluster 504, said at least one optical fiber cluster 504 may be configured electronically to emit a light of a first color when a smartphone is placed in the vessel for recharging, and then here further as enabled, when the smartphone is charged, the at least one optical fiber cluster 504 may be enabled to intermittently emit the light of said first color. Alternatively, when the smartphone is charged, the at least one optical fiber cluster 504 may emit light of a second color. In some embodiments, when the vessel includes at least one optical fiber cluster 504, said at least one optical fiber cluster 504 may emit a light of a first color when an item of electronic jewelry is placed in the vessel for recharging and then in this embodiment where desired, when the item of electronic jewelry is charged, the at least one optical fiber cluster 504 may be enabled electronically to intermittently emit the light of said first color. And in some embodiments thereof, when the item of electronic jewelry is charged, the at least one optical fiber cluster 504 may be configured to emit light of a second color.

Then as to the light-matter interactions here provided as applied to clear and/or colored glass being a transparent and/or translucent physical medium in which, and through which, white light and/or colored light is transmitted to thereby provided ambient illumination and silent notification by electronically-controlled lighted sources, in any of the above discussed glass-blown vessels 540, 594, 616, the molten glass used to form the first layer of glass 618 may include a colorant of a first color, and the molten glass used to form the second layer of glass 620 may also include a colorant of a first color to thereby achieved certain desired light effects resulting from the physical phenonium of colored light interacting with colored glass, where in some cases the colored light is coherent light of a single wavelength, such as that provided by a laser source of light as may deployed by any of the light sources 503 so provided herein. Thus to achieve these desired effects to provide each of ambient illumination, silent notification by illumination, and animation thereof by electronic control, all in a pleasing artistic light show having a prescribed calming effect on a person interacting therewith, the molten glass used to form the first layer of glass 618 may advantageously include colorants of two or more colors, and then similarly, the molten glass used to form the second layer of glass 620 may also include colorants of two or more colors. And then according to these aspects of practicing the referenced methods as per the teachings hereof provided in connection with FIGS. 24A to 24C, 25A to 25C, 26A to 26E, 27A, 27B, and 28A to 28E, the inventors hereof provide a wide variety of illuminated glass vessels 540, 594, 616 made according thereto, where in a basic embodiment thereof such a glass vessel for charging electronic items of jewelry and a smartphone configured to interact therewith, may be comprised of i) a layer of glass 618 formed to include a side surface and a bottom surface 528; ii) at least one light emitting element 502, 503, 504 configured in association with said layer of glass 618; iii) a casing 506 implemented in association with the bottom surface 528 of the layer of glass 618; and iv) electronic circuitry secured inside the casing 506 for charging rechargeable items placed proximate thereto and for activating said at least one light emitting element 502, 503, 504 in association therewith. And then as may be further desired, this basic embodiment of said glass vessel may further comprise any one or more of the features, attributes, or functions as rendered applicable therewith.

And now moving next to FIGS. 31A, 31B, and 31C, which taken together present a flow chart showing the steps of a method of making a stone vessel for electronic jewelry according to yet still additional aspects of this invention, this method as engaging some of the steps of FIGS. 24A, 24B, and 24C; and in companionship therewith, corresponding FIGS. 32A, 32B, and 32C provide a series of perspective pictorials illustrating each of the method steps of FIGS. 31A, 31B, and 31C. Thus here at step 630 in FIG. 31A, a block of stone 632 is provided as shown in FIG. 32A. Next at step 634, a vessel shell, carcass, or casing 636 is formed from the block of stone 632, where the shell 636 includes a top edge 637 that starts to emerge from the block 632 as the material from the center of the block begins to be removed. The stone block 632 is preferably a solid block of natural stone that has a translucent quality when fabricated to a certain thickness. Such preferable natural stone material for these stone vessel shells 636 include a natural stone material such as Onyx, Alabaster, or Makrana marble of the type quarried in and around Agra, Uttar Pradesh, India which may further preferably include some selectedly placed inlayed precious or simi-precious stones as has been well known for hundreds of years by specialized stone artisans working with these materials. In any of these stone sculpted embodiments, the stone vessel as a finished charging vessel for personal electronic items may include gilding or gold leaf where desired to provided further unique characterization to each such a stone vessel made according to these methods as exemplified in the various embodiments and further method steps discussed below in further detail. The forming of the vessel shell 636 at step 634, may be performed by traditional or modern stone sculpting methods, or a combination of traditional and modern techniques, where traditional methods include only hand tools and master crafting techniques specifically designed and developed for stone carving or sculpting as date back hundreds or even thousands of years, while such modern methods for stone and marble fabrication may include CNC machinery using high presser water jets as the sculpting tool.

Next at step 638, the stone shell 636 is inverted and foot holes 640 are formed in in a bottom 641 thereof as illustrated. Here it should be understood that the bottom 641 of the shell 363 as a work-piece-in-process emerges as the last of the interior material is removed from the block 632 during the above shell forming step 634. And then as used in the glass blowing methods above, an oven 516 may be used here at new step 642 to slowly heat the stone shell to a desired temperature before the following first glass blowing step is performed, where since some of the method steps thereof are similarly followed here, common reference numerals will thus be used herein for each of such common steps. Then next here at new step 644, molten glass is prepared for blowing a first layer of glass into the stone shell 636 using a blow pipe 496 as discussed above. The molten glass here, however, may be of a specialized formulation to promote adhering to the inside surface of the stone shell 636 in a permanent desired manner (unlike the above mold plates where a release of the glass from the plated is desired), and similarly, the inside surface of the shell may be lathered, before the first glass blowing step, with an etching solution to dissolve some of the stone material to thereby provide a rough rather than smooth surface so as to better promote mechanical adherence of the first blown glass layer to the inside surface of the stone shell. This rough surface, where desired, may also be achieved by grinding tools of a desired grit factor. On the other hand, the outside surface of the stone shell 636 may be more preferably brought to a very smooth fine polished surface finish by various finishing techniques including hand polishing by various finer and finer grits of stone pumice or buffing rouge, for example, as would be well understood by those of skill in the stone sculpting and stone finishing arts. Then at step 646, a first layer of glass is blown into the stone shell 636, as prepared to receive same, to thereby begin the formation of an illuminated stone vessel according to these teachings here including at this stage of the method a work-piece vessel 648 comprised of the stone shell 636 and a first layer of blown glass.

Then like above at a common step 500, light emitting elements 502 and 504 are applied onto the first glass layer. Here, preferably, like above, the first layer of glass is still in a semi-soft state such that the light emitting elements 502 and 504 may be pressed into the first layer of glass. As illustrated schematically, light emitting element 502 is a strip of LED lights including three light sources or indicator lights 503 numbered 1, 2, and 3, and light emitting element 504, as shown schematically, includes a bundle of optical fibers 504 comprised of individual optical fibers 505 as may be bundled together at one end thereof to form an input end 504a where at the other end of the bundle 504, the individual fibers are loose and free such that they may be placed as desired to form an output end 504b of the bundle 504 as illustrated here and discussed in further detail below as to these stone and glass vessels. Here also, like above, the indictor lights 503 may function in a similar manner as to the indictor lights 174 provided in the electronic charging cases and boxes 165 and 372 discussed above in detail in connection with FIGS. 4A to 4M-2 and 15A and in a similar manner to the indictor lights 406 provided in the above electronic jewelry box 402 as used with traditional and electronic jewelry as discussed in connection with above FIGS. 16A to 16C and thus as such, discussion of this functionality of the indictors lights 503 need not be repeated here.

Next provided in this manufacturing method as a supplied input item, like above, is the electronic circuitry casing or electronics casing 506 here also including the knock-out or marking 507 which marks the location of an electric socket in the casing as discussed below in further detail as to this method. The electronics provided in the casing 506 may be of any desired functionality including at least electronic functionality for controlling at least the light emitting elements 502 and 504 and for providing operability to a charging pad implemented therein for recharging personal electronic items such as smartphones and electronic jewelry. The type of charging pad provide therein is of the type discussed above in detail in connection with FIGS. 4B, 16B, and 16C as implemented in various embodiments across all the charging cases and electronic jewelry boxes hereof as discussed above in FIGS. 2A and 2B, FIGS. 3A and 3B, FIGS. 4A to 4M-2, FIGS. 13A and 13B, FIGS. 13C and 13D, FIGS. 14B to 14F, FIG. 15A, FIGS. 16A to 16C, and FIG. 23, and as further discussed above as included with our blown-glass and now here included with these sculpted-stone and glass illuminated charging vessels for smartphones and electronic jewelry. And as discussed above as to the electronics manufacturer working in collaboration with a traditional jewelry maker to provide the electronic jewelry items hereof, such an electronics manufacturer may also be suited to working in collaboration with the inventor-artisans hereof who have designed and created the blown-glass and sculpted-stone vessels hereof. As such and like above, the particular technical details of the electronic circuitry provided in the casing 506 are considered beyond the intended scope of this disclosure and thus will be provided in companion disclosures as may be filed adjacent hereto.

Thus upon receipt from the electronics manufacture, or prior thereto, the electronics casing 506 may be certified as tested for full functionality as designed before placing same into inventory as an input item to the present method of making these stone and glass vessels as with the above glass-blown vessels.

Moving along now to common step 508 next here in FIG. 31A, the casing 506 is fitted onto the first blown glass layer and at step 510, the casing is electrically connected to the light emitting elements 502 and 504. Here also, an electronics and lights testing may be performed before proceeding to the next step, if desired in the present method. And also, like above, the first glass layer may still be soft when placing the casing 506 thereon or it may be in a cooler more solidified state. Then at step 512, FIG. 31B, a second glass layer is blown into the work-piece vessel 648 to thereby encapsulate the casing 506 and the lighting elements 502 and 504 between the two layers of glass. Then during any of the above steps or the steps hereof following, next as per FIGS. 31B and 32B, the work-piece vessel 648 now including the second layer of blown glass, may be place in the oven 516 under step 518 to heat or cool the stone and glass layers thereof at that time in the method as may be desired to avoid cracking, promote annealing, slowly change from a higher temperature to a lower temperature for further processing, slowly change from a lower temperature to a higher temperature for further processing, or more quickly cool or heat the glass so as to promote a crackled effect in one or both of the glass layers as all of such is well known in the traditional creative glass blowing arts where, however, natural stone shells and encased electronics, like those combined here, may not be known or included within the item there traditionally at hand and thus as such, these aspects of these inventions will require additional techniques and enhanced skills in the artisans handling and processing such like according to detailed specifications provided therewith.

Now with continuing reference to FIGS. 31B and 32B, at common step 520, the work-piece vessel 648 is removed from the oven 516 and a shaping block 522 is placed into the vessel onto the interior surface of the second layer of glass and may be gently pressed there-into and there-against to give a certain final desired shape to the interior shape of the glass vessel here under formation. The shaping block 522 is thusly important here for at least two principal reasons; first, the shaping block 522 shapes the interior of the glass vessel to thereby yield more precise tolerances as to the interior dimensions such that manufactured inserts such as jewelry trays or compartment dividers may be better ensured to fit as intended therewith and then second as to the importance of the shaping block 522 in this method, to provide structural support to the two glass layers inside the stone vessel shell during further processing and final cooling to reach a room-temperature final solidified state. And then as indicated at step 650, this now temporarily-assembled, shaping-block-on-glass-on-stone assembly 652 is inverted and placed on the top surface of a work bench 524. Next so as to provide attractive and functional feet to our sculpted-stone and glass-blown vessel, at step 654 with the vessel here being at a desired temperature for further processing as removed from the oven 516 to achieve same, with the shaping block still in the vessel pressing against the layers of soft glass while atop the work bench 524 as illustrated in the schematic pictorials here at this stage of the present method. So here next at step 654, glass balls 532, like above, in a semi-molten state may then be applied to holes 640 in the stone shell to come in contact with the first layer of glass therein to thereby adhere the glass balls 532 to the first glass layer with the stone shell material adjacent to the holes 640 being pinched there-between. Here the glass-on-stone vessel may be allowed to begin to cool to thereby start approach of the glass layers to a final state of solidification while as is well known in the creative material arts of glass blowing, a torch or blow-torch of the oxyacetylene type or other type may be use to provide localized heating at the location of the feet thorough the stone holes to be applied here as initially semi-molten balls of glass 532. In this manner, a better bonding may be achieved between the semi-molten glass balls 532 and the heated or re-heated localized areas on exterior surface of the first glass layer as exposed in the hole 640 previously formed in the stone shell. Then within a timing tolerance as to the pliability of the applied glass balls 532, at common step 534, the assembly 652 is turned up-right on the work bench 524 and while the glass balls 532 are in a certain desired pliable state, a slight downward pressure as indicted by pressure arrows 536, is applied to the temporary shaping-block-on-glass-on-stone assembly 652 so that the glass balls 532 as feet-to-be may be squeezed or squashed a bit, thus, like above, deforming same into semi-oblong pucks or disks with rounded side edges in an artistic manner to thereby hand form custom feet for each vessel such that when placed in use, the top of the vessel will set level with a night-stand, for example, as illustrated above in FIG. 19 as to the vessel 452 shown therein. And now here again in FIGS. 31B and 32B, at step 656, the assembly is allowed to cool and at a prescribed time, the shaping block 522 is removed from the vessel to thereby present a stone and glass vessel made according to this method such that in a back side 660 thereof, at step 662 an opening 544 may be formed to thereby access an electronics port or socket previously provided in the electronics casing 506 discussed above. Here the opening 544 in the stone and glass vessel 658 and also a corresponding opening in the electronics casing 506 as marked by the knock-out or marking 507, may be achieved by stone, glass, and ceramic grinding tools well know in the art, or may be otherwise pre-formed in the stone shell and then formed in the glass by cutting the glass while in a semi-molten state as like above and so applied here.

Next in FIGS. 31C and 32C at common step 548, after all the above manufacturing steps are performed, particularly those where heat is applied to the vessel as a work-in-process item or work-piece vessel 648, the electronic circuitry contained within the electronics casing is tested using test equipment connected to the socket in the casing via the opening 544 to ensure full functionality thereof. And as would be understood by those of skill in the relevant arts, such a testing step or steps as to the electronic functionality, may be performed at any desired time during fabrication and assembly to ensure the good functioning thereof throughout the steps of this method of making such an item as this stone and glass vessel 658.

Then to prepare for consumer use, at common step 550, FIG. 31C, a cover 552 and a jewelry tray 554 with compartments may be provided as illustrated in FIG. 32C. Then at common step 556, the cover 552 and tray 554 may be fitted to desired tolerances in assembly thereof to be tailored as to a desired level of height and shape for fitting within the interior of the vessel 658 as discussed in further detail herein below in connection with FIGS. 53 to 56. Here, since the shaping block 522, FIG. 32B, was used to form the interior dimensions of the vessel 658 given the shape of the stone shell 636, tailoring needs for the cover 552 and tray 554 will be minimal or even unnecessary when the cover 552 and tray 554 are specified or “spec'd” as to the particular shaping block used in manufacturing. And lastly here, FIG. 32C, like above in FIG. 25C, shows a smartphone 558 and some of the electronic jewelry 560 hereof in dashed line in the bottom interior of the vessel 658 as representing the charging or recharging thereof in accordance with the teachings hereof.

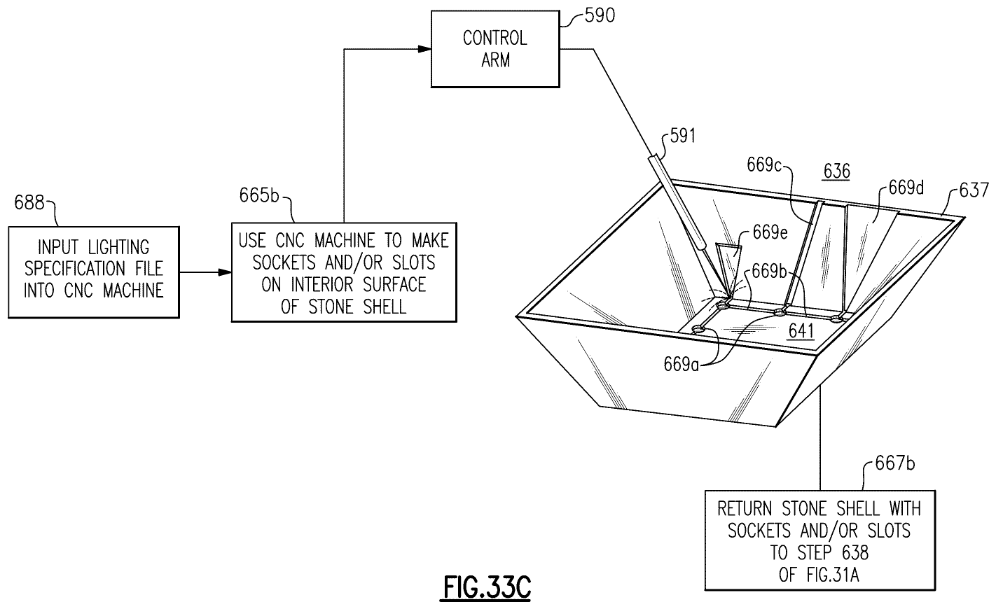

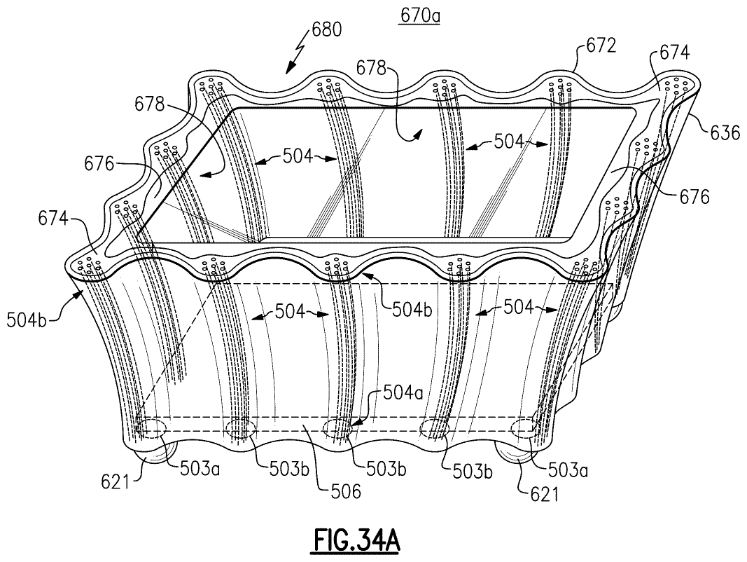

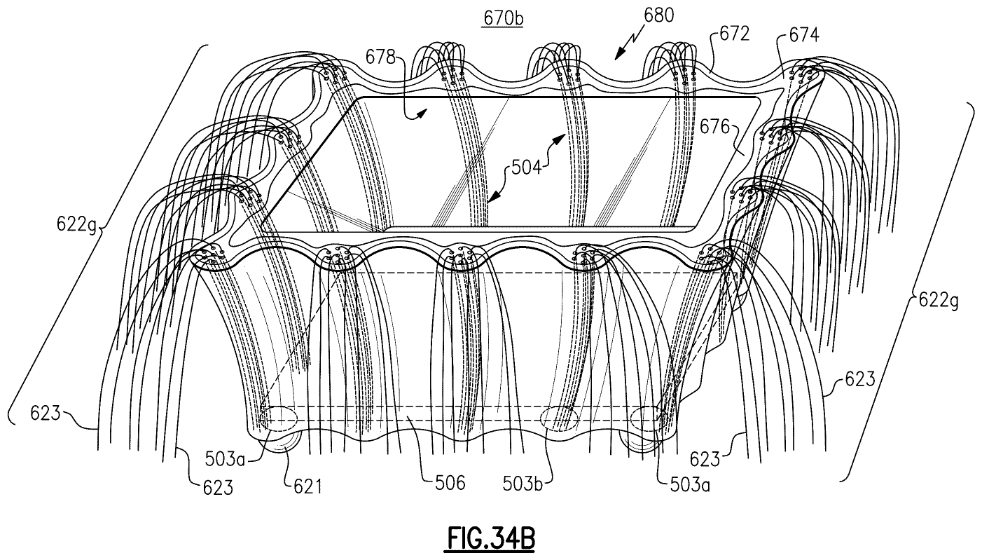

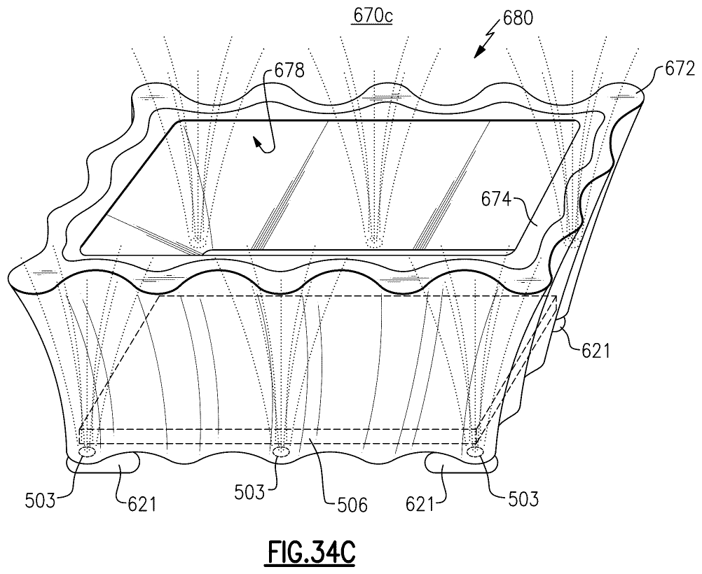

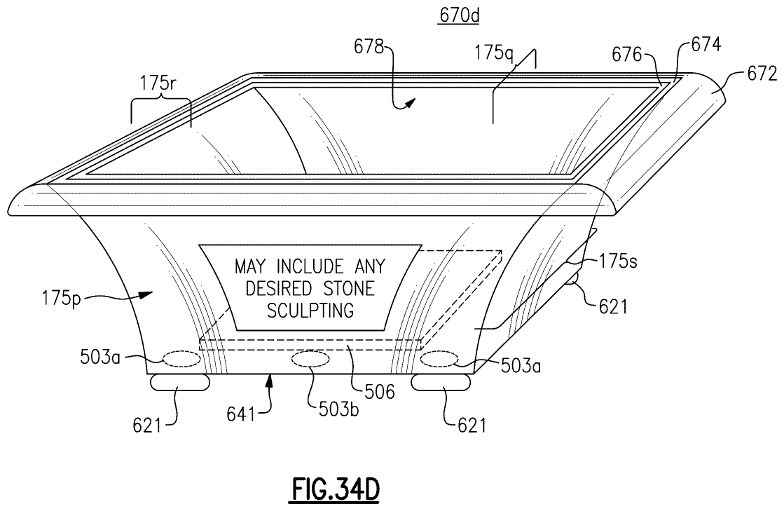

And where desired in a vessel of the type of stone and glass vessel 658 hereof, FIG. 33A is an additional method step 663 and corresponding schematic representation of precious or simi-precious gemstones 664 inlaid into the glass-and-stone vessel 658 made according to certain additional preferred embodiments hereof to thereby provide a unique outside surface associated therewith. Such inlayed gemstones when illuminated with light in a field of white translucent marble, like the Makrana marble of the type from India discussed above, for example, will provide further brilliance and animation to the vessel during use thereof as a recharging station for electronic jewelry that may better comport therewith as to desired style and elegance for selected market segments that may be more attracted thereto as a life-style item rather than as a utilitarian item as based on a cost proposition, while the inventors hereof attempt to provide across any and all such anticipated consumer market segments by providing a wide variety of all such throughout this disclosure.

Thus further as to rendering custom stone vessels akin to the custom glass-on-glass vessel 594 of FIGS. 28A to 28E, here next comes FIG. 33B which provides a subassembly method of receiving photos and operating a CNC machine to make a custom stone casing 636 for the type of illuminated glass-on-stone crafted vessel according to the method discussed above in FIGS. 31A to 32C. Thus here in this subassembly method, at a 581 in common with the above, photos 580 of a beloved family pet are received, then continuing as above in this alternate pet embodiment, at step 582, the images or photos are scanned or otherwise transferred into a digital photo imaging system that includes computer-aided-design or CAD functionality as discussed above, then at common step 584, a three-dimensional or 3D image file of the featured pet is created. Next at common step 586, which may take place at a fabrication location being remotely distanced from where steps 581, 582, and 584 were performed, here at step 586 of this embodiment, the 3D image file of the pet is received, or one such image file of several is selected, and then next at common step 588, the selected 3D image file of the pet is digitally input into the operating system of a CNC machine which, as discussed above, is a type of automated manufacturing machine that includes an operating system having “Computer Numerical Control” or CNC functionality. Then at new step 665a, the CNC machine is used to make a carved or sculpted rendering of the selected image of the pet on a partially prepared stone shell 636 as preliminarily prepared at step 634 of the method discussed above in conjunction with FIGS. 31A and 32A. Here that rendering being a close-up of the face of the canine as illustrated that preferably projects out from the surface of the shell 636. And like above, the CNC machine includes a control arm 590 that has a grinding or other type of stone shaping tool 591 attached thereto that is controlled by the CNC machine to shape, carve, or sculpt the desired rendering of the selected image from the photos. And then as this subassembly is completed, at step 667a, FIG. 33B, the custom sculped stone shell 636 is returned to step 638 of FIG. 31A for further fabrication according to the method thereof. And like above, it should be understood that this subassembly method is not limited to receiving pet photos, but may include receiving any personal photos of any beloved person, place, or thing, which may be further complied along with supporting images from an image library maintained by photo-shop artisans engaged to provide the crafting and curating of such custom or semi-custom image files.

Thus as generally as to the subassembly method of FIG. 33B, the present invention is more particularly directed to a method of making a custom stone shell 636 for making therefrom a customized blown-glass-on-stone illuminated charging vessel 670, said method comprising the steps of i) providing a library of flora and fauna of a particular geographical region; ii) providing a catalogue of national historical architectural treasures associated with said particular geographical region; iii) providing an index of literary works and symbolism associated therewith where said literary works are associated with said particular geographical region; iv) providing an annotated photo album of renown artistic works associated with said particular geographical region; v) providing a software tool that allows selecting images from said library, said catalogue, said index, and said photo album and integrating the selected images into a panel rendering, said software tool then creating an image file of the panel rendering; vi) transferring the image file to a CNC machine and forming on a provided stone shell, a sculpting of the panel rendering to form a sculpted stone shell 636; and vii) using the sculpted stone shell 636 to make a glass-on-stone vessel according to any one or more embodiments of the methods of above FIGS. 31A to 33B, FIG. 33A, and FIGS. 51 to 52B as discussed below. And like above with the glass-on-glass vessels, here also, the software tool may be enabled to intake personal photo images and further allow such personal images to be among those selected for integration into the panel rendering.

And still more particularly as shown in FIG. 33B, the present invention further provides more specifically a method of making a custom stone shell 636 for making therefrom a customized blown-glass-on-stone illuminated charging vessel 670, where this method comprises the steps of i) receiving a collection of personal photo images; ii) creating an image file from the photo images; iii) inputting the image file into a CNC machine and therewith forming on an outside surface of a selected stone shell, a sculpting of an image from the image file to thereby form a sculpted stone shell with the image sculpted thereon; and iv) using the sculpted stone shell 636 with the image sculpted thereon to make a glass-on-stone vessel according to any one or more embodiments of the methods of above FIGS. 31A to 33B, FIG. 33A, and FIGS. 51 to 52B as discussed below, where like above, these photo images may include selected images of a beloved family pet such as the full-breed Shih-Tzu pictured in the photos 580 of FIG. 28A.

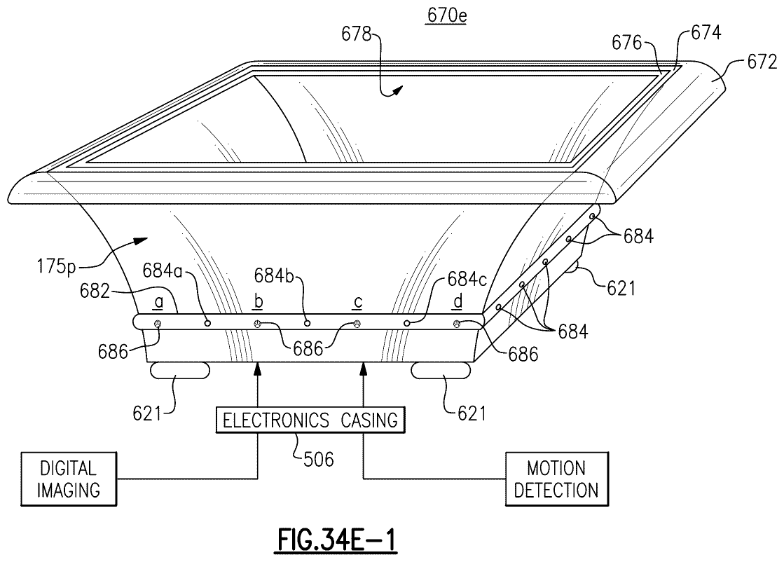

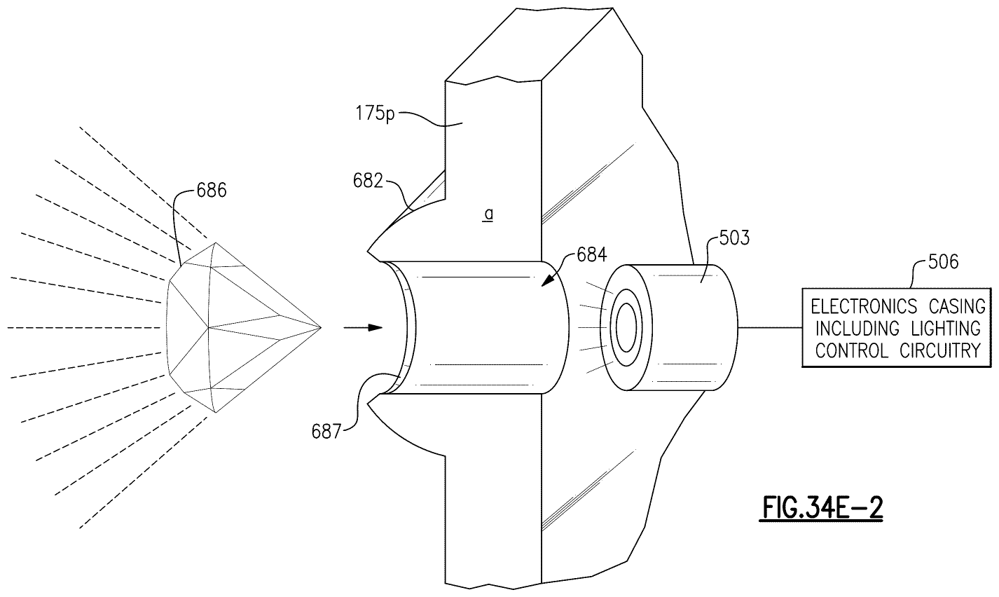

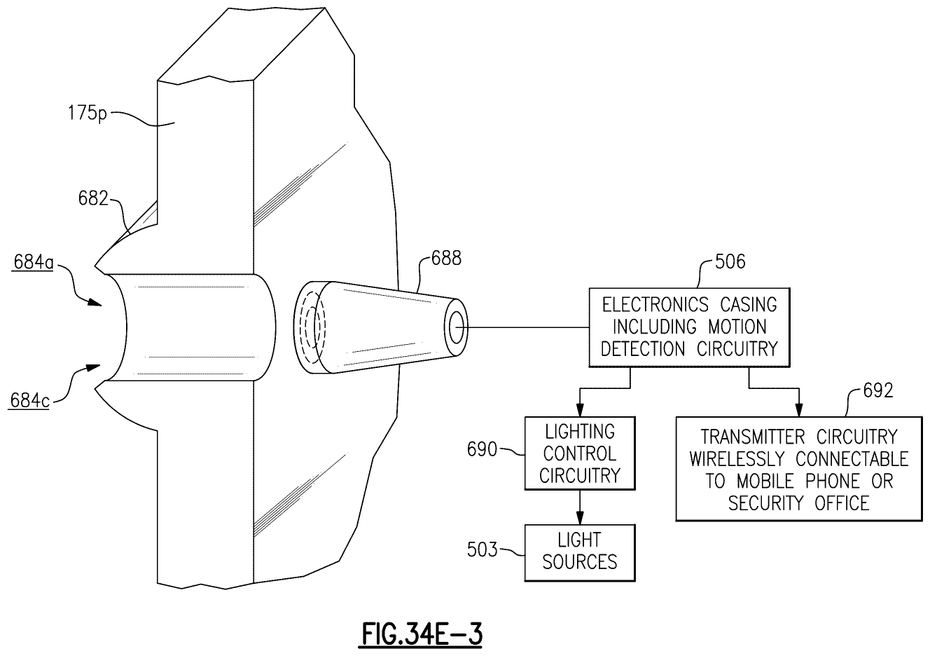

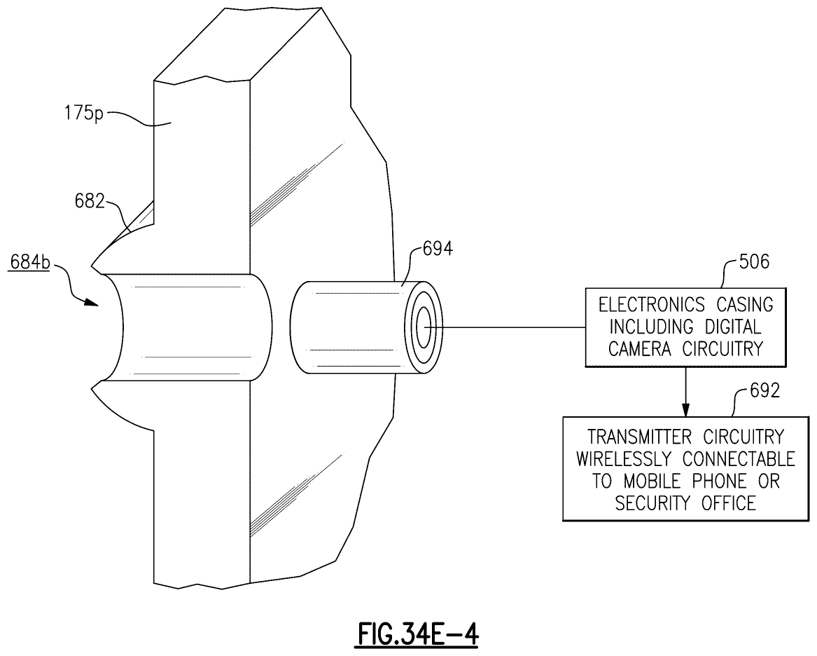

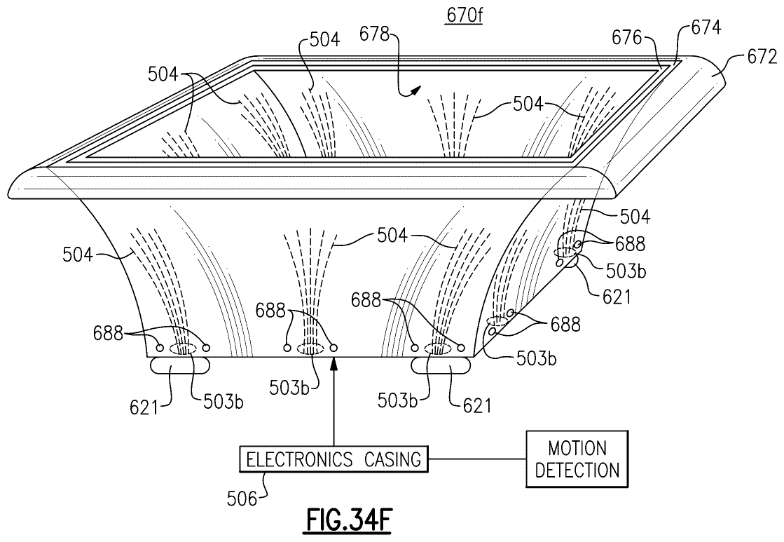

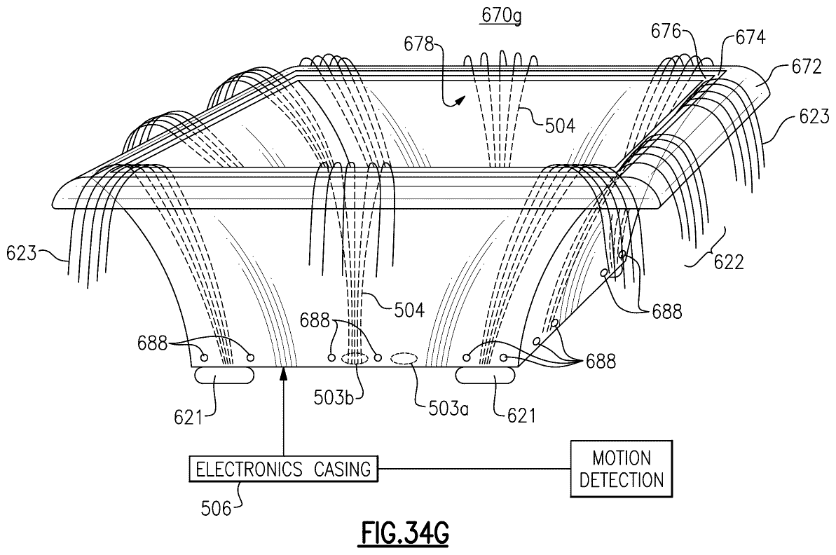

And next in FIG. 33C, the CNC machine may be deployed to form slots or channels on the inside surface of the stone shell 636 before the first layer of glass is blown thereon so as to align the light elements with particularity. Thus here at new step 688, a lighting design specification is input into the operating system of a CNC machine. Next at step 665b, the CNC machine is used to form sockets, slots, grooves and/or channels 669 on the interior surface of the stone shell 636 which was preliminary prepared as a work piece item at step 634 of FIG. 31A. Among the desired forms that may be shaped into the interior surface of the stone shell or casing 636 so as to facilitate the illumination aspects hereof, there are shown here, for example, sockets 669a configured to receive a light source 503, for example; connecting bottom channels 669b configured to align a strip of LED lights 502, for example; a slotted side channel 669c configured to align and confine therein a bundle of optical fibers 504, for example; a full-length fan-shaped side channel 669d configured to align and spread out therein a bundle of optical fibers 504, for example; and a truncated internal fan-shaped channel 669e configured to encapsulate therein a fully encased bundle of optical fibers 504. Here as illustrated, the slotted side channel 669c extends from the bottom 641 of the shell 363, fully up along the inside side surface and through the top edge 637 of the shell 636. Thus since this channel is cut through to the top edge 637 the bundle of optical fibers 504 later placed therein may extend out from the top edge of the shell in a more tightly configured pony-tail style as shown below in FIG. 34B. Similarly, since the full-length fan-shaped side channel 669d is cut through the top edge 637, the bundle of optical fibers 504 later placed therein may fan out and extend up and over from the top edge 637 of the shell 636 in a Pageboy or Angled Bob cut style as discussed above in connection with fringe patterns 622a and 622e shown in FIGS. 30A and 30E, respectively. And finally here as to these subassembly fabrication steps, since the truncated internal fan-shaped channel 669e is configured to encapsulate therein a fully encased shorter-length bundle of optical fibers 504, it should be understood that the height of the truncated internal fan-shaped channel 669e may be set in the design specification so as to terminate for example, a quarter of the way up the side wall, half way up the side wall, three-quarters up the side wall, or just below the top edge 637 of the stone shell 636. Thus in this manner, light emitted from the output end 504b of a bundle 504 place therein may be used as ambient lighting to illuminate any gold leaf or artwork on the corresponding outside surface of the vessel when in use.

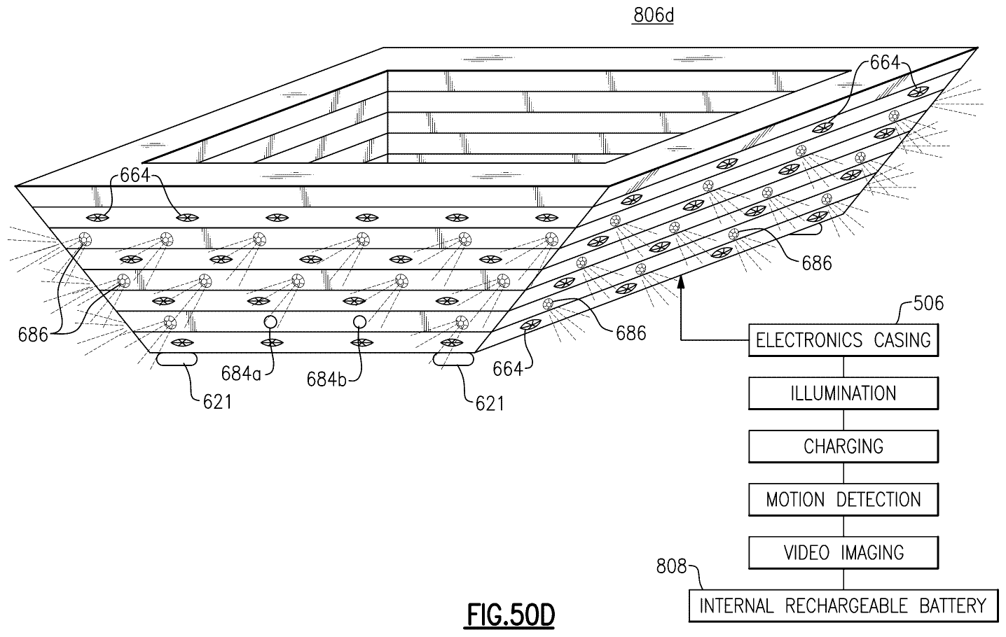

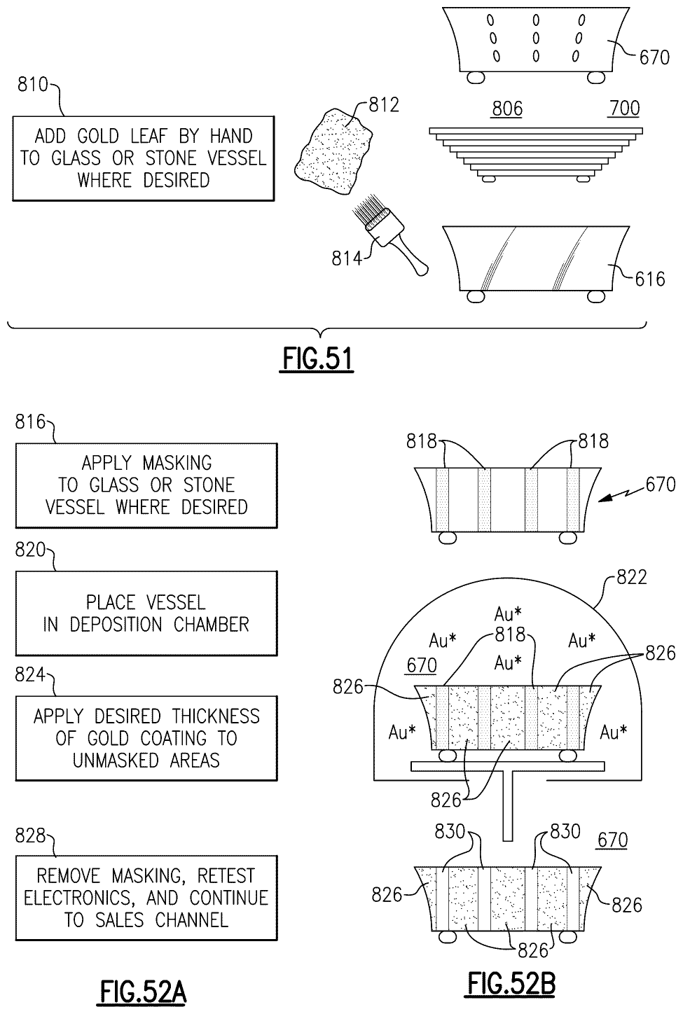

Thus following the above, FIGS. 34A to 34G are perspective pictorial illustrations showing several different glass-in-stone or glass-on-stone vessels 670 made according to various embodiments hereof as discussed above in connection with FIGS. 31A to 33C, in particular, with each including further features, attributes, and variations according to the above and further teaching herein below. Where these vessels 670 include high bias or low bias sculpting of images or shapes, any of these biased segments may be preferably gilded by thinly covering the selected shapes with gold leaf or gold paint, as discussed below in further detail in conjunction with FIGS. 51 to 52B. Thus a further aspect hereof is to provide gold leaf on sculpted stone as illuminated by a controllable LED which illumination gives visual notice regarding an electronic item such as a smartphone or electronic piece of jewelry being of a certain condition or state. In these embodiments, as like with directing the emitted light from an LED or laser source through the inlayed gemstones, which gemstones may be translucent or transmissive to some directed light as in the case of red ruby stones and blue sapphire stones, for example, here when making a glass-on-stone vessel 670 according to a further particularized embodiment of these methods, the gold leaf may be selected of a thickness to be translucent to light so that when the light emitting elements 503 as placed into or on the first layer of glass are directed at the biased sculpted segments of artwork intended for gilding, it may be thus provided that when a personal electronic item is placed in the vessel for charging, or has reached a desired level of charging from the electronic circuitry provided in the bottom of the vessel, such a light source is activated and the gilded segments are thereby illuminated to provide the intended notification to a user of these electronic vessels as configured for use in combination with smartphones and items of electronic jewelry.

Thus according to these extended teachings hereof, in certain highly design and especially curated embodiments of our vessels now including inlayed precious gem stones such as rubies or sapphires on the exterior surface of a white marble stone shell, for example, or gold leaf on sculpted artwork created thereon, as further example, our indicator lights may be directed onto the gemstones or onto the gilded artwork, to provide an illuminated light show giving ruby red color, sapphire blue color, or gold color notification of the electronic state of the recharging of electronic jewelry and/or a smartphone placed therein.

Now as to achieving these desired final embodiments with each illustrated as including an electronic casing 506 as may be provided therein by the above methods of making same, let's next turn first to FIG. 34A, wherein there is provided a glass-on-stone vessel 670a of the above method here show in greater detail as comprising in final assembly, a stone layer or stone wall 672 provided by the stone shell 636, which has applied thereto on an inside surface thereof, a first layer of blown glass 674, which first layer of glass 674 in turn has applied thereto, a second layer of glass 676. As illustrated here in the vessel 670a, the stone shell 636 has been sculped to include scalloped side walls which flow in an undulating pattern around the circumference of the vessel, which when viewed from an inside perspective include lands and valleys as would be understood by those of skill in the mechanical arts. As such, in this embodiment, a bundle of optical fiber 504 has been placed in each valley as shown such that the first layer of blown glass 674 encapsulates each or most of the fibers in the bundle within the first layer 674 with some thereof pressed up against the stone layer or stone wall 672. In this manner, an outer surface of the first glass layer 674 takes the same shape as the interior surface of the stone wall 672, as illustrated, and an inner surface of the first layer of glass 674 may take any form of irregular shape as also illustrated as being so formed during the blowing and applying steps 646 and 500 of FIG. 31A, as discussed above. Then as further illustrated here in FIG. 34A, an outer surface of the second layer of glass 676 takes the irregular shape of the inner surface of the first layer of glass 674, while an interior surface 678 of the second glass layer 676 has a flat or planar face surface as so formed by the shaping block 522 as used in the placing and inverting steps 520 and 650 discussed above in FIG. 31B. In this manner, the vessel 670a is formed and then further as including a top edge 680 comprising and showing or exposing each of the top edges of each of the layers 672 being of stone, and 674 and 676 being of glass. Here, like some prior, the fiber optic bundles 504 do not extend out from the top edge 680 but terminate at their respective output ends 504b, just below the top surface of the top edge 680. And as also shown, source lights 503b are positioned to illuminate the bundles 504 at their respective input ends 504a as so positioned proximate thereto, and source lights 503a are positioned herein to direct light downwardly into the glass feet 621 such that the feet are illuminate and thereby may give the feeling of the vessel floating on a cushion or pillow of light when placed in use on a supporting surface such as a night stand when so activated and illuminated, particularly when the use scenario is a nighttime use in an otherwise dimly lighted room. And here as referenced above, the vessel 670a may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or electronic jewelry as placed in the vessel 670a for recharging when in use as thereby intended.

Next in FIG. 34B, there is provided a perspective view of a glass-on-stone vessel 670b as made according to the above method of FIGS. 31A to 32C here including the pony-tail style fringe pattern 622g with each including a selected number of individual optical fiber extensions 623 as bundled together during the method step 500 of FIG. 31A, which fiber extensions 623 extend out from the top edge 680 of the vessel as shown here and discussed in detail above in FIG. 30G to include movement by touch of hand or motion of air when so formulated and assembled. And like above, this vessel 670b is a three-layer glass-on-stone vessel including the stone layer 672, the first glass layer 674, and the second glass layer 676 having the smooth interior surface 678 formed by the shaping block 522 as used during the above method of making same. And here like above, any desired number of light sources 503 may be included where light sources 503b are positioned to project light into the bundles of optical fiber 504 and the light sources 503a are positioned to illuminate the side walls or the feet 621 to provide a pillow of light at the bottom of the vessel as discussed above and thus not repeated here. And here further, like above, the vessel 670b may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or the electronic jewelry hereof as placed in such a vessel 670b for recharging according to the teaching hereof.

Next in FIG. 34C, there is provided a perspective view of a glass-on-stone vessel 670c as made according to a modified embodiment of the above method of FIGS. 31A to 32C here shown as including internally encased light sources 503 in the absence of any optical fibers as one preferred embodiment thereof. Thus as such, the vessel 670c may not include any optical fibers in a final rendering thereof when so desired, as illustrated here. Thus in those embodiments, the vessel 670c may be illuminated only by the sources 503 and where desired some of all of the outside surface of the stone shell may be covered in a thin layer of gold. This vessel is a two-layer glass-on-stone vessel including the stone layer 672, and here only a single first glass layer 674 which is provided with the smooth interior surface 678 formed by the shaping block 522 as used during the above modified method of making same. And here like above, any desired number of light sources 503 may be included and where desired, any of the above fringe patterns of optical fibers 622 of FIGS. 30A to 30G, or the fully encased bundles 504 of FIG. 30H, may be included here in variations of the vessel 670c as desired where such optical fiber bundles 504 are further provided therewith. And as discussed above in FIG. 33C, the inside surface of the stone shell used to make the vessel 670c may have slots and channels form therein to facilitate the placement of any included optical fibers, optical fiber bundles, and/or light sources associated therewith. And here also, the vessel 670c may advantageously include the electronics casing 506 as positioned in the bottom thereof to enable the desired functionalities of illumination and charging as discussed herein where the electronics casing 506 is configured to control the light sources 503 as well as provide the charging pads for recharging personal electronic items such as a mobile phone or the electronic jewelry hereof when placed in the vessel 670c for recharging according to these aspects hereof.

Still further as to these embodiments with reference next to FIG. 34D, there is provided a perspective view of a glass-on-stone vessel 670d as made according to the above method of FIGS. 31A to 32C here also shown as including internally encased light sources 503 in the absence of any optical fibers as one preferred embodiments hereof and as an option for a desired rendering thereof in such an embodiment of a vessel 670d of this type. This vessel is a three-layer glass-on-stone vessel including the stone layer 672, the first glass layer 674, and the second glass layer 676 which is provided with the smooth interior surface 678 formed by the shaping block 522 as used during the above method of making same. The outside surface of the vessel as formed by the stone shell is smooth and tapered as illustrated with such thus being in the alternative to the above scalloped formations. Thus as a work surface, the front side wall 175h of this type of stone shell may include any desired type of stone carving or sculpting such as, for example, the molded rendering of Pauline Bonaparte as Venus Victrix as discussed above in connection with the glass molded version thereof in FIG. 29G but here rendered in stone by a highly skilled stone sculpting artesian starting with the block of stone 632 of FIG. 32A, and thereon practicing steps 630 and 634 of FIG. 31A. Alternatively where suitable, such stone sculpting may be performed by a CNC machine, or performed by hand using traditional methods of stone sculpting in combination with use of a CNC machine, as like in providing the canine facial rendering on the stone shell 636 discussed in detail above in conjunction with the subassembly method of FIG. 33B. And here too, like above, the vessel 670d may advantageously include the electronics casing 506 as positioned in the bottom thereof to enable the desired functionalities of illumination and charging as discussed herein where the electronics casing 506 is configured to control the light sources 503a and 503b as well as provide the charging pads for recharging personal electronic items such as a mobile phone or the electronic jewelry hereof when placed in such a vessel 670d for recharging according to these aspects hereof.

And as would be readily understood in view hereof, as structurally configured, the vessels 670 include side walls 175 here including a side wall 175p being a front side wall, side wall 175q being a back side wall, side wall 175r being a left-side side wall, and side wall 175s being a right-side side wall as such references may apply to any of the vessels 670 hereof, where from a structural perspective, it may be said that the vessels 670 are formed by side walls 175 and a substantially planar bottom 641 formed integral therewith as may be understood across all common types of glass-on-sculpted-stone vessels 670 disclosed herein. And as with the above glass-on-glass vessels 616 of FIGS. 29A to 29G, these sculped glass-on-stone vessels 670 of FIGS. 34A to 34G may not be limited to including four side walls in a rectangular configuration but may alternatively have three or more straight wall segments or otherwise configured in various desired shapes from round, to heart-shaped, to butterfly-shaped, and any further such, as achieved by providing sculped stone shells from the skilled hands of traditional stone sculpting artisans, or as produced by modern technically sophisticated programming of CNC machines, or as otherwise provided by a combination of these traditional and modern techniques as discussed above in connection with providing the sculped stone shells 636 of FIGS. 32A and 33B.

And here like above, any desired number of light sources 503 may be included and where desired, any of the above fringe patterns of optical fibers 622 of FIGS. 30A to 30G, or the encased bundles 504 of FIG. 30H, may be included here in variations of the vessel 670d as designed and so rendered with one or more optical fiber bundles 504. For example, as illustrated here, light source 503b is positioned to illuminate the front side wall 175h of the vessel 670d which may have artwork sculpted thereon and light sources 503a may be configured to project light downwardly into the feet 621 to provided that pillow of light discussed above.

And as to providing additional electronic functionality as concealed in the bottom of these glass-on-stone embodiments, vessel 670e next discussed with reference to FIG. 34E-1 is an example of a particular embodiment of an illuminated stone charging vessel according to the above teachings but now here further including a hidden digital camera and motion detection functionalities. Like above, this illuminated vessel 670e includes the stone layer 672, the first glass layer 674, and the second glass layer 676 having the smooth interior surface 678 formed by the shaping block 522 as used during the above method of making same. And here like above but not illustrated for purposes of simplicity and further clarity as to the following, the vessel would include the electronic casing as concealed in the bottom thereof and any desired number of light sources positioned to project light where desired. Then next here as to the further intended, there is provided a simple decorative band, beading, or banding 682 which here as illustrated is a half-round molding of the type commonly used in the art of woodworking and finished carpentry. Here however, this half-round band 682 is formed integral with the vessel as sculpted from the same block of stone 632 of FIG. 32A where the artesian or CNC machine or a combination thereof, shaped same according to specifications provided therewith. Next according to this embodiment and the additional method steps associated therewith, a number of holes 684 are drilled, bored, or otherwise formed to extend from the interior of the vessel, through the vessel side walls 175, and then out through the band 682 as illustrated. Next, motion detection and digital camera components as previously provided in association with the electronics casing are here included as positioned behind some of the holes as needed to so function here as intended, where such positioning and assembly take place as additional method steps in the method of making this type of vessel according to these further teachings. And then as to these additional aspects, LED or laser lights may be positioned behind other selected holes 684 as provided by the specifications for making this particular type of vessel. Then where desired, those holes 684 including light sources may have set therein on the exterior of the vessel a precious gemstone such as a ruby, sapphire, or emerald thereby giving red, blue, or green glowing gemstone color respectively which may be animated as a light show by controlling light intensity and on/off timing, for example. Thus here illustrated to show this aspect, at locations a, b, c, and d on the band 682 where holes were formed, now a gemstone such as a cut ruby stone 686 is set in each thereof with a light source positioned there-behind to illuminate the gemstone in a controlled manner. And then when the indicator light positioned there-behind is activated, red color light will emit from the ruby stone thus giving an intended light show that may have notification aspects associated therewith. And further in this embodiment, hole 684a may include a sensor for detecting motion, hole 684b may include a functional digital camera that can provide an image of anything moving in front of the vessel, and like hole 684a, hole 684c may also include a motion detection sensor positioned therein, while hole 684b may have fixed therein, the lens of a hidden camera as configured to take real-time video images and stream them to a mobile phone. And here again, like above, the vessel 670e may advantageously include the electronics casing 506 as positioned in the bottom thereof to enable the desired functionalities of illumination, charging, motion detection, and digital imaging as discussed further below where the electronics casing 506 is configured to control the light sources 503a and 503b as well as provide the charging pads for recharging personal electronic and here illustrated for exemplary purposes as further providing visual imaging and motion detection.

As such, these visual imaging and motion detection aspects hereof will next be discussed in further detail in FIGS. 34E-2, 34E-3, and 34E-4 as implemented in the vessel 670e of FIG. 34E-1, and further discussed below in conjunction with FIGS. 57 and 58 as may be applied generally across all the various vessels hereof.

And here next to provided further details regarding these additional functionalities of the vessel 670e, FIGS. 34E-2, 34E-3, and 34E-4 provide detailed perspective assembly views showing different components and supporting electronic circuitry implemented in the vessel 670e of FIG. 34E-1 as an illustration of such components, circuitry, and functionality that may be so implemented in any of the charging vessels hereof. Thus first in FIG. 34E-2, there is shown a cut-away section of the front side wall 175p of the vessel 670e of FIG. 34E-1, and in the decorative band 682 there is shown further a cut-away section of a selected hole 684. Here, the outside facing end of the hole 684 is provided with a bevel 687 so that a precious gemstone like the illustrated cut ruby 686 may be permanently set therein by methods well know of the arts of setting gemstones in metal or stone. Then next according to more modern facilitations, a light source 503 is fitted into the back side or interior side of the hole 684, and as discussed above, during fabrication and assembly, the light source 503 is operably connected to the electronic casing 506 that includes lighting control circuitry. Here then when in use, the lighting control circuitry is programmed to activate the light source 503 is a prescribed manner as to color, intensity, and duration to thereby provide a light show that may be notification of an event such as the charging of a mobile phone, charging of electronic jewelry, or otherwise activated by motion detection as a type of interactive light show. The light source 503 may be any suitable light source such as an LED that provides white light or colored light, or the source 503 may be a laser light source that provides coherent light of a single wave length. And as understood in the arts of providing precious gemstones, each type of gemstone such as a ruby-red, emerald-green, or sapphire-blue has a particular color that may be specified particularly with optical instruments such that the naturally exhibited or expressed color of the gemstone may be expressed as an RGB value, and then each value thereof associated with a wavelength of EM radiation in the visible spectrum. Thus with such specifications provided in the assembly hereof, the LED or laser light source may be tuned as to its wavelengths or wavelength to more preferably interact with a particular gemstone so that the light-matter interaction between the directed light from the source 503 through a gemstone like the ruby gemstone 686 shown here in FIG. 34E-2 for exemplary purposes, may provide a more brilliant or vivid light show given the diffraction or refraction of the selected light through the gemstone in further view of its type of cut and number of facets associated therewith.

Then next as shown in FIG. 34E-3, in the holes designated 684a and 684c in the decorative band 682, a motion detector or sensor 688 may be advantageously implemented to thereby provide either or both of notification or interaction by illumination and property security by wireless reporting. Thus according to these further aspects of this invention, during fabrication and assembly, any number of motion detectors 688 may be provided in a vessel hereof a specified. Each of the detectors 688 are then connected to the electronics casing 506 which in these embodiments further includes motion detection circuitry which is operatively associated with specified lighting control circuitry 690 which in turn controls at least some of the light sources 503. Thus here in use, when a person duly associated with the vessel comes in proximity to the motion detector 688 as so configured in the vessel, the motion and control circuitry will active at least one light source 503 for a set time duration to thereby give a form of personification to the vessel whereby the vessel acknowledges the presence of a person in proximity thereto by having at least one of its light sources turned on or otherwise change state from an off-state to an on-state for a set time duration. And as to providing security features to these vessels, the electronic casing 506 may further include transmitter circuitry 692 which may be set in a security mode such that when an unauthorized person comes in proximity with the vessel as detected by the motion detector 688, an alarm message or notification is wirelessly sent by the transmitter circuitry 692 to a mobile phone or security office.

And then finally here as to further security features that may be included in any of the jewelry vessels, boxes, or cases hereof, FIGS. 34E-1 and 34E-4 taken together show a digital camera 694 installed in the hole 684b as situated here in this embodiment in the decorative band 682 integrally formed in the stone shell comprising the front side wall 175p of the three-layer glass-on-stone vessel 670e. Then here similarly as with the above motion sensor in security mode, digital camera circuitry provided in the electronics casing 506 may be operatively associated with the transmitter circuitry 692 to thereby wirelessly live-stream a video image from the digital camera 694 to an authorized mobile phone or security office.

And now moving on to certain of these aspects of motion detection as configured herein for illumination and animation purposes to thereby give the vessel a type of life-like ability to silently interact with a duly intended user thereof, FIG. 34F provides a perspective view of a vessel 670f made according to the above methods which is a three-layer glass-on-stone vessel here similarly including the stone layer 672, the first glass layer 674, and the second glass layer 676 having the smooth interior surface 678 formed by the shaping block 522 as used during the above method of making same. And here like above, any desired number of light sources 503 may be included where here light sources 503b are positioned to project light into their respective bundles of optical fiber 504 as shown, and other lights sources, not shown here for simplicity, may be positioned, for example, to illuminate the side walls between the bundles or the feet 621 to give the effect of having the vessel 670f floating on a cloud of light (which light may be of any desired color) when the vessel is placed in use on a supporting surface such as a nightstand, particularly when the use scenario is nighttime use in an otherwise dimly lighted room. And then here to achieve the above-referenced animation associated with bodily movement proximate the vessel, a motion detector or sensor 688 may be associated with a corresponding bundle 504 such that when a person walks by the vessel, one or more of the motion detectors 688 that detect such movement, cause a corresponding indicator light 503b to activate by emitting light for a prescribed time period and in a prescribed manner as to light color, intensity, and/or on/off speed such that the movement proximate the vessel is notified by an indicator light 503 so configured in combination with a motion detection sensor 688. Thus in this manner, it may be said that the vessel acknowledges the presence of a user thereof by providing a timed light show that may be preferably of just a few seconds in duration such that same is co-temporal with the timing and proximity of the bodily movement relative to the standing placement of the vessel in use. And here as per the above teachings, the vessel 670f may advantageously include the electronics casing 506 as positioned in the bottom thereof to enable the desired functionalities of illumination, charging, motion detection for light animation where the electronics casing 506 is configured to control the light sources 503 as well as provide the charging pads for recharging personal electronic and here illustrated for exemplary purposes as further providing motion detection for activating certain illumination aspects hereof as discussed above.

And finally here as to these various embodiments, FIG. 34G provides a perspective view of a vessel 670g made according to the above methods which is also a three-layer glass-on-stone vessel here similarly including the stone layer 672, the first glass layer 674, and the second glass layer 676 having the smooth interior surface 678 formed by the shaping block 522 as used during the above method of making same. And here like above, any desired number of light sources 503a and 503b may be provided as described above with some thereof positioned to direct light into a corresponding bundle of optical fiber 504 or otherwise illumine the side wall or feet. And here for additional animation and interaction, the optical fiber extensions 623 forming a fringe 622 as extending out from between the layers of glass 674 and 676, are in this embodiment made of a sufficient length and strength such that by gentle touch of hand or movement of air, move or otherwise oscillate through a cycle of motion with decreasing amplitude, which when such motion is caused by touch of hand by a person proximate thereto, the motion detectors 688 will detect the presence of the person and thereby trigger a light source to turn on and therewith direct light into the bundle 504 and out the end of the extensions 623. Thus in this manner, it may be said by personification that the vessel responds to both the presence and touch of a person proximate thereto, particularly when the extensions 623 are configured to drape below the sensors 688 such that movement of both the proximate person and the extensions case a light source to trigger to an on state that is otherwise configured to be in an off state. And here too like above in the vessel 670f, the vessel 670g may advantageously include the electronics casing 506 as positioned in the bottom thereof to enable the desired functionalities of illumination, charging, motion detection for light animation where the electronics casing 506 is configured to control the light sources 503a and 503b as well as provide the charging pads for recharging personal electronic and motion detection for activating certain illumination aspects as discussed above.

Thus generally across FIGS. 30A to 30H and 31A to 34G, the present invention is more particularly directed to a method of making a natural stone jewelry box 670 having light display notification and recharging functionality for use with personal electronic items 558, 560, where one principal embodiment of this method comprises the steps of i) selecting a block of stone 632 having a desired translucence property; ii) forming a stone vessel or shell 636 from the block of stone, said stone vessel 636 having an outside surface, an inside surface, and a thickness therebetween that allows some light to pass therethrough; iii) blowing molten glass into the stone vessel to form a first layer of glass 674 on the inside surface of the stone vessel or shell 636; iv) applying at least one light emitting element 502, 503, 504 onto the first layer of glass 674 in a selected location; and v) blowing molten glass onto the first layer of glass to form a second layer of glass 676 to thereby encapsulate said at least one light emitting element between the first and second layers of glass. In all of these embodiments, the stone vessel 636 has a bottom area 641 with a corresponding outside surface and inside surface, and the further steps of providing a ceramic casing 506 having electronic circuitry secured therein, and fitting the ceramic casing 506 onto the inside surface of the bottom area 641 may be advantageously applied. And in all embodiments thereof, the further step of connecting the at least one light emitting element 502, 503, 504 to the electronic circuitry in the ceramic casing 506 before the step of blowing molten glass to form the second layer of glass 676, may be also desired and thus so included. And for guarding against heat degradation, the ceramic casing 506 may be formed from a high-temperature ceramic material having heat resistant properties that protect the electronic circuitry from the temperatures associated with the step of blowing molten glass onto the ceramic casing, and/or heating or cooling the vessel as a work-piece item in an oven during the process of making same. Thus as such, this method may further include the step of placing the stone vessel into an oven 516 to cool so the glass layers 674 and 676 avoid cracking while cooling down from the higher temperature associated with blowing molten glass, to a lower temperature for further processing. And in any of these embodiments with the electronics casing 506, the electronic circuitry may advantageously include circuitry for charging rechargeable batteries when placed in the vessel 670 during use thereof, and further as related thereto, the electronic circuitry may also include circuitry for providing power to the at least one light emitting element 502, 503, 504 which may be comprised from a light emitting diode 503, a strip of light emitting diodes 502, an optical fiber 505, a bundle of optical fibers 504, laser light, or any desired combination of two or more thereof.

Then as such, with these provisions and steps performed in accordance with the teaching hereof, a glass-on-stone vessel 670 of any of the types shown in FIGS. 34A to 34G, or further variations thereof, may be made and delivered for use as intended. And thus in use, the vessel 670 may be used to charge a mobile phone 558, to charge electronic jewelry items 560, or used to charge both a mobile phone 558 and electronic jewelry items 560 placed therein together at the end of a day's use of any such mobile phone or electronic jewelry item.

And as would be further understood in view of the present disclosure and teachings hereof, a two-layer or three-layer glass-on-stone vessel of the type 670 discussed in connection with FIGS. 31A to 34G may further comprise any one or more of the features, attributes, or functions of any of the two-layer glass-on-glass vessels 540, 594, and 616 previously discussed in connection with FIGS. 24A to 29G as rendered applicable therewith. For example, the photo images 580 of FIG. 28A may be received, converted to an image file, sent to a CNC machine, and then an image of the photographed pet thereby sculpted in stone on the front outer face of the stone shell 636 first introduced above at step 634 of the method of FIGS. 31A to 32C. Similarly, any of the textures from Table 1 of FIG. 26C or patterns of Table 2 from FIG. 26D, may be provided on the outer surface of the stone shell 636, and the color of the indicator or illumination lights and glass layers thereof may be so informed by same in rendering any glass-on-stone vessel of the types made according to the methods of FIGS. 31A to 32C and hereafter related as further discussed below. And further, rather than glass molded renderings of renown artwork on a glass-on-glass vessel as rendered by a specially cut front molding plate, such like Pauline Bonaparte as Venus Victrix on the vessel 616g of FIG. 29G, a counterpart stone sculpture thereof may be readily provided on the front side of the glass-on-stone vessel 670d of FIG. 34D as discussed above in connection therewith. And thus as such, those such variations as may be readily adopted from the above glass-on-glass discussions and applied to these sculpted glass-on-stone vessels, should not need repeating here.

Thus moving on to illuminating these glass-on-stone vessels, the method of FIGS. 31A to 32C may be provided such that the at least one light emitting element includes a predetermined number of light sources 503 with each thereof being associated with a corresponding cluster of optical fibers 504 bundled together at an input end 504a thereof, and then is some preferred embodiments thereof, the step of forming the stone vessel or shell 636 from the block of stone 632 further comprises forming at least one side wall including a scalloped pattern having repeating lands and channels, such as those shown in FIGS. 34A to 34C. And then where desired, the step of applying the at least one light emitting element onto the first layer of glass 674 may further comprise pressing a selected corresponding cluster of optical fibers 504 into a corresponding side wall channel before the step of blowing molten glass to form the second layer of glass 676. Then herewith, each cluster of optical fibers 504 may be contained within the first and second layers of glass 674 and 676, as shown in FIG. 34A, for example, or as each vessel 670 includes a side wall with an upper edge 680, then during assembly step 500 of FIGS. 31A and 32A, any selected number of fibers from at least one cluster of optical fibers 504 may each extend out from the upper edge 680 as represented by one of the bundles 504 in the fringe 622g of FIG. 34B, for example, and in a further variation thereof, a selected number of fibers 503 from each cluster of optical fibers 504 may extend out from the upper edge 680 thereby forming a corresponding number of extension segments 623, as shown in the fringe 622 of the vessel 670g in FIG. 34G, for example. Then for additional animation by lighted movement of individual fiber extension 623 where such lighted movement may be provided in silence resulting from the absence of any electronic beeping sounds from anything in the electronic casing, at least some of the extension segments 623 are of sufficient length to be movable by touch of hand or movement of air directed thereon as shown and discussed, for example, in conjunction with the extensions 623 on the vessel 670b of FIG. 34B. And then where desired, during the assembly process, all of the extension segments 623 may be of a sufficient length to be movable by touch of hand or movement of air directed thereon. And as depending on the cut style of the extensions, all of the extension segments 623 may be of substantially the same length, or the extension segments 23 may be of randomly differing lengths as discussed in FIGS. 30A to 30G and applied in the methods of FIGS. 31A to 32C to produce the vessels 670b and 670g of types discussed in FIGS. 34B to 34G, for example. Or alternatively, the extension segments 623 of any of these glass-on-stone vessels 670 may be cut to form a repeating fringe pattern of the type in FIG. 30B.

And as discussed below in detail in conjunction with FIGS. 51, 52A, and 52B, in making any of the vessels 670 according to the method of FIGS. 31A to 32C, such method may in comprise the further step of applying gold leaf onto at least a portion of the outside surface of any one or more of the glass-on-stone vessels 670 as selected for same. This gold leaf may be applied by placing the vessel in a deposition chamber, and then depositing a semi-transparent layer of gold onto at least a portion of the outside surface of the vessel. And then depending on the desired placement of gold leaf as considered in view of the placement of the light sources 503, some of the outside surface of the stone vessel may be masked before the steps of placing the stone vessel in the deposition chamber and depositing the semi-transparent layer of gold onto the outside surface thereof. Then by specification during the assembly process, particularly at step 500, FIG. 31A, the step of applying the at least one light emitting element onto the first layer of glass 674 may include placing the at least one light emitting element to direct light onto the portion of the outside surface of the vessel as specified to have the semi-transparent layer of gold. This portion may be large or small and when small, may be formed in a repeating or random pattern as by the masking step with a light source provided for illumination of each portion of the vessel that has such a semi-transparent gold coating. Or alternatively, this step may include depositing the semi-transparent layer of gold onto a substantial portion of the outside surface of the vessel, when then the placement of light sources is less critical as to illumination of the gold layer thereby provide a light show by golden glow which may have any features of notification as discussed above. And in a preferred embodiment thereof, so as to cover essentially the entire outside surface of the vessel in semi-transparent gold, the step of depositing the semi-transparent layer of gold onto the outside surface of the stone vessel may include masking only a bottom portion of the stone vessel to prevent the deposition of gold thereto. Here as to fabrication, the step of placing the stone vessel in the deposition chamber may be performed before blowing molten glass to form the first layer of glass 674 such that the artisan placing the light sources 503 at step 500, FIG. 31A, may see the gold thereon or alternatively, the step of placing the stone vessel in the deposition chamber may be performed after blowing molten glass to form the second layer of glass 676 thus allowing the light sources 503 to be illuminated during a test step where then the masking may be applied to the vessel as so illuminated to thereby selected where gold layering may be desired or not desired in that specific vessel.

Thus with the above method steps at hand, a wide variety of illuminated three-layer glass-on-stone vessels 670 may be provided as demonstrated by the vessel 670a or FIG. 34A, vessel 670b of FIG. 34B, vessel 670d of FIG. 34D, vessel 670e of FIG. 34E, vessel 670f of FIG. 34F, and vessel 670g of FIG. 34G, which further may include any of the fringe patterns 622 formed by the extensions 623 from FIGS. 30A to 30G, and/or including the internally encapsulated fiber bundles 504 of FIG. 30H. Further illuminated artwork and gold leaf or semi-transparent gold layers may be applied, and inlayed precious gemstones such a rubies, emeralds, and/or sapphires may be illuminated by light to augment the notifications by light signals into ambient space when in use.

And still further, the present invention is also directed to a method of making a natural stone jewelry box having light display notification and recharging functionality for use with personal electronic items, this method comprising the steps of i) selecting a block of stone 632 having a desired translucence property; ii) forming a stone vessel or shell 636 from the block of stone, said stone vessel or shell 636 having an outside surface, an inside surface, and a thickness therebetween that allows some light to pass therethrough; iii) positioning at least one light emitting element 502, 503, 504 at a selected location along the inside surface of said stone vessel or shell 636; iv) providing a ceramic casing 506 having electronic circuitry secured therein and fitting the ceramic casing 506 onto an inside bottom area 641 of said stone vessel or shell 636; and v) blowing molten glass onto the inside surface of the stone shell 636 to form a layer of glass 674 to thereby encapsulate said at least one light emitting element 502, 503, 504 and said ceramic casing 506 between the inside surface of the stone vessel or shell 636 and the layer of glass 674, as made according to an alternate embodiment of the method of FIGS. 31A to 31C to thereby produce a two-layer glass-on-stone vessel 670c of the type discussed in connection with FIG. 34C. Here like above, the stone shell 636 has the bottom area 641 that includes an outside bottom surface and an inside bottom surface and to provide the electronic functionally as desired, this method also includes the further step of connecting the at least one light emitting element 502, 503, 504 to the electronic circuitry in the ceramic casing 506 before the step of blowing molten glass to form the layer of glass 674. And like above in the three-layer embodiment, here in the two-layer version, the ceramic casing 506 is preferably formed from a high-temperature ceramic material having heat resistant properties that protect the electronic circuitry from the temperatures associated with the step of blowing molten glass onto the ceramic casing 506. And similarly in this two-layer embodiment, this method may further comprise the step of placing the glass-on-stone vessel 648 as a work piece in process into an oven 516 to cool so the single glass layer 674 avoids cracking while cooling down from the higher temperature associated with blowing molten glass, to a lower temperature for further processing. And to provide the desired recharging functionality, here also in the two-layer embodiment, the electronic circuitry includes circuitry for charging rechargeable batteries when placed in the glass-on-stone vessel 670 during use thereof, and the electronic circuitry further includes circuitry for providing power to the at least one light emitting element 502, 503, 504 where these elements may be comprised from a light emitting diode 503, an optical fiber 505, and optical fiber bundle 503 including laser light for as a light source of coherent light of a single wave length, or any desired combination thereof.

Thus with the above method steps at hand, a wide variety of illuminated two-layer glass-on-stone vessels 670 may be provided as demonstrated by the vessel 670c or FIG. 34C which further may include any of the fringe patterns 622 formed by the extensions 623 from FIGS. 30A to 30G, and/or include the internally encapsulated fiber bundles 504 of FIG. 30H. Further illuminated artwork and gold leaf or semi-transparent gold layers may be applied to these two-layer embodiments, like above, and also inlayed precious gemstones such a rubies, emeralds, and/or sapphires may be illuminated by light to augment the notifications by providing controlled light signals into ambient space when in use.

And then is use, any of these two-layer vessels, like above, may be used to charge a mobile phone 558, used to charge any of the electronic jewelry items 560 hereof, or used to charge both a mobile phone 558 and electronic jewelry items 560 placed therein together, as illustrated in FIG. 32C. And generally here, it should be understood, without repeating the drawing figures unnecessarily, that any of these two-layer vessels may further comprise any one or more of the features, attributes, or functions provided in the above three-layer glass-on-stone embodiments or any of the above two-layer glass-on-glass embodiments as rendered applicable therewith.

And thus for purposes of particularity and distinctiveness as to describing same, any of these two-layer glass-on-stone vessels of the type 670c of FIG. 34C, may more specifically be provided wherein the at least one light emitting element 502, 503, 504 includes a predetermined number of light sources 502 with each thereof being associated with a corresponding cluster of optical fibers 504 bundled together at an input end thereof 504a. And then the step of forming the stone vessel from the block of stone 632 may further comprise forming at least one side wall including a channel 669 formed therein on the inside surface of the stone vessel or shell 636, as discussed in FIG. 33C. Then here, the step of positioning the at least one light emitting element along the inside surface of the stone shell 636 may further comprise positioning a selected corresponding cluster of optical fibers 504 into a corresponding side wall channel 669 before the step of blowing molten glass to form the layer of glass 674. And where preferred, the channel is fan-shaped 669d, 669e having a neck portion proximate the bottom area of the stone shell and extending upwardly therefrom in a widening configuration and in the case of the truncated fan-channel 669e, each corresponding cluster of optical fibers 504 is contained within the layer of glass 674. In alternate embodiments, the side wall includes an upper edge 637, 680 and a selected number of fibers 505 from at least one cluster of optical fibers 504 each extend out from the upper edge, or a selected number of fibers 505 from each cluster of optical fibers 504 each extend out from the upper edge thereby forming a corresponding number of extension segments 623. And then further where desired, at least some of the extension segments 623 are of sufficient length to be movable by touch of hand or movement of air directed thereon, or all of the extension segments 623 are of sufficient length to be movable by touch of hand or movement of air directed thereon. In some embodiments thereof, all of the extension segments 623 are of substantially the same length, as shown in FIG. 30A, or the extension segments 623 are of randomly differing lengths as shown in FIG. 30C, or still here, like above, the extension segments may be cut to form a repeating fringe pattern, as in FIG. 30B. And then further where desired, the making of any of these two-layer stone vessels may further include the step of applying gold leaf onto at least a portion of the outside surface of the stone vessel 670 after all prior steps or onto the stone shell 636 as a work piece in process. Then as to these embodiments, the further step of placing the stone vessel or shell in a deposition chamber may be provided to thereby perform the step of depositing a semi-transparent layer of gold onto at least a portion of the outside surface of the stone vessel or shell as discussed in detail below in conjunction with FIGS. 52A and 52B. And like above, here also, some of the outside surface of the stone vessel 670 or shell 636 may be masked before the steps of placing the stone vessel or shell in the deposition chamber and depositing the semi-transparent layer of gold onto the outside surface thereof. And as discussed in the development of a particular lighting design, the step of positioning the at least one light emitting element includes placing the at least one light emitting element to direct light onto the portion of the outside surface of the stone vessel having the semi-transparent layer of gold. Or alternatively without masking or limited masking, then the step of depositing a semi-transparent layer of gold may be directed to providing the layer of gold onto a substantial portion of the outside surface of the stone vessel or stone shell in process. And in the limited case of masking, then the step of depositing a semi-transparent layer of gold onto the outside surface of the stone vessel may include masking only a bottom portion of the stone vessel to prevent the deposition of gold thereto. And then depending on process preferences, the step of placing in the deposition chamber is performed on the stone shell 636 before blowing molten glass there-into to form the layer of glass 674, or the step of placing in the deposition chamber is performed on the stone vessel 670 after fitting the lighting elements and blowing molten glass to form the layer of glass 674.

Thus with these additional method steps at hand, a further variety of illuminated two-layer glass-on-stone vessels of the type of vessel 670c of FIG. 34A may be provided by practicing a modified embodiment of the method of FIG. 31A to 31C where step 512 of blowing the second glass layer may not be included as necessary.

And then with this modified embodiment of the method of FIGS. 31A to 31C practiced in combination with at least some of the subassembly steps of FIG. 33C, the present invention is further directed to a method of making a natural stone jewelry box having light display notification and recharging functionality for use with personal electronic items, where this method comprises the steps of i) selecting a block of stone 632 having a desired translucence property; ii) forming a stone vessel, casing, or shell 636 from the block of stone 632, said stone shell 636 having an outside surface, an inside surface, and a thickness therebetween that allows some light to pass therethrough; iii) forming at least one socket or slot 669 into a selected inside bottom or side location of said stone shell 363; iv) positioning at least one light emitting element 502, 503, 504, 505 into a corresponding socket or slot 669 as formed into the selected inside bottom or side location of said stone vessel to accommodate the positioning thereof; and v) blowing molten glass onto the inside surface of the stone vessel to form a layer of glass to thereby encapsulate said at least one light emitting element within its corresponding socket or slot between the inside surface of the stone vessel 636 and the layer of glass 674. Then here as desired, this method may include the further step of forming at least one channel 669c, 669d, or 669e, or any combination thereof, on the inside surface of the stone shell 636 extending upwardly from a corresponding socket or slot 669a or 669b to contain therein at least one optical fiber 505 or an optical fiber bundle 504 as implemented in association with a corresponding light emitting element. Thus such vessels may be provided with each thereof including an electronic casing 506 as fitted in the bottom of the vessel and connected to the one or more light sources further including therein electronic charging pads, such that when in use the stone vessel may be provided as a jewelry box used to charge a mobile phone 558, used to charge electronic jewelry items 560 of the type hereof, or so used to charge both a mobile phone 558 and electronic jewelry items 560 placed therein together. And as such, in view of all of the above, such a two-layer, glass-on-stone vessel of the type 670c of FIG. 34C, may further comprise any one or more of the features, attributes, or functions recited below in claims 143 to 285 as rendered applicable therewith while here excluding repetitions of recurrent subject matter or repeating portions thereof.

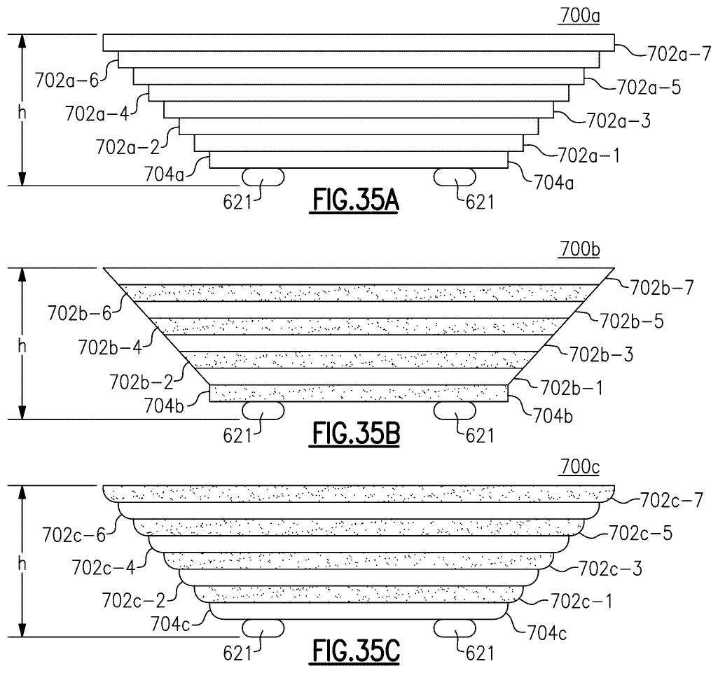

Now moving along from the above with same at hand, the inventors hereof further provide an inverted stairstep stone vessel and related methods of making and using same for use as a finished illuminated charging vessel for personal electronic items. Thus next in FIGS. 35A, 35B, and 35C there are presented side elevation views of three different embodiments of our horizontally-layered, laminated, or stratified stone vessel 700 formed from individual frame segments 702 and a bottom panel 704 where vessel 700a has an inverted stairstep outside surface, vessel 700b has a smooth outside surface with a square-cut bottom panel 704b, and vessel 700c has a quarter-round edging detail on the outside surface of each frame segment 702c and the bottom panel 704c. More particularly now with regard to each of these laminated vessels, vessel 700a of FIG. 35A includes seven consecutive frame segments referenced 702a-1 to 702a-7 and a bottom panel 704a each having square-cut out-side edges as illustrated. And as represented by the absence of any cross-hatching or stippling in the components 702a and 704a, each of the frame segments 702a and the bottom panel 704a are preferably cut from the same slab of stone material or otherwise preferably cut from consecutive slabs from the same quarry block. Vessel 700a of FIG. 35A also includes feet 621 that are glass-blown feet of the type discussed above in regard to the two-layer glass-blown vessels 616 of FIGS. 29A to 29G and also of the type discussed above in the two-layer and three-layer glass-on-stone vessels 670 of FIGS. 34A to 34G. And as indicated by the dimensional reference h, the vessel 700a has a standing height h that extends from the bottom of the feet 621 to the top surface of the top-most frame segment 702a-7.

Vessel 700b of FIG. 35B as illustrated also includes seven consecutive frame segments as here referenced 702b-1 to 702b-7 and the bottom panel 704b. And as further illustrated, the frame segments 702b-1 to 702b-7 after assembly are shaped in process to form a smooth outside surface while the bottom panel 704b has retained square-cut outside edges as shown. Further, stippling on the bottom panel 704b and alternate even-numbered frame segments 702b-2, 702b-4, and 702b-6 represents that these elements are cut from a slab of stone material of a first type while the absence of stippling on the odd-numbered frame segments 702b-1, 702b-3, 702b-5, and 702b-7 represents that these components are cut from a slab of stone material of a second type. For example, the bottom panel 704b and even-numbered frame segments 702b-2, 702b-4, and 702b-6 may be cut from a slab of Makrana marble from India that is black in color, while the odd-numbered frame segments 702b-1, 702b-3, 702b-5, and 702b-7 may then be cut from a slab of Makrana marble from India that is white in color. Thus in a finished assembly of such an embodiment, the vessel 700b would comprise alternating horizontal layers of black color marble and white color marble where as may be desired, only the white color layers are illuminated to further accentuate contrast between the layers when in use. And like above, here also, the vessel 700b includes glass-blown feet 621. And as indicated by the dimensional reference h, the vessel 700b has a standing height h that extends from the bottom of the feet 621 to the top surface of the top-most frame segment 702b-7.

Vessel 700c of FIG. 35C as illustrated, like the two above, also includes seven consecutive frame segments as here referenced 702c-1 to 702c-7 and the bottom panel 704c. And as further illustrated, the frame segments 702c-1 to 702c-7 and the bottom panel 704c before assembly are shaped in process to form quarter-round bottom side edging as illustrated. Further, stippling on alternate odd-numbered frame segments 702c-1, 702c-3, 702c-5 and 702c-7, represents that these elements are cut from a slab of stone material of a first type while the absence of stippling on the bottom panel 704c and the even-numbered frame segments 702c-2, 702c-4, and 702c-6 represents that these components are cut from a slab of stone material of a second type. For example, the bottom panel 704c and even-numbered frame segments 702c-2, 702c-4, and 702c-6 may be cut from a slab of Makrana marble from India that is white in color, while the odd-numbered frame segments 702c-1, 702c-3, 702c-5, and 702c-7 may then be cut from a slab of Makrana marble from India that is black in color. Thus in a finished assembly of such an embodiment, the vessel 700c would comprise alternating horizontal layers of white color marble and black color marble where as may be desired, only the white color layers are illuminated to further accentuate contrast between the layers when in use. And like above, here also, the vessel 700c includes glass-blown feet 621, and then as such, since the bottom panel 704c is white in color with then some degree of desired translucence, light sources may be positioned in the bottom of the vessel to direct light through the bottom panel and into the glass-blown feet 621 such that, like as discussed above, may thereby give a visual feeling that the vessel is floating on a cushion or pillow of light when placed in use on a supporting surface such as a nightstand, particularly during nighttime use or where otherwise placed in more dimly lighted ambient surroundings. And like above as here indicated again by the dimensional reference h, the vessel 700c has a standing height h that extends from the bottom of the feet 621 to the top surface of the top-most frame segment 702c-7 as shown.

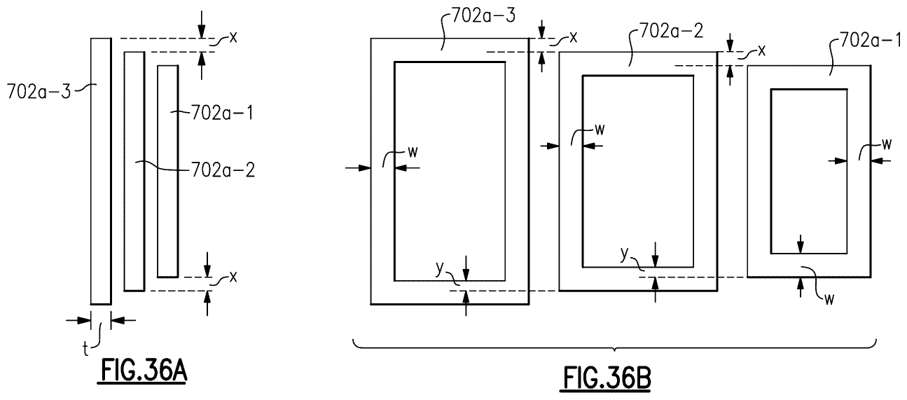

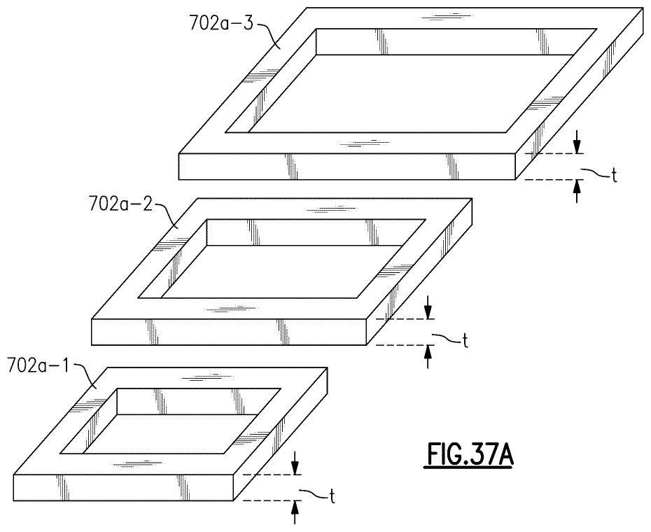

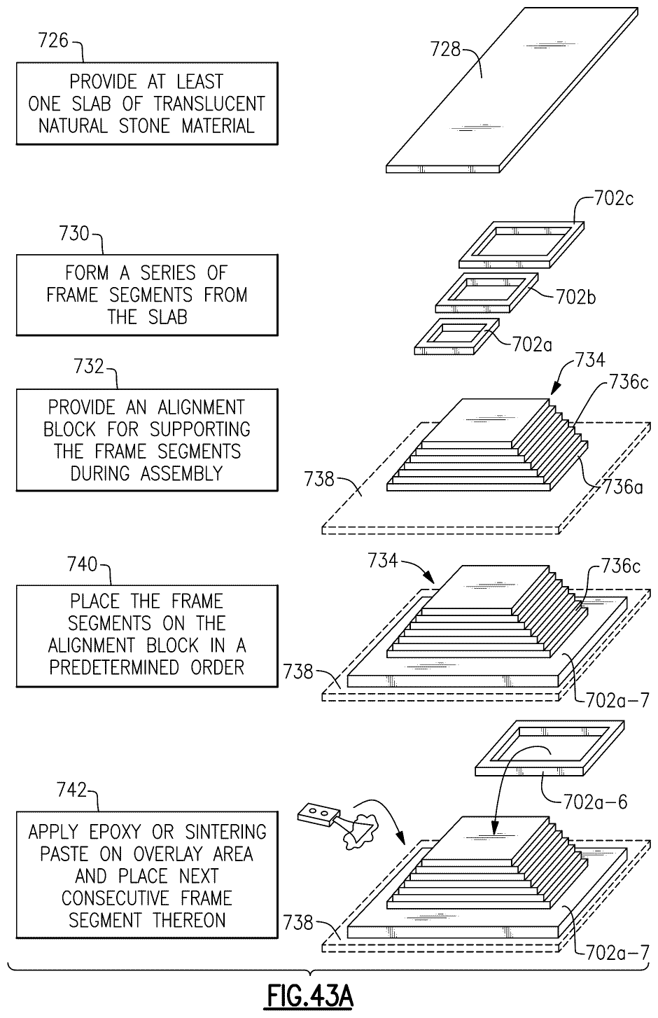

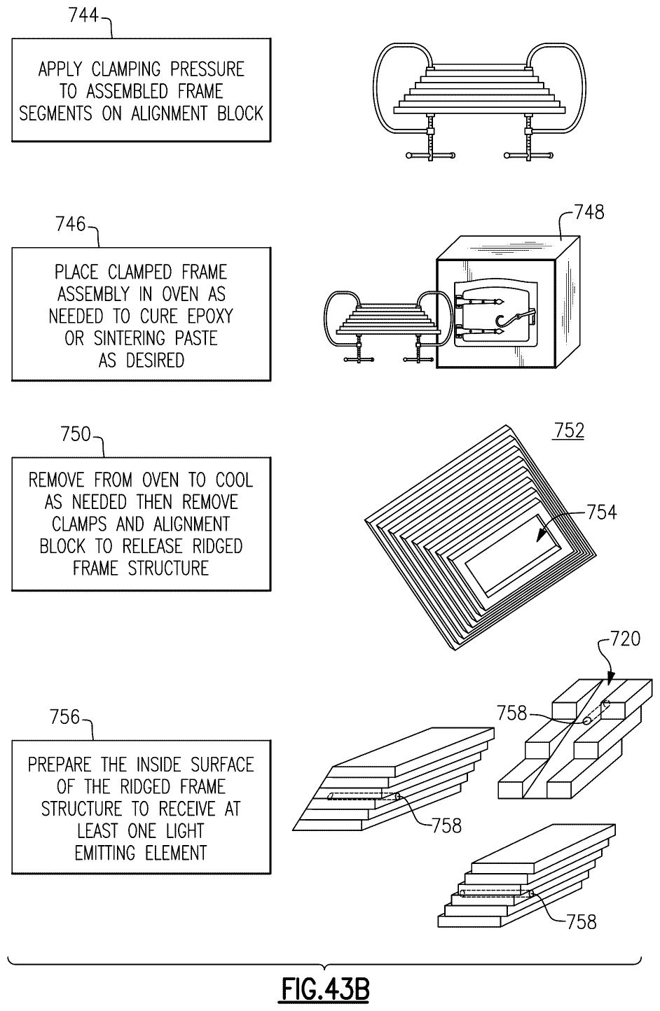

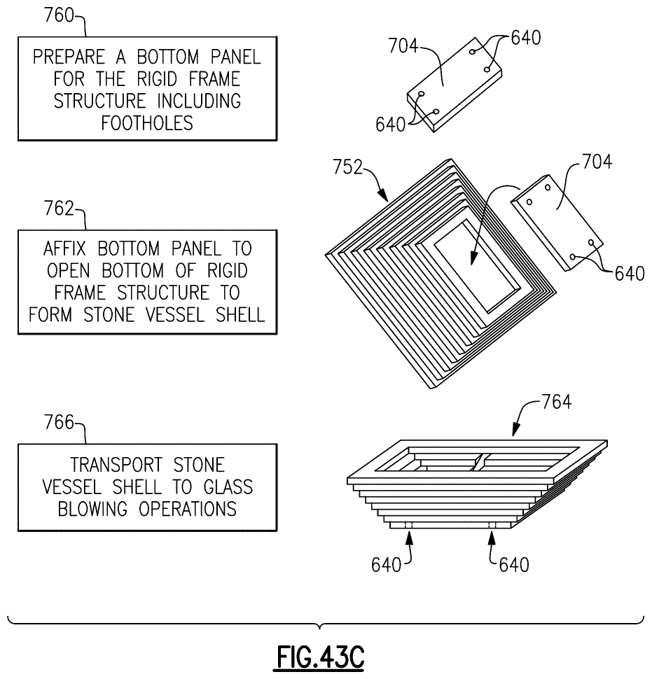

Process steps for fabricating and assembling vessels of the type 700a, 700b, and 700c of FIGS. 35A, 35B, and 35C, respectively, will be discussed in further detail below in FIGS. 43A to 49 while next in FIGS. 36A to 42C, various subassembly configurations of such vessels 700 will be presented so as to more particularly provide details appertaining thereto prior to discussing the various methods of making same.

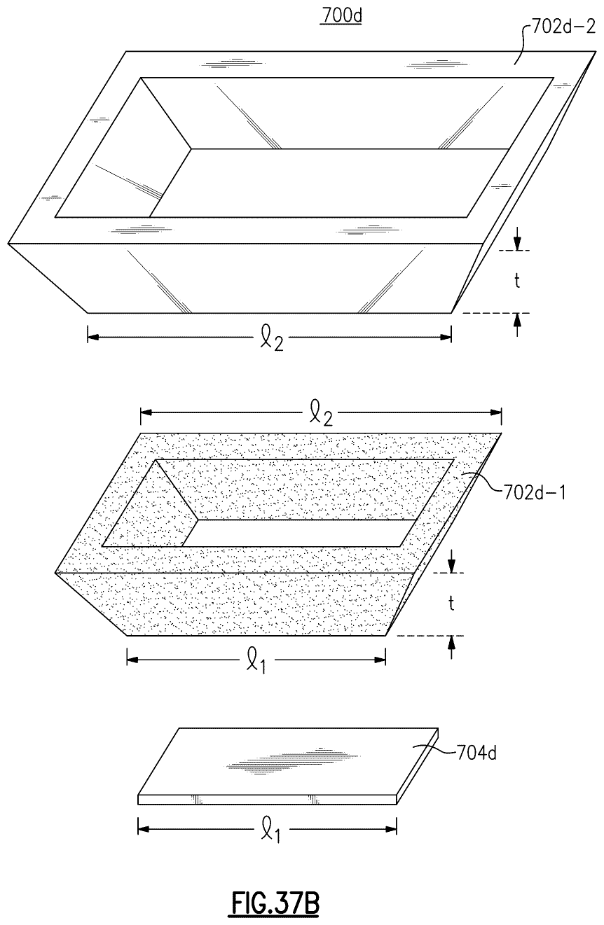

Thus next in FIG. 36A, there is shown a side view of three consecutive frame segments 702a from the stone vessel 700a of FIG. 35A here including, for example, individual frame segments 702a-1, 702a-2, and 702a-3 before assembly thereof, with each thereof having a thickness t. As illustrated, each consecutive frame segment is sized to have an overhang x as compared to an adjacent frame segment with this overhang x having a variable dimension as to length and then sized for each embodiment as desired and as may depend on the thickness of the slab used to make the segments and the desired total number of frame segments used to make same. Similarly, as shown in FIG. 36B, each consecutive frame segment 702a has an overlap y with adjacent frame segments such that in assembly, each frame segment is supported by the overlap area of the frame segment thereunder which then forms a stack of supported frame segments where here in the vessel 700a of FIG. 35A, the stack is formed by the indicated seven individual frame segments 702a-1 to 702a-7, while further in final assembly, the stack of frame segments is supported structurally by the bottom panel 704a, which in turn is supported mechanically by the feet 621 that position the outside surface of the bottom panel 704a at a desired or predetermined height above a supporting surface when in use. Here in certain preferred embodiments, the thickness of the frame segments t may be on the order of two or three centimeters or otherwise in the range of between about one-quarter of an inch (¼″) to about two inches (2″) thick. In other embodiments as may be desired with two or more frame segments and a desired standing height of between five inches (5″) and twenty inches (20″), for example, the frame segments may be on the order of two inches (2″) thick to about five inches (5″) or six inches (6″) thick. Thus in the case of a desired standing height of sixteen inches (16″) using one-quarter inch (¼″) thick frame segments 702, such an embodiment of a vessel 700 of this configuration would include about 56 to 62 frame segments given a standing height of sixteen inches (16″) with the feet 621 at between one-half inch (½′) to about two inches (2″) in height. In other embodiments, for example, where the thickness of the frame segments 702 is about two centimeters (2 cm) and the desired standing height of the vessel is specified at about seven inches (7″), with the feet 621 having a height of about one and one-half inches (1.5″), then seven frame segments would be needed to construct such an embodiment of a vessel 700 so configured. And further generally as to the sizing of the frame segments for various certain embodiments hereof, the width w of the frame segments may be on the order of one-half inch (½″) to about three inches (“3”) with the overhang x then being at about one-half inch (½″), and the overlap width y being at about three-quarter inch (¾″) to about one and one-half inches (1.5″) which may vary as needed to accommodate various interior and exterior surface configurations as further discussed below. Or otherwise dimensions x and y may approach their respective limits when in other embodiments hereof, the frame segments are thicker sculpted or tapered frame segments (about 2.5″ or greater in thickness) as formed from starting block material (discussed below in FIG. 37B), rather than relatively thinner frame segments (between about ¼″ to 2″ in thickness) as formed like here in FIGS. 36A and 36B from starting with slab material being at a standard of 2 cm or 3 cm in thickness.

FIG. 37A is a perspective assembly view of the frame segments 702a-1, 702a-2, and 702a-3 of FIGS. 36A and 36B which as illustrated may be on the order of 2 cm or 3 cm in thickness t as a typical standard of delivery for polished stone slabs from various quarries across the globe, while FIG. 37B is a perspective view of an unassembled vessel 700d having two frame segments 702d-1 and 702d-2 and a bottom panel 704d. Here as illustrated, the frame segments 702d-1 and 702d-2 are beveled or tapered in shape and may be each on the order of between about two and a half inches (2.5″) to five inches (5″) in thickness as formed, shaped, or otherwise sculpted from thick slab panels or block material as a starting work piece. As such, here in the vessel 700d of FIG. 37B, the top frame segment 702d-2 has a bottom length 12 while the bottom frame segment 702d-1 has a top length also of l₂ such that in assembly, the overhang x is essentially zero while the overlap dimension y is at essentially one-hundred percent of the frame width w. In a similar manner, the bottom frame segment 702d-1 has a bottom length l₁ while the bottom panel 704d has a length also of l₁ such that in assembly, the vessel 700d has a bottom detail such as that shown in above FIG. 35B. And here also in the vessel 700d, like above, the top frame segment 702d-2 and the bottom panel 704d may be formed from a block of Makrana marble from India that is white in color, while the bottom frame segment 702d-1 may then be formed from a block of Makrana marble from India that is black in color where light sources may then be positioned in the bottom of the vessel to illuminate the feet through the panel 704d.

And as should be readily understood in view of the above-discussed, in assembly when the top frame segment 702d-2, the bottom frame segment 702d-1, and the bottom panel 704d are affixed together as a rigid singular structure, a stone shell of the type of stone shell 636 shown in FIG. 32A is thereby formed and thus as such, such a two-color, stratified stone shell may be run through the process steps of FIGS. 31A to 31C and then further through any of the process steps of FIGS. 33A to 33C, as may be desired therewith.

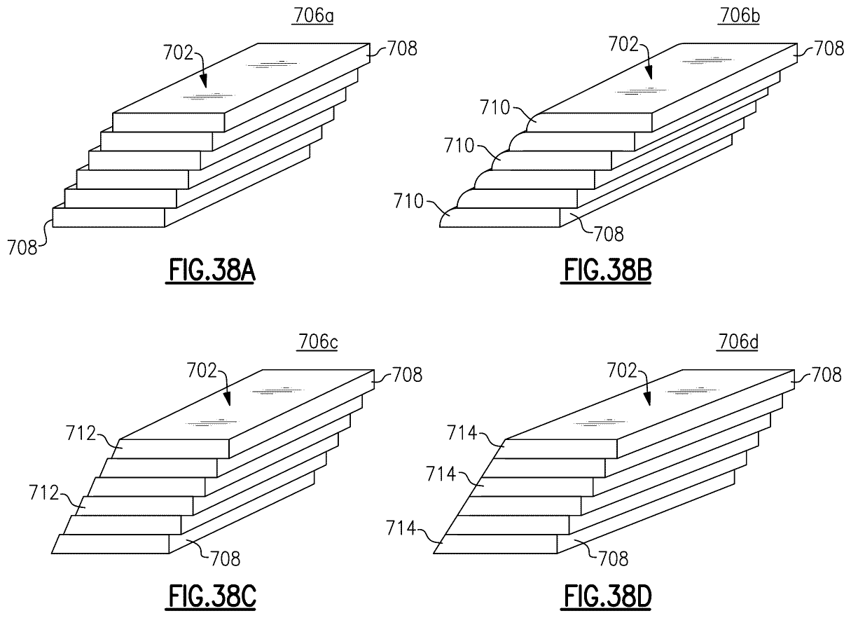

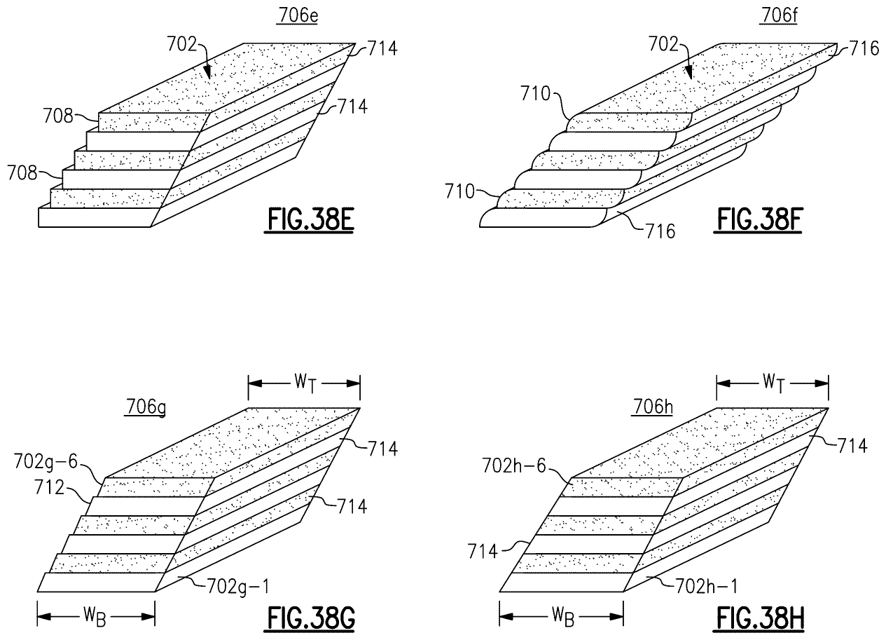

FIGS. 38A, 38B, 38C, and 38D are perspective detail views showing the outside inverted step surface of the stone vessel 700a of FIG. 35A with four different inside surface formulations, respectively. As such, FIG. 38A shows a stack of frame segments 706a that represents one of the side walls of the vessel 700a where the outside edge of each of the frame segments 702 has a square-cut formation or edge treatment 708 and the inside edge of each frame segment 702 also has such a square-cut edge treatment 708. Next, FIG. 38B shows a representative stack of frame segments 706b where the outside edge of each of the frame segments 702 has the square-cut edge treatment 708 while the inside edge of each frame segment 702 has a quarter-round top edge treatment 710. Then next in FIG. 38C, there is shown a representative stack of frame segments 706c where the outside edge of each of the frame segments 702 has the square-cut edge treatment 708 while the inside edge of each frame segment 702 has a louvered edge treatment 712. And then finally in FIG. 38D, there is shown a representative stack of frame segments 706d where the outside edge of each of the frame segments 702 has the square-cut edge treatment 708 while the inside edge of each frame segment 702 has a beveled or smooth edge treatment 714 such that each of the inside edges from a smooth planar surface within the vessel as illustrated.

FIG. 38E is a detailed perspective view of a stack of frame segments 706e representing a portion of a side wall of the stone vessel 700b of FIG. 35B having a smooth outside surface and a stepped inside surface where each of the frame segments 702 has has a beveled edge treatment 714 on the outside edge to thereby render the outside surface of the vessel smooth as in the vessel 700b of FIG. 35B where here in the stack 706e, the square-cut edge treatment 708 on the inside edge of each of the frame segments 702. And next, FIG. 38F is a detailed perspective view of a stack of frame segments 706f representing a portion of a side wall of the stone vessel 700c of FIG. 35C having the quarter-round top edge treatment 710 inside and a quarter-round bottom edge treatment 716 on the outside of the vessel as illustrated.

FIG. 38G is a detailed perspective view of stack of frame segments 706g representing a portion of a side wall of the stone vessel 700b of FIG. 35B having a smooth outside surface and a louvered inside surface formed by the louvered edge treatments 712 where each consecutive frame segment 702g starting at the bottom with frame segment 702g-1 to the top with frame segment 702g-6, has a progressively shorter width w such that the width WB at the bottom of the vessel is greater than the width WT at the top of the vessel. And similarly in FIG. 38H as represented by the stack of frame segments 706h, where the vessel is provided with a smooth surface on the inside and outside by the beveled edge treatments 714, each consecutive frame segment 702h starting at the bottom with frame segment 702h-1 to the top with 702h-6, has a progressively shorter width w such that the width WB and the bottom of the vessel is greater than the width WT at the top of the vessel. In this manner, the vessel 700 thereby fabricated and procured will have an art deco architectural feel given the slanting smooth interior and exterior side walls that taper in thickness from thicker at the bottom to thinner at the top.

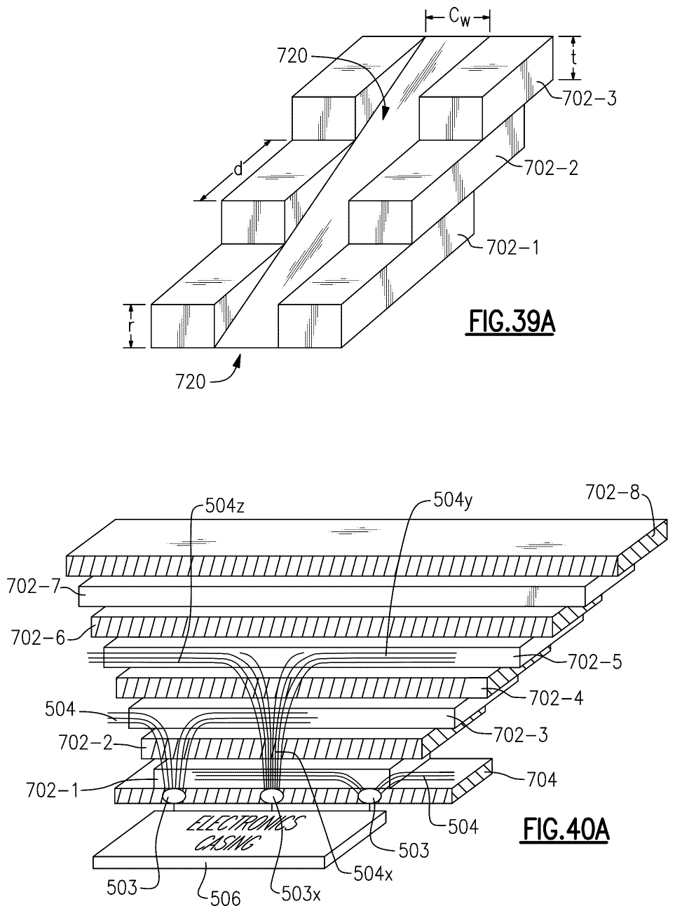

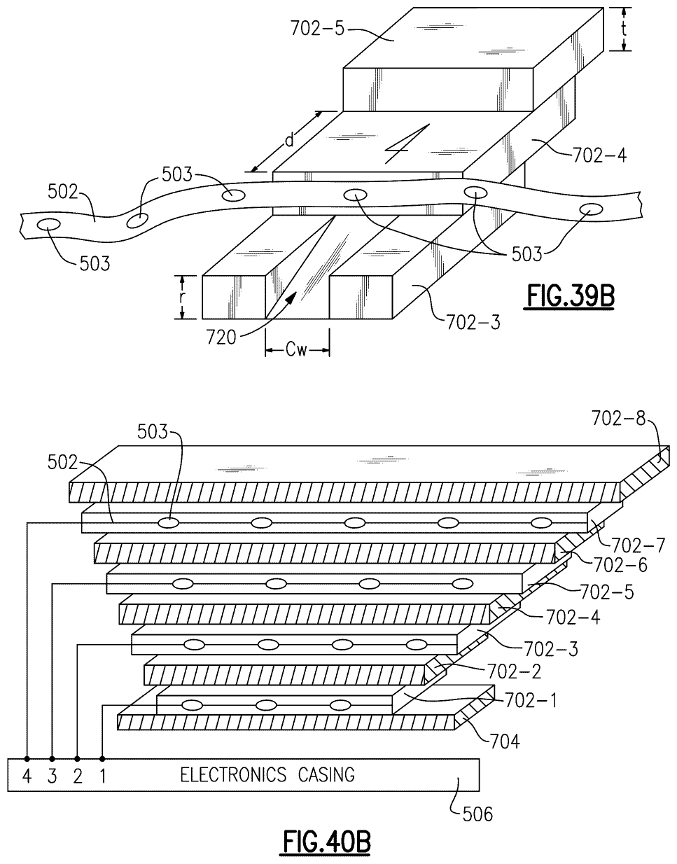

Next in FIGS. 39A to 42C, various subassemblies for facilitating illumination of the types of vessels 700 of FIGS. 35A to 35C will be discussed before proceeding to the below methods of making such illuminated vessels 700. As such, FIG. 39A is a detail perspective partial view of the inside surface of a laminated or stratified vessel 700 having a channel 720 formed therein for receiving a bundle of optical fibers according to certain of the above teachings hereof. The channel 720 has a channel width C_{w} that cuts up through several frame segments 702-1, 702-2, and 702-3, as illustrated. Each frame segment 702 when viewed from the interior has a raise dimension r and a depth d which dimensions r and d may be sized to efficiently accommodate the various lighting elements that will be associated therewith. Here then as to the geometry, the step rise r on the frame segments 702 inside of the vessel may be equal in length to the thickness t of the frame segments 702 on the outside of the vessel or the length of the raise may be rendered short than the thickness t during inside fabrication of the vessel as desired to accommodate the various lighting elements intended for application thereto. Thus next in FIG. 39B, an LED strip 502 of light sources 503 is deployed horizontally along the back side of the frame segment 702-4 as illustrated where when in process, the LED strip 502 will have applied thereof a layer of blown glass to thereby encapsulate the LED strip 502 between the blown glass and inside surface of the stone frame segments 702. Channel 720 as illustrated here terminates at frame segment 702-3 to provided where needed a pathway, trench, or conduit for an electrical connection from the LED strip 502 down to the electronics casing as provided in the bottom of the vessel.

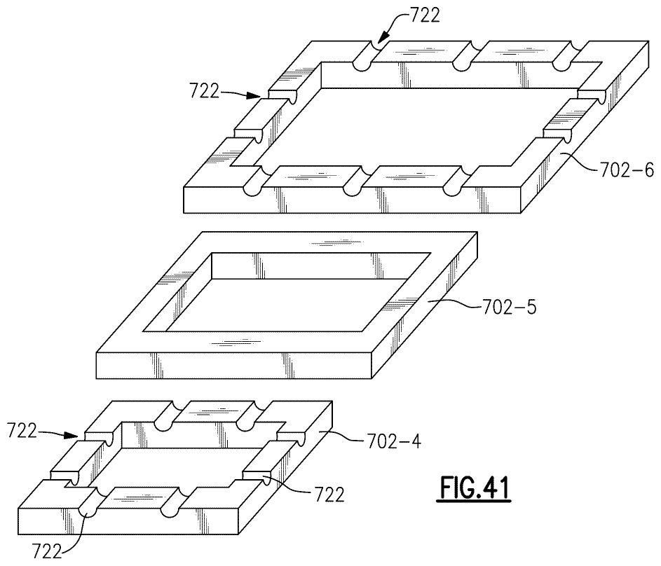

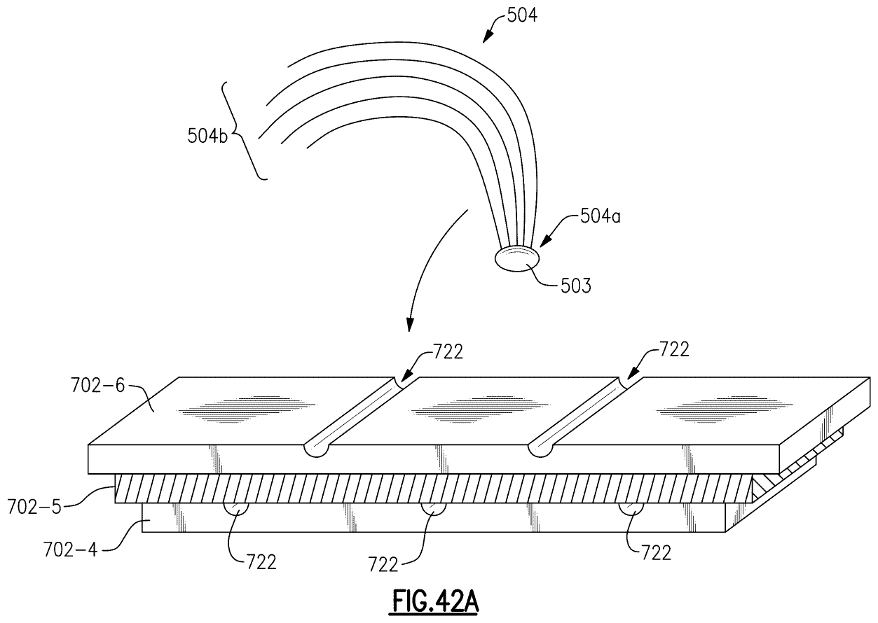

FIG. 40A is a partial perspective view of three fiber optic bundles deployed horizontally on the inside surface of a stone vessel of the type hereof with each respective bundle associated with a corresponding light emitting element. More particularly, light source 503x is associated with optical fiber bundle 504x with extends up to frame segment 702-5 where in assembly it has been split or parted into two oppositely directed sub-bundles 504y and 504z that when illuminated will have a common light source 503x. Here the splitting of a single bundle into two sub-bundles is for illustration purposes and thus it may be readily understood that such is not intended to be so limit but in various embodiments hereof, a single bundle with a corresponding light source at its input end may be in this manner divided during assembly into any number of desired sub-bundles and directed along any number of different frame segments as desired in rendering differing variations in the design of illuminated vessels of this type herein so provided by the inventors hereof.

FIG. 40B is a view similar to FIG. 40A wherein as an alternative to fiberoptic bundles, strips 502 of light emitting sources 503 are deployed horizontally along selected inside steps or louvers of a stone vessel of the type hereof. And as commonly illustrated in both of FIGS. 40A and 40B, the representative vessel shown therein includes a bottom panel 704 and alternate frame segments 702-2, 702-4, 702-6, and 702-8 that may be of a darker color or otherwise less transmissive to light as indicated by the cross hatching thereon, while the odd numbered frame segments 702-1, 702-3, 702-5, and 702-7 without cross hatching are provide in a natural stone and color that is transmissive to at least some light such that the fiberoptic bundles 504 and light strips 502 are thus applied thereto in assembly so that in use those frame segments are specifically illuminated as desired and so intended.

FIG. 41 is a view similar to FIG. 37 where half-round channels 722 for receiving a respective fiberoptic bundle have been formed in selected individual frame segments 702-4 and 702-6 before the assembly thereof as here illustrated.

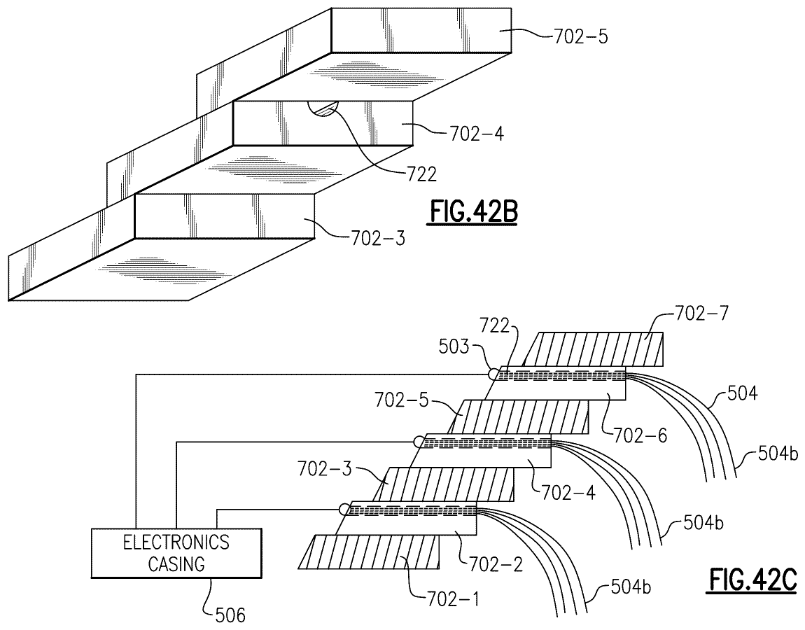

FIGS. 42A, 42B, and 42C are partial detailed schematic views of the light channels 722 of FIG. 41 as formulated for receiving a corresponding fiberoptic bundle 504. Thus here in assembly as each frame segment is stack atop the next consecutive frame segment, as represented in FIG. 42A, a respective bundle of optical fibers 504 may be place in a corresponding channel 722. Alternatively, the stone shell of frame segments may be first formed as a solitary structure as represented in FIG. 42B, and then thereafter but before the step of blowing a layer of glass into the vessel, the fiberoptic bundles 504 may be threaded through a corresponding channel 722. Thus according to either of these sub-assembly methods, the configuration of FIG. 42C may be thereby achieved where each channel 722 is a through-channel having threaded therethrough a corresponding bundle of optical fibers 504 which may each extend out from the exterior side wall surface of the vessel in a pony-tail style as illustrated such that their respective output ends 504b may be illuminated and may also be movable by touch of hand as discussed above. Here in the alternative, rather than being positioned in the bottom of the vessel, each light source 503 may be preferably poised or positioned adjacent the backside of a correspond frame segment as illustrated here and then encapsulated in that position when the layer of glass is applied thereto. And as discussed above, each of the light sources 503 are operatively connected to the electronics casing 506 as schematically shown here to thereby provide the desired light show and notifications by illumination as discussed and described above in detail and thus as such need not be repeated here.

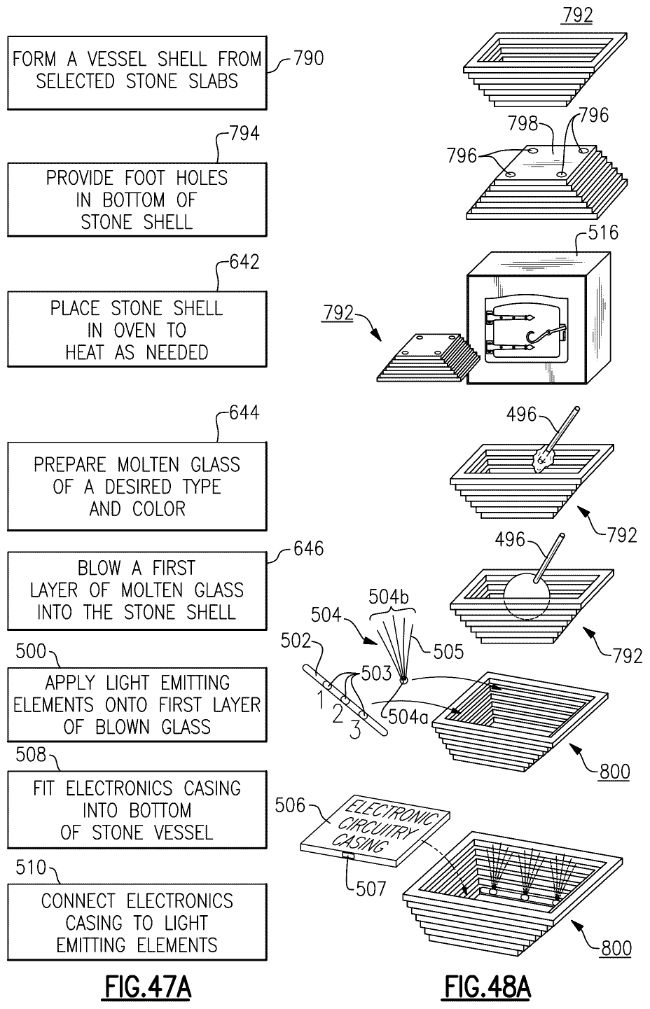

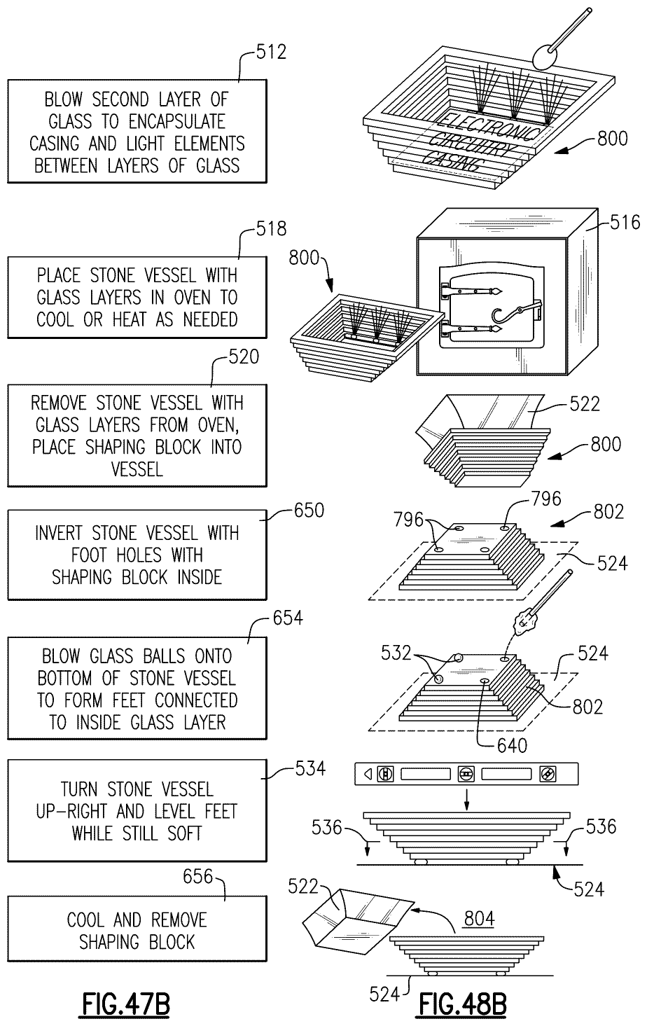

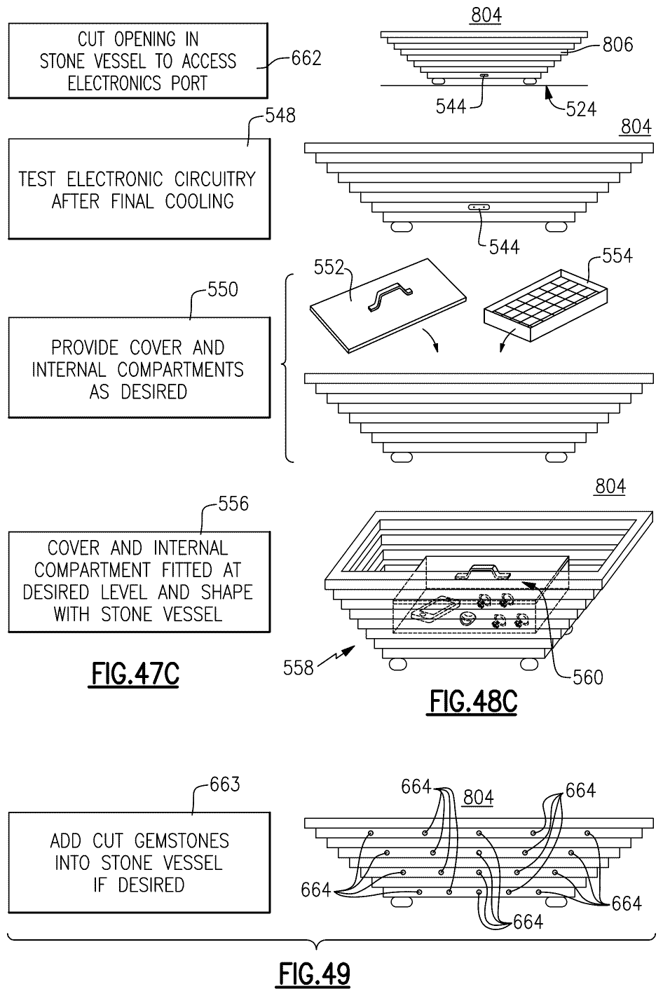

Now in regard to methods of making the type of stratified stone vessels 700 discussed above in detail as to various structural aspects thereof, FIGS. 43A, 43B, and 43C taken together provide a flow chart and pictographic illustrations of a series of steps of a method of making a horizontally-layered stone shell according to still further aspects of this invention. Thus at step 726, a stone slab 728 is provided and to accommodate the illimitation aspects hereof, the stone slab 728 is selected to have a desired translucent property so that some light may pass there through. The stone slab 728 may be preferably of a natural material such as onyx, alabaster, or marble or be of a more modern manufacture of stone material as specified for use herein such as crushed quartz formed into slabs by use of non-opaque resins or binders. Next at step 730, a series of consecutive frame segments 702a, 702b, 702c are formed where each of the frame segments are sized within the series to have an overhang x and an overlap y as discussed above in FIGS. 36A and 36B. The desired number of frame segments for any particular shell is dependent on the desired standing height h as discussed in connection with FIGS. 35A to 35C, and the thickness t of each frame segment as discussed in above in FIGS. 36A and 37B. Then back here again in FIG. 43A, at step 732, an alignment block 734 is provided. The alignment block 734 is comprised of a series of sheet segments 736 that each correspond in size to the inside opening of each of the consecutive frame segments and a support panel 738 shown here in dashed-line to distinguish it from the sheet segments 736 of the alignment block 734 and from the stone frame segments 702 shown here in these assembly illustrations. The support panel 738 is preferably sized just slightly larger in its length and width than the corresponding outside dimensions of the first frame segment 702 to be placed thereon. As such, at a next step 740, the first frame segment 702 is placed on the alignment block 734. Here for purposes of illustration, the first frame segment placed on the block is frame segment 702a-7 discussed above in FIG. 35A. Thus as would now be understood in view hereof, the top most frame segment 702a-7, 702b-7, or 702c-7 comprising the vessels 700a, 700b, and 700c, respectively, as shown in FIGS. 35A to 35C, for example, is the first frame segment to be placed on the alignment block in contact with the support panel 738 in the assembly thereof since the vessel or stone shell here is assembled up-side-down relative to its orientation while in use. Then next at step 742 epoxy or a specially formulated stone sintering paste is applied to the exposed side of the first frame segment 702a-7 and then the next consecutive frame segment 702a-6, here in making the vessel 700a of FIG. 35A, is placed on the frame segment 702a-7 as illustrated. As such, steps 740 and 742 are repeated for each consecutive frame segment until the last of the consecutive frame segments have been so assembled. Then at step 744, FIG. 43B, clamping pressure is applied to the frame segments and the alignment block as illustrated. Then next at step 746 the clamped assembly is placed in an oven 748 as may be desired to cure the epoxy or otherwise activate the stone sintering paste to affix the frame segments together to thereby form a singular rigid structure with an open bottom 754. Thus as such, at step 750, after the assembly has been removed from the oven and allowed to cool to a desired working temperature, the alignment block is removed from the assembly to thereby provide such a singular rigid structure 752 with the open bottom 754 as illustrated here at step 750 which at this stage of the method, the structure 752 is a work piece item in process. Then next at step 756, the inside surface is prepared to receive at lease one light emitting element or otherwise may be smoothed or shaped in any desired manner to achieve an inside surface of any desired formation as described above such as forming inside channels 720 or through-holes 758 for accommodating a specific lighting design plan or specification for any particular vessel under assembly. Then once all the interior fabrication on the structure 752 is completed as facilitated by the open bottom 754, at step 760 in FIG. 43C, a bottom panel 704 is prepared to preferably include foot holes 640 as discussed above in corresponding method step 638 of FIG. 31A. In other embodiments hereof, the bottom panel 704 may preferably not include any foot hole at this stage of the process so that such may be fabricated at a later time as discussed below. The next here in FIG. 43C at step 762, the bottom panel 704 is affixed to the stone shell 752 by epoxy glue or stone sintering paste and heated in the oven 748, FIG. 43B, as may be desired or otherwise needed the cure the epoxy glue or activate the sintering paste. Then once the bottom panel 704 is affixed in a secure and rigid manner with the open-bottom work piece structure 752, a finished stone shell 764 is thereby achieved and as such at step 766, the stone shell 764 may be delivered to a facility for glass blowing operations as provided below in detailed discussion in connection with the method of FIGS. 47A to 47C.

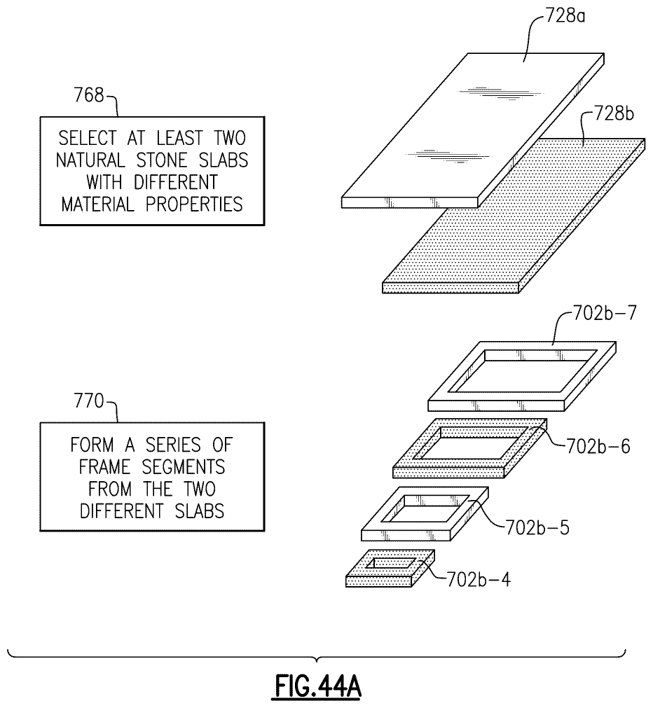

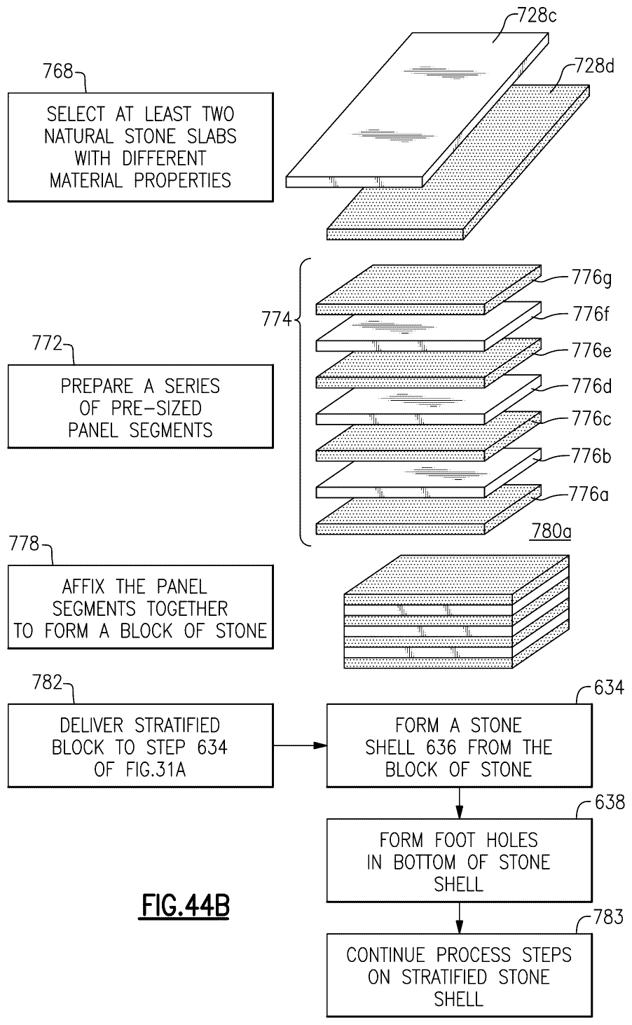

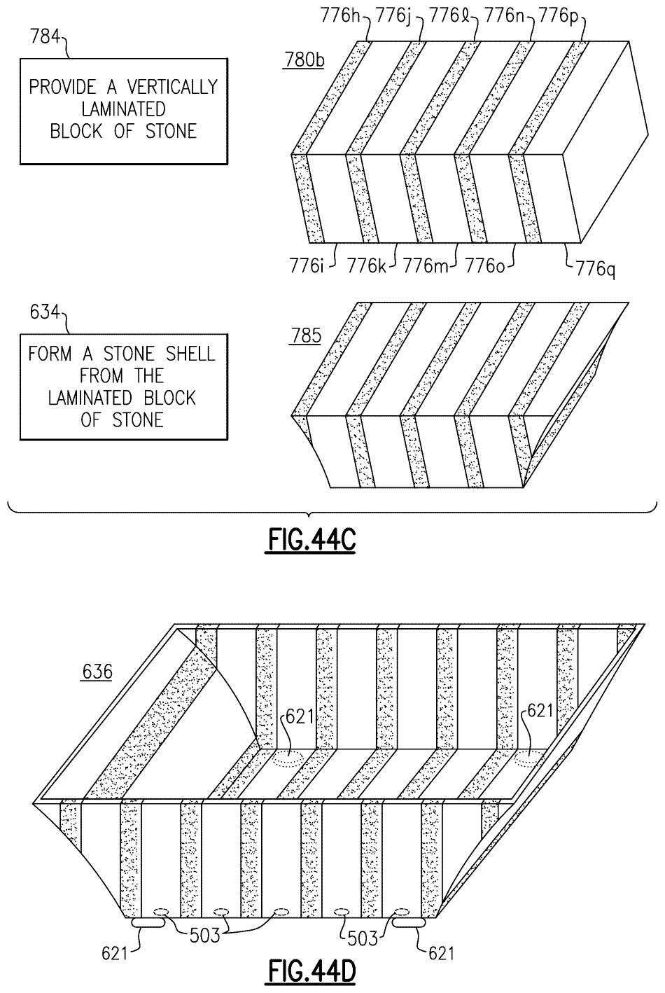

As an adjunct to the above method of FIGS. 43A to 43C, the horizontally-layered shell 752 may be formed from two different types of stone material. Thus FIG. 44A presents flow-chart steps and illustrates pictorially additional initial steps that may be included in the above method to make a horizontally layered stone vessel from two types of natural stone having different material properties where here such vessels are of the types 700b and 700c presented in above FIGS. 35B and 35C. Thus here in FIG. 44A at step 768, at least two different stone slabs 728a and 728b are selected such that each slab has different material properties as to thickness, translucency, and/or color, for example. Then at step 770, a series of frame segments are formed from the two different slabs 728a and 278b here shown as alternating frame segment 702b-4 as formed from slab 728b, frame segment 702b-5 as formed from slab 728a, frame segment 702b-6 as formed from slab 728b, and finally here for purposes of illustration, frame segment 702b-7 as formed from slab 728a which correspond in assembly to the frame segments with common reference comprising the vessel 700b in FIG. 35B wherein the outside surface thereof has been smoothed in fabrication rather than retained in an inverted stair-step fashion.

Next as an alternative to using frame segments to form a horizontally-layered stone shell of two different types of stone material, FIG. 44B flow-charts and illustrates pictorially a subassembly method for making a laminated stone block 780a from two types of natural stone slabs 728c and 728d having different material properties, where the laminated stone block 780a here in FIG. 44B may then be used in place of the block of stone 632 as starting with step 630 of FIG. 31A for further processing according to the above sculpting method of FIGS. 31A to 31C. Thus here again in FIG. 44B, at step 768 at least two stone slabs 728c and 728d each with different material properties from the other are selected as desired and as to thickness, they may of the same thickness or two different thicknesses. Then at step 772, a series or stack 774 of pre-sized panel segments 776 is prepared by fabrication and assembly thereof. As illustrated here in this example, the stack 774 is comprised of panel segments 776a, 776c, 776e, and 776g as formed from slab 728d, and panel segments 776b, 776d, and 776f as formed from slab 728c. Then after a respective stack of panel segments 774 is formed according to the particular specifications for a vessel to be provided therefrom, each of the panel segments 776 in the stack 774 are affixed together by epoxy or sintering paste under pressure with heat where needed or desired, to form therefrom a block of stone 780 which as illustrated here includes alternating horizontal layers in the block 780a or simply by changing the orientation of the block in fabrication when so configured, may include alternating vertical layers to thereby make a vertically-layered stone shell or vessel as may be desired in some alternate embodiments hereof, as shown below in FIGS. 44C and 44D. Then here as the last step 782 of this subassembly process, the completed layered or stratified block 780a is delivered to step 634 of FIG. 31A, where the common step 634 is applied thereto to form a stone shell from the laminated block of stone 780a, common step 638 is performed to form the foot holes in the bottom of the shell to facilitate formation of the glass-blown feet, and then here as per step 783, the remainder of needed steps along with any further optional or supplemental steps of FIG. 31A to 33C may be applied to the horizontally or vertically layered stone shell as desired. Thus for purposes of further detail and illustration, step 630 of FIG. 31A may here in FIG. 44C include the step 784 of providing a vertically laminated block of stone 780b as formed by the subassembly steps of FIG. 44B. Here the block of stone 780b includes panel segments 776h, 776j, 776l, 776n and 776p as formed from stone material of a first thickness and desired material properties assembled with alternating panel segments 776i, 776k, 776m, 776o, and 776q as formed from stone materiel of a greater thickness, for example, with different material properties than the former panel segments, with at least the later panel segments having a desired level of translucence for allowing some light to pass there-through when formed into a thinner stone shell. Then at common step 634 in FIG. 44C, a vertically layered or laminated stone shell 785 begins to take shape and with the completion thereof, a finished shell of the form 636 as shown in FIG. 44D may thus be provided in a manner akin to forming such a stone shell 636 as discussed in detail in connection in FIG. 32A at common step 634 of FIG. 31A. And then to make a vessel 670 of the types discussed in FIGS. 34A to 34G, the remainder of needed steps along with any further optional or supplemental steps of FIG. 31A to 33C may be applied to the vertically layered stone shell 636 of FIG. 44D where light sources 503 to be provided therewith may be advantageously positioned in any desired location among the vertical stone segments as illustrated here for exemplary purposes, with an electronics casing to be provided therewith by step 508 of FIG. 31A, and then glass blown feet 621 to be further provided therewith according to steps 650, 654, and 534 of FIG. 31A to thereby provide a vertically laminated illuminated glass-on-stone charging vessel of any of the types discussed in detail in FIGS. 34A to 34G.

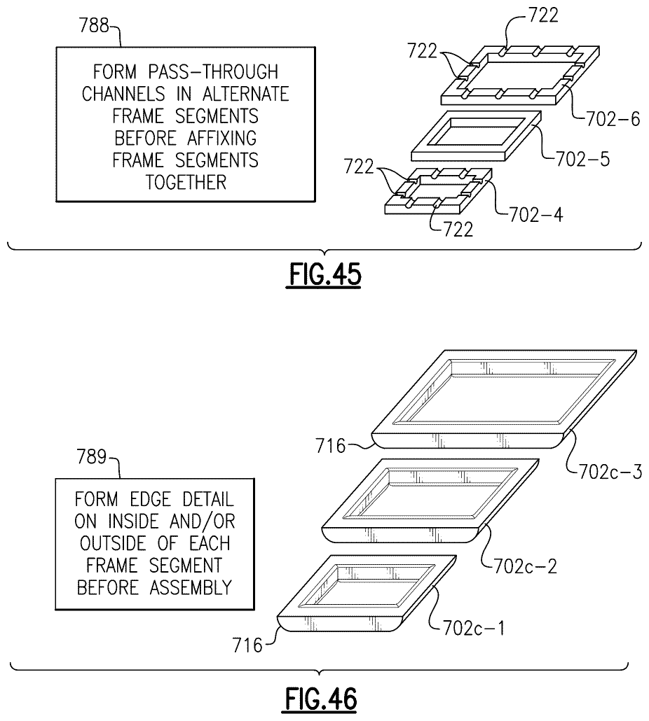

FIGS. 45 and 46 flow-chart and illustrate pictorially additional steps that may be used in making a horizontally-layered stone vessel of the types discussed in connection with FIGS. 43A to 43C and FIG. 44A. Thus at step 788 of FIG. 45, pass-through channels are formed in selected frame segments prior to assembly thereof. Here, the pass-through channels are in the form of the half-round channels 722 formed in alternated frame segments 702-4 and 702-6 as discussed above in FIGS. 4I and 42C. And then at step 789 of FIG. 46, edge details may be formed on the frame segments prior to assembly thereof into a stone shell for further processing. Here illustrated are beveled edges on the inside top surface of the frame segments and the quarter-round edge detail 716 on the bottom outside edge of each consecutive frame segment 702c-1, 702c-2, and 702c-3 which outside edge detail 716 may be seen in the stack of frame segments 706f in FIG. 38F and in the assembled frame segments of vessel 700c of FIG. 35C.

Embodiments of making a laminated stone shell. Now with general reference again to FIGS. 35A to 42C and FIGS. 43A to 46, certain aspects of the present invention are more particularly directed to a method of making a stone shell for use as a jewelry box, said method comprising the steps of i) selecting a at least one slab of natural stone material 728 having a desired translucence property; ii) forming a series of frame segments 702 from the at least slab of natural stone 728, each frame segment 702 having an overlay portion y and an overhang portion x; affixing overlay portions of consecutive frame segments 702 together to form a rigid structure 752 having an inside surface and an outside inverted stepped surface formed by the overhang portion x of each consecutive frame segment 702; and preparing the inside surface of the rigid structure 752 to receive at least one light emitting element, 502, 503, 504. The method may further advantageously include the further step of affixing a bottom panel 704 to the rigid structure 752 formed by the consecutive frame segments 702 as shown at step 762 of FIG. 43C. Here, the bottom panel 704 may be formed from the same at least one slab of natural stone material 728. Alternatively where desired, the step of selecting at least one slab of naturel stone material may preferably include selecting two slabs of natural stone material 728, step 768 of FIG. 44A, including a first slab 728a having a first translucence property and a second slab 728b having a second translucence property. There therewith, the consecutive frame segments 702 in said series of frame segments alternately formed from said first and second slabs 728a and 728b as provided at step 770 of FIG. 44A. And still further where desired, the step of selecting at least one slab of naturel stone material 728 may include selecting three slabs of natural stone material including a first slab having a first translucence property, a second slab having a second translucence property, and a third slab having a third translucence property. The like above, here with three different slabs 728, the consecutive frame segments 702 in said series of frame segments may be alternately formed from said first, second, and third slabs, and then with such at hand, the consecutive frame segments 702 in said series of frame segments may be randomly formed from said first, second, and third slabs. And as an extension thereof, where desired for more variety, the step of selecting at least one slab of naturel stone material 728 includes selecting four slabs of natural stone material including a first slab having a first translucence property, a second slab having a second translucence property, a third slab having a third translucence property, and a fourth slab having a third translucence property. Then here like above, the consecutive frame segments 702 in the series of frame segments may be alternately formed from said first, second, third, fourth slabs; or the consecutive frame segments 702 in said series of frame segments may be randomly formed from said first, second, third, and fourth slabs. And still further, the step of selecting at least one slab of naturel stone material 728 may include selecting a random number of slabs of natural stone material where the consecutive frame segments 702 in said series of frame segments are randomly formed from said random number of slabs of natural stone material. In any of these embodiments, the consecutive frame segments 702 may be affixed to one another under heat and pressure using a ceramic sintering paste formulated for use with natural stone, or the consecutive frame segments 702 may be affixed to one another using an epoxy glue formulated for use with natural stone, as described above in detail at step 742 of FIG. 43A. And where heat application is not needed or desired, then the consecutive frame segments 702 may be affixed to one another under pressure only using an epoxy glue formulated for use with natural stone. And then when a heat application is desired, the consecutive frame segments 702 are affixed to one another under heat and pressure using an epoxy glue formulated for use with natural stone. As to the illumination features hereof, the step of preparing the inside surface of the rigid structure 752 to receive at least one light emitting element 502, 503, 504 may include forming a smooth inside surface in the rigid structure 752, or partially removing some of the material forming the inside surface of the rigid structure 752. Then thereafter when desired and needed in the process, the bottom panel 704 may be formed from any one of said selected slabs of natural stone material 728 and affixed to the ridged structure 752 by any of the sintering or glueing techniques recited above.

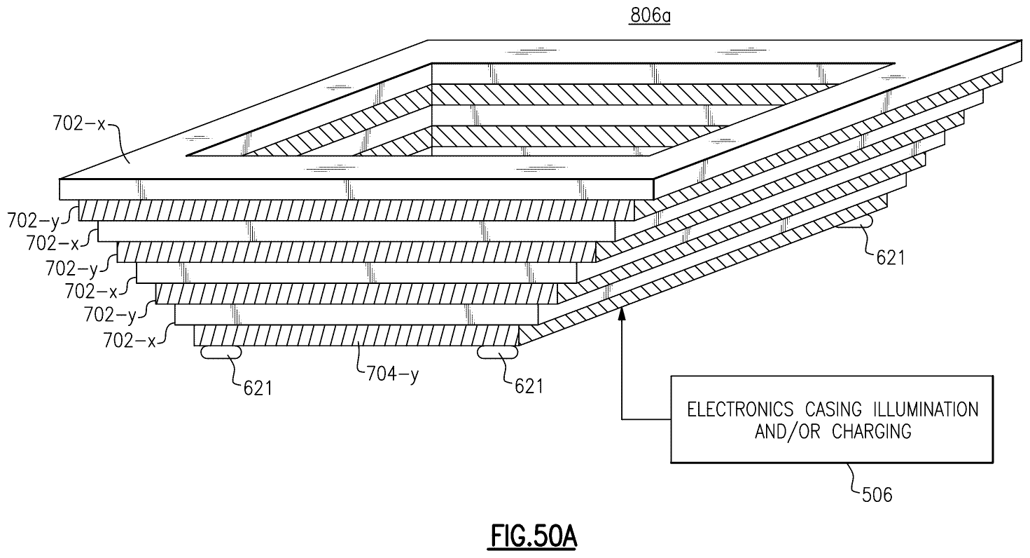

And now moving next to FIGS. 47A, 47B, and 47C, which taken together present a flow chart showing the steps of a method of making a horizontally-layered, laminated, or stratified glass-on-stone vessel for charging electronic jewelry, this method engaging some of the method steps of FIGS. 24A, 24B, and 24C as well as some of the steps from the methods of FIGS. 31A, 31B, and 31C; and in companionship therewith, corresponding FIGS. 48A, 48B, and 48C herein provide a series of perspective pictorials illustrating each of the method steps of FIGS. 47A, 47B, and 47C.

Then here to begin at step 790 in FIG. 47A, there is provided a horizontally-layered, laminated, or stratified vessel shell, carcass, or casing 792 as formed from selected stone slabs. The stone shell 792 may be formed by the above method of FIGS. 43A to 43C, the above method of FIG. 44B, or any other such methods as may be similarly provided. Next at step 794, in the case where foot holes 640 as per FIG. 43C were not provided, then here foot holes 796 are formed in the bottom 798 of the shell 792. And then as used in the glass-on-stone blowing methods above, an oven 516 may be used here at common step 642 to slowly heat the stone shell 792 to a desired temperature before the following glass blowing step is performed, and thus since some of the above method steps are similarly followed here, common reference numerals will be used herein for each of the common steps as for making the above glass-on-glass vessels 616 and glass-on-stone vessels 670. Then next here in FIG. 47A at common step 644, molten glass is prepared for blowing a first layer of glass into the stone shell 792 using a blow pipe 496 as discussed above. The molten glass here, like above, may be of a specialized formulation to promote adhering to the inside surface of the stone shell 792 in a permanent desired manner, and similarly, the inside surface of the shell may be lathered, before the first glass blowing step, with an etching solution to dissolve some of the stone material to thereby provide a rough rather than smooth surface so as to better promote mechanical adherence of the first blown glass layer to the inside surface of the stone shell 792. This rough surface, where desired, may also be achieved by grinding tools of a desired grit factor. On the other hand, the outside surface of the stone shell 792 may be more preferably brought to a very smooth fine polished surface finish by various finishing techniques including hand polishing by various finer and finer grits of stone pumice or buffing rouge, for example, as would be well understood by those of skill in the stone sculpting and stone finishing arts. Then at common step 646, a first layer of glass is blown into the stone shell 792, as prepared to receive same, to thereby begin the formation of an illuminated horizontally-layered stone vessel according to these teachings here including at this stage of the method a work-piece vessel 800 comprised of the laminated stone shell 792 and a first layer of blown glass.

Then like above at a common step 500, light emitting elements 502 and 504 are applied onto the first glass layer. Here, preferably, like above, the first layer of glass is still in a semi-soft state such that the light emitting elements 502 and 504 may be pressed into the first layer of glass. As illustrated schematically, light emitting element 502 is a strip of LED lights including three light sources or indicator lights 503 numbered 1, 2, and 3, and light emitting element 504, as shown schematically, includes a bundle of optical fibers 504 comprised of individual optical fibers 505 as may be bundled together at one end thereof to form an input end 504a where at the other end of the bundle 504, the individual fibers are loose and free such that they may be placed as desired to form an output end 504b of the bundle 504 as illustrated here and discussed in further detail below as to these laminated stone and glass vessels. Here also, like above, the indictor lights 503 may function in a similar manner as to the indictor lights 174 provided in the electronic charging cases and boxes 165 and 372 discussed above in detail in connection with FIGS. 4A to 4M-2 and FIG. 15A and in a similar manner to the indictor lights 406 provided in the above electronic jewelry box 402 as used with traditional and electronic jewelry as discussed in connection with above FIGS. 16A to 16C and thus as such, discussion of this functionality of the indictors lights 503 need not be repeated here.

Next in this manufacturing method as a supplied input item, like above, there is provided the electronic circuitry casing or electronics casing 506 here also including the knock-out or marking 507 which marks the location of an electric socket in the casing as discussed above in detail as to related methods and common components. The electronics provided in the casing 506 may be of any desired functionality including at least electronic functionality for controlling at least the light emitting elements and sources 502, 503, and 504 and for providing operability to a charging pad implemented therein for recharging personal electronic items such as smartphones and electronic jewelry. The type of charging pad provide therein is of the type discussed above in detail in connection with FIGS. 4B, 16B, and 16C as implemented in various embodiments across all the charging cases and electronic jewelry boxes hereof as discussed above in FIGS. 2A and 2B, FIGS. 3A and 3B, FIGS. 4A to 4M-2, FIGS. 13A and 13B, FIGS. 13C and 13D, FIGS. 14B to 14F, FIG. 15A, FIGS. 16A to 16C, and FIG. 23, and as further discussed as included with the above glass-on-glass vessels and the above glass-on-stone sculpted-stone vessels, and now here included with these laminated-stone and glass illuminated charging vessels for smartphones and electronic jewelry. And as discussed above as to the electronics manufacturer working in collaboration with a traditional jewelry maker to provide the electronic jewelry items hereof, such an electronics manufacturer may also be suited to working in collaboration with the inventor-artisans hereof who have designed and created these blown-glass, sculpted stone, and laminated stone vessels. As such and like above then, the particular technical details of the electronic circuitry provided in the casing 506 are considered beyond the intended scope of this disclosure and thus will be provided in companion disclosures as may be filed adjacent hereto.

Thus upon receipt from the electronics manufacture, the electronics casing 506 may be certified as tested for full functionality as designed before placing same into inventory as an input item to the present method of making these laminated stone and glass vessels as with the above glass-on-glass and glass-on-sculpted-stone vessels.

Moving along now to common step 508 next here in FIG. 47A, the casing 506 is fitted onto the first blown glass layer and at common step 510, the casing is electrically connected to the light emitting elements 502, 503, and 504 as per the particular lighting design for a specified vessel hereof. Here also, an electronics and lights testing step may be performed before proceeding to the next step. And also, like above, the first glass layer may still be soft when placing the casing 506 thereon or it may be in a cooler more solidified state. Then at common step 512, FIG. 47B, a second glass layer is blown into the work-piece vessel 800 to thereby encapsulate the casing 506 and the lighting elements 502, 503, and 504 between the two layers of glass. Then during any of the above steps or the steps hereof following, next as per FIGS. 47B and 48B, the work-piece vessel 800 now including the second layer of blown glass, may be place in the oven 516 under step 518 to heat or cool the stone and glass layers thereof at that time in the method as may be desired to avoid cracking, promote annealing, slowly change from a higher temperature to a lower temperature for further processing, slowly change from a lower temperature to a higher temperature for further processing, or more quickly cool or heat the glass so as to promote a crackled effect in one or both of the glass layers as all of such is well known in the traditional creative glass blowing arts where, however, natural stone shells and encased electronics, like those combined here, may not be known or included within the item there traditionally at hand and thus as such, these aspects of these inventions will require additional techniques and enhanced skills in the artisans handling and processing such like according to detailed specifications provided therewith.

Now with continuing reference to FIGS. 47B and 48B, at common step 520, the work-piece vessel 800 is removed from the oven 516 and a shaping block 522 is placed into the vessel onto the interior surface of the second layer of glass and may be gently pressed there-into and there-against to give a certain final desired shape to the interior shape of the glass vessel here under formation. The shaping block 522 is thusly important here for at least two principal reasons; first, the shaping block 522 shapes the interior of the glass vessel to thereby yield more precise tolerances as to the interior dimensions such that manufactured inserts such as jewelry trays or compartment dividers may be better ensured to fit as intended therewith and then second as to the importance of the shaping block 522 in this method, to provide structural support to the two glass layers inside the stone vessel shell during further processing and final cooling to reach a room-temperature final solidified state. And then as indicated at common step 650, this now temporarily-assembled, shaping-block-in-glass-on-stone assembly 802 is inverted and placed on the top surface of a work bench 524. Next so as to provide attractive and functional feet this vessel, like above, at common step 654, the vessel here is at a desired temperature for further processing as removed from the oven 516 to achieve same, with the shaping block still in the vessel pressing against the layers of soft glass while atop the work bench 524 as illustrated in the schematic pictorials here at this stage of the present method. So then here next at step 654, glass balls 532, like above, in a semi-molten state may then be applied to holes 796 in the stone shell to come in contact with the first layer of glass therein to thereby adhere the glass balls 532 to the first glass layer with the stone shell material adjacent the holes 796 being pinched there-between. Here the glass-on-stone vessel may be allowed to begin to cool to thereby start the approach of the glass layers to a final state of solidification while, as is well known in the creative material arts of glass blowing, a torch or blow-torch of the oxyacetylene type or other type may be used at this time to provide localized heating at the location of the feet thorough the stone holes so initially semi-molten balls of glass 532 adhere better to the first glass layer inside the vessel. In this manner, a better bonding may be achieved between the semi-molten glass balls 532 and the heated or re-heated localized areas on exterior surface of the first glass layer as exposed in the hole 796 previously formed in the stone shell. Then within a timing tolerance as to the pliability of the applied glass balls 532, at common step 534, the assembly 802 is turned up-right on the work bench 524 and while the glass balls 532 are in a certain desired pliable state, a slight downward pressure as indicted by pressure arrows 536, is applied to the temporary shaping-block-in-glass-on-stone assembly 802 so that the glass balls 532 as feet-to-be may be squeezed or squashed a bit, thus, like above, deforming same into semi-oblong pucks or disks with rounded side edges in an artistic manner to thereby hand form custom feet for each vessel such that when placed in use, the top of the vessel will set level with a night-stand, for example, as illustrated above in FIG. 19 as to the vessel 452 shown therein. And now here again in FIGS. 47B and 48B, at common step 656, the assembly is allowed to cool and at a prescribed time, the shaping block 522 is removed from the vessel to thereby present a laminated stone and glass vessel 804 made according to this method. Then next in FIGS. 47C and 48C, in a back side 660 of the vessel 804, at step 662, an opening 544 may be formed to thereby access an electronics port or socket previously provided in the electronics casing 506 discussed above. Here the opening 544 in the laminated stone and glass vessel 804 and also a corresponding opening in the electronics casing 506 as marked by the knock-out or marking 507, may be achieved by stone, glass, and ceramic grinding tools well know in the art, or may be otherwise pre-formed in the laminated stone shell 792 and then formed in the glass in process by cutting the glass while in a semi-molten state as like above and so applied here. Then at common step 548, after all the above manufacturing steps are performed, particularly those where heat is applied to the vessel as a work-in-process item or work-piece vessel 800, the electronic circuitry contained within the electronics casing is tested using test equipment connected to the socket in the casing via the opening 544 to ensure full functionality thereof. And as would be understood by those of skill in the relevant arts, such a testing step or steps as to the electronic functionality, may be performed at any desired time during fabrication and assembly to ensure the good functioning thereof throughout the steps of this method of making such an item as this laminated glass-on-stone vessel 804.

Then to prepare for consumer use, at common step 550, FIG. 47C, a cover 552 and a jewelry tray 554 with compartments may be provided as illustrated in FIG. 48C. And at common step 556, the cover 552 and tray 554 may be fitted to desired tolerances in assembly thereof to be tailored as to a desired level of height and shape for fitting within the interior of the vessel 804 as discussed in further detail herein below in connection with FIGS. 53 to 56. Here, like above, since the shaping block 522, FIG. 47B, was used to form the interior dimensions of the vessel 804 given the shape and interior formation of the laminated stone shell 792, tailoring needs for the cover 552 and tray 554 will be minimal or even unnecessary when the cover 552 and tray 554 are specified or “spec'd” as to the particular shaping block used in manufacturing. And lastly here, FIG. 48C, like above in FIG. 25C, shows a smartphone 558 and some of the electronic jewelry 560 hereof in dashed line in the bottom interior of the vessel 804 as representing the charging or recharging thereof in accordance with the teachings hereof.

And where desired in a laminated glass-on-stone vessel 804 hereof, FIG. 49 shows common additional method step 663 and corresponding schematic representation of precious or simi-precious gemstones 664 inlaid into the vessel 804 made according to these certain additional preferred embodiments hereof to thereby provide a unique outside surface associated therewith. Such inlayed gemstones when illuminated with light in a field of white translucent marble, like the Makrana marble of the type from India discussed above, for example, will provide further brilliance and animation to the vessel during use thereof as a recharging station for electronic jewelry that may better comport therewith as to desired style and elegance for selected market segments that may further desire such artistic features integrated into such a utilitarian article of manufacture.

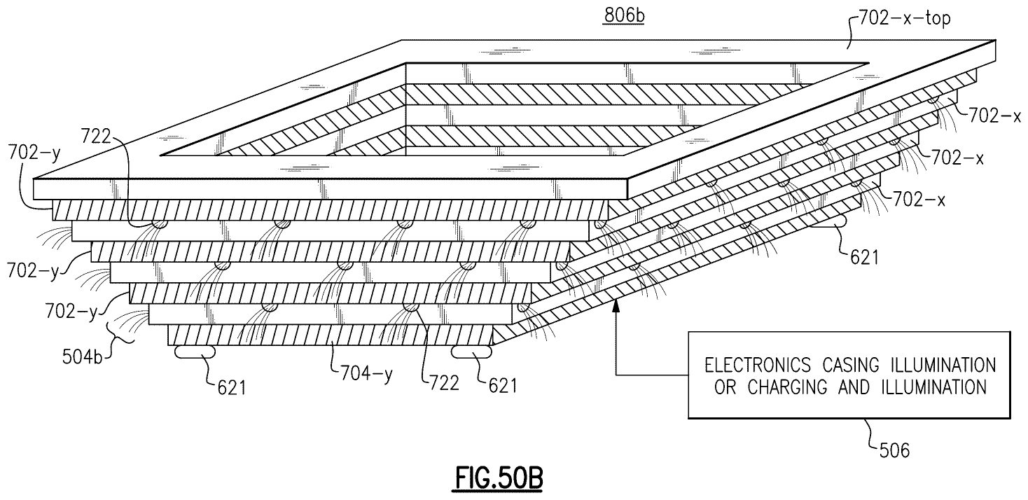

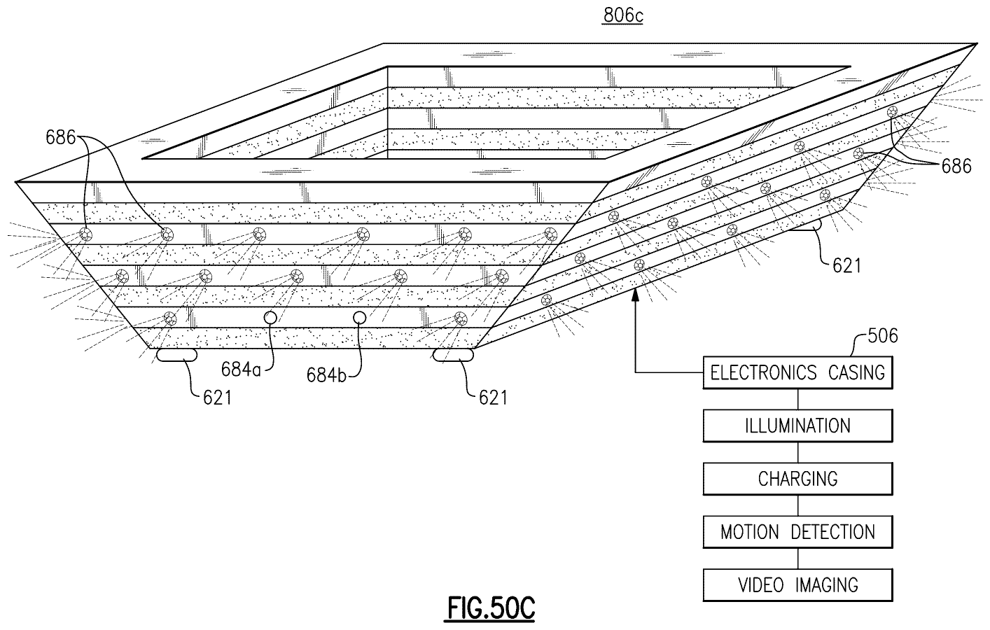

FIGS. 50A to 50D are perspective illustrations showing four different horizontally-layered natural stone jewelry vessels 806 that may be made according to corresponding methods hereof. Each of the vessels 806a and 806b, FIGS. 50A and 50B, respectively, are shown with smooth inside surfaces and outside surfaces that are inverted square-cut, stair-stepped in formation as discussed above, and thus as such may be provided by first providing the frame segments of FIGS. 36A and 36B as then fabricated by the method of FIGS. 47A to 47C. Next in contrast thereto, each of the two vessels 806c and 806d, FIGS. 50C and 50D, respectively, are shown with smooth inside surfaces and outside surfaces, rather than stair-stepped, and thus as such may be provided by either first providing the frame segments of FIGS. 36A and 36B as then fabricated by the method of FIGS. 47A to 47C, or may otherwise be provided by starting from a laminated block of stone 780, rather than frame segments, as shown in FIG. 44B as then further fabricated into such a finished vessel 806c or 806d by the sculpting method of FIGS. 31A to 31C.

Thus with the above at hand and now as to the vessel 806a of FIG. 50A, there are provided alternating frame segments 702-x and 702-y where the frame segments 702-x are cut from a stone slab of a first type while the frame segments 702-y and the bottom panel 704-y are cut from a stone slab of a second type, as discussed above. Also shown supporting the vessel 806a are the glass-blown feet 621 as formed by the above subassembly process steps. And so that the vessel 806a may have at least some of the electronic functionalities hereof, the vessel 806a is provided with the electronic casing 506 as per the method of making same, so that illumination, recharging, or both illumination and recharging may be provided therewith as according to the various embodiments and teachings hereof.

Next as to the vessel 806b of FIG. 50B, there are similarly provided alternating frame segments 702-x and 702-y where the frame segments 702-x are cut from a stone slab of a first type while the frame segments 702-y and the bottom panel 704-y are cut from a stone slab of a second type, as discussed above. As further illustrated in this example embodiment, the frame segments 702-x, excluding the top-most frame segment 702-x-top, included the half-round light channels 722 which each include a bundle of optical fibers with their respective out-put ends 504b extending out from the end of the channel 722 as illustrated. Thus in this manner, with light sources provided in the bottom of the vessel, a light show, illumination, and notification-by-illumination may be further provided as discussed above. And like above, the glass-blown feet 621 are provided to support the bowl of the vessel above a surface such as the top surface of a night stand when in use. And further as discussed above, each of the feet 621 may have provided in association therewith, a respective light source that directs light downwardly into its corresponding foot such that in use, there is thereby provided the feeling of the vessel floating on a cushion or pillow of light when placed in use on a supporting surface such as a night stand, particularly during nighttime use or as used in an otherwise dimly lighted room. And so that the vessel 806b may have at least some of the electronic functionalities hereof, the vessel 806b is here provided with the electronic casing 506 as per the method of making same, so that illumination, recharging, or both illumination and recharging may be provided therewith as according to the various embodiments and teachings hereof.

Then next in FIG. 50C, the vessel 806c may be formed from frame segments or from a laminated block of stone each comprising two different types of stone material such as alternating black and white horizontal layers of marble as discussed above. Here the vessel 806c is provided with ruby gemstones 686 that have positioned there-behind a light source 503, as in FIG. 34E-2, so the illumination aspects hereof are thereby enhanced as to light show features as discussed above in detail in connection with FIG. 34E-2 and thus not repeated here. In addition thereto, motion detection and video imaging may be provided in the vessel 806c as shown here in FIG. 50C via the holes 684a and 684b, respectively, as discussed above in detail in connection with FIGS. 34E-3 and 34E-4.

And finally as to these particular exemplary embodiments, the vessel 806d of FIG. 50D is similarly formed from frame segments or from a laminated block of stone each comprising two different types of stone material such as alternating black and white horizontal layers of marble. Here the vessel 806d is also provided with ruby gemstones 686 that have positioned there-behind a light source 503, see FIG. 34E-2, so the illumination aspects hereof are thereby enhanced as to light show features as discussed above in detail in connection with FIG. 34E-2, and thus not repeated here. And further as illustrated, alternate layers of the vessel 806d are provided with inlaid semi-precious stones 664 as a decorative feature that may be illuminated as discussed above in method step 663 of FIG. 49. And also like above in FIG. 50C, motion detection and video imaging may be provided in the vessel 806d as shown here in FIG. 50D vis the holes 684a and 684b, respectively, as per the details of FIGS. 34E-3, and 34E-4. And then further here, a rechargeable battery 808 may be provided in conjunction with the electronics casing 506 so that vessel 806d may operate in a cordless manner with such a cordless feature further discussed below in connection with the illuminated flower vase of FIG. 57B.

FIG. 51 charts and illustrates an additional method step 810 that may be included in making any of the glass or stone vessels 616, 670, 700, 806 hereof where gold leaf is applied by hand. As such, at step 810 a traditional sheet of gold leaf 812 and a traditional gilding brush 814, typically of squirrel hair, are provided and then on an area of the vessel where gilding is desired, an application of sizing, sizing glue, sizing adhesive, or gilding adhesive is applied thereto, and then gold leaf is applied to the sizing as would be known by those of skill in the traditional gilding arts.

To better control the desired thickness of the gold layer so that same is semi-transparent and will thereby allow some directed light to pass there-through, FIGS. 52A and 52B chart and illustrate additional method steps that may be included in making any of the glass or stone vessels hereof where a thin gold (Au) coating or layer is applied by vapor deposition. Thus here at step 816, a sculped stone vessel 670, as an example of any of the above, has applied thereto masking 818 where gold is not desired. Here the masking 818 is shown as a series of stripes but such masking 818 may be applied in any desired pattern or formation. Then at step 820, the vessel 670 is placed in a deposition chamber 822 and at step 824 a desired thickness of gold coating 826 is applied or deposited onto the vessel and as such the gold coating 826 adheres to the outside surface of vessel in the areas without masking. Then at step 828, the masking is removed and a gilded vessel is thereby provided where here, the vessel 670 includes striped areas 830 without gold that may be illuminated, and wider stripped areas where the gold coating 826 has been applied which gold-coated areas may also be illuminated with any desired number and type of upwardly directed light sources provided in the bottom of the vessel. In this manner, when light is directed through the areas with gold coating 826 which have been controlled in the making thereof to be of a semi-transparent thickness, the light-gold interaction will provided a special type of light show that may have notification aspects associated therewith, where such notification may include indicating when a mobile phone or item of electronic jewelry has been full charged, or otherwise used as a silent light-show wake-up alarm, or in non-notification uses, may be deployed simply as a night-light when dimly lighted or deployed as a lamp fixture providing some desired level of ambient lighting during any time of the day or night.

Embodiments of making an illuminated glass-on-stone vessel. Now with general reference again to the methods of FIGS. 31A to 32C and FIGS. 47A to 52B, as to making the types of glass-on-stone vessels 670 of FIGS. 34A to 34G, the types of vessels 700 of FIGS. 35A to 35C, and the types of vessels 806 of FIGS. 50A to 50D, certain aspects and features of these inventions are further directed to a method of making a natural stone jewelry box or jewelry vessel having light display notification and recharging functionality for use with personal electronic items, said method comprising the steps of i) selecting a stone shell 670, 806 made according to any one of the methods hereof; ii) blowing molten glass into the stone shell 670, 806 to form a first layer of glass 674 on the inside surface of the stone shell; iii) applying at least one light emitting element, 502, 503, 504 onto the first layer of glass 674 in a selected location; and iv) blowing molten glass onto the first layer of glass 674 to form a second layer of glass 676 to thereby encapsulate said at least one light emitting element 502, 503, 504 between the first and second layers of glass 674 and 676. Here by inherent physical properties, the stone shell 670, 806 has a bottom or bottom area 641,798 with a corresponding outside surface and inside surface. And to provided desired electronic functionally, this method includes the further step of providing or obtaining an electronics casing 506 having circuitry secured therein for performing or otherwise providing such desired electronic functionally. And thus with the electronics casing 506 at hand, this method further includes step of fitting the casing 506 onto the inside surface of a respective bottom area 641 or 798 of the vessel under assembly. The with same so situated, the method further includes the step of connecting the at least one light emitting element 502, 503, 504 to the electronic circuitry in the electronics casing 506 before the step of blowing molten glass to form the second layer of glass 676. And as may be preferably desired in certain embodiments, the electronics casing 506 is formed from a high-temperature ceramic material having heat resistant properties that protect the electronic circuitry from the temperatures associated with the step of blowing molten glass onto the ceramic casing. And where needed or desired, this method may further comprise the step of placing the stone vessel into an oven to cool so the glass layers avoid cracking while cooling down from the higher temperature associated with blowing molten glass, to a lower temperature for further processing. And then to provided the desired recharging functionality, the electronic circuitry hereof includes circuitry for charging rechargeable batteries when placed in the vessel during use thereof, and as to the illumination aspects hereof, the electronic circuitry further includes circuitry for providing power to the at least one light emitting element 502, 503, 504 which may be comprised of a light emitting diode, an optical fiber, laser light, or a combination thereof. Thus by these methods there are provided various embodiments of an illuminated stone jewelry box or jewelry vessel that may be used to charge a mobile phone such as represented by the mobile phone 558, FIGS. 32C and 48C, or used to charge electronic jewelry items such as represented by the jewelry items 560, FIGS. 32C and 48C, or otherwise used to charge both a mobile phone and electronic jewelry items placed therein together. And then still further as to the illumination aspects hereof, the at least one light emitting element includes a predetermined number of light sources 503 with each thereof being associated with a corresponding cluster of optical fibers 504 bundled together at an input end 504b thereof. And thus to achieve same in assembly, the step of applying the at least one light emitting element onto the first layer of glass 674 further may comprise pressing a selected corresponding cluster of optical fibers 504 into a corresponding side wall channel before the step of blowing molten glass to form the second layer of glass 676 wherein each cluster of optical fibers 504 is contained within the first and second layers of glass 674 and 676. The in final assembly, where the side wall includes an upper edge 680 and a selected number of fibers from at least one cluster of optical fibers each extend out from the upper edge 680. And in a variation thereof, a selected number of fibers 505, FIG. 32A, from each cluster of optical fibers 504 extends out from the upper edge thereby forming a corresponding number of extension segments 623, as shown for example in FIG. 34B. The here like above, at least some of the extension segments 623 are of sufficient length to be movable by touch of hand or movement of air directed thereon, or where desired, all of the extension segments 623 are of sufficient length to be movable by touch of hand or movement of air directed thereon. In sone thereof as may be desired or preferred, all of the extension segments 623 may be of substantially the same length, or the extension segments 623 may be of randomly differing lengths, or the extension segments 623 are cut to form a repeating fringe pattern like fringe pattern 622b of FIG. 30B, for example. And then as to further features where desired, this method may further include the step of applying gold leaf onto at least a portion of the outside surface of the stone shell as provided in FIG. 51. Or where the gold is desired to be of a thinness such that some light may pass there-through, then the step of placing the stone vessel in a deposition chamber 822 and depositing a semi-transparent layer of gold (Au) onto at least a portion of the outside surface of the stone vessel may be provided according to illustrated steps of FIGS. 52A and 52B. Then here as indicated, some of the outside surface of the stone vessel may be masked 818 before the steps of placing the stone vessel in the deposition chamber 822 and depositing the semi-transparent layer of gold onto the outside surface thereof. Then to achieve coordination in the lighting design, the step of applying the at least one light emitting element onto the first layer of glass 674 may include placing the at least one light emitting element to direct light onto the portion of the outside surface of the stone vessel having the semi-transparent layer of gold. And when more gold surface is desired, then the step of depositing a semi-transparent layer of gold onto a substantial portion of the outside surface of the stone vessel may be provided. And for full coverage of essentially the full viewable exterior stone surface such of such a vessel, the step of depositing a semi-transparent layer of gold onto the outside surface of the stone vessel may include providing only a bottom portion of the stone vessel as being masked to prevent the deposition of gold thereon. And then as to order of process steps, the step of placing the stone vessel in the deposition chamber 822 may be performed before blowing molten glass to form the first layer of glass 674, or the step of placing the stone vessel in the deposition chamber 822 may be performed after blowing molten glass to form the second layer of glass and 676. And thus by these methods and optional steps where desired, a wide variety of illuminated glass-on-stone vessels according to the teaching hereof may be advantageously provided for use as an artistic stone jewelry box in the recharging of electronic jewelry as also provided herein.

And for further animation, any of these vessels, whether glass-on-glass or glass-on-stone, may be provided wherein the electronic circuitry provided in the electronics casing 506 includes a motion detector 688, FIG. 34E-3, so that when a person moves into close proximity to the vessel, said at least one light emitting element is activated. And then as to the security aspects hereof, any such vessel may be further provided wherein the electronic circuitry provided in the electronics casing 506 includes the motion detector 688 and a transmitter so that when an unauthorized person moves into close proximity to the vessel, said transmitter promptly sends an alarm signal to a property custodian.

Now moving on to additional features for the vessels hereof, FIG. 53 is a perspective illustration of a segmented insert assembly or jewelry tray 554a particularly configured in this example to be used in conjunction with any of the horizontally-layered stone jewelry boxes hereof such as provided by the laminated vessel 700a of FIG. 35A with the square-cut, stair-step interior surface 708 as shown in FIG. 38A. Here the jewelry tray 554a is divided into separate individual compartments with a smartphone compartment 831 and several jewelry compartments 832. In some preferred embodiments, the jewelry tray is made from a desired hard wood such an American Cherry or Walnut, Brazilian Jatoba, Honduran Mahogany, Gaboon or African Ebony, English Holly, or Canadian Maple, to list as few as to desired type and color which may be curated specifically to the textures and colors of each of the vessels paired therewith.

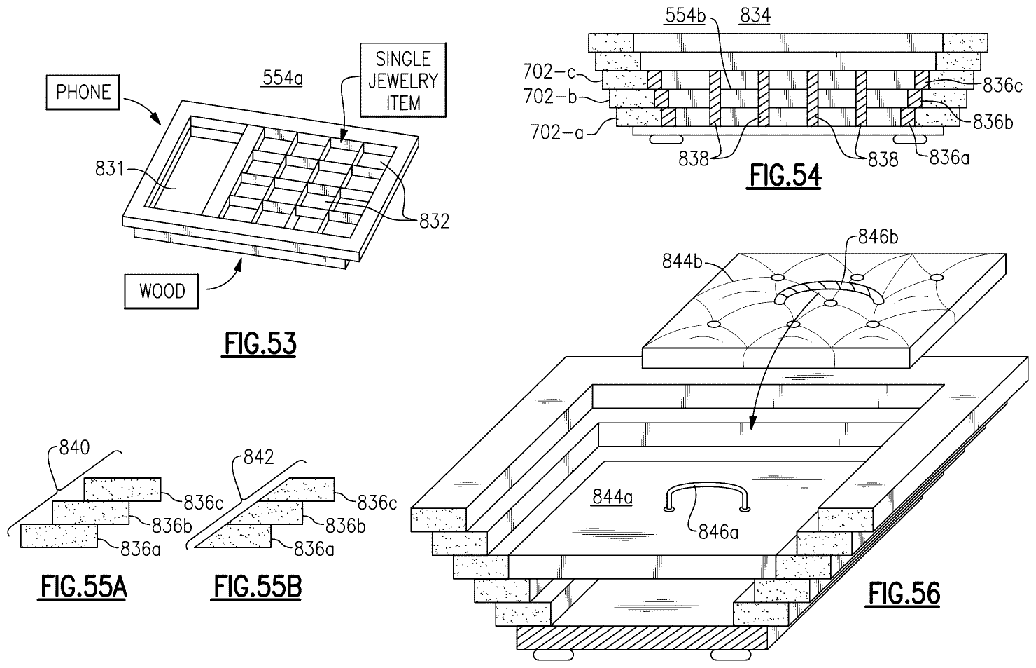

FIG. 54 is a sectional elevation view of a segmented insert assembly or jewelry tray 554b of the type of FIG. 53 shown in a horizontally-layered stone jewelry box or vessel 834 of the type provided by the laminated stone vessel 700a of FIG. 35A with the square-cut, stair-step interior surface 708 as shown in FIG. 38A. Here the tray 554b is formed from three wooded frame segments 836a, 836b, and 836c, which conformed in shape and fit to the inside surface of the vessel 834 as formed by the three corresponding stone frame segments 702-a, 702-b, and 702-c, as illustrated. The tray 554b also includes interior divider slats 838 configured to form the individual compartments in the tray which when made of wood are assembled by joinery that would be well understood by those of skill in the woodworking arts. Thus in use, the wooden tray 554b sits in register in a mating stair-step manner within the bottom interior volume of the vessel created by the inside edges of the three consecutive stone frame segments 702-a, 702-b, and 702-c, as shown.

FIGS. 55A and 55B are schematic representations of two different inside surface formations that may be utilized in making the segmented insert assembly 554b of FIG. 54. As such, the inside surface of the tray 554b may be of a stair-step configuration 840 as shown in FIG. 55A, or it may be of a smooth configuration 842 as shown in FIG. 55B.

FIG. 56 is a cut-away perspective view of a layered stone jewelry box hereof showing two different types of top covers 844a and 844b that may be provided therewith. The top cover 844a as shown may be made of wood in any desired species of wood as preferably used to make a corresponding tray 554 that would be paired therewith. And to easily access the items inside the tray during use, the top cover is provided with a handle 846a that may preferably be made from a precious metal such as gold (Au) or silver (Ag) as plated on nickel (Ni) or otherwise made of brass or bronze. The top cover 844b is an example of an upholstered cover that has internally a structural element of any suitable rigid or semi-rigid board material preferably with some thickness of soft padding thereon applied and then a fabric covering such a colored or raw silk that may be tufted with a handle 846b here show as a rope handle that may be formed from gold (Au) or silver (Ag) thread as intertwined with colored natural hemp material or otherwise intertwined with more modern manufactured materials such as polyester or nylon.

Next shown in FIG. 57A, is a schematic elevation view of a container 850 as an exemplary representation of any of the cases, boxes, vessels, and other such herein below, whether for electronic jewelry and/or mobile phones, or otherwise used, showing in accompanying block diagram a corresponding electronics casing 506 and a variety of options of various additional electronic functionalities that may be included therewith. And like the vessel 670e of FIG. 34E-1 discussed above, the container 850 here in FIG. 57A may be advantageously provided with holes 684 where depending on the desired embodiment of a container 850 as configured from these elements, a hole 684x may be configured to facilitate the functionality of motion detection, or where desired a hole 684y may be configured to facilitate digital imaging, or where further desired a hole 684z may include a set gemstone, like the ruby stone 686 of FIG. 34E-2, with a light source positioned there behind. The container 850 is then fitted with the electronics casing 506 in a suitable manner as discussed above wherein the electronic casing 506 may be provided with electronic circuitry to facilitate any of the options presented here or any further desired options in addition thereto. Thus when using the options of FIG. 57A to configure a particular embodiment of the container 850 according to any combination of the teachings provided herein, an illuminated cordless fruit bowl having motion detection with an internal rechargeable battery may be provided, or a cordless illuminated flower vase with internal rechargeable battery may be provided, or still further, for example, an illuminated soup tureen may be provided which may be corded as to power supply given the energy needed to keep soup warm on a side board during service of a meal, wherein rather than having charging pads for recharging personal electronic items, the electronics casing 506 is provided with a heating element to keep the soup at a desired warm temperature. And thus in such a warm temperature application, such a vessel or container may more preferably comprise an interior layer of ceramic or fired clay and then an outer layer of blown glass as may be illuminated by the teaching hereof. And now returning to the jewelry boxes and illuminated charging vessels as the principal illuminated containers hereof, an electronics casing provided therein may include tilt detection, such that when an unauthorized person picks up the jewelry box or illuminated vessel during a theft scenario, for example, a tilt detector sends a signal to a transmitter, which in turn sends a signal to an authorized smartphone or security office as described above at step 692 of FIG. 34E-3.

Thus next as an illustrated example of using the options presented above in FIG. 57A to render a particular embodiment of a container 850, FIG. 57B provides a perspective view of a rechargeable illuminated flower vase 852 as an embodiment of such a container as configured by combining selected teachings hereof and including selected options from the functional features presented in the block diagram of FIG. 57A. Thus as such, the illuminated flower vase 852 includes a top opening 853 having a top edge 854, an electronics casing 506 configured for the functionalities hereof positioned in the bottom of the vase as illustrated, a rechargeable battery 856 in association therewith, and a number of optical fiber bundles 504 with extension segments 858 which are provided in a manner similar to the fringe pattern 622g of FIG. 30G but rather than extending out from the top edge 854, are configured here in this embodiment to extend out from a knot or nub 860 through a hole 862 provided therein during assembly and after the bundle of fiber is threaded there-through, the hole 862 in the knot 860 is filled with molten glass from the inside out thereby encapsulating a corresponding segment of the bundle within the knot 860 during fabrication thereof. And further as indicated in the top edge 854, the vase 852 includes a first glass layer 618 and a second glass layer 620 like the layers of glass discussed above in the glass-on-glass vessels 616 of FIGS. 29A to 29E. As such, it should be readily understood in view hereof, that the vase 852 here in FIG. 57B may be made according to the method of FIGS. 24A to 24C as adapted hereto for making same. And like the decorative band 682 in the glass-on-stone vessel 670e of FIG. 34E-1, the vase 852 here includes a decorative band 682 which includes motion detectors 688 that may be implemented therein as discussed above. And thus as illustrated in conjunction with length segment BT that for purposes of discussion here extends along the length of a fiber bundle on left side of vase, from the base or bottom B of the vase to the tip T of a respective fiber bundle 504 at the output end 504b thereof. Thus along length segment A, the length of the bundle 504 corresponding thereto is encapsulated between the layers of glass 618 and 620 and in a similar manner, along length segment B, the length of the bundle 504 corresponding thereto is encapsulated within the knot 860, and then as illustrated, the length of the bundle 504 corresponding to length segment C is a pony-tail style bundle of extensions that are free of any mechanical confinement or encasing such that when configured with sufficient length and material composition, may be movable by touch of hand or gentle force of air applied thereto as discussed above with similar such embodiments. And further according to the above teachings relating to illumination of the various embodiments of the vessels hereof, the vase 852 here in FIG. 57B includes light sources 503b with each thereof positioned at the input end 504a of a corresponding optical fiber bundle 504. And like above as discussed in detail, and thus briefly recited here, as to operational functionality, the motion detectors 688 and light sources 503b are connected to control circuitry in the electronics casing 506 such that when a respective motion detector 688 detects motion in proximity thereto, such as a person walking passed the vase while positioned in use on a table top, for example, the control circuitry will activate a respective light source to thereby illuminated the bundle associated therewith for a certain duration of time, such as to personify the vase with animation in giving notice to the passer-by that it has detected their proximity and in recognition thereof provides for them a pleasing light show of limited duration, as powered in a cordless manner by the rechargeable batter 856 provide therein. And thus as would be well understood by those of skill in the electrical arts, a charging pad 864 as configured to fit in register with the base of the vase may be provided to recharge the battery 856 such that the vase may then be placed for operation in any desired location for use without the need to contend with providing accommodation for an electrical cord when so situated in such a desired location. And as to functionality and artistic composition related thereto, in this embodiment, the decorative band 682 provides a natural demarcation point such that anything provided, contained, encased, or encapsulated at or below the band 682 may be considered as so provided, contained, encased, or encapsulated in a lower part of the vase, and anything provided, contained, encased, or encapsulated above the decorative band 682 may be considered as so provided, contained, encased, or encapsulated in an upper part of the vase, for purposes of clarity in discussion and description hereof. Thus in the fabrication of the vase 852, the lower part of the vase may preferably be made from opaque glass material or darkly colored glass so that, for example, the bottom cuttings or roots of any flowers or plant material placed therein may not be seen and similarly any electronic components such as conductors connecting the electronics casing to the motion detectors or other such, and any lighting components situated in the lower part of the vase may also not be seen by an observer of the vase when looking at same from an exterior perspective. And in contrast therewith, the upper part of the vase 852 is thus preferably made from clear glass or more lightly colored glass such that any plant material contained by the vase in the upper part of the vase is viewable by an observer along with the segments of optical fibers in each bundle that are encapsulated between the glass layers in the upper part of the vase. Thus in this manner, the view of the plant material in the upper part of the vase, such as plant stalks or flower stems, is comingled with the viewable segments of optical fibers and thus when the fibers are illuminated by a person moving in proximity to the vase, as by motion detection thereof, the person may experience a pleasing sensation that light energy is comingled with the plant material thereby giving an additional life-like animation to the plant material particularly as the individual optical fibers may be configured to appear as comingled stem-like material when taken in the same view as any flower stems in the upper part of the vase. And further as would be understood in view of the above discussions, upwardly directed light sources may be provided as encapsulated in the lower part of the vase just below the decorative band 682, thus encased in the opaque lower glass, preferably, for ambient lighting of the vase without bundles of optical fiber associated therewith, or in various alternative embodiments or as combined therewith, the optical bundles may terminate at the level of the band 682 and thus are as such contained only in the lower part of the vase, or some or all of the fibers of any particular bundle may extend upwardly into the upper part of the vase but terminate with their output ends as being encapsulated within the clear glass layers with out any extension as shown in FIG. 30H and the vessel 670f of FIG. 34F discussed above, and then in such embodiments, the individual optical fibers may extend upwardly into close proximity to the top edge 854 of the vase as in the vessel 670a of FIG. 34A. And in still other embodiments, it may be preferred to have the individual fibers extend up through the top edge 854 of the vase 852 as pony-tail style extensions as shown in the vessel 670b of FIG. 34B or otherwise in any of the fringe patterns 622 of FIGS. 30A to 30C, for example.

FIG. 57C is a simplified block diagram of the features selected from the options hereof to specify the functionalities of a rechargeable illuminated flower vase 852 of the type presented in FIG. 57B which may be used as a general specification in collaboration with an electronics partner engaged to provide the light, motion, power, and electronic control functionalities thereof with the details thereof being beyond the intended scope hereof and thus reserved as the subject matter of subsequent disclosures as filed adjunct hereto.

Embodiments of an illuminated flower vase. Now with particular reference to FIGS. 57B and 57C in view of FIGS. 34E-1 to 34E-3 and as further referenced below, the inventors hereof also provide an illuminated glass vase 852 comprising: i) a first layer of blown glass 618 formed to include a bottom and at least one side wall with each thereof having an outside surface and an inside surface; ii) a second layer of blown glass 620 applied to the inside surface of the first layer of blown glass; iii) an electronics casing 506 positioned on the inside surface of the bottom of the first layer of glass 618 and encapsulated therein by the second layer of blown glass 620, said electronics casing 506 including control circuitry; iv) at least one light source 503 encapsulated between the first layer of blown glass 618 and the second layer of blown glass 620 and operably associated with said control circuitry; v) at least one motion detector 688 operably associated with said control circuitry; and vi) a rechargeable battery 856 for providing power to said at least one light source 503 and said at least one motion detector 688 to thereby illuminate the at least one light source 503 in a prescribed manner when the motion detector 688 detects a motion in proximity thereto. Here as described, the at least one light source 503 may be associated with a bundle of optical fibers 504. In some of these embodiments, each optical fiber 505 in the bundle of optical fibers 504 may be fully encapsulated within the first and second layers of blown glass 618, 620. In other embodiments, a segment A of the optical fiber bundle 504 is encapsulated between the first and second layers of blown glass 618 and 620, and at least some of the optical fibers in the bundle 504 have extension segments 858 that extend out from the outside surface of the at least one side wall of the first glass layer 618. In other embodiments, a segment of the optical fiber bundle A is encapsulated between the first and second layers of blown glass 618 and 620, and at least some of the optical fibers 505 in the bundle have extension segments that extend up and over a top edge 854 of the vase 852, as shown in FIG. 29G, for example. In any of these embodiments with extension segments 858, at least some of the extension segments 858 may be of sufficient length and composition to be moveable by touch of hand or force of air as applied thereto. And as to overall shape of the vase 852, the at least one side wall may include three side walls and the vase 852 is thereby configured in a corresponding type of triangular form, or the at least one side wall may include four side walls and the vase 852 is thereby configured in a corresponding type of square or rectangular form, or the at least one side wall is circular in shape and the vase 852 is thereby configured in a corresponding type of rounded form are here illustrated in FIG. 57B and not repeated in illustration of the above different shapes. And further in view of the above generally regarding illumination of the boxes and vessels hereof, the at least one light source 503 here in the vase 852 may preferably include a plurality of light sources. In these embodiments, some of the plurality of light sources 503 may be each associated with a corresponding bundle of optical fibers 504, or alternatively each of the plurality of light sources 503 may be associated with a bundle of optical fibers 504. In any of these embodiments, any number of optical fiber bundles 504 may be fully encapsulated within the first and second layers of blown glass, 618, and 620. In other embodiments, any number of optical fiber bundles 504 may be encapsulated between the first and second layers of blown glass 618 and 620 and at least some thereof may have at least some extension segments 858 that extend out from the outside surface of the at least one side wall of the first glass layer 618. And where desired, any number of optical fiber bundles 504 may be encapsulated between the first and second layers of blown glass, 618 and 620, and at least some thereof may then have at least some extension segments that extend up and over the top edge 854 of the vase 852. And like above, here also, at least some of the extension segments 858 may be of sufficient length and composition to be moveable by touch of hand or force of air as applied thereto. And then as to providing animation to the vase 852 by motion and illumination when the at least one motion detector 688 detects motion proximate the vase 852, the control circuitry activates each of the light sources 503 associated with a corresponding bundle of optical fibers 504 to thereby provide light along the fibers for a fixed duration of time. And in extended embodiments thereof, the at least one motion detector 688 may include two motion detectors 688 where each thereof is associated with a different light source 503 so that when a respective motion detector 688 detects motion proximate the vase 852, the control circuitry activates the light source 503 associated therewith to thereby provide illumination for a fixed duration of time. And then still further given a sufficient sizing of the vase 852, the at least one motion detector 688 may include a plurality of motion detectors 688 where each thereof is associated with a light source 503 that is associated with a bundle of optical fibers 405 so that when a respective motion detector 688 detects motion proximate the vase 852, the control circuitry activates the light source 503 associated therewith to thereby illuminate its corresponding bundle of optical fibers 504 for a fixed duration of time. And thus with same at hand when the at least one motion detector 688 includes a plurality of motion detectors 688 being equal in number to the number of light sources 503 in said plurality of light sources, each of said motion detectors 688 may then be associated with a different light source 503 so that when a respective motion detector 688 detects motion proximate the vase 852, the control circuitry activates the light source 503 associated therewith to thereby illuminate the vase 852 for a fixed duration of time. And then like above, in these embodiments with multiple motion detectors and light sources, as to shape of the vase, the at least one side wall may include three side walls and the vase 852 is thereby configured in a corresponding type of triangular form, or the at least one side wall may include four side walls and the vase 852 is thereby configured in a corresponding type of rectangular form, or still further, the at least one side wall may be circular in shape and the vase 852 is thereby configured in a corresponding type of rounded form as illustrated in FIG. 57B.

Next more generally as to the illuminated vessels provided herein, FIG. 58A is a simplified block diagram of additional functionalities that may be provided in the electronics casing of FIG. 57A as may be implemented in any illuminated jewelry vessel hereof, for example, whereby there is provided an architectural mode of operation 866 and a security mode of operation 868. In the architectural mode 866, the motion detection functionality is directed to illumination features as discussed above, while in the security mode 868, the motion detection, along with tilt detection and video imaging as may be further provided separately or combination therewith, is directed to providing wireless notification of a possible intruder during a theft or robbery scenario. Thus in use, when an owner of such a vessel departs their home for the day, they may use their mobile phone as paired with the vessel to switch the vessel from the architectural mode 866 to the security mode 868. And like above, since the electronic software details relating to these modes of operation are beyond the intended scope hereof they are thus reserved as the subject matter of subsequent disclosures filed adjunct hereto.

FIG. 58B is a simplified block diagram and corresponding pictorial rendering that may be used to specify the functionally of an illuminated charging vessel 870, here being representative of any of the vessels hereof, deployed as including a silent wake-up light alarm functionally as selected from the options provided above in FIG. 57A. Here the vessel 870 includes at least one light source 503 and a charging pad 872 as provided in the electronic casing 506 for charging a mobile phone 873 over night. In this use scenario, at the end of a day's use, the mobile phone 873 is place in the vessel 870 where a wake time has been entered into the phone in the usual manner. Then over night, the charging pad 872 recharges the phone 873 and at the designated wake-up time, the one of more light sources 503 are silently illuminated to thereby give a level of ambient lighting next to the then sleeping user such that a typical user after a night's sleep may detect as a night-light turned on as positioned close to their sleeping head when placed on a near by night stand, and thus wake up more gently than as may be compared to beeping sound or music that would otherwise be provided by a typical wake-up alarm. In an alternate embodiment thereof, each of the nightlights 503a, 503b, 503c, and 503d may be illuminated in a consecutive manner over a certain time period, such as fifteen minutes, for example, such that the slowly increasing ambient lighting provided by each consecutive light source when illuminated in turn may emulate the sun rising in an otherwise fully darkened room. And still further, one of more of the light sources 503 may have a variable intensity, as controlled by a dimmer function in the control circuitry, such that each light source separate from any other may transition from an off-state, to an on-state, as triggered by the wake-up function in the phone, with the on-state being initially at a very low light intensity which then gently increases in light intensity over a set time period of about fifteen minutes to again individually, consecutively, or otherwise collectively, emulate the rising sun in an otherwise fully darkened room.

Additional aspects here illustrated in the vessel 870 include at least one side wall 874, here including four such side walls or side wall segments comprising front and back, and left and right side walls configured together in a rectangular form with a corresponding rectangular bottom portion 876, as illustrated. The vessel 870 if a two-layer vessel have a first rigid layer 878 and a second rigid layer 880 which may be comprised of glass, stone, or any other suitable rigid material wherein the first rigid layer 878 has a desired translucence property such that some light when directed thereon on from an encased light source 503, may pass there-through and be visible to an observer of the vessel when so illumined as discussed above. And as discussed above in various related embodiments, the vessel 870 illustrated here is shown with three exemplary bundles of optical fiber 504 including bundle or cluster of optical fibers 504i where all the individual fibers thereof are fully encapsulated or encased between and within the rigid layers 878 and 880, a bundle 504j wherein some of the individual fibers thereof are fully encased, and some thereof have extension segments 886b that extend out from the side wall 874 as illustrated here in FIG. 58B in a manner akin to the extensions 858 discussed above in connection with the illuminated flower vase 852 of FIG. 57B. And then back here in FIG. 58B, the bundle 504k on the back wall 874 is an example of where all the individual fibers in the bundle have extension segments 886a that extend up from and over into the exterior space of the vessel container portion 871 from a top edge 888 of the vessel 870 as illustrated.

Embodiments of an illuminated charging and wake-up vessel. Now with particular reference to FIG. 58B as to the various elements, components, features, and functionalities presented therein, the present invention is thereby further directed to an illuminated charging and wake-up vessel 870, comprising i) a container portion 871 having at least one side wall 874 and a bottom 876, said container portion 871 being formed from a first rigid layer 878 and a second rigid layer 880 with said first rigid layer 878 being formed from a translucent material; ii) an electronics casing 506 positioned in the bottom 876 of the container portion 871 being encapsulated therein between the first and second rigid layers 878 and 880, said electronics casing 506 including control circuitry 882 and a charging pad 872 for changing a mobile phone 873 when placed in the container portion 871; iii) receiving circuitry 884 in operable engagement with said control circuitry 882, said receiving circuitry 884 configured to receive a wake-up signal from the mobile phone 873; and iv) at least one light source 503 encapsulated between the first and second rigid layers 878 and 880 and associated operably with said control circuitry 882 such that when said receiving circuitry 884 receives a wake-up signal from the mobile phone 873, said control circuitry 882 switches said at least one light source 503 from an off-state to an on-state. And in a preferred embodiment thereof, at least one light source 503 includes a plurality of light sources 503a, 503b, 503c, and 503d. As for further such as to illumination, at least one of the plurality of light sources such as 503a may be associated with a bundle of optical fibers 504j, and in further variation thereof, some of the plurality of light sources 503 are each associated with a corresponding bundle of optical fibers 504, or all of light sources 503 are each associated with a bundle of optical fibers. And where desired, any number of optical fiber bundles 504 may be fully encapsulated within the first and second rigid layers 878 and 880 as shown with bundle 504i, or any number of optical fiber bundles 504 may be encapsulated between the first and second rigid layers 878 and 880 and at least some thereof have at least some extension segments 886a that extend up and over a top edge 888 of the vessel 870 with the bundle 504k, or still, any number of optical fiber bundles like bundle 504j may be encapsulated between the first and second rigid layers 878 and 880 and at least some thereof have at least some extension segments 886b that extend out from an outside surface of the at least one side wall 874 of the container portion 871. And like above, at least some of the extension segments 886a or 886b may be of sufficient length and composition to be moveable by touch of hand or gentle force of air as applied thereto. Then as to functionality, the on-state may include an initial lower-power on-state that transitions to a higher-power on-state over a set time period such that the at least one light source 503 in response thereto transitions from a corresponding lower brightness to a higher brightness over said set time period, and then for a typical application, the set time period may be within the range of 5 minutes to 30 minutes, and preferably in some embodiments, the set time period is about 15 minutes. For assurance as to being awoken, in any of the above embodiment, at the end of said set time period, the mobile phone 873 may be set to give a sounded wake-up alarm. And as to further regarding illumination, the on-state of each of said plurality of light sources 503 may include an initial lower-power on-state that transitions to a higher-power on-state over a set time period such that each light source 503 is thereby configured to transition from a corresponding lower brightness to a higher brightness over said set time period. And in each embodiment hereof as desired, when said receiving circuitry 884 receives a wake-up signal from the mobile phone 873, said control circuitry 882 may switch each of said plurality of light sources 503 to its on-state at the same time, or alternatively, when said receiving circuitry 884 receives a wake-up signal from the mobile phone 873, said control circuitry 882 switches each of said plurality of light sources 503 to its on-state consecutively one after the other. Then in any of these embodiments, the set time period may also be within the range of 5 minutes to 30 minutes, and preferably set in a typical case at about 15 minutes. And then like above, in these embodiments also, at the end of said set time period, the mobile phone may give a sounded wake-up alarm. And in a particular embodiments hereof, the set time period from a first of the consecutive on-states to a last of the consecutive on-states is within the range of 5 minutes to 30 minutes, with same being preferably set at about 15 minutes, and then hereto, at the end of said set time period, the mobile phone 873 may give a sounded wake-up alarm. Then as to shape and geometric configuration, the at least one side wall 874 may include four side walls and the vessel 870 is thereby configured in a corresponding type of square or rectangular form, or the at least one side wall 874 is circular in shape and then the vessel 870 is thereby configured in a corresponding type of rounded form, or still, the at least one side wall may be heart-shaped and the vessel 870 is then configured in a corresponding type of heart-shaped form, or lastly as an example here, the at least one side wall 874 may be butterfly-shaped and then the vessel 870 is thereby configured in a corresponding type of butterfly-shaped form. Thus there are provided according to the teachings hereof, several embodiments of an illuminated charging and wake-up vessel of the type of vessel 870 shown and discussed above in conjunction with FIG. 58B in view of various features, aspects, functionalities, and uses presented in the disclosure hereof.

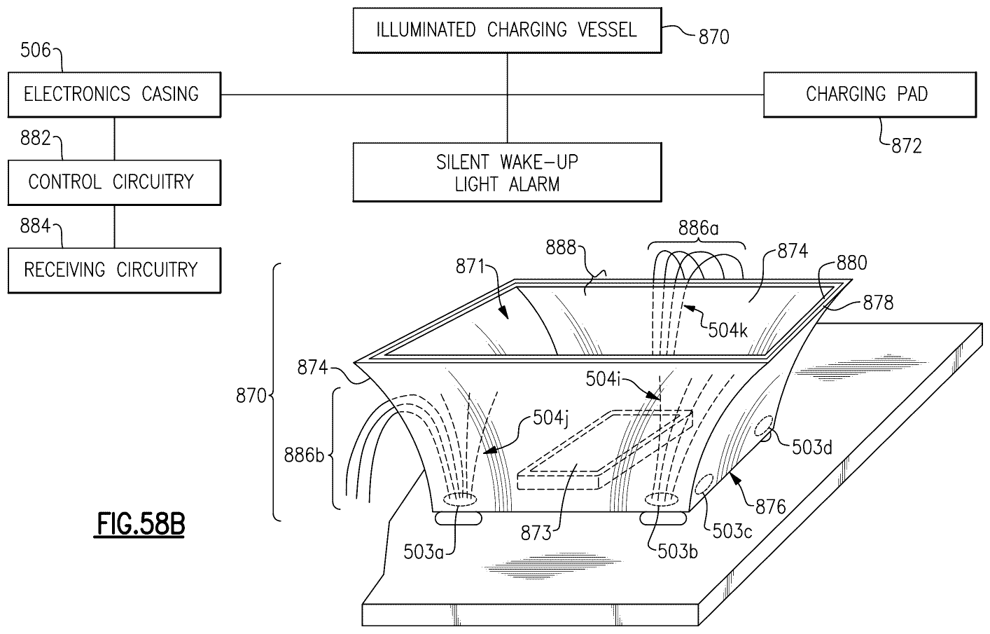

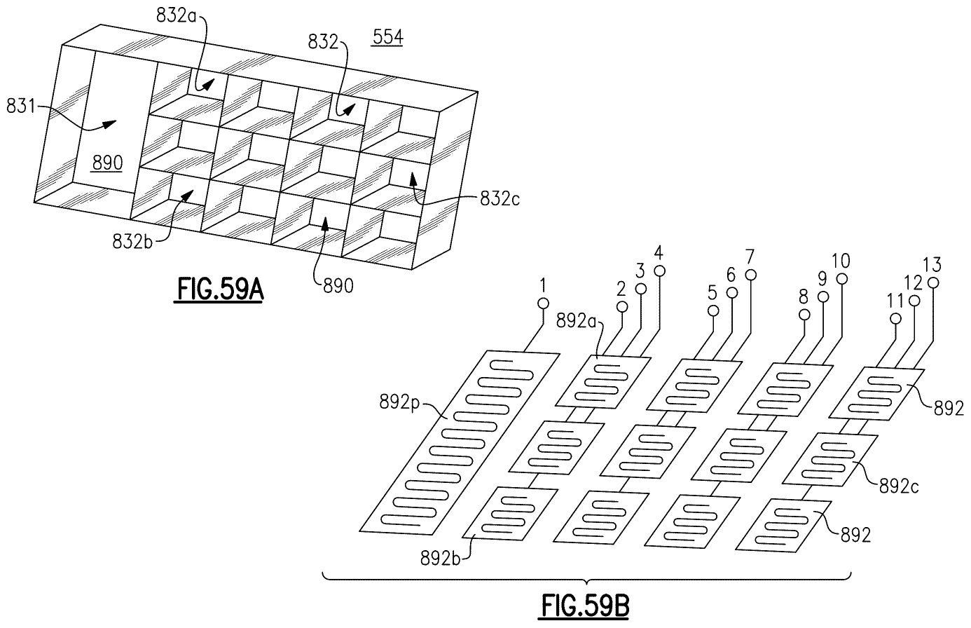

Next moving on the FIG. 59A, there is provided a perspective top view of a representative segmented insert assembly 554 of the type presented in detail in conjunction with FIGS. 53 to 55B including a phone charging compartment 831 and several electronic jewelry charging compartments 832 and as discussed above, each of the compartments 831 and 832 has an open bottom reference here at 890. Then in corresponding fashion, FIG. 59B is a schematic perspective assembly view of a number of individual charging pads 892 each associated with a corresponding light source where the charging assembly is configured to comport with the design of the segmented insert assembly 554 of FIG. 59A, and in use is provided in a corresponding electronics casing such as any of the electronics casings 506 shown in FIGS. 29A to 29G, FIGS. 34A to 34G, and FIGS. 50A to 50D, for example. As such, the charging pad 892p is configured to recharge a mobile phone such as any of the mobile phones discussed herein, and is sized to sit in register just under the phone compartment 831 with the open bottom 890 such that when in use, the phone contacts the inside bottom surface of a glass layer and EM energy produced by the coil in the pad charges the rechargeable battery in the phone. In a similar manner, in a fully assembled vessel including an insert assembly or tray 554, charging pad 892a sits in register with jewelry compartment 832a, charging pad 892b sits in register with jewelry compartment 832b, charging pad 892c sits in register with jewelry compartment 832, and so on for the remaining jewelry compartments 832. As indicated above, each charging pad 892 may be associated with a light source as discussed herein across several embodiments where here there may be provided thirteen different light sources numbered 1 to 13 with each thereof uniquely and separately associated with one of the individual charging pads 892 in this embodiment. Thus here for example, light source 1 is associated with charging pad 892p and thus may give illuminated notification regarding the status of a phone placed thereon as contained with phone compartment 831, light source 2 may be associated with charging pad 892a and thus may give illuminated notification regarding the status of any electronic jewelry item placed thereon as contained within jewelry compartment 832a, light source 4 may be associated with charging pad 892b and thus may give illuminated notification regarding the status of any electronic jewelry item placed thereon as contained within jewelry compartment 832b, and lastly for illustration purposes here, light source 12 may be associated with charging pad 892c and thus may give illuminated notification regarding the status of any electronic jewelry item placed thereon as contained within jewelry compartment 832c.

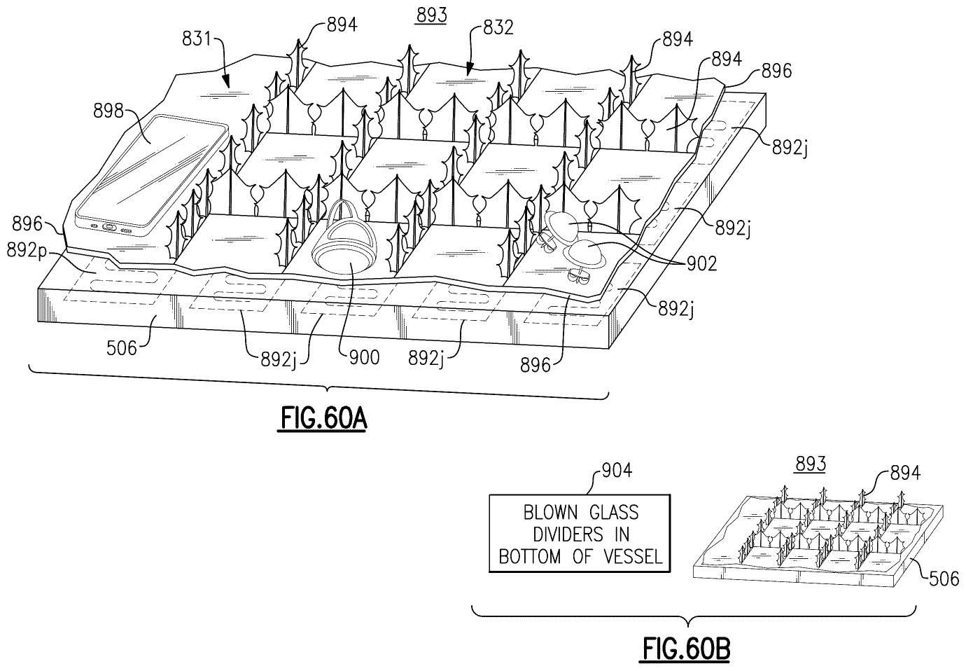

In any of the above glass-on-glass or glass-on-stone illumined charging vessels according to the various teachings, embodiments, and methods hereof, it may be desired to have glass blown dividers in the bottom of the vessel for separating electronic jewelry items from each other along with a clearly defined charging location for a personal mobile phone. As such, the inventors hereof provide the following glass blown divider assembly that may be used in place of any of the insert assemblies or jewelry trays 554 discussed above in conjunction with FIGS. 25C, 32C, 48C, and 53 to 55B. Thus next, FIG. 60A provides a break-away perspective view of an alternate bottom embodiment for any of the illuminated vessels hereof showing a glass-blown jewelry tray 893 including glass-blown divider elements 984 formed integral with the blown-glass bottom of the vessel here illustrated as glass layer 896. Thus also shown therewith is an electronic casing 506 including therein as show here in dashed line, charging pads 892 including a charging pad 892p for the exemplary phone 898 and individual charging pads 892j corresponding to separate compartments 832 in the jewelry tray 893 for recharging, for example, an electronic ring 900 and a pair of electronic earrings 902 as illustrated here for exemplary purposes as being representative of any of the electronic jewelry items hereby provided above. And then as further illustrated here, the glass-blown divider elements 894 take the form of individual leaves of an English Holly tree or shrub, Ilex aquifolium, that have been cut in half while in a semi-molten state and applied to the glass layer 896 during fabrication thereof, and then when cooled to a solidified state become the rigid jewelry tray dividers show here. And like with any of the glass-blown elements discussed above, these glass-blown elements 894 may be of colored glass such as green and while and may be up lit by light sources positioned therefor in the bottom of the vessel. And as world be apparent in view hereof, the glass-blown divider elements 894 are not limited to being of the form of holly shrub leaves cut in half, but may take any desired organic or botanical form including flower petals such as cherry blossom pedals, or rose petals, other type of leaves, intertwining vines, or such like, or may take other abstracted forms such as roping, fencing, or any other such abstracted or geometric forms as may be desired.

And thus in view of the features of FIG. 60A, any of the glass-on stone, or glass-on-glass vessels hereof may further include glass-blown divider elements 894 positioned in the bottom thereof to form a phone compartment 831 and at least one jewelry compartment 892 with each such compartment having associated therewith a charging pad 892 provided in operable conjunction with the control circuitry in a corresponding electronics casing.

Thus in fabrication of such a glass-blown jewelry tray 893 of FIG. 60A, here next in FIG. 60B there is provided a subassembly method step 904 illustrating the making of a glass-blown jewelry tray of the type discussed in detail above in FIG. 60A as configured atop an electronics casing 506. As such, the step 904 of making a glass-blown divider in the bottom of any of the glass-on-glass or glass-on stone vessels hereof may be included as a desired step in any of the above glass-blowing methods.

As expressed above in various manner, across various points along the way so far herein, one principal aspect of this invention is to provide life-like characteristics to the glass and stone charging vessels hereof. This animation is achieved not by robotic moving parts, and the noise that may be associated therewith, but by light of different colors, different intensities, and by natural gentle movement of optical fibers that extend aloft the top edge of the vessels as described herein above. Many non-human objects are animated by various and sundry means but few are so animated to provide a stationary utilitarian article fully cloaked in aesthetic detail such as our illuminated electronic jewelry vessel having a type of animation that subtly and gently communicates with the user of such an item. Thus the intent of the inventors hereof as to this aspect of the present invention is to provide an illuminated blown-glass or sculpted-stone-with-blown-glass electronic charging vessel made from natural material, having molded or sculpted thereon themes from nature such as botanicals, or inlaid or inlayed therein further natural materials such as semi-precious stone, that may be further bejeweled with precious gemstones, and that may then even further be adorned with gold leaf, all of which is then animated by the mystical beauty of electro-magnetic radiation in the visible range that we call “light”; which when provided by our sun, gives all life to mother Earth.

Embodiments of illuminated two-layer glass-on-glass vessels. Now with particular reference again to FIGS. 24A to 28E, FIGS. 29A to 30H, and FIGS. 59A to 60B, in view of FIGS. 34E-2 to 34E-4, as referenced below, the inventors hereof provide a glass vessel, of the type of vessel 540, 594, 616 described above, said glass vessel being for use as a jewelry box having light display notification and recharging functionality for personal electronic items placed therein, and said glass vessel comprising i) a first layer of blown glass 618 formed to include a bottom 528 and at least one side 175 wall with each thereof having an outside surface and an inside surface; ii) a second layer of blown glass 620 applied to the inside surface of the first layer of blown glass 618; iii) an electronics casing 506 positioned on the inside surface of the bottom 528 of the first layer of glass 618 and encapsulated therein by the second layer of blown glass 620, said electronics casing 506 including control circuitry and at least one charging pad 892; and iv) a plurality of light sources 503 encapsulated between the first layer of blown glass 618 and the second layer of blown glass 620, each of said plurality of light sources 503 operably connected to the control circuitry to thereby illuminate the glass vessel in a prescribed manner. Then to achieve certain aspects hereof relating to motion detection, this glass vessel may further include at least one motion detector 688 and motion detection circuitry operably connected thereto and to the control circuitry wherein when motion is detected by the at least motion detector 688, the control circuitry activates at least one of the plurality of light sources 503. And in further embodiments thereof, the glass vessel may also include transmitter circuitry 692 operably connected to the control circuitry wherein when motion is detected by the at least one motion detector 688, the transmitter circuitry 692 sends a wireless alarm signal to a remote device such as a mobile phone or such other remote device as located at a security office. And in still further embodiments hereof, the glass vessel may further include the transmitter circuitry 692 and a digital camera 694 operably connected to the control circuitry, the transmitter circuitry 692 then configured to live-stream a wireless video signal to a remote device such as a mobile phone or other remote device as located at a security office. And in certain embodiments thereof, including at least one digital camera 694 and digital camera circuitry operably connected thereto and to the control circuitry, when motion is detected by at least one motion detector 688 included therewith, the transmitter circuitry 692 then sends a live-stream wireless video signal to a to a mobile phone or remote device a remote device located at a security office.

In any of the above glass vessels, the at least one of the plurality of light sources 503 may be associated with a bundle of optical fibers 504, or some of the plurality of light sources 503 may be each associated with a corresponding bundle of optical fibers 504, or still, each of the plurality of light sources 503 may be associated with a bundle of optical fibers 504. In any of these embodiments, any number of optical fiber bundles 540 may be fully encapsulated within the first layer of blown glass 618 and the second layer of blown glass 620. Alternatively, any number of optical fiber bundles 504 may be encapsulated between the first layer of blown glass 618 and the second layer of blown glass 620 and at least some thereof have at least some extension segments 622, 623 that extend up and over a top edge 624 of the vessel. And where desired, at least some of the extension segments 622, 623 may be of sufficient length and composition to be moveable by touch of hand or force of air as applied thereto.

Then as to motion detection and illumination, any of the above glass vessels may be provided wherein at least one of the plurality of light sources 503 is associated with a bundle of optical fibers 504 and then when the at least one motion detector 688 detects motion proximate the vessel, the control circuitry activates the at least one of the plurality of light sources 503 to thereby provide light along the fibers of the bundle 504 in a prescribed manner for a fixed duration of time. And in a similar manner, when some of the plurality of light sources 503 are each associated with a corresponding bundle of optical fibers 504 and each corresponding bundle of optical fibers 504 is associated with a respective motion sensor 688 then when a respective motion detector 688 detects motion proximate the vessel, the control circuitry activates an associated light source 503 to thereby provide light along the corresponding fibers in the bundle 504 in a prescribed manner for a fixed duration of time. And still, in the embodiments where each of the plurality of light sources 503 is associated with a bundle of optical fibers 504, and each corresponding bundle of optical fibers 504 is associated with a respective motion sensor 688, then when a respective motion detector 688 detects motion proximate the vessel, the control circuitry activates an associated light source 503 to thereby provide light along the corresponding fibers in the bundle 504 in a prescribed manner for a fixed duration of time. In any of there embodiments where desired, any number of optical fiber bundles 504 may be fully encapsulated within the first layer of blown glass 618 and the second layer of blown glass 620. And then when desired as a variation thereof, any number of optical fiber bundles 504 may be encapsulated between the first layer of blown glass 618 and the second layer of blown glass 620 and then at least some thereof have at least some extension segments 622, 623 that extend up and over the top edge 624 of the vessel. And like above, in these embodiments with extension segments 622, 623, at least some of the extension segments 622, 623 may be of sufficient length and composition to be moveable by touch of hand or force of air as applied thereto.

And then as to geometric configurations, any of the above glass vessels may be provided wherein said at least one side wall 175 includes four side walls and the vessel is thereby configured in a corresponding type of square or rectangular form, or said at least one side wall is circular in shape and the vessel is thereby configured in a corresponding type of rounded form, or when more complex configurations are desired, the glass vessel may be provided wherein said at least one side wall 175 is heart-shaped and the vessel is thereby configured in a corresponding type of heart-shaped form, or said at least one side wall 175 is butterfly-shaped and the vessel is thereby configured in a corresponding type of butterfly-shaped form.

Embodiments of illuminated three-layer glass-on-stone vessels. Now with particular reference again to FIG. 32A, FIGS. 34A to 34G, FIG. 43C, FIGS. 44B to 44D, FIG. 48A, and FIGS. 59A to 60B, as referenced below, the inventors hereof provide a three-layer glass-on-stone vessel for use as a jewelry box having light display notification and recharging functionality for personal electronic items placed therein, said three-layer vessel comprising i) a stone shell 636, 764, 792 formed to include a bottom 641, 704, 798 and at least one side wall 175 with each thereof having an outside surface and an inside surface; ii) a first layer of blown glass 674 applied to the inside surface of the stone shell taking the form thereof and thereby presenting a corresponding inside surface in the formation thereof; iii) a second layer of blown glass 676 applied to the inside surface of the first layer of blown glass 674; iv) an electronics casing 506 positioned on the inside surface of a bottom of the first layer of glass 674 and encapsulated therein by the second layer of blown glass 676, said electronics casing 506 including control circuitry and at least one charging pad 892; and v) a plurality of light sources 503 encapsulated between the first layer of blown glass 674 and the second layer of blown glass, each of said plurality of light sources operably connected to the control circuitry to thereby illuminate the vessel in a prescribed manner. And then as to providing motion detection, this three-layer vessel may further include at least one motion detector 688, FIG. 34E-3, and motion detection circuitry operably connected thereto and to the control circuitry wherein when motion is detected by the at least motion detector 688, the control circuitry activates at least one of the plurality of light sources 503. And then in further embodiments as to certain security features hereof. This three-layer vessel may further include transmitter circuitry 692 operably connected to the control circuitry wherein when motion is detected by the at least one motion detector, the transmitter circuitry sends a wireless alarm signal to a remote device such as a mobile phone or such other remote device located at a security office. And like the above glass vessel, the three-layer vessels here may further include the transmitter circuitry 692 and a digital camera 694, FIG. 34E-4, operably connected to the control circuitry, the transmitter circuitry 692 configured to live-stream a wireless video signal to a remote device such as a mobile phone or other remote device located at a security office. Then when the vessel is place in a security mode and includes at least one digital camera 692 and digital camera circuitry operably connected thereto and to the control circuitry wherein when motion is detected by the at least one motion detector 688, the transmitter circuitry 692 sends a live-stream wireless video signal to a remote device such as a mobile phone or other such remote device located at a security office.

And like above in the glass vessel, here also in these embodiments as to illumination, the three-layer vessel may be provided wherein at least one of the plurality of light sources 503 is associated with a bundle of optical fibers 504, or wherein some of the plurality of light sources 503 are each associated with a corresponding bundle of optical fibers 504, or further wherein each of the plurality of light sources 503 is associated with a bundle of optical fibers 504. Then in any of these embodiments, any number of optical fiber bundles 504 may be fully encapsulated within the first layer of blown glass 674 and the second layer of blown glass 676, or any number of optical fiber bundles 504 may be encapsulated between the first layer of blown glass 674 and the second layer of blown glass 676 where at least some thereof have at least some extension segments 622, 623 that extend up and over a top edge 680 of the vessel. And then in any of these embodiments with extension segments 622, 623, at least some of the extension segments 622, 623 may be of sufficient length and composition to be moveable by touch of hand or force of air as applied thereto. And then when desired further, where in any of these vessels at least one of the plurality of light sources 503 is associated with a bundle of optical fibers 504, then when the at least one motion detector 688 detects motion proximate the vessel, the control circuitry activates the at least one of the plurality of light sources 503 to thereby provide light along the fibers in the bundle 504 in a prescribed manner for a fixed duration of time. Alternatively, wherein some of the plurality of light sources are each associated with a corresponding bundle of optical fibers 504 and each corresponding bundle of optical fibers 504 is associated with a respective motion sensor 688, then when a respective motion detector 688 detects motion proximate the vessel, the control circuitry activates an associated light source 503 to thereby provide light along the corresponding fibers in in the bundle 504 in a prescribed manner for a fixed duration of time. And still further in the alternative, when each of the plurality of light sources 503 is associated with a bundle of optical fibers 504, and each corresponding bundle of optical fibers 504 is associated with a respective motion sensor 688, then when a respective motion detector 688 detects motion proximate the vessel, the control circuitry activates an associated light source 403 to thereby provide light along the corresponding fibers in the correspond bundle 504 in a prescribed manner for a fixed duration of time. In any of these embodiments, any number of optical fiber bundles 504 may be fully encapsulated within the first layer of blown glass 674 and the second layer of blown glass 676, or any number of optical fiber bundles 504 may be encapsulated between the first layer of blown glass 674 and the second layer of blown glass 676 and at least some thereof have at least some extension segments 622, 623 that extend up and over a top edge 680 of the vessel. Then like above, in any of these embodiments with extension segments 622, 623 at least some of the extension segments 622, 623 may be of sufficient length and composition to be moveable by touch of hand or force of air as applied thereto.

Then as to shape and geometric configurations, any or these three-layer vessels may be provided wherein said at least one side wall 175 includes four side walls and the vessel is thereby configured in a corresponding type of square or rectangular form, or said at least one side wall 175 is circular in shape and then the vessel is thereby configured in a corresponding type of rounded form, or further, said at least one side wall 175 is heart-shaped and the vessel is thereby configured in a corresponding type of heart-shaped form, or still further, said at least one side wall 175 is butterfly-shaped and the vessel is thereby configured in a corresponding type of butterfly-shaped form.

Embodiments of illuminated two-layer glass-on-stone vessels. Now with reference again to FIG. 32A, FIG. 33C, FIGS. 34A to 34G, FIG. 43C, FIGS. 44B to 44D, FIG. 48A, and FIGS. 59A to 60B, as for two-layer glass-on-stone vessels wherein the second layer of glass 676 is not utilized as discussed above, and here again otherwise referenced below in the same manner, the inventors hereof provide a two-layer glass-on-stone vessel for use as a jewelry box having light display notification and recharging functionality for personal electronic items placed therein, said two-layer vessel comprising i) a stone shell 636, 764, 792 formed to include a bottom 641, 704, 798 and at least one side wall 175 with each thereof having an outside surface and an inside surface; ii) a layer of blown glass 674 applied to the inside surface of the stone shell 636, 764, 792 thereby presenting a corresponding inside surface in the formation thereof; iii) an electronics casing 506 positioned on the inside surface of the bottom 641, 704, 798 of the stone shell 636, 764, 792 and encapsulated therein by the layer of blown glass 674, said electronics casing 506 including control circuitry and at least one charging pad 892; and iv) a plurality of light sources 503 encapsulated between the layer of blown glass 674 and the inside surface of the stone shell 636, 764, 792, each of said plurality of light sources 503 operably connected to the control circuitry to thereby illuminate the vessel in a prescribed manner. And then to facilitate illumination, the two-layer vessel may further comprise at least one socket or slot 669 formed into a selected inside bottom or side location of said stone shell 636, 764, 792 wherein each of said at least one sockets or slots 669 is configured to align a corresponding lighting or electrical element. And then like above as to motion detection, this two-layer vessel may further include at least one motion detector 688, FIG. 34E-3, and motion detection circuitry operably connected thereto and to the control circuitry wherein when motion is detected by the at least motion detector 688, the control circuitry activates at least one of the plurality of light sources 503. And for security applications, the two-layer vessel may further include transmitter circuitry 692 operably connected to the control circuitry wherein when motion is detected by the at least one motion detector 688, the transmitter circuitry 692 sends a wireless alarm signal to a remote device such as a mobile phone or other such remote device located at a security office. And still further as to security, where desired, the two-layer vessel may further include the transmitter circuitry 692 and a digital camera 694, FIG. 34E-4, operably connected to the control circuitry, the transmitter circuitry 692 then further configured to live-stream a wireless video signal to a remote device such as a mobile phone or other remote device as located at a security office. And then in application, when the two-layer vessel includes at least one digital camera 694 and the digital camera circuitry operably connected thereto and to the control circuitry, then when motion is detected by the at least one motion detector 688, the transmitter circuitry 692 may send a live-stream wireless video signal to a remote device such as a mobile phone or other remote device as located at a security office.

And like above as to illumination, here further in various embodiments of this two-layer vessel, at least one of the plurality of light sources 503 may be associated with a bundle of optical fibers 504, or some of the plurality of light sources 503 may be each associated with a corresponding bundle of optical fibers 504, or still further, each of the plurality of light sources 503 may be associated with a bundle of optical fibers 504. Then in any of these embodiments, any number of optical fiber bundles 504 may be fully encapsulated within the layer of blown glass and the inside surface of the stone shell 636, 764, 792. Alternatively, any number of optical fiber bundles 504 may be encapsulated between the layer of blown glass and the inside surface of the stone shell 636, 764, 792 and at least some thereof have at least some extension segments 622, 623 that extend up and over a top edge 680 of the vessel. Here also, at least some of the extension segments 622, 623 may be of sufficient length and composition to be moveable by touch of hand or force of air as applied thereto. And then as to motion and illumination for animation, wherein at least one of the plurality of light sources 503 is provided in association with a bundle of optical fibers 504, then when the at least one motion detector 688 detects motion proximate the vessel, the control circuitry activates the at least one of the plurality of light sources 503 to thereby provide light along the fibers of the bundle in a prescribed manner for a fixed duration of time. Alternatively, in embodiments hereof wherein some of the plurality of light sources 503 are each associated with a corresponding bundle of optical fibers 504 and each corresponding bundle of optical fibers is associated with a respective motion sensor 688, then when a respective motion detector 688 detects motion proximate the vessel, the control circuitry activates an associated light source 503 to thereby provide light along the corresponding fibers in the corresponding bundle 504 in a prescribed manner for a fixed duration of time. And still in alternative versions thereof, wherein each of the plurality of light sources 503 is associated with a bundle of optical fibers 540, and each corresponding bundle of optical fibers 504 is associated with a respective motion sensor 688, then when a respective motion detector 688 detects motion proximate the vessel, the control circuitry activates an associated light source 503 to thereby provide light along the corresponding fibers in the associated bundle 504 in a prescribed manner for a fixed duration of time. And then in any of these embodiments with motion detection and fiber optical bundles 504, like above, any number of optical fiber bundles 504 may be fully encapsulated within the layer of blown glass and the inside surface of the stone shell 636, 764, 792. Alternatively, any number of optical fiber bundles 504 may be encapsulated between the layer of blown glass and the inside surface of the stone shell 636, 764, 792 and at least some thereof have at least some extension segments 622, 623 that extend up and over a top edge 680 of the vessel. And where provided at least some of the extension segments 622, 623 may be of sufficient length and composition to be moveable by touch of hand or force of air as applied thereto.

And here also as to shape and geometric configuration, at least one side wall 175 may include four side walls and the vessel is thereby configured in a corresponding type of square or rectangular form, or said at least one side wall 175 may be circular in shape and the vessel is thereby configured in a corresponding type of rounded form, or still said at least one side wall 175 may be heart-shaped and the vessel is thereby configured in a corresponding type of heart-shaped form, or still further, said at least one side wall 175 may be butterfly-shaped and the vessel is thereby configured in a corresponding type of butterfly-shaped form.

Stone Shell Embodiments. In the above glass-on-stone embodiments, the stone shell 636 may be formed from a block of stone 626 as shown in FIG. 32A. Alternatively, the block of stone 636 may be formed from a series of panel segments 776 as shown and described above in detail in FIG. 44B. In these embodiments, the panel segments 776 may be formed from a single slab of stone such as 728c, or the panel segments 776 may be formed from at least two different slabs of stone such as 728c and 728d, as shown in FIG. 44B. Alternately, the stone shell as used in any of the above two-layer or three-layer glass-on stone vessels may be of the type of stone shell 764 as made by the method of FIGS. 43A to 43C as formed from a series of frame segments 702. Here when so used, the frame segments 702 may be formed from a single slab of stone, such as the slab of stone 728 shown and discussed in conjunction with FIG. 43A, or the frame segments 702 may be formed from at least two different slabs of stone such as slabs 728a and 728b as shown and discussed above in detail in connection with FIG. 44A. In any of these stone shells, the outside surface of the stone shell may include sculpted features as shown and described above in regard to the stone shell 636 of FIG. 33B. And where desired, the outside surface of any of these stone shells may includes inlaid gemstones such as rubies, emeralds, or sapphires, for example, as positioned to have directed there-through light from at least one of the plurality of light sources 503, as shown and discussed in FIGS. 34E-1 and 34E-2 and the glass-on-stone vessels 806c and 806d shown respectively in FIGS. 50C and 50D. Further, the outside surface of any of these stone shells, where desired, may include inlaid semi-precious stone features 664 as described in the method step 663 of FIG. 49 and described in the vessel 806d of FIG. 50D. And further where desired in combination with any of the above features, any of these stone shells may include a semi-transparent layer of gold located in an area to have directed there-through at least some light from at least one of the plurality of light sources 503, as described and illustrated in method of FIGS. 52A and 52B as applied to the exemplary vessel thereof.

Now the following discussion will next be directed to applying the above teachings to expanded areas of application in the fields of architectural design, building security, residential lighting design, and home security.

Thus in FIG. 61 presents a flow-chart with pictographic illustrations of principal steps of making an illuminated flat glass panel by applying certain teaching hereof to the arts of making sheet glass for windows, doors, partitions, and walls. As such, at step 910, a glass sheet or panel 912 is provided. The glass panel 912 may be formed by modern float glass methods or may be formed by more tradition methods such as hand-blown English reamed glass using a tubular form with a hinged opening. Then at step 914, optical fiber bundles 916 are applied to the glass panel 912. The bundles 916 may be of any desired length and preferably fit within the outside parameter of the panel 912. And as further indicated, each of the bundles of optical fiber includes a number of individual optical fibers 917. As such, each of the bundles 916 has an input end 916a and an output end 916b, and each of the respective input ends 916a is optically connected to a light source 918 that is provided within a suitable housing 920 with each thereof having a separate power supply cord 921. In fabrication, the panel 912 may be cool and fully solidified before the bundles 916 are applied thereto, or the panel 912 may be in a heated semi-molten state where then each of the fibers 917 of each bundle 916 is pressed into the soft glass forming the sheet or panel 912. The next at step 922, a second sheet or panel of glass 924 is applied to the first sheet 912 to encapsulate the optical fibers 917 between the two layers of glass 912 and 924 to form a finished panel 926 that may be illuminated when power is suppled to the respective power supply cords 921. Then at step 927, the finished panel 926 is tested for operating functionality.

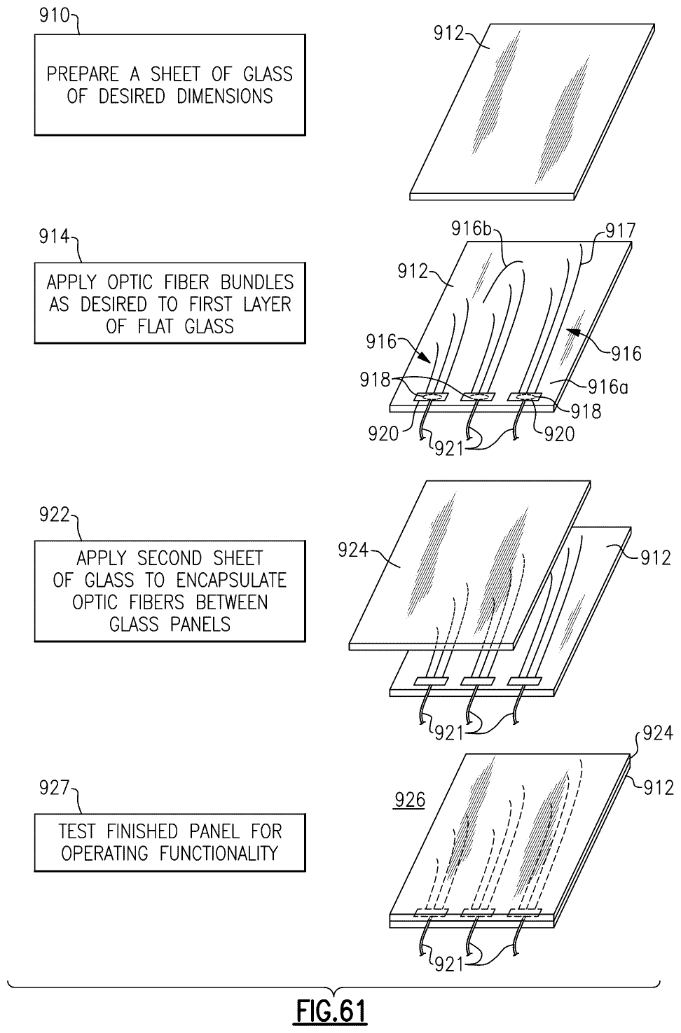

FIG. 62A is an elevation view of a representative partition wall assembly 928 made with the illuminated glass panels 926 hereof showing in an accompanying block diagram certain electronic functionalities that may be provided therewith by applying selected teachings here-from to the arts of architectural design and building security. First, motion detectors 929 are provided in the assembly 928 for each panel 926, and a connection from the motion detectors is provided to control circuitry that is provided with an architectural mode 930 and a security mode 932. Thus is use in the architectural mode 930, the assembly 928 may be placed in a hotel lobby or the first floor of an office building, for example, and as people pass by the assembly, the motion detectors 929 will detect such motion and then illuminate the light sources 918, FIG. 61, in each panel 926 for a certain duration of time. In this manner, the illumination of the panels is reflective of the motion of people in the hotel lobby or the office building first floor. In the security mode 932, such detected motion may be indicative of an unauthorized person in proximity thereto depending on the time of day and other such factors. Here then, one advantage of the assembly 928 as a security device is that such is not apparent by casual observation.

FIG. 62B is a perspective view of a certain office building that has provided therein the illuminated glass panels 926 with each thereof having at least one bundle of optical fibers 916 as described above. Then where desired, each and every window of such a building may be provided with such a panel, with each panel then further associated with one or more motion detectors. The motion detector or detectors for each panel may be place in any location such as the room adjacent the window, the lobby of the building, or any such combination to thereby give animated life to the window by illumination that is reflective of human activity and motion within the building. In addition thereto, the windows may be illuminated with the colors of a local sports team, or be activated during a political season as indicative of votes being cast and counted in an election thereby in any such case of use giving board public notice of such events.

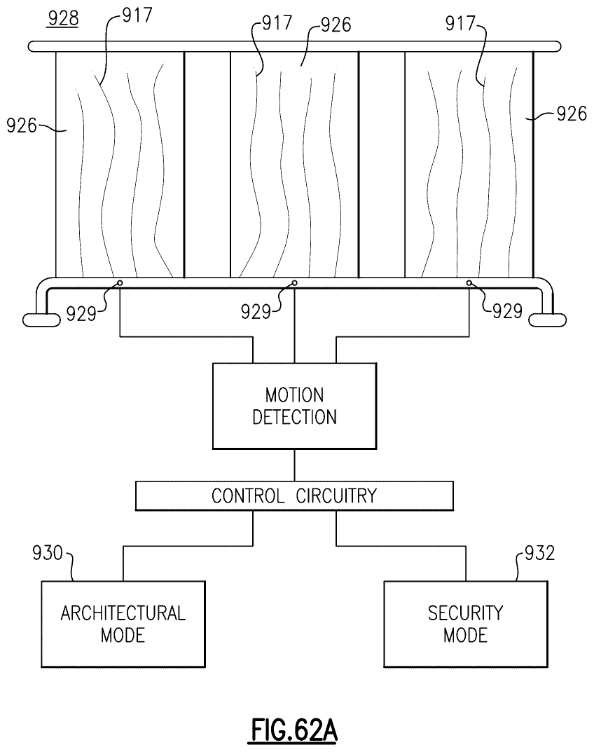

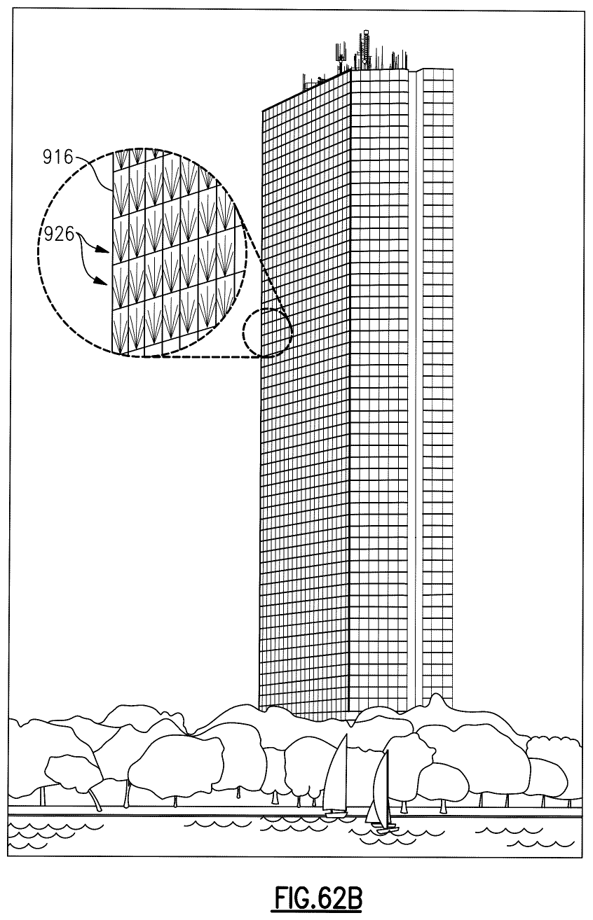

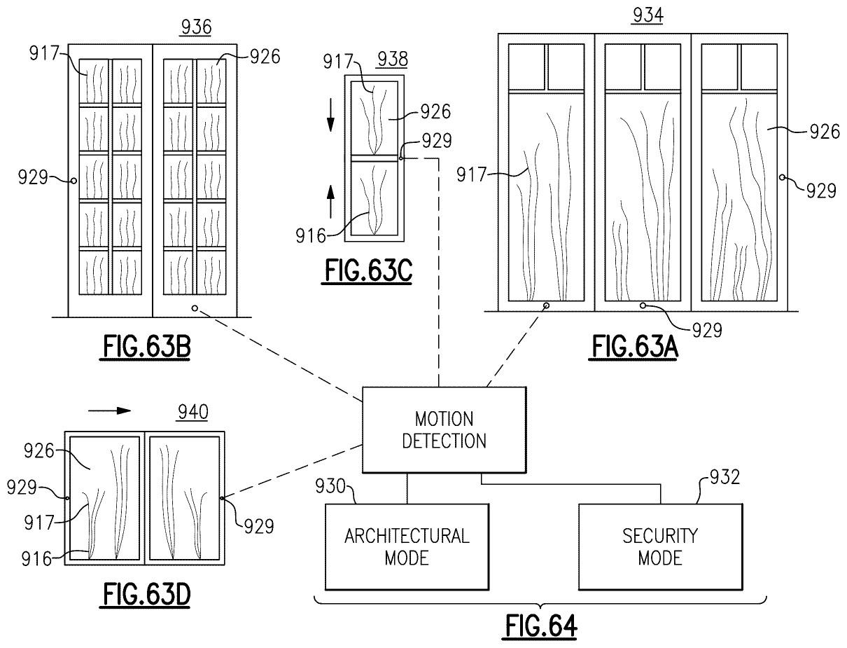

FIGS. 63A to 63D are elevation views of various doors and windows provided with the illuminated glass panels 926 hereof as applied to the arts of residential design and home security. As such, in FIG. 63A there is shown a three-panel set of sliding doors 934, FIG. 63B shows a pair of French doors 936, FIG. 63C shows a typical double-hung window 938 that may be used in a bedroom or bathroom, and FIG. 63D shows a two-panel sliding window 940 that may also be used in a bedroom or kitchen for example. Each of these windows and doors include the illuminated or “illumified” panels hereof and associated with each thereof is at least one motion detector 929 as illustrated. Thus in use in an architectural mode 930, FIG. 64, each fiber 917 in each of the bundles 916 will be illuminated where there is motion in close proximity to a respective motion detector 929. For example, during an evening or daytime party in a home including such, as guests mingle and move about the home, the windows and doors will be illuminated for short intervals of time with such being indicative of the motion in the party. In a security mode 932, FIG. 64, the light sources may be illuminated as being indicative of an unauthorized person in proximity to the home. The light may discourage such an unauthorized person from proceeding with their intentions and the motion detectors as integrated with control circuitry and transmitter circuitry may also then rely such unauthorized presence to a security office such as discussed above.

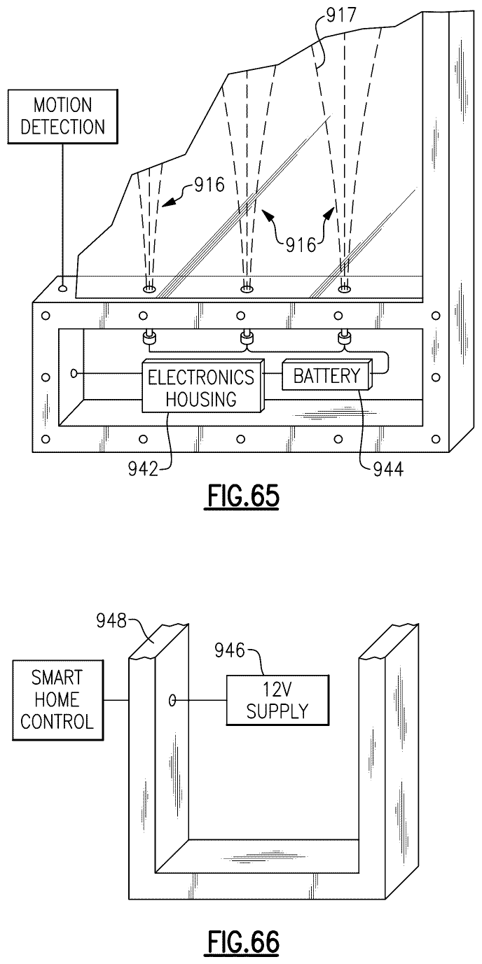

FIG. 65 is a detailed cut-away view of an illuminated or illumified door of the type in FIGS. 63A and 63B herein showing an example of providing an electronics assembly for operation thereof. Thus here within the frame of the door, an electronic housing 942 is supplied to operate the light bundles and motion detection which may be powered by an internal battery 944 or low voltage line supply 946, FIG. 66, that may be provided through the door or window jamb 948 as illustrated.

Thus as provided in FIGS. 61 to 66, the present invention is further directed to a method of making a glass panel assembly for use as an architectural item and a security item, said method comprising the steps of providing i) a first layer of flat glass 912 in a semi-molten state or solidified state; ii) providing a cluster or bundle of optical fibers 916 that are bundled together at an input end 916a thereof; iii) applying the cluster of optical fibers 916 onto the first layer of glass; iv) forming a second layer of glass 924 onto the first layer of glass 912 to encapsulate the cluster of optical fibers 916 between the first and second layers of glass 912 and 924; v) operatively associating at least one light emitting element 918 with the input end 916a of the cluster of optical fibers 916; and vi) providing electronic circuitry including control circuitry, a power supply, and a motion detection component 929 with each thereof being operatively associated with said at least one light emitting element 918 so that, in use, when a person comes into proximity to a glass panel 926 as detected by the motion detection component 929, the control circuitry activates said at least one light emitting element 918 to thereby create a desired light display. In this assembly, the electronic circuitry may further include a transmitter that sends a silent alarm signal to a property custodian when the control circuitry is placed in a security mode 932. Then in application, window package 938 or 940 may be provided as including the above glass panel assembly, or a door package 934 or 936 may be provided as including the above glass panel assembly. And then when such door or window packages are provided such may be implemented in a residential or commercial building structure.

Moving on now to another application of the teaching hereof, FIG. 67 is a schematic perspective view of a multi-mode glass chandelier 950 that may be provided hereunder by applying selected teachings here-from to the arts interior lighting. The chandelier 950 is shown here with along with an accompanying block diagram representing various electronic components and functionalities that may be included therewith. As such, the chandelier 950 includes traditional light assemblies 952 and branches, arms, or elements 954 which each include at least one optical fiber bundle 956 according to the teachings hereof. The chandelier 950 also includes a base portion or base assembly 958 that conceals an electronics housing or casing 960 that is powered by a battery 962 that may be rechargeable when line voltage is supplied or otherwise powered by a low voltage line supply 964 provided through or via a center hanging assembly 966 as suspended from a ceiling location in a typical manner. The base assembly 958 is here further provided with motion detectors 929 that are operably associated with control circuitry in the electronics housing 960 such that when a person walks in close proximity to the chandelier, the motion detectors 929 will detect their presence and then even when the chandelier is otherwise switched off so that the traditional light elements 952 are in an off-state, the low voltage line voltage or battery will power the light bundles in the arms or elements 954 for a short fix period of time to thereby animate the chandelier such that it gives this illuminated acknowledgement of a person in proximity thereto. In addition, the chandelle may serve as a night light such that in an otherwise dark room, the presence of a person in the middle of the night may be gently illuminated with a soft and low light provided by the fiber bundles 956 in the arms or elements 954.

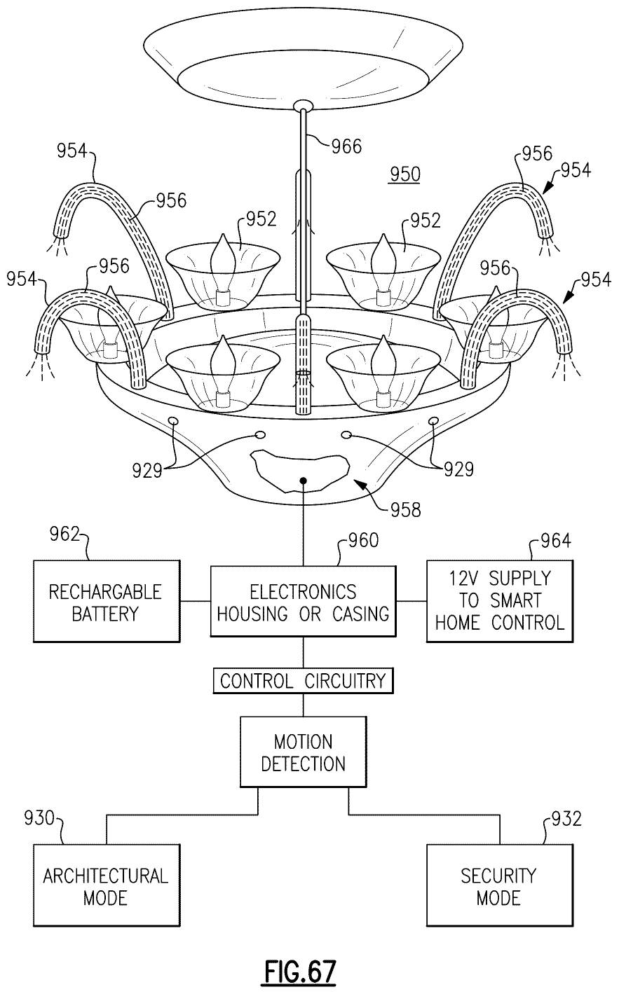

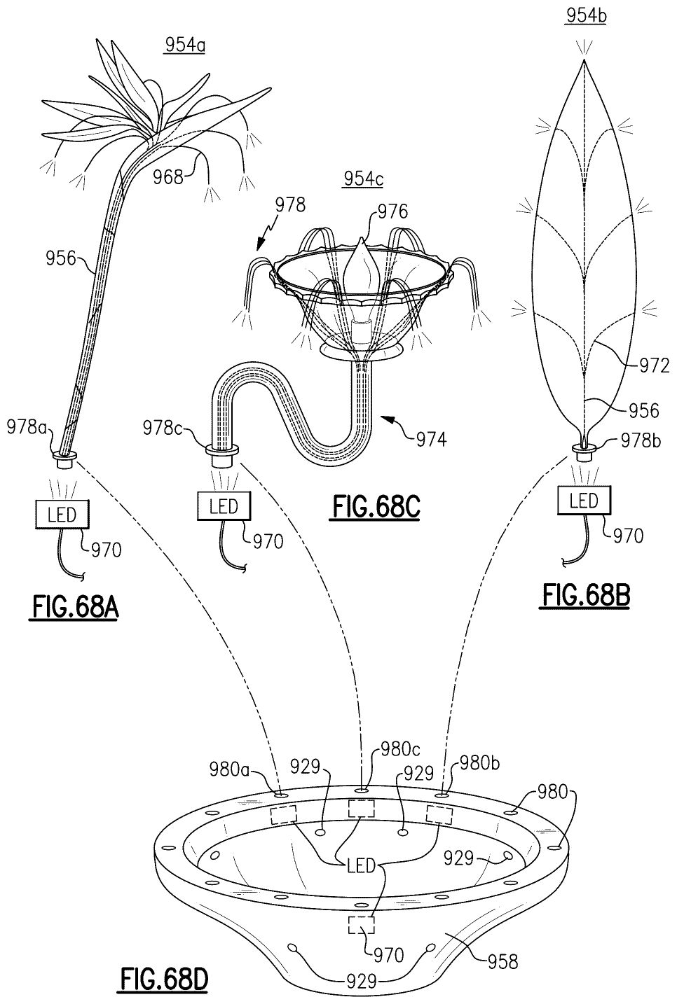

FIG. 68A is an illustration of a glass-blown element 954 for use with the type of chandelier 950 of FIG. 67 here configured in the form of a bird of paradise flower and stalk and as such referenced 954a. The lighting element 954a is provided with a bundle of light-emitting optical fibers 956 partially encased therein and partially extending therefrom as including extension segments 968. In view of related disclosure herein provided above, the extension segments 968 include an output end that when illuminated by a light source such as the LED light source 970 shown here provides some light output therefrom.

FIG. 68B is an illustration of a glass-blown element for use with the type of chandelier 950 of FIG. 67 configured in the form of a single bird of paradise leaf 954b having light-emitting optical fibers fully encased therein. Here, the encased bundle 956 is configured to follow the veins in the leaf where some thereof may be comprised of a single fiber 972 as illustrated.

FIG. 68C is an illustration of a glass-blown element for the type hereof for use with the chandelier 950 of FIG. 67 configured in the form of a goose-neck branch or arm 974 having a traditional flame-tip light bulb 976 and a bundle of light-emitting optical fibers 956 partially encased therein and partially extending therefrom in a pony-tail style of extension segments 978.

FIG. 68D is a schematic illustration of the electronic base assembly 958 according to this invention as provided for the type of chandelier 950 of FIG. 67 configured to support the glass-blown lighting elements of FIGS. 68A, 68B, and 68C as illustrated. Here each of the elements 954a, 954b, and 954c includes a plug member 978 that fits into a corresponding socket 980 such that in assembly, plug 978a on element 954a fits into socket 980a, plug 978b on element 954b fits into socket 980b, plug 978c on element 954c fits into socket 980c, and so on around the base 958 until the chandelier is completed as desired.

Thus in this manner as provided in FIGS. 67 to 68D, there is further hereby provided by the inventors hereof a multimode chandelier 950 comprising i) a base assembly 958 configured to receive a plurality of lighting elements; ii) a first plurality of lighting elements 952, 976 being connectable to a wall switch; iii) a second plurality of lighting elements 954 with each thereof including a bundle of optical fibers 956; iv) an electronics housing 960 provided in association with the base assembly 958, said electronics housing 960 including control circuitry for controlling illumination of said second plurality of lighting elements 954; v) at least one light source 970 associated with said second plurality of lighting elements 954, said at least one light source 970 being concealed within the base assembly 958 and operably associated with said control circuitry; and vi) at least one motion detector 929 provided in the base assembly 958 and operably associated with said control circuitry such that when motion is detected by said at least one motion detector 929, said control circuitry activates said at least one light source 970 to thereby provide light along at least one of the bundles of optical fiber 956 as provided in a corresponding one of said second plurality of lighting elements 954.

While this invention has been described in detail with reference to certain preferred embodiments, it should be appreciated that the present invention is not limited to those precise embodiments. Rather, in view of the present disclosure which describes the current best modes for practicing the inventions hereof, many modifications and variations would present themselves to those of skill in the respective arts hereof without departing from the scope and spirit of these invention. The scope of the present inventions is, therefore, indicated by the following claims rather than by the foregoing description. All changes, modifications, and variations coming within the meaning and range of coverage of the claims are to be considered within their scope.

1-251. (canceled)

252. A method of making a natural stone jewelry box having light display notification and recharging functionality for use with personal electronic items, said method comprising the steps of:
selecting a block of stone having a desired translucence property;
forming a stone vessel or shell from the block of stone, said stone vessel having an outside surface, an inside surface, and a thickness therebetween that allows some light to pass therethrough;
positioning at least one light emitting element at a selected location along the inside surface of said stone vessel;
providing a ceramic casing having electronic circuitry secured therein and fitting the ceramic casing onto an inside bottom area of said stone vessel; and
blowing molten glass onto the inside surface of the stone vessel to form a layer of glass to thereby encapsulate said at least one light emitting element and said ceramic casing between the inside surface of the stone vessel and the layer of glass.

253-256. (canceled)

257. The method according to claim 252 wherein the electronic circuitry includes circuitry for charging rechargeable batteries when placed in the stone vessel during use thereof.

258-264. (canceled)

265. The method according to claim 252 wherein the at least one light emitting element includes a predetermined number of light sources with each thereof being associated with a corresponding cluster of optical fibers bundled together at an input end thereof.

266. The method according to claim 265 wherein the step of forming the stone vessel from the block of stone further comprises forming at least one side wall including a channel formed therein on the inside surface of the stone vessel.

267. The method according to claim 266 wherein the step of positioning the at least one light emitting element along the inside surface of the stone vessel further comprises positioning a selected corresponding cluster of optical fibers into a corresponding side wall channel before the step of blowing molten glass to form the layer of glass.

268-278. (canceled)

279. The method according to claim 252 further comprising the step of depositing a semi-transparent layer of gold onto at least a portion of the outside surface of the stone vessel.

280-364. (canceled)

365. A method of making a laminated stone shell for use as a jewelry box, said method comprising the steps of:
selecting at least one slab of natural stone material having a desired translucence property;
forming a series of frame segments from the at least one slab of natural stone, each frame segment having an overlay portion and an overhang portion;
affixing overlay portions of consecutive frame segments together to form a rigid structure having an inside surface and an outside inverted stepped surface formed by the overhang portion of each consecutive frame segment; and
preparing the inside surface of the rigid structure to receive at least one light emitting element.

366-384. (canceled)

385. The method of claim 365 further comprising:
blowing molten glass into the stone shell to form a first layer of glass on the inside surface of the stone shell;
applying at least one light emitting element onto the first layer of glass in a selected location; and
blowing molten glass onto the first layer of glass to form a second layer of glass to thereby encapsulate said at least one light emitting element between the first and second layers of glass to form a natural stone jewelry vessel having light display notification and recharging functionality for use with personal electronic items.

386. (canceled)

387. The method according to claim 385 including the further step of providing or obtaining an electronics casing having electronic circuitry secured therein.

388. The method according to claim 387 including the further step of fitting the electronics casing onto an inside surface of a bottom area of the stone shell.

389. The method according to claim 388 including the further step of connecting the at least one light emitting element to the electronic circuitry in the electronics casing before the step of blowing molten glass to form the second layer of glass.

390. (canceled)

391. (canceled)

392. The method according to claim 385 wherein the electronic circuitry includes circuitry for charging rechargeable batteries when placed in the vessel during use thereof.

393. (canceled)

394. (canceled)

395. The method according to claim 385 wherein the natural stone jewelry vessel is configured as a stone jewelry box.

396. (canceled)

397. (canceled)

398. The method according to claim 395 wherein the stone jewelry box is configured to be used to charge either or both a mobile phone and electronic jewelry items placed therein together.

399. (canceled)

400. The method according to claim 385 wherein the at least one light emitting element includes a predetermined number of light sources with each thereof being associated with a corresponding cluster of optical fibers bundled together at an input end thereof.

401-588. (canceled)

589. An illuminated charging and wake-up vessel, comprising:
a container portion having at least one side wall and a bottom, said container portion being formed from a first rigid layer and a second rigid layer with said first rigid layer being formed from a translucent material;
an electronics casing positioned in the bottom of the container portion being encapsulated therein between the first and second rigid layers, said electronics casing including control circuitry and a charging pad for changing a mobile phone when placed in the container portion;
receiving circuitry in operable engagement with said control circuitry, said receiving circuitry configured to receive a wake-up signal from the mobile phone; and
at least one light source encapsulated between the first and second rigid layers and associated operably with said control circuitry such that when said receiving circuitry receives a wake-up signal from the mobile phone, said control circuitry switches said at least one light source from an off-state to an on-state.

590. The illuminated wake-up vessel according to claim 589 wherein said at least one light source includes a plurality of light sources.

591. The illuminated wake-up vessel according to claim 590 wherein at least one of the plurality of light sources is associated with a bundle of optical fibers.

592-594. (canceled)

595. The illuminated wake-up vessel according to claim 591 wherein any number of optical fiber bundles is encapsulated between the first and second rigid layers and at least some thereof have at least some extension segments that extend up and over a top edge of the vessel.

596. (canceled)

597. (canceled)

598. The illuminated wake-up vessel according to claim 589 wherein said on-state includes an initial lower-power on-state that transitions to a higher-power on-state over a set time period such that the at least one light source in response thereto transitions from a corresponding lower brightness to a higher brightness over said set time period.

599-629. (canceled)
